SEQUENCE LISTING (26 sequences)

SEQ ID NO: 1 - protein, 120 aa, synthetic construct [sequence omitted]

SEQ ID NO: 2 - protein, 107 aa, synthetic construct [sequence omitted]

SEQ ID NO: 3 - protein, 8 aa, synthetic construct
WYGQGFYA

SEQ ID NO: 4 - protein, 8 aa, synthetic construct
WGYDYFYA

SEQ ID NO: 5 - protein, 8 aa, synthetic construct
WGGDCRYA

SEQ ID NO: 6 - protein, 8 aa, synthetic construct
WGDNGFYA

SEQ ID NO: 7 - protein, 8 aa, synthetic construct
WGNDGFSA

SEQ ID NO: 8 - protein, 8 aa, synthetic construct
WGGPGFYA

SEQ ID NO: 9 - protein, 8 aa, synthetic construct
WWGSGFYA

SEQ ID NO: 10 - protein, 8 aa, synthetic construct
WHGTGFYA

SEQ ID NO: 11 - protein, 8 aa, synthetic construct
RGGDYFYA

SEQ ID NO: 12 - protein, 8 aa, synthetic construct
WHGYGFYA

SEQ ID NO: 13 - protein, 8 aa, synthetic construct
WGGDFGYA

SEQ ID NO: 14 - protein, 8 aa, synthetic construct
WGGDGFQA

SEQ ID NO: 15 - protein, 8 aa, synthetic construct
WVGDGFIA

SEQ ID NO: 16 - protein, 8 aa, synthetic construct
WHGDGTYA

SEQ ID NO: 17 - protein, 8 aa, synthetic construct
WGCDGFSA

SEQ ID NO: 18 - protein, 8 aa, synthetic construct
WVGDDFYA

SEQ ID NO: 19 - protein, 8 aa, synthetic construct
SGGDSFYA

SEQ ID NO: 20 - protein, 8 aa, synthetic construct
WGGDGFRA

SEQ ID NO: 21 - protein, 8 aa, synthetic construct
WGTDGVYA

SEQ ID NO: 22 - protein, 8 aa, synthetic construct
WGGDGFTT

SEQ ID NO: 23 - protein, 20 aa, synthetic construct
ARNDQEGHILKMFPSTWYVC

SEQ ID NO: 24 - protein, 121 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARHGNYYYYYGMDVWGQGTTVTVSS

SEQ ID NO: 25 - protein, 121 aa, synthetic construct
QVQLVQSGAEVKKPGSSVKVSCKASGGTSNNYAISWVRQAPGQGLEWMGGISPIFGSTAYAQKFQGRVTISADIFSNTAYMELNSLTSEDTAVYFCARHGNYYYYSGMDVWGQGTTVTVSS

SEQ ID NO: 26 - DNA, 12 bp, synthetic construct
  source 1..12: mol_type = other DNA
tattattgcgcg

(19) United States
(12) Patent Application Publication
(10) Pub. No.: US 2023/0268026 A1
(43) Pub. Date: Aug. 24, 2023

(54) DESIGNING BIOMOLECULE SEQUENCE VARIANTS WITH PRE-SPECIFIED ATTRIBUTES

(71) Applicant: ABSCI CORPORATION, Vancouver, WA (US)

(72) Inventors: Roberto Spreafico, Vancouver, WA (US); Goran Rakocevic, Vancouver, WA (US); Ariel Schwartz, Vancouver, WA (US); Joshua Meier, Vancouver, WA (US)

(21) Appl. No.: 18/046,849

(22) Filed: Oct. 14, 2022

Related U.S. Application Data
(60) Provisional application No. 63/398,222, filed on Aug. 15, 2022.
(60) Provisional application No. 63/339,450, filed on May 7, 2022.
(60) Provisional application No. 63/338,433, filed on May 4, 2022.
(60) Provisional application No. 63/320,067, filed on Mar. 15, 2022.
(60) Provisional application No. 63/297,679, filed on Jan. 7, 2022.
(60) Provisional application No. 63/338,398, filed on May 4, 2022.

(51) Int. Cl. G16B 20/30; G06N 3/08; G16B 20/20; G16B 40/20

(52) U.S. Cl. CPC G16B 20/30; G06N 3/08; G16B 20/20; G16B 40/20

(57) ABSTRACT

A computing system includes a processor and a memory having stored thereon a trained machine-learned model and instructions that, when executed by the one or more processors, cause the computing system to process a biomolecule sequence variant to predict binding characteristics, identify biomolecule sequence variants of interest; and provide the biomolecule sequence variants of interest as output. A computer-implemented method for training a machine learning model to identify biomolecule sequence variants of interest includes generating biomolecule sequence variants, receiving screening data; and training the machine learning model to predict binding characteristics of an input biomolecule sequence variant. A computing system includes a processor; and a non-transitory computer-readable media having stored thereon a machine-learned model trained using training data and instructions that, when executed by the one or more processors, cause the computing system to: process one or more input biomolecule sequence variants; and provide a predicted naturalness characteristics as output.

### CROSS-REFERENCE TO RELATED APPLICATIONS

This application claims the benefit of U.S. Provisional Application 63/398,222 filed on Aug. 15, 2022. This application claims the benefit of U.S. Provisional Application 63/339,450 filed on May 7, 2022. This application claims the benefit of U.S. Provisional Application 63/338,398 filed on May 4, 2022. This application claims the benefit of U.S. Provisional Application 63/338,433 filed on May 4, 2022. This application claims the benefit of U.S. Provisional Application 63/320,067 filed on Mar. 15, 2022. This application claims the benefit of U.S. Provisional Application 63/297,679 filed on Jan. 7, 2022. The priority applications are hereby incorporated by reference in their entireties.

### INCORPORATION BY REFERENCE OF MATERIAL SUBMITTED ELECTRONICALLY

The Sequence Listing, which is a part of the present disclosure, is submitted concurrently with the specification as an XML file. The name of the XML containing the Sequence Listing is “57548_SubSeqlisting.xml”, which was created on Jan. 4, 2023 and is 68,563 bytes in size. The subject matter of the Sequence Listing is incorporated herein in its entirety by reference.

### BACKGROUND

Monoclonal antibodies may exhibit suboptimal binding affinity towards their target antigen. Affinity may be improved by “maturation” of the antibody sequence, often by combinatory mutagenesis of CDRs. However, the combinatory mutational space is so large it would take a considerable amount of time and resources to exhaustively probe by experimental methods. Therefore, wet lab screening solutions (enhanced antibody affinity) may be inefficient and time consuming.

Biological drug discovery is a complex combinatorial challenge. The number of possible monoclonal antibody CDR variants exceeds the number of atoms in the universe. Traditional antibody screening approaches explore a small sequence space (e.g., hundreds to thousands of variants), often resulting in drug candidates with poor binding affinities, developability concerns, and poor immunogenicity profiles. Biological drug discovery fails too often. Specifically, despite billions of dollars of investment every year, only an estimated 4% of drug leads succeed in their journey from discovery to launch. Even worse, only 18% of drug leads that pass preclinical trials eventually pass Phase I and II trials, suggesting the large majority of drug candidates are unsafe or ineffective. While much of this failure rate is attributable to incomplete understanding of the underlying biology and pathology, insufficient drug lead optimization contributes to a large number of failures.

Traditional antibody screening approaches can only explore small sequence spaces, which may constrain results to sequences that confer suboptimal properties such as insufficient binding affinity, developability limitations and poor immunogenicity profiles. In contrast, deep mutagenesis coupled with screening or selection allows for the exploration of a larger antibody sequence space, potentially yielding more and better drug leads. However, deep mutagenesis comes with its own challenges. For example, most mutations degrade the binding affinity of a given antibody rather than improving it, which reduces screening efficiency. Moreover, the combinatorics of the antibody sequence variant space grows exponentially with mutational load (i.e., the number of mutations simultaneously introduced into each sequence variant) and quickly exceeds the capacity of experimental assays by orders of magnitude.

Still further, the development of a candidate biomolecule (e.g., antibody) into a therapeutic drug is a complex process with a high degree of risk. This risk is often due to numerous challenges in production, formulation, efficacy, and adverse reactions. Modeling these risks has been a tremendous challenge for the industry due to the difficulty in obtaining the relevant data, particularly at scale.

In addition, most antibody screening approaches are limited to screening only one property at a time, restricting simultaneous optimization of drug potency and developability. Simultaneous, rather than sequential, optimization of antibody properties is a more advantageous therapeutic strategy, because improvement of a single property may negatively impact other properties. Strategies that concurrently co-optimize all properties can yield better therapeutic products. Deep neural networks are an emerging tool for overcoming the limitations of experimental screening capacity. The general approach involves training a model on a small amount of experimental data and applying it to predict which sequences, are most likely to improve the measured trait. Several promising approaches have been proposed, but only a limited number of in silico predictions from these models have been validated in the lab. While sufficient as a proof of principle, such demonstrations are limited for practical design by the shortcomings of the screening platforms used to generate training data: binary (rather than continuous) readouts with limited throughput. Overall, this curbs the quantitative accuracy of the models and their ability to extrapolate to higher mutational loads.

An additional concern for antibody screening approaches is that the improvement of binding affinity can come at the cost of decreased developability and immunogenicity properties. This issue would remain unaddressed by machine learning models trained without regard for other properties.

Thus, techniques are needed that enable more exploration of the correct sequences, by coupling high-throughput experimental biology with machine learning to improve upon conventional biomolecule screening approaches.

### SUMMARY

In one aspect, a computing system for identifying biomolecule sequence variants of interest, the computing system includes (a) one or more processors; and (b) one or more non-transitory computer-readable media having stored thereon (i) a machine-learned model trained using training data, wherein the training data includes one or more training biomolecule sequence variants, each having a respective measured binding characteristic representing the ability of each to bind to a corresponding respective binding partner, and wherein the machine-learned model is configured to output a predicted biomolecule binding characteristic of an input biomolecule sequence variant; and (ii) instructions that, when executed by the one or more processors, cause the computing system to: (1) process the one or more biomolecule sequence variants with the machine-learned model to generate one or more predicted binding characteristics, each corresponding to a respective one of the one or more biomolecule sequence variants; (2) analyze the one or more predicted binding characteristics to identify one or more biomolecule sequence variants of interest from among the sequence variants, each of the one or more biomolecule sequence variants of interest having a respective one or more desired properties; and (3) provide the one or more biomolecule sequence variants of interest as an output.

In another aspect, a computer-implemented method for training a machine learning model to identify biomolecule sequence variants of interest includes (1) generating one or more biomolecule sequence variants by programmatically mutating a reference biomolecule; (2) receiving screening data including a ranking the biomolecule sequence variants according to one or more training binding characteristics; and (3) training the machine learning model using the received screening data to predict one or more desired binding characteristics of an input biomolecule sequence variant.

In another aspect, a computing system for predicting a naturalness of a biomolecule sequence variant, the computing system includes one or more processors; and one or more non-transitory computer-readable media having stored thereon: a machine-learned model trained using training data, wherein the training data includes one or more training biomolecule sequence variants, and wherein the machine-learned model is configured to output a respective predicted naturalness characteristic of one or more biomolecule sequence variants; and instructions that, when executed by the one or more processors, cause the computing system to: (i) process one or more input biomolecule sequence variants with the machine-learned model to generate a respective predicted naturalness characteristic for each of the one or more input biomolecule sequence variants; and (ii) provide at least one of the predicted naturalness characteristics as output.

In yet another aspect, a computing system for predicting the naturalness of a biomolecule sequence variant, the computing system includes one or more processors; and one or more non-transitory computer-readable media having stored thereon: a machine-learned model trained using training data, wherein the training data includes one or more training biomolecule sequence variants, and wherein the machine-learned model is configured to output a respective predicted naturalness characteristic of one or more biomolecule sequence variants; and instructions that, when executed by the one or more processors, cause the computing system to: (1) process one or more input biomolecule sequence variants with the machine-learned model to generate a respective predicted naturalness characteristic for each of the one or more input biomolecule sequence variants; and (2) provide at least one of the one or more predicted naturalness characteristics as output.

In some embodiments, the present disclosure provides methods for generating training data. In one embodiment, a method for generating training data for a machine learning model is provided comprising: a) expressing a biomolecule variant library in host cells; b) measuring: (i) expression levels and (ii) affinity values to a binding partner of interest of two or more biomolecule variants expressed in (b); c) sorting the host cells into a distribution of cell subpopulations based on the measured expression levels and measured affinity values; thereby collecting cells across an affinity distribution; d) sequencing the biomolecule variants expressed from the collected cells of (c); e) calculating an enrichment score for each sequenced biomolecule variant, wherein said enrichment score and said biomolecule variant sequence is capable of training a machine learning model capable of performing sequence-based affinity predictions.

In another embodiment, an aforementioned method is provided wherein the library of biomolecule variants is generated by randomly mutating a nucleic acid encoding a reference biomolecule. In another embodiment, an aforementioned method is provided wherein the library of biomolecule variants is generated by random mutagenesis, error-prone PCR mutagenesis, oligonucleotide-directed mutagenesis, cassette mutagenesis, shuffling, saturation mutagenesis, homology-directed mutagenesis, Activation Induced Cytidine Deaminase (AID) mediated mutagenesis, or transposon mutagenesis. In still another embodiment, an aforementioned method is provided wherein the library of biomolecule variants comprises at least 104-107 unique biomolecule variant sequences. In yet another embodiment, an aforementioned method is provided wherein the library of biomolecule variants are displayed on the host cell surface. In another embodiment, an aforementioned method is provided wherein the library of biomolecule variants are expressed and retained in the host cell cytoplasm.

In another embodiment, an aforementioned method is provided wherein the host cells are Escherichia coli cells. In yet another embodiment, an aforementioned method is provided wherein Escherichia coli cells are Escherichia coli 521 cells. In another embodiment, an aforementioned method is provided wherein the Escherichia coli cells comprises one or more or all of: a) an alteration of gene function of at least one gene encoding a transporter protein for an inducer of at least one inducible promoter; b) a reduced level of gene function of at least one gene encoding a protein that metabolizes an inducer of at least one inducible promoter; c) a reduced level of gene function of at least one gene encoding a protein involved in biosynthesis of an inducer of at least one inducible promoter; d) an altered gene function of a gene that affects the reduction/oxidation environment of the host cell cytoplasm; e) a reduced level of gene function of a gene that encodes a reductase; f) at least one expression construct encoding at least one disulfide bond isomerase protein; g) at least one polynucleotide encoding a form of DsbC lacking a signal peptide; and/or h) at least one polynucleotide encoding Ervlp.

In still another embodiment, an aforementioned method is provided wherein step (c) optionally additionally measures one or more of binding specificity, biological activity, stability, and/or solubility of the expressed biomolecule variants.

In yet another embodiment, an aforementioned method is provided wherein affinity is quantified by measuring binding dissociation constant (KD) of a biomolecule variant to the binding partner of interest. In one embodiment, the binding partner of interest is a fluorescently labeled antigen.

In still another embodiment, an aforementioned method is provided wherein expression level of the biomolecule variants is quantified by measuring anti-IgG-binding capacity. In another embodiment, an aforementioned method is provided wherein expression level of the biomolecule variants is quantified using an anti-IgG antibody conjugated to a fluorophore. In yet another embodiment, an aforementioned method is provided wherein expression level of the biomolecule variants is quantified by measuring a non-antigen binding capacity.

The present disclosure also provides, in some embodiments, an aforementioned method wherein the measuring in step (c) and sorting in step (d) comprises a fluorescence-activated cell sorting (FACS) assay. In another embodiment, an aforementioned method is provided optionally further comprising measuring binding affinity of the sequenced biomolecule variants prior to calculating an enrichment score. In one embodiment, the binding affinity is measured using an assay selected from the group consisting of a Surface Plasmon Resonance (SPR) based binding assay, Biolayer Interferometry and/or flow cytometry derived binding curves.

In another embodiment, an aforementioned method is provided wherein the sequencing of step (e) is obtained by a method selected from the group consisting of deep sequencing, next generation sequencing, Long read nanopore sequencing, Single Molecule Real-Time long read sequencing (pacbio). In another embodiment, the sequencing of step (e) is obtained by a method selected from the group consisting of deep sequencing, next generation sequencing, Long read nanopore sequencing, Single Molecule Real-Time long read sequencing (pacbio). In another embodiment, an aforementioned method is provided wherein wherein nucleic acids encoding the biomolecule variants are modified prior to sequencing to comprise barcode sequences comprising unique molecular identifiers (UMIs).

The present disclosure also provides, in one embodiment, an aforementioned method wherein the biomolecule variants are selected from a group consisting of a monoclonal antibody, a bispecific antibody, a multispecific antibody, a humanized antibody, a chimeric antibody, a camelised antibody, a single domain antibody, a single-chain Fvs (ScFv), a single chain antibody, a Fab fragment, a F(ab′) fragment, a disulfide-linked Fvs (sdFv), or an anti-idiotypic (anti-Id) antibody. In another embodiment, an aforementioned method is provided wherein wherein the biomolecule variants are selected from a group consisting of a monoclonal antibody, a bispecific antibody, a multispecific antibody, a humanized antibody, a chimeric antibody, a camelised antibody, a single domain antibody, a single-chain Fvs (ScFv), a single chain antibody, a Fab fragment, a F(ab′) fragment, a disulfide-linked Fvs (sdFv), or an anti-idiotypic (anti-Id) antibody. In yet another embodiment, an aforementioned method is provided wherein the biomolecule variants are selected from a group consisting of a peptide, a polypeptide, a protease, an oxidoreductase, a transferase, a hydrolase, a lyase, an isomerase, a ligase, an enzyme, an antibody, a cytokine, a chemokine, a nucleic acid, a metabolite, a small molecule (<1 kDa) and a synthetic molecule.

In still another embodiment, a method for generating training data for a machine learning model is provided comprising: a) expressing a biomolecule variant library in host cells; b) measuring: (i) expression levels and (ii) affinity values to a binding partner of interest of two or more biomolecule variants expressed in (b); c) sorting the host cells into a distribution of cell subpopulations based on the measured expression levels and measured affinity values; thereby collecting cells across an affinity distribution; d) isolating nucleic acids encoding the biomolecule variants from the collected host cells of (c), amplifying said nucleic acids using selective rolling circle amplification (sRCA), and sequencing nucleic acids encoding the biomolecule variants; and e) calculating an enrichment score for each sequenced biomolecule variant, wherein said enrichment score and said biomolecule variant sequence is capable of training a machine learning model capable of performing sequence-based affinity predictions.

### BRIEF DESCRIPTION OF THE DRAWINGS

FIG. 1 shows an exemplary computing environment for performing the present techniques, according to some aspects.

FIG. 2 shows an exemplary computer-implemented method for training one or more machine-learned model to identify one or more biomolecule sequence variants of interest, according to some aspects.

FIG. 3A depicts a computer-implemented method of operating a trained machine-learned model to identify one or more biomolecule sequence variants of interest, according to some aspects.

FIG. 3B depicts a computer-implemented method of training a machine learning model to identify biomolecule sequence variants of interest, according to some aspects.

FIG. 4A shows an exemplary data flow diagram depicting training and predicting of biomolecule sequence variants of interest, that may correspond to FIG. 2, according to some aspects.

FIG. 4B depicts an example block flow diagram for performing the assay of FIG. 4A, according to some aspects. Strains expressing unique antibody sequence variants may be added to fix and permeabilized cells, and probes may be added.

FIG. 4C depicts an example affinity prediction chart, according to some aspects.

FIG. 4D depicts an example affinity prediction validation chart, according to some aspects.

FIG. 4E depicts exemplary denoised data charts, according to some aspects.

FIG. 4F depicts an exemplary conceptual diagram depicting naturalness training and prediction, according to some aspects.

FIG. 4G depicts exemplary naturalness score validation charts, according to some aspects.

FIG. 4H depicts exemplary naturalness/developability correlation charts, according to some aspects.

FIG. 4I depicts exemplary naturalness and immunogenicity correlation charts, according to some aspects.

FIG. 4J depicts exemplary naturalness and mutational load correlation charts, according to some aspects.

FIG. 4K depicts exemplary charts of affinity prediction improvement when enriching with naturalness data, according to some aspects.

FIG. 4L depicts exemplary conceptual diagrams of in silico sequence variant generation and optimization, according to some aspects.

FIG. 4M depicts an exemplary chart of affinity prediction from trastuzumab, according to some aspects.

FIG. 4N depicts exemplary affinity prediction charts from different parent antibodies, according to some aspects.

FIG. 4O depicts exemplary visualizations of optimizing for affinity and naturalness, according to some aspects.

FIG. 4P depicts another example affinity prediction chart, including a comparison of binding affinity measurements, according to some aspects.

FIG. 4Q depicts an example affinity prediction chart, including a comparison of binding affinity measurements made by SPR and model predictions, trained on SPR data while holding out all points with affinity higher than wild-type Trastuzumab, according to some aspects.

FIG. 5A depicts an exemplary AI-augmented antibody optimization diagram 500, according to some aspects.

FIG. 5B depicts a Fluorescence-Activated Cell Sorting (FACS) and Next-Generation Sequencing (NGS) method of binning antibody variants based on affinity, according to some aspects.

FIG. 6A depicts an exemplary workflow proof-of-concept diagram, according to some aspects.

FIG. 6B depicts predictive performance of a model trained on qaACE scores of variants from 90% of trast-1, evaluated on a 10% holdout data set, according to some aspect.

FIG. 6C depicts a comparative analysis of replicate qaACE measurements and qaACE scores predicted from models trained on individual qaACE replicates, according to some aspects.

FIG. 6D depicts a comparison of ACE scores measured by two replicate FACS sorts, according to some aspects.

FIG. 6E depicts an all-vs-all comparison of ACE scores measured by one of two replicate FACS sorts against ACE scores predicted by models trained only with data from one of the two replicates, according to some aspects.

FIG. 6F depicts a correlation between qaACE affinity score and log-transformed SPR K_{D} measurements, according to some aspects.

FIG. 6G depicts predictive performance against a hold-out set uniformly distributed with respect to binding affinity, according to some aspects.

FIG. 7A depicts predictions from a model trained on SPR-measured −log₁₀ K_{D} values, according to some aspects.

FIG. 7B depicts comparative analysis of replicate −log₁₀ K_{D} measurements and −log₁₀ K_{D} predicted from models trained on individual SPR replicates, according to some aspects.

FIG. 7C depicts predictions from a model trained on log₁₀ kₒₙ values, according to some aspects.

FIG. 7D depicts predictions from a model trained on −log₁₀ k_{off} values, according to some aspects.

FIG. 7E depicts a comparison of −log₁₀ K_{D} values measured by two SPR experiments, according to some aspects.

FIG. 7F depicts an all-vs-all comparison of −log₁₀ K_{D} values measured by one of two replicate SPR experiments against −log₁₀ K_{D} values predicted by models trained only with data from one of two replicates, according to some aspects.

FIG. 7G depicts prediction of −log₁₀ K_{D} using SPR training data alone or supplemented by ACE measurements, according to some aspects.

FIG. 7H depicts a comparison of log₁₀ kₒₙ values measured by two SPR experiments, according to some aspects.

FIG. 7I depicts a comparative analysis of replicate log₁₀ k measurements and log₁₀ kₒₙ predicted from models trained on individual SPR replicates, according to some aspects.

FIG. 7J depicts an all-vs-all comparison of log₁₀ kₒₙ values measured by one of two replicate SPR experiments against log₁₀ kₒₙ values predicted by models trained only with data from one of the two replicates, according to some aspects.

FIG. 7K depicts a comparison of −log₁₀ k_{off} values measured by two SPR experiments, according to some aspects.

FIG. 7L depicts a comparative analysis of replicate −log₁₀ k_{off} measurements and −log₁₀ k_{off} predicted from models trained on individual SPR replicates, according to some aspects.

FIG. 7M depicts an all-vs-all comparison of −log₁₀ k_{off} values measured by one of two replicate SPR experiments against −log₁₀ k_{off} values predicted by models trained only with data from one of the two replicates, according to some aspects.

FIG. 7N depicts a 90:10 train:hold-out split of ACE scores from the trast-1 dataset, according to some aspects.

FIG. 7O depicts a 10-fold cross-validation with −log₁₀ K_{D} values from the trast-2 dataset, according to some aspects.

FIG. 7P depicts a scatter plot of random model embeddings relative to binding affinity, no pre-training and no fine-tuning, according to some aspects.

FIG. 7Q depicts a scatter plot of model embeddings relative to binding affinity, with no pre-training and fine-tuning with binding affinity data using the trast-2 dataset, according to some aspects.

FIG. 7R depicts a scatter plot of model embeddings relative to binding affinity, with pre-training using OAS-derived sequences and no fine-tuning, according to some aspects.

FIG. 7S depicts a scatter plot of model embeddings relative to binding affinity, with pre-training using OAS derived sequences and fine-tuning with binding affinity data using the trast-2 dataset, according to some aspects.

FIG. 8A depicts a density plot of predicted (Design) and measured (Validation) binding affinities of 50 sequences designed to span about 2 orders of magnitude of KDs (set A), according to some aspects.

FIG. 8B depicts a density plot of predicted (Design) and measured (Validation) binding affinities of 50 sequences designed to bind HER2 more tightly than parental trastuzumab (set B), according to some aspects.

FIG. 8C depicts an empirical distribution function (ECDF) of the measured (Validation) binding affinities of the 50 sequences from design set B, wherein lines indicate the measured −log₁₀ K_{D} of trastuzumab (or deviations by −0.1 or −0.5 log), according to some aspects.

FIG. 8D depicts a density plot of binding affinities from set B as predicted by a model trained with a full trast-2 dataset as in FIG. 8B, (Design, original predictions) or as re-predicted (Design, predictions with KD-capped training) by a model trained on a trast-2 dataset version depleted of any variant binding more strongly than parental trastuzumab (Training, KD-capped), according to some aspects.

FIG. 8E depicts a scatterplot of predicted (design) and measured (validated) −log₁₀ K_{D} values, wherein the data refers to design set A of FIG. 8A, according to some aspects.

FIG. 8F depicts a scatterplot of measured (validated) −log₁₀ K_{D} values in individual SPR replicates, wherein the data refers to design set A of FIG. 8A, according to some aspects.

FIG. 8G depicts a scatterplot of predicted (design) and measured (validated) −log₁₀ K_{D} values, wherein the data refers to design set B of FIGS. 8B-8D, according to some aspects.

FIG. 8H depicts a scatterplot of measured (validated) −log₁₀ K_{D} values in individual SPR replicates, wherein the data refers to design set B of FIGS. 8B-8D, according to some aspects.

FIG. 8I depicts a chart of model predictions for variants with desired binding properties relative to naive library screening, according to some aspects.

FIG. 9A depicts an illustration of the combinatorial mutagenesis strategy of the trast-3 dataset: up to triple mutants in 20 positions (10 in CDRH2, 10 in CDRH3) of trastuzumab, screened using ACE, according to some aspects.

FIG. 9B depicts predictive performance of a model trained on the trast-3 dataset, with 20% of data in the hold-out set, according to some aspects.

FIG. 9C depicts models trained on up to triple mutants were validated against a hold-out set of up to triple mutants, and against hold-out sets of quadruple and quintuple mutants, extrapolating predictions to a higher mutational load than seen in the training set, according to some aspects.

FIG. 9D depicts a line plot showing model accuracy on a common hold-out validation set across different training set sizes, wherein: (i) shaded regions indicate standard deviations across folds; (ii) for each training subset size, respective performance of the OAS-pretrained model and a randomly-initialized model are shown, each trained using subsets of the high-fidelity trast-3 dataset or a low-fidelity version of the dataset; and (iii) under each subset size is included an indication of a fraction of training data used, the size of the training dataset, and the percent of the complete mutational space covered by the training subset, according to some aspects.

FIG. 9E depicts performance of modeling with randomized ACE scores (trast-3 dataset), according to some aspects.

FIG. 9F depicts extrapolation of predictions to higher mutational loads for quadruple mutants (trast-3 dataset), according to some aspects.

FIG. 9G depicts extrapolation of predictions to higher mutational loads for quintuple mutants (trast-3 dataset), according to some aspects.

FIG. 9H depicts a plot depicting that the effects of individual mutations can vary strongly with the presence of other mutations for ranges of incremental effects (minimum to maximum) on predicted binding affinity from a model trained on the trast-3 dataset upon each individual substitution across all possible single mutants of trastuzumab, according to some aspects.

FIG. 9I depicts a plot depicting that the effects of individual mutations can vary strongly with the presence of other mutations for ranges of incremental effects (minimum to maximum) on predicted binding affinity from a model trained on the trast-3 dataset upon each individual substitution across all possible double mutants of trastuzumab, according to some aspects.

FIG. 9J depicts sequence logo plots illustrating the composition of high-affinity variants of trastuzumab, according to some aspects.

FIG. 9K depicts a heatmap illustrating epistatic effects across all possible pairs of substitutions, according to some aspects.

FIG. 10A depicts predicted binding affinities for single mutants from a model trained on the trast-3 dataset, wherein (i) positions holding mutations comprised CDRH2 (10 positions starting with R55) and CDRH3 (10 positions starting with W107); (ii) the reference trastuzumab sequence is highlighted with crosses; and (iii) mutations at each position include all possible substitutions with natural amino acids except cysteine, sorted alphabetically (i.e., X∈[A, D, E, F, G, H, I, K, L, M, N, P, Q, R, S, T, V, W, Y]), according to some aspects.

FIG. 10B depicts predicted binding affinities for double mutants from a model trained on the trast-3 dataset, wherein (i) positions holding mutations comprised CDRH2 (10 positions starting with R55) and CDRH3 (10 positions starting with W107); (ii) the reference trastuzumab sequence is highlighted with crosses; and (iii) mutations at each position include all possible substitutions with natural amino acids except cysteine, sorted alphabetically (i.e., X∈[A, D, E, F, G, H, I, K, L, M, N, P, Q, R, S, T, V, W, Y]), according to some aspects.

FIG. 10C depicts regression performance of models trained with 10% of the CR 9114 dataset, according to some aspects.

FIG. 10D depicts regression performance of models trained with 1% of the CR 9114 dataset, according to some aspects.

FIG. 10E depicts regression performance of models trained with 0.1% of the CR 9114 dataset, according to some aspects.

FIG. 10F depicts regression performance of mixture models trained with 10% of the CR 9114, according to some aspects.

FIG. 10G depicts regression performance of mixture models trained with 1% of the CR 9114, according to some aspects.

FIG. 10H depicts regression performance of mixture models trained with 0.1% of the CR 9114, according to some aspects.

FIG. 11A depicts language models pre-trained with antibody repertoire that sequences can be leveraged to compute the naturalness of an antibody sequence conditioned on a given species, wherein naturalness scores were investigated for association with four antibody properties, according to some aspects.

FIG. 11B depicts immunogenicity using Anti-Drug Antibody (ADA) responses to humanized clinical-stage antibodies reported by Marks et al. [28] (n=97), according to some aspects.

FIG. 11C depicts developability failures as predicted by the Therapeutic Antibody Profiler (TAP) for round 3-enriched phage display hits from the Gifford library [29] (n=882), according to some aspects.

FIG. 11D depicts expression levels in HEK-293 cells (mg/L) of clinical-stage humanized antibodies from Jain et al. [30] (n=67), according to some aspects.

FIG. 11E depicts naturalness density plots for 6,710,401 trastuzumab variants split by mutational load, wherein dashed lines correspond to the naturalness of the parental trastuzumab sequence, according to some aspects.

FIG. 11F depicts a correlation between naturalness and antibody immunogenicity, according to some aspects.

FIG. 11G depicts naturalness scores of clinical-stage humanized antibodies used in the immunogenicity analysis of FIG. 11B, according to some aspects.

FIG. 11H depicts naturalness scores of round 3-enriched phase display hits from the Gifford library (n=882) used in a developability analysis with the Therapeutic Antibody Profiler (TAP) as in FIG. 11C, according to some aspects.

FIG. 11I depicts naturalness scores of trastuzumab triple mutants from the combinatorial space from which the trast-3 dataset was sampled (n=6710401) used in a developability analysis with TAP as in FIG. 11H, according to some aspects.

FIG. 11J depicts naturalness scores of clinical-stage humanized antibodies used in the analysis of HEK-293 expression titer as in FIG. 11D, according to some aspects.

FIG. 11K depicts naturalness scores corresponding to FIG. 11I and TAP-predicted developability failures for trastuzumab triple mutants from the combinatorial space from which the trast-3 dataset was sampled (n=6710401), wherein P-values may be computed using the Jonckheere-Terpstra trend test for binary data, according to some aspects.

FIG. 11L depicts a density map of the complete trast-3 search space, according to some aspects.

FIG. 11M depicts a density map of the variants with predicted ACE scores higher than trastuzumab, according to some aspects.

FIG. 12A depicts a diagram in which each line tracks the average predicted qaACE score of the best 100 sequences observed across the evolutionary trajectory, and shaded regions indicate the standard deviation, according to some aspects.

FIG. 12B depicts a diagram of average naturalness of the best 100 sequences observed across the evolutionary trajectory, wherein shaded regions indicate the standard deviation, according to some aspects.

FIG. 12C depicts a diagram of qaACE and naturalness scores of the best 100 sequences determined through three search strategies: Genetic Algorithm, Exhaustive Search, and Random Search; wherein dashed lines indicate the scores predicted for trastuzumab; and purple dashed lines indicate maximum scores predicted across the entire combinatorial space, according to some aspects.

FIG. 12D depicts a histogram showing the first generation where each of the top 100 sequences observed along the evolutionary trajectory was identified, according to some aspects.

FIG. 13A depicts a representative parent gating for all ACE sorts, according to some aspects.

FIG. 13B depicts a specific expression and collecting gating for each ACE library sort, according to some aspects.

FIG. 14 depicts a flow chart 1400 with the number of sequences filtered out and retained after each pre-processing step, according to some aspects.

FIG. 15A depicts output of a grid search across hyperparameter values performed on a pilot data set, according to some aspects.

FIG. 15B depicts output of a grid search across hyperparameter values performed on a subset of the pilot dataset of FIG. 15A containing 500 randomly selected sequences.

FIG. 16 depicts a chart depicting performance of models trained on ACE+SPR data with different ACE:SPR loss ratios, according to some aspects.

FIG. 17 depicts graphs of hyperparameter optimization for XGBoost baseline on a pilot dataset, according to some aspects.

FIG. 18A depicts a density plot of naturalness distributions for different sequence groups, according to some aspects.

FIG. 18B depicts a diagram of the relationship between sequence spaces, according to some aspects.

### DETAILED DESCRIPTION

The present disclosure addresses the need for an artificial intelligence (AI) and machine learning (ML) model that is trained using the mapping between antibody sequence variants and experimental measurements (e.g., binding affinities, pH, and other data types). As described herein, once trained, the model is able to predict the binding affinities of unseen sequence variants. The present techniques include deep contextual language models which, combined with high-throughput and low-throughput binding affinity data, may predict binding affinities of unseen antibody sequence variants spanning a K_{D} range of several (e.g., four) orders of magnitude. The present techniques enable measuring the “naturalness” of biomolecule (e.g., antibody) sequence variants, a widely-applicable metric shown herein to be associated with downstream issues related to drug developability and immunogenicity. The present techniques may accelerate and improve biomolecule (e.g., antibody) engineering, and increase the success rate of practical applications (e.g., developing antibody drug candidates).

A major challenge for constructing accurate machine-learning models is the scarcity of appropriate large-scale training datasets. Directed evolution platforms are well-suited for this as they rely on the linking of biological sequence data (DNA, RNA, protein) to a phenotypic output. In fact, it has long been proposed to use ML models trained on data generated by mutagenesis libraries as a means to guide protein engineering. In recent years, access to deep sequencing and parallel computing has enabled the construction of deep learning models capable of predicting molecular phenotype from sequence data. Deep learning incorporates multiple hidden layers to decipher relationships buried in large, high-dimensional data sets, such as the millions of reads gathered from a single deep sequencing experiment. Well trained models can then be used to make predictions on completely unseen and novel variants. This application of model extrapolation lends itself perfectly to protein engineering because it provides a way to interrogate a much larger sequence space than what is physically possible. Here we address this problem by combining deep mutational scanning and a bacterial display system to generate a training dataset for A ML model to learn sequence-function relationships.

An activity-specific cell-enrichment (ACE) assay that identifies host cells that express active gene product of interest (e.g., biomolecules, as used herein) rather than inactive material, has been described in WO 2021/146626, incorporated herein in relevant part. Active gene product can be distinguished from inactive material by the ability of active gene product to specifically bind a binding partner molecule, or by the ability of gene product to participate in a chemical or enzymatic reaction, as examples. The presence of properly formed disulfide bonds in a polypeptide gene product is an indication that it is correctly folded and presumptively active. In the cell-enrichment methods, active gene product of interest is detected by utilizing an appropriate labeling complex that specifically binds to active gene product of interest, such as a labeled antigen if the gene product of interest is an antibody or Fab; or a labeled ligand if the gene product of interest is a receptor or a receptor fragment, where the ligand specifically binds to an active conformation of the receptor; or a labeled substrate or a labeled substrate analog if the gene product of interest is an enzyme, as examples. For any gene product of interest, if there is an available antibody or antibody fragment that specifically binds to the active gene product and not to inactive gene product, that antibody or antibody fragment can be used to label the active gene product of interest when attached to a detectable moiety.

A key strength of ACE is its ability to screen tens of thousands of “units of variation” in a single run. However, ongoing AI efforts applied to drug discovery add additional requirements to wet lab-only screenings, which impose additional optimization of ACE to generate datasets suitable for AI. Wet lab-only screenings aimed at selecting top performing variants do not require stringent quantitativeness from an assay. Indeed, the iterative nature of such screenings is such that hits from the n−1 step are rescreened in step n, effectively weeding out n−1 false positives. Moreover, wet lab screenings are often tuned to selecting only a desired population of interest (for example, higher affinity variants), and as such the assay does not have to be quantitative over a large dynamic range of the parameter of interest (for example, antibody affinity). However, AI models for predicting quantitative predictions benefit from quantitative sequence variant training data. As such, quantitative sequence variant training data need to be accurate for the model to produce meaningful predictions down the line. The present disclosure addresses these needs and shortcomings.

The present disclosure provides, in various embodiments, an augmentation of the qaACE assay-quantitative affinity qaACE (“qaACE”), as a method for sampling the affinity of antibody variants at high throughput using flow cytometry and next generation sequencing to generate a qaACE score that correlates with KD. The main goal of this method is to generate highly quantitative high throughput training data for an AI model to perform sequence-based affinity predictions. This method can be applied to any antibody format, mabs, fabs, scFv, scFAB, VHH, nanobody etc. and could conceivably be applied to other binding drug formats as well.

In one embodiment, the first step in the qaACE process is to generate a mutationally diverse antibody library, that evenly sample the sequence space around the starting point antibody molecule. This library contains variants that span a range in mutational distance from the original sequence.

In some embodiments including the Examples herein, the method provides a flow cytometry read out of an antibody, expressed in SoluPro E. coli, binding to a fluorescently labeled antigen probe. In the qaACE assay, setting expression of the antibody molecule is normalized such that a change in fluorescent signal in a cell will be due to different affinities of the expressed antibody variants in the cells binding to the fluorescent antigen probe. This normalization is accomplished via a generic target molecule probe that will bind to all variants and whose signal will be in an orthogonal fluorescent channel to the antigen probe. In this setting we show that the fluorescent signal of a variant is proportional to the measured KD of an antibody variant within a range. Given this proportionality, using FACS, cells containing antibody variants can be sorted that span a range (e.g., a distribution) of affinities.

After sorting across a range of affinity values with gating across the library population distribution, the cell material is sequenced and quantified for the prevalence of observed variants across the affinity gates (bins, tubes). Using the quantifications, an enrichment score is calculated for each variant. The enrichment scores generated via qaACE are an ideal data type for AI modeling purposes because of the accuracy and throughput.

In one exemplary workflow, the present disclosure provides a qaACE assay that comprises some or all of the following general steps:
1) Generation of an antibody or other drug molecule library for screening through qaACE expressed in a host cell such as SoluPro E. coli.
2) Identification of an antigen or binding partner probe that is fluorescently labeled for use in, for example, FACS via initial cytometry development process.
3) Use of generic probe to the target molecule variants that will allow for detection of expression level within a cell. This expression signal is used to gate a uniformly expression population to disambiguate affinity and expression signal related to epitope binding signal.
4) Sorting of cells across the affinity distribution.
5) Sequencing of cells sorted across the affinity distribution.
6) During the sequencing DNA barcodes or UMIs may be added via PCR amplification of the region of interest. These UMIs will enable absolute quantification of variants retrieved from the gates.
7) Generation of affinity correlated enrichment scores for each observed variant.
8) AI model training using enrichment score and antibody variant sequence.

As described herein, the present disclosure provides a method for generating highly quantitative high-throughput training data for a ML model to perform, for example, sequence-based affinity predictions. In some embodiments of the present disclosure, sequences of a highly diverse library of biomolecule variants, which are expressed in, or on the surface of, host cells, serve as input to an experiment (e.g., an assay to determine expression and/or affinity, among other readouts). In some embodiments, the variants are sorted into a plurality of bins based on high throughput measurements of binding affinity values (KD) which are normalized for variant expression levels and variant sequences in each bin are obtained and tallied by deep DNA sequencing. In some embodiments, the method then outputs a plurality of enrichment scores which correlate the KD across the full experimental affinity distribution, (i.e. from non-binders, low and high binders) and sequence information of every biomolecule variant in each bin. The enrichment scores generated via qaACE assay of the present disclosure are an ideal data type for AI modeling purposes because of their accuracy and throughput. The combined method of obtaining affinity and sequence data of biomolecule variants is accordingly referred to herein as the quantitative affinity Activity-specific Cell Enrichment (qaACE) assay.

As used herein, the term “quantitative affinity Activity-specific Cell Enrichment or qaACE assay” refers to a high throughput assay for obtaining affinity and sequence data of biomolecule variants (U.S. Provisional Application No. 63/371,474, filed Aug. 15, 2022, incorporated by reference in its entirety).

As used herein, the term “affinity distribution” refers to the distribution of KD values for antigen binding to all possible sequence variants in the randomized library of biomolecule variants. A comparison to the KD value of the reference biomolecule gives an indication whether the variants bind with a higher or lower affinity.

The present techniques demonstrate the capability to improve the binding affinity of an antibody to its target antigen using deep contextual language models and quantitative, high-throughput experimental binding affinity data. We show that models can quantitatively predict binding affinities of unseen antibody variants with high accuracy, providing the ability to perform drug screenings in silico, ultimately augmenting the accessible sequence space by orders of magnitude. In this sense, the trained learner fulfills the role of a general surrogate to the black-box problem of assigning a functional annotation from sequence alone. Novel variants with defined properties can be consistently designed by using models as oracles for a variety of frameworks trained on the protein fitness landscape. We confirm predictions and consequent designs in the lab, with a much higher success rate than would be attained with traditional screening.

The present deep contextual language models include large language models (e.g., for antibody engineering using high-quality binding affinity measurements of Trastuzumab sequence variants) that are capable of predicting binding affinities of unseen sequence variants spanning one or more (e.g., four) orders of magnitude with high accuracy, resulting in the ability to perform drug screenings entirely in silico. Here, by introducing natural antibody sequences into our language models, the present techniques are able to “characterize the naturalness” of any given sequence for a host species. Empirical study has shown that high naturalness scores are associated with improved immunogenicity and developability metrics, thereby highlighting the importance of simultaneously optimizing multiple antibody properties during drug lead screening. To address this task, we present a genetic algorithm for the extremely efficient identification of sequences with both strong binding affinity and high naturalness.

These models may be used to identify variants with improved binding affinity and to confirm that many (e.g., 76%) of those variants have higher binding affinity than wild-type Trastuzumab based on precise wet lab screening. These developments in combining deep contextual language models with in silico screening enable greatly accelerated antibody lead optimization and improve the therapeutic potential of antibody candidates.

As will be appreciated by those of skill in the art, a much larger mutational space can be explored, since in silico modeling is much faster and cheaper than wet lab experiments. As provided herein, in some aspects, the model performs quantitative predictions of binding affinity (expressed as K_{D}—i.e., the model is a regressor) as opposed to the most recently published state of the art (Mason et al., Nat Biomedical Engineering, 2021, 5, 600-612) which can only perform qualitative predictions (binders vs. non-binder, i.e., in Mason, the model is a rudimentary classifier). By virtue of screening in silico, exploration of a far greater sequence space is possible as compared to using wet lab methods. The present techniques may include wet lab aspects (e.g., for model training) that greatly accelerate the generation of highly accurate training data. The AI-assisted workflow described herein thus provides higher yield and better results with less effort.

As will be appreciated by those of ordinary skill in the art, while the present disclosure provides various embodiments related to antibody-antigen binding properties, the model and methods described herein can be applied to any biomolecule including, for example, a protein, nucleic acid, receptor, ligand and the like, wherein the biomolecule is capable of binding or otherwise interacting with a binding partner (which, in some embodiments is the same or different type of biomolecule). “Biomolecule sequence variants of interest” thus refers to, in some embodiments, variations (e.g., mutations) of a sequence of a biomolecule (such as an antibody or antibody fragment) as described herein. The present techniques include generating mutated sequences in silico and subsequently synthesizing those sequences in a laboratory setting.

Embodiments of the present disclosure provide compositions and methods for using a model to, for example, identify an antibody sequence that will confer a higher binding affinity (e.g., to its antigen binding partner). The model may be an artificial neural network. In one embodiment, the neural network architecture is a transformer-encoder model, as described in RoBERTa (Liu et al. 2019 arXiv) and “Attention is all you need” (Vaswani et al. 2017 arXiv). The RoBERTa architecture belongs to the “transformers” family of neural networks, primarily used in natural language processing. Those of ordinary skill in the art will appreciate that RoBERTa refers to the entire training setup, not exclusively the model architecture (the architecture is a transformer encoder). The fine architecture (such as number of hidden layers, size of embeddings, etc.) may be parameterized according to a number of alternative RoBERTa configurations, and experimental results indicate comparable performance. As such, the fine-grained architecture is likely not a prime factor in performance. Alternative transformer and non-transformer deep neural network architectures may yield similar performances. Alternative non-deep-neural-network machine learning algorithms might yield similar or slightly inferior performances.

In some aspects, the core architecture of the models is the RoBERTa model (e.g., its PyTorch implementation within the Hugging Face framework (Wolf, T., Debut, L., Sanh, V., Chaumond, J., Delangue, C., Moi, A., Cistac, P., Rault, T., Louf, R., Funtowicz, M., et al. Huggingface's transformers: State-of-the-art natural language processing. arXiv preprint arXiv:1910.03771, 2019). The trunk of the model may contained a number (e.g., 16) of hidden layers, with a number (e.g., 12) of attention heads per layer, and a hidden layer size (e.g., 768). The regression tasks may include one or more hidden layers of a given size (e.g., 768), followed by a projection layer with the required number of outputs. For example, the total size of the model may be 114 million parameters.

As discussed below, the present techniques may include binding affinity models that are first pre-trained (e.g., on immunoglobulin sequences) in a self-supervised regime (e.g., using the Observed Antibody Space database (OAS)). The immunoglobulin chains may be represented by a token encoding the species from where the biomolecule (e.g., antibody) was derived, followed by a concatenation of complementarity-determining regions (CDRs), defined, e.g., by using the union of IMGT (Lefranc, M.-P., Pommié, C., Ruiz, M., Giudicelli, V., Foulquier, E., Truong, L., Thouvenin-Contet, V., and Lefranc, G. Imgt unique numbering for immunoglobulin and t cell receptor variable domains and ig superfamily v-like domains. Developmental & Comparative Immunology, 27(1):55-77, 2003) and Martin (Abhinandan, K. and Martin, A. C. Analysis and improvements to kabat and structurally correct numbering of anti-body variable domains. Molecular immunology, 45(14): 3832-3839, 2008) labeling schemes (e.g., with separator tokens between them). For example, as discussed below, the present techniques may include receiving data (e.g., OAS database) including unpaired immunoglobulin chains and excluded studies (e.g., those whose samples were also a part of another study present in the database, studies originating from immature B cells, B cell-associated cancers, etc.). In some aspects, the present techniques may include further filtering the sequences that fail a number of quality checks, and/or extracting desired chain representations (e.g., Extended CDR or Near Full), and/or de-duplicating the resulting sequences across the entire database. The present techniques may further include filtering out sequences that were only observed once in a single study, as shown in the following table, depicting datasets and training configurations for respective pretraining tasks:

| | | DATA | BATCH | TRAINING |
|---|---|---|---|---|
| REPRESENTAITON | CHAIN | SET SIZE | SIZE | STEPS |
| EXTENDED CDR | HEAVY | 139,187,988 | 12,288 | 130000 |
| EXTENDED CDR | LIGHT | 2,262,795 | 9,216 | 48200 |
| NEAR FULL | HEAVY | 150,202,903 | 6,656 | 200000 |
| NEAR FULL | LIGHT | 2,459,027 | 6,656 | 40000 |

Antibody variants, including sequence variants (e.g., within a region or regions of a reference antibody) are described in detail herein. A sequence variant is a sequence deviating from the reference/wild type sequence by one or more mutations. Mutations are often introduced in CDRs (identified according to any common definition such as IMGT, Martin, Kabat, Chothia, etc) but may in principle be introduced in the framework as well. The present techniques include intentionally “mutating” sequences to generate sequence variants thereof.

The data and predictions provided by the present disclosure have been generated using Fabs, but alternative scaffolds such as mAbs, scFvs, VHHs, etc., as well as heavy chain vs light chain, are possible with a very similar modeling approach.

As described herein, in one embodiment the model is first pre-trained using natural antibody sequencing data from multiple species, including human, mouse and camelid among others (e.g., any sequence data relating to any suitable species now known or later developed). Pre-training is performed, in one embodiment, using a masked language model objective: some positions in the antibody sequence are randomly masked and the model is tasked with predicting which amino acid was present at the masked position (classification task). By doing so, the model gains an understanding of the “grammar” governing antibody sequences (i.e. it gets an understanding of “naturalness” and/or “humanness”), which makes it more efficient to later fine-tune the model using affinity data. Pre-training does not require labeled data: only antibody sequences are necessary, without knowledge of their antigen specificity or other properties. The requirement is only that these sequences are natural sequences. These sequences were sourced from the Observed Antibody Space (OAS), a database published by the University of Oxford's Oxford Protein Informatics Group (OPIG). OAS does not contribute novel sequences: it is an aggregator of data sourced from multiple publications. However, it does re-annotate the raw data from such disparate sources with a unified pipeline (Kovaltsuk, et al., J Immunol, 2018, 201 (8) 2502-2509). OAS re-annotation is convenient but likely not a prime factor in modeling performance. Similarly, aggregation of data from multiple studies is convenient but no single study is likely essential for the modeling performance.

The present disclosure provides that, in some embodiments, pre-training improves affinity predictions when proprietary affinity data is limiting. As the size of proprietary affinity datasets increases, the benefit of pre-training decreases. As such, pre-training is, in one embodiment, characterized as optional, while still emphasizing the benefit of pre-training in “low-N” settings (i.e. small affinity datasets). This concept is well-established in protein engineering (Biswas, S., Khimulya, G., Alley, E. C. et al. Low-N protein engineering with data-efficient deep learning. Nat Methods 18, 389-396 (2021). https://doi.org/10.1038/s41592-21-01100-y), but no demonstration specific to antibodies has been made to date. The distinction between generic protein engineering and antibody engineering is important because not all machine learning methods developed for proteins work with antibodies. As an example, AlphaFold2 can predict protein complexes, but cannot predict antibody-antigen interactions. (Evans, R., et al., bioRxiv 2021.10.4.463034). Additionally, in some embodiments, pre-training is beneficial for reasons unrelated to low-N settings, such as the ability to make predictions for variants with high mutational burden using training data from variants with lower mutational burden only (among others).

After pre-training (or random initialization, in embodiments wherein pre-training is not performed), the model may be trained (i.e., fine-tuned) using affinity data generated using a workflow encompassing primary screening, for example using a high-throughput, quantitative, activity-based method (WO/2021/146626) and targeted rescreening with Carterra LSA SPR (high accuracy) (carterra-bio.com/Isa/). In other embodiments, other display technologies are used (e.g. yeast display, mRNA display, phage display, ribosome display, etc.) in a deep mutational scanning (DMS) setting (Kyrin, R., et al., Trends in Pharmacological Sciences, 2021, ISSN 0165-6147). In still other embodiments, Carterra LSA SPR can be replaced with low-throughput/traditional SPR, BLI or similar techniques. While data collection is described herein in two steps to maximize throughput (primary screening) and accuracy (secondary rescreening), in some embodiments the disclosure provides either of the two steps to generate training data. Because affinity data is antigen-specific, the model is also antigen-specific. For a new antibody/antigen pair, pre-training is not repeated, but affinity data collection and model fine-tuning is repeated.

Upon model pre-training and fine-tuning, the model can make affinity predictions for unseen sequence variants. For low-combinatorial space (for example, triple mutants in two CDRs), the number of sequence combinations is sufficiently small to be tackled by exhaustively predicting the affinity of every possible sequence variant.

For highly combinatorial space, the number of sequence combinations is too large to be exhaustively predicted computationally. In that case, two solutions are possible as provided by the present disclosure: (1) turning a predictive model into a generative model using Plug and Play language models (Dathathri et al., arXiv:1912.02164 [cs.CL]), but several other generative strategies have been published (see, e.g., Zachary Wu, Kadina E. Johnston, Frances H. Arnold, Kevin K. Yang, Protein sequence design with deep generative models, Current Opinion in Chemical Biology, Volume 65, 2021, Pages 18-27, ISSN 1367-5931, https://doi.org/10.1016/j.cbpa.2021.4.004.(https://www.sciencedirect.com/science/article/pii/S13 6759312100051X)); and (2) coupling model predictions with more traditional optimization techniques, such as genetic algorithms and the like (e.g., simulated annealing). Still further methods are available to those of ordinary skill in the art (see, e.g., “Controllable Neural Text Generation,” https://lilianweng.github.io/lil-log/2021/01/02/controllable-neural-text-generation.html (retrieved 1/7/2022)).

The methods and models provided by the present disclosure can be used for numerous purposes. In one embodiment, the methods and models provided by the present disclosure are used for affinity maturation of weakly binding antibodies, including commercial antibodies. Such antibodies may be weak binders because of humanization of animal-derived antibodies, de novo hits from library screenings, poor target immunogenicity (e.g. mammalian antigens), or other reasons.

In one embodiment, the methods and models provided by the present disclosure are used for simultaneous affinity maturation towards two or more antigens. Such antigens might be homologous proteins belonging to different species, often one being human and the other(s) being a non-human species, e.g., cynomolgus monkey. Engineering an antibody to bind to the same antigen from different species enables in vivo testing during development. Alternatively, the antigens might be variants of the same protein. An example is in infectious diseases, where certain variants might escape antibody binding, thereby abrogating therapeutic efficacy. Restoring affinity towards escape variants without compromising affinity towards non-escape variants is valuable to endow an antibody with broad neutralizing activity. Alternatively, the antigens might be distinct members of the same family. This is valuable when multiple members of the same family must be engaged or neutralized, either because therapeutic potency increases or because there is functional redundancy across family members such that engaging/blocking a single member is ineffective.

In another embodiment, the methods and models provided by the present disclosure are used for affinity maturation for the same antigen under different conditions. For example, such conditions might involve varying the pH, which might change in different microenvironments, thereby affecting binding.

While the emphasis of affinity maturation is often on increasing binding affinity as much as possible, the model and methods described herein make quantitative predictions, enabling, for example, (i) reducing, rather than increasing, affinity; (ii) engineering affinity to be within defined lower and upper bounds, e.g., to facilitate clearing of the antibody in vivo and/or to limit engagement or blockade of the target antigen when side effects are present; and (iii) when performing multi-antigen affinity maturation, the goal might not necessarily be enhancement for all antigens—it may be desirable to increase affinity towards one or more antigen(s) while decreasing/abrogating affinity towards one or more other antigen(s) (For example, this might be advantageous when engaging/blocking an antigen providing therapeutic benefit, while engaging/blocking a related antigen leads to toxicity. Similarly, one might want to enhance affinity against the specific target, while reducing/abrogating non-specific binding to a related but undesired antigen).

While the emphasis is on affinity maturation of antibodies against antigens, in some embodiments the same strategy can be applied to any protein-protein interaction. This might benefit from performing model pre-training with a protein database such as Uniref90 rather than or in addition to the OAS, depending on the nature of the two interactants. For example, a cytokine sequence might be engineered to increase/decrease binding affinity towards receptors. Similarly, next-generation antibody scaffolds as well as antibody mimetic scaffolds (such as DARPins) can also be engineered. In another example, the Fc region of an antibody might be engineered to increase/decrease binding for specific Fc receptors using the same strategy described here for the variable region affinity for antigens. In other aspects, the model may be pre-trained on multiple databases such as OAS and Uniref90, either in a combined form or sequentially.

In one embodiment, the model requires training data specific for the pairwise interaction being optimized (i.e. affinity data of antibody sequence variants against a single antigen). A novel pairwise interaction of interest will require a new training dataset specific for that interaction. However, the model architecture, the model pre-training and the workflow (from data generation all the way to model prediction) remain the same. While the model is in one embodiment as described herein used with training data specific for the pairwise interaction being optimized (i.e. affinity data of antibody sequence variants against a single antigen), the following embodiments are also provided herein: (a) The antigen (and not the antibody) is mutated, while the antibody (and not the antigen) is fixed. This is useful to predict, for example, which antigen variants are likely to escape the antibody; (b) Library-on-library screening and training, where both the antibody and the antigen are simultaneously mutagenized (and not just the antibody). This is useful, for example, when engineering an antibody against antigen variants (for example, escape variants in infectious diseases) without having to screen for a predetermined/fixed set of variants. This is also useful when insight about the paratope/epitope residues is needed; (c) A library of antibody sequences which is no longer variants (i.e. one or few mutations away) of a reference sequence, but rather a randomized CDR(s) sequence(s) library in a selected antibody scaffold. This would enable the model to learn about multiple unrelated binders to the same antigen. Distinct binders might target distinct antigenic sites/epitopes; (d) A library of antigens which are no longer variants of a reference sequence, but rather randomized peptides, either linear or conformational/cyclic. This would enable the model to learn about which motifs (linear or 3D) bind to an antibody sequence; (e) A (library-on-library) combination of the previous two points, which would enable the model to learn near-universal relationships between arbitrary antibody sequences and arbitrary structural motifs (captured by the peptide library). This might ultimately enable de novo in silico antibody design upon specifying an epitope of choice in terms of an alphabet of structural motifs, and then asking the model to generate a sequence with affinity against such structural motifs.

In one embodiment of the present disclosure, affinity is rendered numerically as K_{D} (or single-number surrogates/correlates of K_{D}). As K_{D} results from association and dissociation constants (Kₐ and K_{d}) and the same K_{D} can result from different combinations of Kₐ and K_{d}, the model can be tasked with predicting Kₐ and K_{d} rather than K_{D}. This is useful when specific association/dissociation rates are desired, as opposed to overall affinity.

While in one embodiment the focus is antigen affinity, the model provided herein maps sequences to numerical features. As such, the same modeling strategy (including pre-training with natural antibody sequences) can be used to predict any quantitative outcome for which there is sufficient training data. The decision to deploy a model to predict a different numerical property of an antibody does not depend on the modeling strategy, which is invariant, but on the assay throughput, which should be sufficient to generate enough data for the fine-tuning step. It is important to note that, unlike affinity, most other properties of an antibody are not specific for a given antigen, but depend exclusively on the sequence of the antibody. This is the case, for example, of biophysical/developability properties of an antibody such as solubility, viscosity, etc. On the one hand, this means that training data acquired for a project may be consolidated with training data acquired for a different project, even if these two projects concern different antibodies/antigens. On the other hand, it means that generalizable predictions require training data spanning a greater sequence variation than just local variation around a few reference/wild type sequences.

As used herein, the term “developability” refers to the feasibility of molecules to successfully progress from discovery to development via evaluation of their physicochemical properties. The term “developability” may also include concepts related to the ability of a molecule (e.g., an antibody) to bind to a desired target molecule and other considerations (e.g., feasibility of manufacture, stability in storage, and absence of off-target stickiness). As used herein, “binding partner” refers to a molecule with which another molecule forms a physical interaction. For example, the binding partner of an antibody is its antigen. As used herein, the term “binding characteristic” includes but is not limited to an equilibrium dissociation constant (K_{D}) that is a metric measuring binding affinity, a dissociation constant (K_{d}) and an association constant (Kₐ). K_{D} may be defined as the concentration of ligand, which half the ligand binding sites on the protein are occupied in the system equilibrium. It may be calculated by dividing a rate constant (K_{off}) that says the same for a given pair of proteins and ligands, by a the rate at which a forward reaction is taking place while a protein ligand complex is formed (Kₒₙ).

The biophysical/developability properties of the present techniques represent significant advantageous improvements over conventional techniques. In particular, the careful preprocessing and preparation of data in the present techniques, especially in those aspects of the present techniques that use model pre-training and/or model fine-tuning, significantly improve over those conventional methods that are based on random model initialization. Specifically, the sequences generated by the present modeling techniques have inherently better developability properties because the models are informed by natural immune repertoires, being trained on antibodies that actually exist in humans. Thus, the results of the modeling is better and more developable than any results based on a randomized approach could be.

Those of ordinary skill in the art will appreciate that finding high affinity biomolecules that relate to sequences not found in humans or other animals, and are thus not developable, is a possible, if not likely outcome. Sequence identification is a multi-variate problem, wherein affinity binding is but one variable. Finding high affinity antibodies that suffer from developability issues (poor solubility, excessive viscosity, human immunogenicity, etc.) are major research and development roadblocks that are overcome by the present techniques. These roadblocks are particularly common when phage display techniques are used.

Still, while the present modeling techniques may be biased toward “humanness” (i.e., sequences that are more similar to those found in humans, and thus more likely developable) this is not to say that the present techniques cannot output a sequence that appears non-natural. Indeed, in some cases, the strength of affinity of an unnatural biomolecule may override the penalties for unnaturalness resulting from pretraining data.

The present techniques are highly sensitive, so much so, in fact, that experimental error/noise generated during binding assay may affect modeling outputs in some ranges. For example, when considering a range of predictions (e.g., between 0.1 picomolar to 0.2 picomolar) experimental error introduced during assay may cause sequential experimental runs of trained models to generate results having different orders (e.g., the top two affinity variants may be transposed). This may prevent relative ranking of variants by affinity in some cases. However, as discussed herein, the present techniques still represent a significant advantageous improvement over conventional techniques, which are limited to binary classification, whereas the present techniques are quantitative and generally enable mathematical operations (e.g., ranking, sorting, averaging, limiting, etc.) across orders of magnitude.

The following references describe various aspects of protein engineering and modeling: Protein engineering using machine learning (Biswas et al.; and Evans et al.); Antibody affinity engineering using machine learning (Mason et al., and Hanning et al., Trends Pharm Sciences, 2021, doi.org/10.1016/j.tips.2021.11.10); Generative NLP models (Dathathri et al); Antibody sequencing data (Kovaltsuk et al.); Model architecture (Liu et al. 2019 arXiv, arXiv:1907.11692); and Machine learning guided polypeptide design (WO2021026037A1 and WO2020167667A1).

The present in silico screening aspects provide many important advantages over wet lab assays, including dramatically higher throughput and multi-objective optimization. The present techniques demonstrate the capability of deep learning models to accurately predict binding affinity over several (e.g., four) orders of magnitude of K_{D}, and that models may include implicit understandings of sequence naturalness, providing a strong measure of various developability measures. The present techniques represent advantageous practical steps towards enhanced in silico antibody design for therapeutic applications.

### Exemplary Computer-Implemented Machine Learning Training and Operation

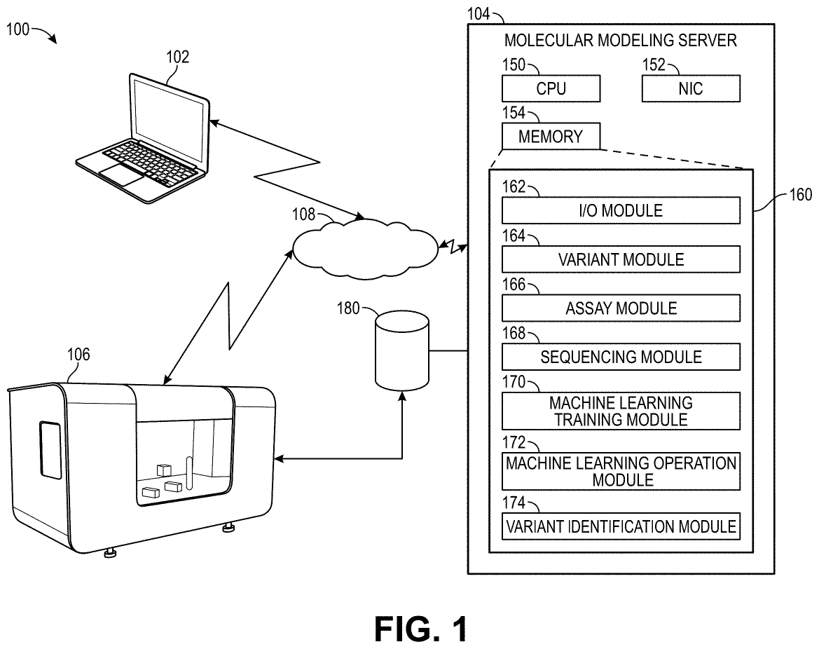

FIG. 1 depicts an exemplary computing environment 100 for training and/or operating one or more machine learning (ML) models, according to some aspects. The environment 100 includes a client computing device 102, a molecular modeling server 104, an assay device 106 and an electronic network 108. Some embodiments may include a plurality of client devices 102, a plurality of molecular modeling servers 104, and/or a plurality of assay devices 106. Generally, the one or more molecular modeling servers 104 operates to perform training and operation of full or partial in silico molecular modeling as described herein.

The client computing device 102 may be an individual server, a group (e.g., cluster) of multiple servers, or another suitable type of computing device or system (e.g., a collection of computing resources). For example, the client computing device 102 may be any suitable computing device (e.g., a server, a mobile computing device, a smart phone, a tablet, a laptop, a wearable device, etc.). In some embodiments, one or more components of the client device 102 may be embodied by one or more virtual instances (e.g., a cloud-based virtualization service) and/or may be included in a respective remote data center (e.g., a cloud computing environment, a public cloud, a private cloud, hybrid cloud, etc.). The client computing device 102 includes a processor and a network interface controller (NIC). The processor may include any suitable number of processors and/or processor types, such as CPUs and one or more graphics processing units (GPUs). Generally, the processor is configured to execute software instructions stored in a memory. The memory may include one or more persistent memories (e.g., a hard drive/solid state memory) and stores one or more set of computer executable instructions/modules. For example, the executable instructions may receive and/or display results generated by the server 104.

The client computing device 102, may include a respective input device and a respective output device. The respective input devices may include any suitable device or devices for receiving input, such as one or more microphone, one or more camera, a hardware keyboard, a hardware mouse, a capacitive touch screen, etc. The respective output devices may include any suitable device for conveying output, such as a hardware speaker, a computer monitor, a touch screen, etc. In some cases, the input device and the output device may be integrated into a single device, such as a touch screen device that accepts user input and displays output. The NIC of the client computing device may include any suitable network interface controller(s), such as wired/wireless controllers (e.g., Ethernet controllers), and facilitate bidirectional/multiplexed networking over the network between the client computing device 102 and other components of the environment 100.

The molecular modeling server 104 includes a processor 150, a network interface controller (NIC) 152 and a memory 154. The molecular modeling server 104 may further include a data repository 180. The data repository 180 may be a structured query language (SQL) database (e.g., a MySQL database, an Oracle database, etc.) or another type of database (e.g., a not only SQL (NoSQL) database). In some aspects, the data repository 180 may comprise file system (e.g., an EXT filesystem, Apple file system (APFS), a networked filesystem (NFS), local filesystem, etc.), an object store (e.g., Amazon Web Services S3), a data lake, etc. The data repository 180 may include a plurality of data types, such as pretraining data sourced from public data sources (e.g., OAS data) and fine-tuning data. Fine-tuning data may be proprietary affinity data that is sourced from a quantitative assay ACE, Carterra, or any other suitable source.

The server 104 may include a library of client bindings for accessing the data repository 180. In some embodiments, the data repository 180 is located remote from the molecular modeling server 104. For example, the data repository 180 may be implemented using a RESTdb.IO database, an Amazon Relational Database Service (RDS), etc. in some aspects. In some aspects, the molecular modeling server 104 may include a client-server platform technology such as Python, PHP, ASP.NET, Java J2EE, Ruby on Rails, Node.js, a web service or online API, responsive for receiving and responding to electronic requests. Further, the molecular modeling server 104 may include sets of instructions for performing machine learning operations, as discussed below, that may be integrated with the client-server platform technology.

The assay device 106 may be a Surface Plasmon Resonance (SPR) machine, for example, such as a Carterra SPR machine. The device 106 may be physically connected to either the molecular modeling server 104 or the data repository 180, as depicted. The device 106 may be located in a laboratory, and may be accessible from one or more computers within the laboratory (not depicted) and/or from the molecular modeling server 104. The device 106 may generate data and upload that data to the data repository 180, directly and/or via the laboratory computer(s). The assay device 106 may include instructions for receiving one or more sequences (e.g., mutated sequences) and for synthesizing those sequences. The synthesis may sometimes be performed via another technique (e.g., via a different device or via a human). In some aspects, the device 106 may be configured not as a device, but as an alternative assay that can measure protein-protein interactions as listed in other sections of this application. For example, the device 106 may instead be configured as a suite of devices/workflows, including plates and liquid handling. In general, the device 106 may be substituted with suitable hardware and/or software optionally including human operators to generate affinity data.

The network 108 may be a single communication network, or may include multiple communication networks of one or more types (e.g., one or more wired and/or wireless local area networks (LANs), and/or one or more wired and/or wireless wide area networks (WANs) such as the Internet). The network 108 may enable bidirectional communication between the client computing device 102 and the molecular modeling server 104, for example.

The processor 150 may include any suitable number of processors and/or processor types, such as CPUs and one or more graphics processing units (GPUs). Generally, the processor 150 is configured to execute software instructions stored in the memory 154. The memory 154 may include one or more persistent memories (e.g., a hard drive/solid state memory) and stores one or more set of computer executable instructions/modules 160, including an input/output (VO) module 162, a variant module 164, an assay module 166, a sequencing module 168, a machine learning training module 170, a machine learning operation module 172; and a variant identification module 174.

Each of the modules 160 implements specific functionality related to the present techniques, as will be described further, below. The modules 160 may store machine readable instructions, including one or more application(s), one or more software component(s), and/or one or more APIs, which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. In some embodiments, a plurality of the modules 160 may act in concert implement a particular technique. For example, the machine learning operation module 172 may load information from one or more other models prior to, during and/or after initiating an inference operation. Thus, the modules 160 may exchange data via suitable techniques, e.g., via inter-process communication (IPC), a Representational State Transfer (REST) API, etc. within a single computing device, such as the molecular modeling server 104. In some embodiments one or more the modules 160 may be implemented in a plurality of computing devices (e.g., a plurality of servers 104). The modules 160 may exchange data among the plurality of computing devices via a network such as the network 108. The modules 160 of FIG. 1 will now be described in greater detail.

Generally, the I/O module 162 includes instructions that enable a user (e.g., an employee of the company) to access and operate the molecular modeling server 104 (e.g., via the client computing device 102). For example, the employee may be a software developer who trains one or more ML models using the ML training module 170 in preparation for using the one or more trained ML models to generate outputs used in an antibody modeling project. Once the one or more ML models are trained, the same user (or another) may access the molecular modeling server 104 via the I/O module to cause the molecular modeling process to be initiated. The I/O module 162 may include instructions for generating one or more graphical user interfaces (GUIs) (not depicted) that collect and store parameters related to biomolecular modeling, such as a user selection of a particular reference protein, biomolecule, binding partner, etc. from a list stored in the data repository 180.

The variant module 164 may include computer-executable instructions for generating one or more mutated sequence variants based on a one or more reference biomolecules. For example, the user may be able to parameterize the variant module 164 using the I/O module 162 to selectively alter the manner in which reference biomolecule mutations are performed, and the use may repeatedly perform mutations, each of which the variant module 164 may store in the data repository 180 using a set of mutation storage instructions. Thus, the user may, via the I/O module 162, retrieve a previously run parameterized mutated sequence variant, or load the results of that mutation.

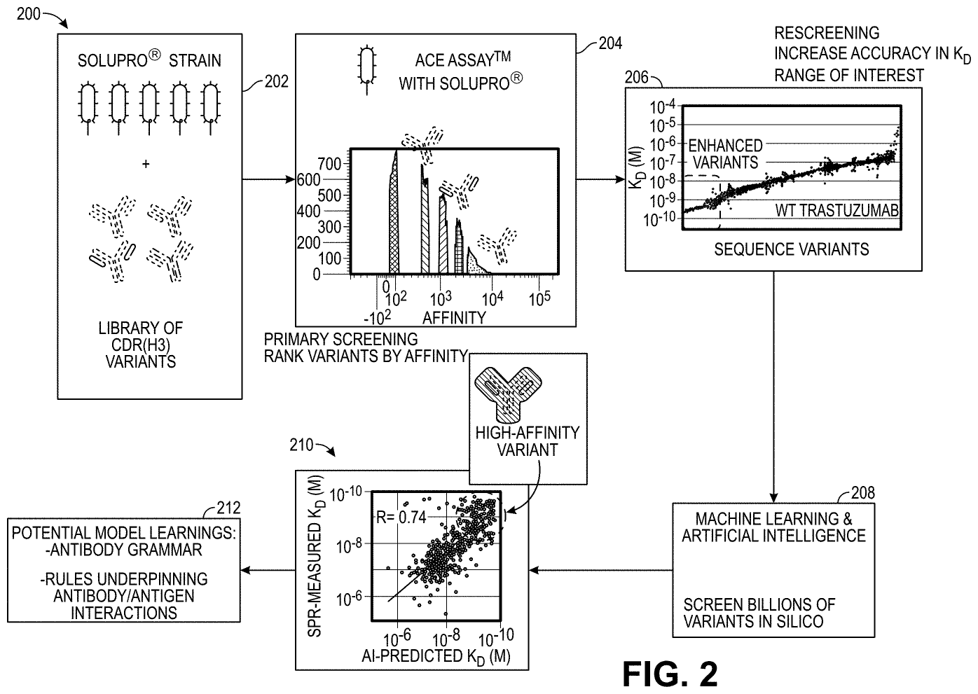

The assay module 166 may include computer-executable instructions for retrieving/receiving one or more synthesized mutated variants (e.g., via the memory 154 and/or via the data repository 180, when stored) and for controlling the assay machine 106. For example, the assay module 166 may include instructions for causing the assay machine 106 to analyze the synthesized mutated variants. The assay module may include instructions for determining binding kinetics and for performing next-generation sequencing, to determine measured binding affinity, as shown in FIG. 2. The assay module 166 may store the determined measured binding affinity in the data repository 180 in association with the one or more mutated variants, such that another module/process (e.g., the sequencing module 168) may retrieve the variant, along with its measured binding affinity and other related data.

The sequencing module 168 may include computer-executable instructions for manipulating genetic sequences and for transforming data generated by the assay module 166 and its operation of the assay machine 106, in some aspects. The sequencing module 168 may store transformed assay data in a separate database table of the electronic data repository 180, for example. The sequencing module 168 may also, in some cases, include a software library for accessing third-party data sources, such as OAS.

### Exemplary Computer-Implemented Machine Learning Model Training and Model Operation

In general, a computer program or computer based product, application, or code (e.g., the model(s), such as machine learning models, or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 150 (e.g., working in connection with the respective operating system in memory 154) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

In some aspects, the computing modules 160 may include a ML model training module 170, comprising a set of computer-executable instructions implementing machine learning training, configuration, parameterization and/or storage functionality. The ML model training module 170 may initialize, train and/or store one or more ML models, as discussed herein. The trained ML models and their weights/parameters may be stored in the data repository 180, which is accessible or otherwise communicatively coupled to the molecular modeling server 104.

For example, the ML training module 170 may train one or more ML models (e.g., an artificial neural network (ANN)). One or more training data sets may be used for model training in the present techniques, as discussed herein. The input data may have a particular shape that may affect the ANN network architecture. The elements of the training data set may comprise tensors scaled to small values (e.g., in the range of (−1.0, 1.0)). In some aspects, a preprocessing layer may be included in training (and operation) which applies principal component analysis (PCA) or another technique to the input data. PCA or another dimensionality reduction technique may be applied during training to reduce dimensionality from a high number to a relatively smaller number. Reducing dimensionality may result in a substantial reduction in computational resources (e.g., memory and CPU cycles) required to train and/or analyze the input data.

In general, training an ANN may include establishing a network architecture, or topology, adding layers including activation functions for each layer (e.g., a “leaky” rectified linear unit (ReLU), softmax, hyperbolic tangent, etc.), loss function, and optimizer. In an aspect, the ANN may use different activation functions at each layer, or as between hidden layers and the output layer. A suitable optimizer may include Adam and Nadam optimizers. In an aspect, a different neural network type may be chosen (e.g., a recurrent neural network, a deep learning neural network, etc.). Training data may be divided into training, validation, and testing data. For example, 20% of the training data set may be held back for later validation and/or testing. In that example, 80% of the training data set may be used for training. In that example, the training data set data may be shuffled before being so divided. Dividing the dataset may also be performed in a cross-validation setting, e.g., when the data set is small. Data input to the artificial neural network may be encoded in an N-dimensional tensor, array, matrix, and/or other suitable data structure. In some aspects, training may be performed by successive evaluation (e.g., looping) of the network, using labeled training samples. The process of training the ANN may cause weights, or parameters, of the ANN to be altered. The weights may be initialized to random values. The weights may be adjusted as the network is successively trained, by using one or more gradient descent algorithms, to reduce loss and to cause the values output by the network to converge to expected, or “learned”, values. In an aspect, a regression may be used which has no activation function. Therein, input data may be normalized by mean centering, and a mean squared error loss function may be used, in addition to mean absolute error, to determine the appropriate loss as well as to quantify the accuracy of the outputs.

In some aspects, the ML training module 170 may include computer-executable instructions for performing ML model pre-training, ML model fine-tuning and/or ML model self-supervised training. Model pre-training may be known as transfer learning, and may enable training of a base model that is universal, in the sense that it can be used as a common grammar for all antibody sequences, for example. The term “pretraining” may be used to describe scenarios wherein a second training may occur (i.e., when the model may be “fine-tuned”). Transfer learning refers to the ability of the model to leverage the result (weights) of a first pre-training to better initialize the second training, which may otherwise require a random initialization. The second training, i.e., fine-tuning, may be performed using proprietary affinity data as discussed herein. The technique of combining pre-training and fine-tuning advantageously boosts performance, in that the result of the training on affinity data performs better after pre-training training (e.g., using natural antibody sequences from OAS as described) than when no pre-training is performed. Model fine-tuning may be performed with respect to given antibody-antigen pairs, in some aspects. ML model self-supervised learning may be performed to endow the model with an understanding of the antibody grammar during pre-training.

Generally, an ML model may be trained as described herein using a supervised, semi-supervised or unsupervised machine learning program or algorithm. The machine learning program or algorithm may employ a neural network, which may be a convolutional neural network, a deep learning neural network, transformer, autoencoder and/or a combined learning module or program that learns in two or more features or feature datasets (e.g., structured data, unstructured data, etc.) in a particular areas of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques (e.g., generative algorithms, genetic algorithms, etc.).

In some aspects, an ML algorithm or techniques may be chosen for a particular input based on the problem set size of the input. In some aspects, the artificial intelligence and/or machine learning based algorithms may be based on, or otherwise incorporate aspects of one or more machine learning algorithms included as a library or package executed on server(s) 104. For example, libraries may include the TensorFlow based library, the Pytorch library (e.g., PyTorch Lightning), the Keras libraries, the Jax library, the HuggingFace ecosystem (e.g., transformers, datasets and/or tokenizer libraries therein), and/or the scikit-learn Python library. However, these popular open source libraries are a nicety, and are not required. The present techniques may be implemented using other frameworks/languages.

Machine learning may involve identifying and recognizing patterns in existing data (e.g., binding affinity) in order to facilitate making predictions, classifications, and/or identifications for subsequent data (such as using the trained models to predict variants having high binding affinity). Machine learning model(s), may be created and trained based upon example data (e.g., “training data”) inputs or data (which may be termed “features” and “labels”) in order to make valid and reliable predictions for new inputs. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output.

For example, the ML training module 170 may analyze labeled data at an input layer of a model having a networked layer architecture (e.g., an artificial neural network, a convolutional neural network, a deep neural network, etc.) to generate ML models. The training data may be, for example, sequence variants labeled according to affinity. During training, the labeled data may be propagated through one or more connected deep layers of the ML model to establish weights of one or more nodes, or neurons, of the respective layers. Initially, the weights may be initialized to random values, and one or more suitable activation functions may be chosen for the training process, as will be appreciated by those of ordinary skill in the art. The ML training module 170 may include training a respective output layer of the one or more machine learning models. The output layer may be trained to output a prediction. For example, the ML models trained herein are able to predict binding affinities of unseen sequence variants by analyzing the labeled examples provided during training. In some embodiments, the binding affinity may be expressed as a real number (e.g., in a regression analysis). In some embodiments, the binding affinity may be expressed as a boolean value (e.g., in classification). In some aspects, multiple ANNs may be separately trained and/or operated. For example, an individual model may be fine-tuned (i.e., trained) based on a pre-trained model, using transfer learning, for a plurality of different antibody-antigen pairs.

In unsupervised or semi-supervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model is generated. In the present techniques, semi-supervised learning may be used, inter alia, for natural language processing purposes and to learn a grammar of antibody sequences using an objective, such as a masked language model objective. Supervised learning and/or unsupervised machine learning may also comprise retraining, relearning, or otherwise updating models with new, or different, information, which may include information received, ingested, generated, or otherwise used over time. In various aspects, training the ML models herein may include generating an ensemble model comprising multiple models or sub-models, comprising models trained by the same and/or different AI algorithms, as described herein, and that are configured to operate together.

Once the model training module 170 has initialized the one or more ML models, which may be ANNs or regression networks, for example, the model training module 170 trains the ML models by inputting labeled data into the models (e.g., antibody variants labeled by affinity) The trained ML model may be expected to provide accurate affinity predictions given antibody variant inputs previously unseen by the model (i.e., not used during training).

The model training module 170 may divide the labeled data into a respective training data set and testing data set. The model training module 170 may train the ANN using the labeled data. The model training module 170 may compute accuracy/error metrics (e.g., cross entropy) using the test data and test corresponding sets of labels. The model training module 170 may serialize the trained model and store the trained model in a database (e.g., the data repository 180). Of course, it will be appreciated by those of ordinary skill in the art that the model training module 170 may train and store more than one model. For example, the model training module 170 may train an individual model for each antibody-antigen pair. It should be appreciated that the structure of the network as described may differ, depending on the embodiment.

In some aspects, the computing modules 160 may include a machine learning operation module 172, comprising a set of computer-executable instructions implementing machine learning loading, configuration, initialization and/or operation functionality. The ML operation module 172 may include instructions for storing trained models (e.g., in the electronic data repository 180, as a pickled binary, etc.). Once trained, a trained ML model may be operated in inference mode, whereupon when provided with de novo input that the model has not previously been provided, the model may output one or more predictions, classifications, etc. as described herein. In an unsupervised learning aspect, a loss minimization function may be used, for example, to teach a ML model to generate output that resembles known output (i.e., ground truth exemplars).

Once the model(s) are trained by the model training module 170, the model operation module 172 may load one or more trained models (e.g., from the data repository 180). The model operation module 172 generally applies new data that the trained model has not previously analyzed to the trained model. For example, the model operation module 172 may load a serialized model, deserialize the model, and load the model into the memory 154. The model operation module 172 may load new molecular variant data that was not used to train the trained model. For example, the new molecular data may include antibody sequence data, antigen sequence data, etc. as described herein, encoded as input tensors. The model operation module 172 may apply the one or more input tensor(s) to the trained ML model. The model operation module 172 may receive output (e.g., tensors, feature maps, etc.) from the trained ML model. The output of the ML model may be a prediction of the affinity associated with the input sequences. In this way, the present techniques advantageously provide a means of quantitatively estimating molecular affinity that is far more accurate and data rich than conventional industry practices. An advantage is that measuring these molecular affinities is time consuming and expensive, as it needs to be done in the lab. By using ML, the present techniques need only perform lab measurements to generate the training set, and then can predict unmeasured sequence variants/KD pairs in a relatively inexpensive and fast manner due to in silico performance, rather than requiring continued use of the wet lab.

The model operation module 172 may be accessed by another element of the molecular modeling server 104 (e.g., a web service). The ML operation module 172 may pass its output to the variant identification module 174 further processing/analysis. Alternatively, the variant identification module 174 may receive results stored by the ML operation module 172 in the electronic data repository 180. For example, the variant identification module 174 may evaluate the output of the ML operation module 172 using a set of rules, to identify one or more variants of interest (e.g., those that have highest binding, lowest binding, or other properties as discussed herein). The variant identification module 174 may include further instructions for providing the one or more sequence variants of interest as an input (e.g., via an email, as a visualization such as a chart/graph, as an element of a GUI in a computing device such as the client computing device 102, etc.). In some embodiments, a user may interact with the ML model during training and/or operation using a command line tool, an Application Programming Interface (API), a software development kit (SDK), a Jupyter notebook, etc.

Regarding the modules 160, it will be appreciated by those of ordinary skill in the art that in some aspects, the software instructions comprising the module 160 may be organized differently, and more/fewer modules may be included. For example, one or more of the modules 160 may be omitted or combined. In some aspects, additional modules may be added (e.g., a localization module). In some embodiments, software libraries implementing one or more modules (e.g., Python code) may be combined, such that, for example, the ML training module 170 and ML operation module 172 are a single set of executable instructions used for training and making predictions. In still further examples, the modules 160 may not include the assay module 166 and/or the sequencing module 168. For example, a laboratory computer and/or the assay device 106 may implement those modules, and/or others of the modules 160. In that case, assays and sequencing may be performed in the laboratory to generate training data that is stored in the data repository 180 and accessed by the server 104.

### Exemplary Computer-Implemented Methods

### Model Training

FIG. 2 depicts an exemplary data flow block diagram of a computer-implemented method 200 for training a machine learning model to predict binding of a previously-unseen sequence variant, according to some aspects of the present techniques.

As discussed above, the present techniques may involve a one-step or two-step training procedure. Either of these techniques may include model training using a limited number of data points generated in a wet lab, to construct and train one or models that can predict variants having desired properties (e.g., high affinity). As noted, in some aspects, the training process involves both pre-training using human antibody sequences and fine-tuning using affinity data. At training time, training data (e.g., KD measurements of specific sequence variants) comes from wet lab experiments/assays performed on synthesized variant sequences. The method 200 may perform pre-training and/or fine-tuning. Using both pre-training and fine-tuning has been shown empirically to provide the best performance. Using only fine-tuning provides the second-best performance, and using pre-training only provides the third best performance. Specifically, the method 200 may include receiving screening data including a ranking of the biomolecule sequence variants according to one or more training binding characteristics (block 204). The training binding characteristics may include rankings according to affinity, and may be performed by one or more “wet lab” binding assays of the synthesized biomolecule sequence variants. For example, the assays may involve activity-based screening techniques, SPR techniques and/or others, as discussed herein.

In some aspects, the method 200 may include receiving rescreening data corresponding to the biomolecule sequence variants to amplify/improve the training binding characteristics, and further training the machine learning model using the rescreening data to improve model accuracy (block 206). For example, the rescreening data may increase accuracy in a K_{D} range of interest. It should be noted that the method 200 may include generating a graph of the measured binding characteristics (e.g., the measured binding affinity) to provide a visual demonstration of the relative affinity of each sequence as shown at block 206. Information determined by the assays such as binding kinetics and next-generation sequencing may be received at block 206.

The method 200 may include creating an AI/ML model and training that machine-learned model using the received screening training data to predict one or more desired binding characteristics of an input biomolecule sequence variant (block 208). The training data may include the assayed synthesized biomolecule sequence variants from block 204 and/or block 206, wherein each one has a respective measured binding characteristic (e.g., affinity) representing the ability of each one to bind to a corresponding respective binding partner biomolecule (e.g., an antigen when the biomolecule sequence relates to an antibody, and an antibody when the biomolecule sequence relates to an antigen). As discussed, the training at block 208 may apply transfer learning, wherein a generalized/universal (i.e., pre-trained) model endowed with knowledge of antibody grammar is used in conjunction with a fine-tuning step that involves specific antibody-antigen training based on affinity data. The method 200 may include cross-validating the machine learned model and generating one or more coefficients (e.g., Pearson correlation coefficient) between measured and predicted K_{D}, as discussed below with respect to Example 1 (block 210).

Through the training process of method 200, the method 200 may adjust weights of the machine-learned model, so that the model learns the rules underpinning antibody/antigen interactions. Thus, as discussed in the next section, the trained model may reliably predict biomolecule binding characteristics of input biomolecule sequence variants that the model has not previously seen (block 212). The previously unseen (i.e., novel or simulated) biomolecule sequence variants input to the trained model may be generated via the process of block 202, or may come from another source altogether.

In some aspects, model training (e.g., the model training of the method 200) may enable trained model(s) to predict of one or more antibody variant characteristics, e.g., affinity, using only a limited amount of total possible variant/mutational space to train the model. The variant space may refer to a combinatorial search space of biomolecule (e.g., antibody/antigen, etc.) variants, as discussed herein. For example, in some aspects, 10% of the variant space may be used to train the model. Empirical testing has shown that using a lower percentage of the total possible variant space still provides accurate predictions. Thus, in some aspects, less than 10%, less than 9%, less than 8%, less than 7%, less than 6%, less than 5%, less than 4%, less than 3%, less than 2%, or less than 1% of the total possible variant space may be used to train the model. Percentages of less than 0.5%, less than 0.4%, less than 0.3%, less than 0.2%, or less than 0.1% of the total possible variant space may be used and still achieve a Pearson R value of greater than 0.6.

It will be appreciated by those of ordinary skill in the art that achieving a high correlative value, while using a relatively miniscule proportion of the mutational space for training data (e.g., 0.3%) represents a significant improvement, and advantageously enables the present techniques to be used even in scenarios where limited computational power is available (e.g., via mobile devices, wearable devices, etc.). This also advantageously enables the present techniques to perform training and retraining quickly, thereby reducing latency and improving the performance of applications that train and operate the disclosed models. Finally, the relatively low training data requirements advantageously reduce the amount of training data that must be stored.

### Model Inference

The present techniques may include using the above-described trained ML model(s) to make predictions in silico to obtain silico-predicted KDs. At a high-level, this is akin to “simulating” an experiment in silico because the only way to measure KDs in the lab is to indeed perform assays, sequencing etc. However, in silico, the present techniques advantageously benefit from not needing to simulate or determine every single step in order to get KDs. Rather, the AI learns the relationship between sequence variants and KD and is able to make KD predictions for unseen variants. These predictions are comparable to lab-based experiments, even though the ML model never explicitly simulates an experiment, an assay, or a sequencing run, but rather is able to output predicted KDs given novel (i.e., previously unseen) sequence variants.

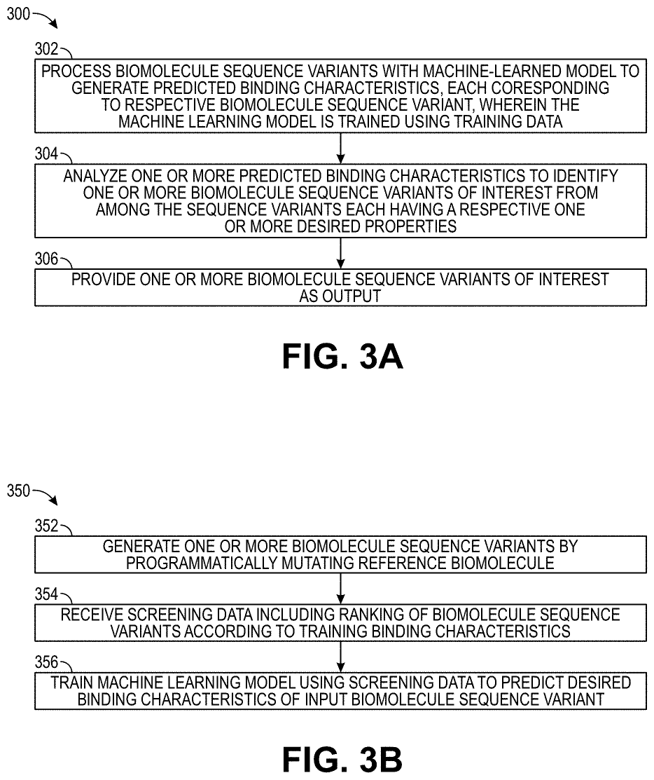

FIG. 3A depicts a computer-implemented method 300 of operating a trained machine-learned model to identify one or more biomolecule sequence variants of interest, according to some embodiments. The method 300 may include receiving one or more simulated/unseen biomolecule sequence variants. The method 300 may generate the simulated biomolecule sequence variants via mutation, as discussed herein, in some aspects. For example, training data may be generated in the wet lab, and one or more ML models trained as discussed above. The method 300 may then include using the trained ML model to make predictions of KDs on arbitrary sequences varied in silico. For example, the method 300 may include changing amino acids in silico and inputting those sequences into the trained ML model, to obtain KD after these changes. In some aspects, this process may be optimized, wherein the task of giving a sequence variant and predicting KD using the model (i.e., inference/prediction) is repeated until a sequence variant with a desired predicted KD is found (i.e., optimization).

Input to the trained model may be generated using a suitable generative technique (e.g., a generative adversarial technique). Both generation and optimization have the same objective: to provide the trained model with a KD and to obtain from it a sequence. Generation attempts to do that directly, by running ML algorithms in a direction opposite to inference. Optimization instead leverages classical inference, and it continues running inference until a sequence with desired KD is found. Optimization is more efficient than an exhaustive search of every possible sequence variant, which would be inefficient and in some cases impossible. An example of a generative technique is plug-and-play, as discussed above, whereas an example of optimization is a genetic algorithm.

After the machine-learned model is trained, the method 300 may include receiving the not previously seen biomolecule sequence variants discussed herein. Of course, the method 300 may just as well receive a previously seen variant (e.g., one used during training). The method 300 may include processing the one or more previously unseen (e.g., simulated) antibody sequence variants with the machine-learned model to generate one or more predicted binding affinities, each corresponding to a respective one of the one or more previously unseen (e.g., simulated) antibody sequence variants (block 302). Specifically, as discussed herein, the machine-learned model may have been pre-trained using a masked language model objective that has an understanding of the grammar governing antibody sequences. Alternatively, or in addition, the machine-learned model may have been fine-tuned using affinity data, such that the model weights have been updated with affinity measurements. The previously unseen antibody sequence variants may be generated using a mutation technique as discussed herein, e.g., by mutagenesis of a reference biomolecule (e.g., an antibody, antigen, etc.).

For example, given an antibody sequence, the machine-learned model may generate a list of predicted binding affinities, wherein each one is associated with a respective antigen. The method 300 may further include analyzing the one or more predicted binding characteristics to identify one or more biomolecule sequence variants of interest from among the simulated sequence variants, each of the one or more biomolecule sequence variants of interest having a respective one or more desired properties. The desired properties may include aspects such as upper/lower bounds of predicted binding affinity, and many others, as discussed herein.

As discussed herein, the variant identification module 174 may include computer-executable instructions for analyzing the output of the machine-learned model to identify one or more biomolecule sequence variants of interest (block 304). For example, as discussed herein, the properties of interest in the one or more variants of interest may include one or more of the following: (i) an increase in at least one predicted binding affinity of the variant of interest; (ii) a decrease in at least one predicted binding affinity of the variant of interest; (iii) an upper bound of at least one predicted binding affinity of the variant of interest; (iv) a lower bound of at least one predicted binding affinity of the variant of interest; (v) an increase in affinity toward a first antigen of a first predicted binding affinity of the variant of interest and a decrease in affinity toward a second antigen of a second predicted binding affinity of the variant of interest; (vi) ability of a cytokine sequence of a variant of interest to increase or decrease binding affinity towards receptors; (vii) suitability of a variant of interest for use as a next-generation antibody scaffold and/or antibody mimetic scaffold; (viii) ability of a variant of interest in an Fc region of an antibody to bind to an Fc receptor; or (ix) a developability of the variant of interest as indicated by tolerability upon administration.

The method 300 may include providing the one or more biomolecule sequence variants of interest as an output (block 306). For example, the method 200 may cause the variants of interest to be stored in an electronic database (e.g., the electronic database of FIG. 1), displayed in a display screen (e.g., the display of the computing device 102 of FIG. 1), or otherwise transmitted to a user (e.g., via email).

In some aspects, training of the ML model may include a fixed antibody and a mutated antigen. In that case, inference may include an antigen search space (i.e., inputting previously unseen antigens). In the fixed antibody-mutated antigen case, the antibody sequence is constant, and the antigen sequence can vary. However, even when varying the antigen sequence, the antigen may be the same, merely mutated at some residues. And the model may output binding affinities. Thus, the search universe in this setting includes the binding affinities for all the antigen variants, in the sequence space of all possible/desired antigen variants. In the fixed antigen-mutated antibody case, the antigen sequence is constant, and the antibody sequence can vary. However, even when varying the antibody sequence, the antibody may be the same, merely mutated at some residues. And the model may output binding affinities. Thus, the search universe in this setting includes the binding affinities for all the antibody variants, in the sequence space of all possible/desired antibody variants.

In other aspects, training of the ML model may include a fixed antigen and a mutated antibody. In that case, inference may include an antibody search space (i.e., inputting previously unseen antibodies). In still further aspects, training of the ML model may include respective mutation of an antigen and an antibody. In that case, inference may include an antigen and antibody search space (i.e., inputting previously unseen antigens and antibodies).

FIG. 3B depicts a computer-implemented method 350 of training a machine learning model to identify biomolecule sequence variants of interest, according to some aspects. The method may be performed by a computer, such as the molecular modeling server 102 of FIG. 1, in some aspects.

The method 350 may include generating one or more biomolecule sequence variants by programmatically mutating a reference biomolecule (block 352). Herein, the term “programmatic” or “programmatically” means according to a method or system (e.g., via a computer-implemented method, via a computer program, via a computing system, etc.). The mutation may be performed according to the principles discussed herein. For example, the method 350 may include generating an antibody library that evenly samples a sequence space around a starting point antibody molecule. The method 350 may perform the mutation in silico, i.e., by a processor executing computer-executable instructions (e.g., by the CPU 150 of FIG. 1 using the variant module 164 of the molecular modeling server 102).

The method 350 may include receiving screening data including a ranking of the biomolecule sequence variants according to one or more training binding characteristics (block 354). As discussed, the screening of the present techniques may include wet lab screening (e.g., using qaACE) and/or in silico screening. The method 350 may include training the machine learning model using the screening data to predict one or more desired binding characteristics of an input biomolecule sequence variant (block 356).

In some aspects, the method 350 may include receiving rescreening data corresponding to the biomolecule sequence variants to amplify the one or more training binding characteristics; and further training the machine learning model using the rescreening data to improve accuracy of the machine learning model. In some aspects, the training binding characteristics of the method 350 may include binding affinity (KD). In some aspects, the screening data of the method 350 may be received from one or both of (i) a human experimenter, and (ii) an assay device. In some aspects, the one or more biomolecule sequence variants of the method 350 include an antibody or an antigen.

### Exemplary Data Flow Diagram

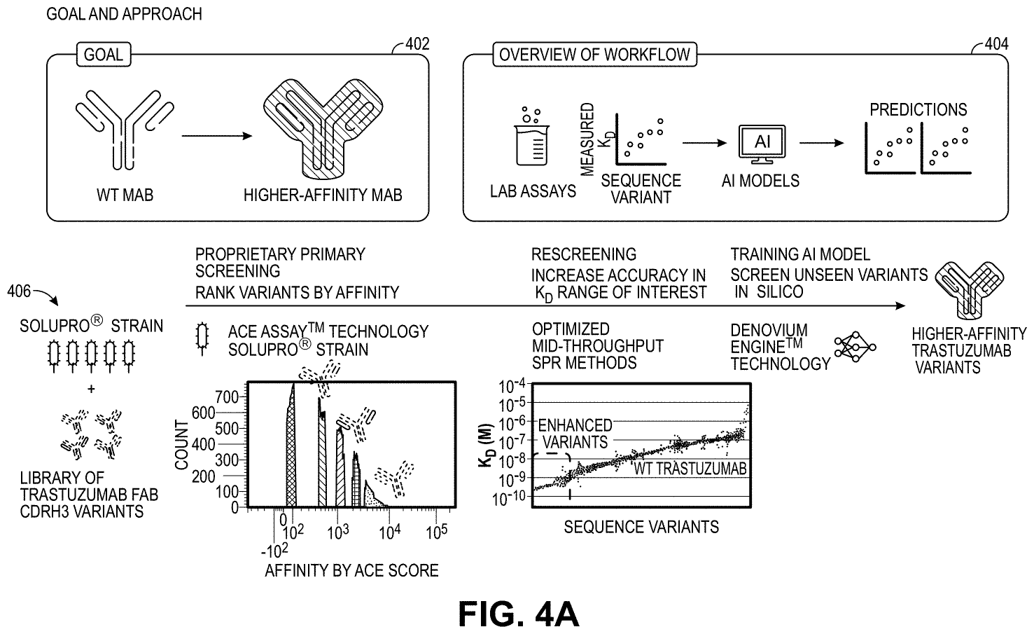

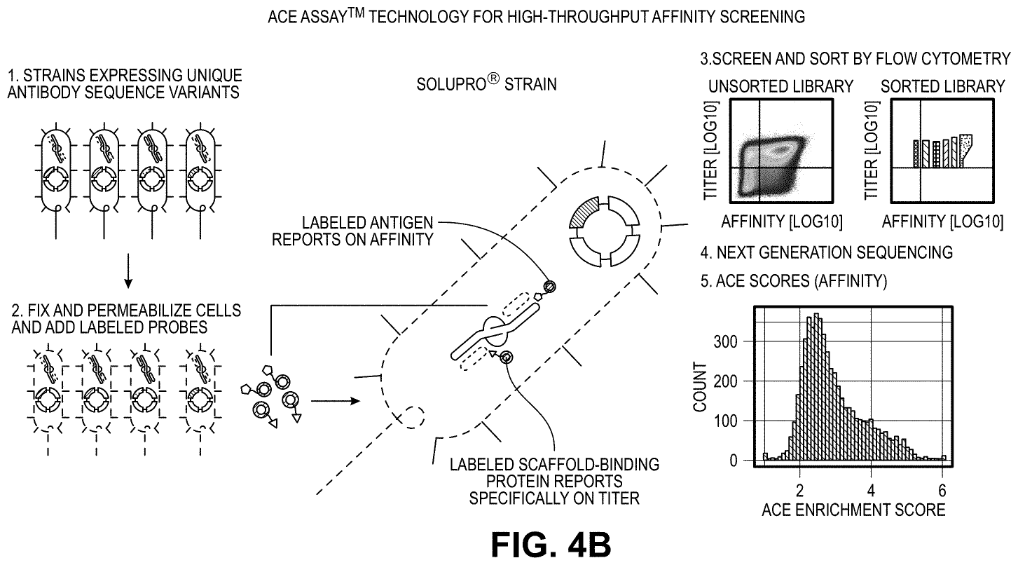

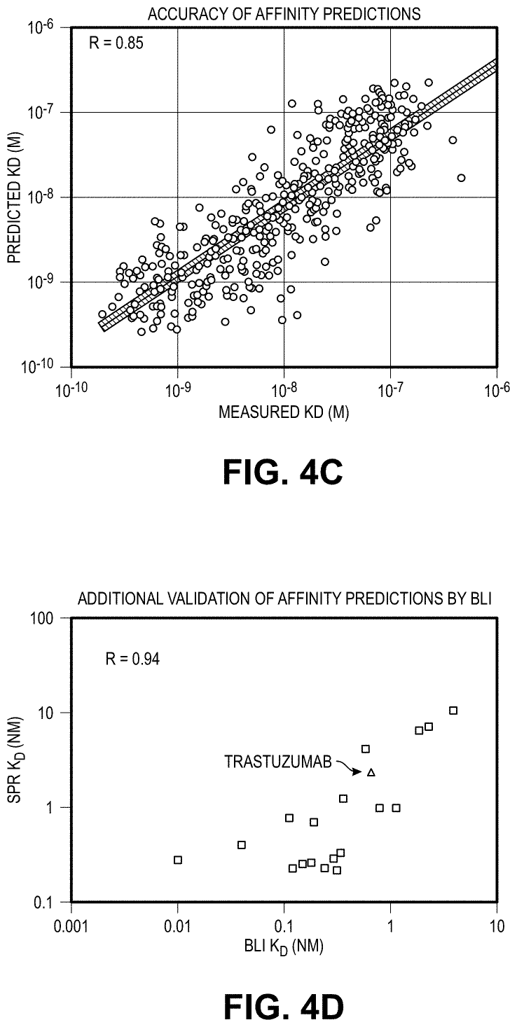

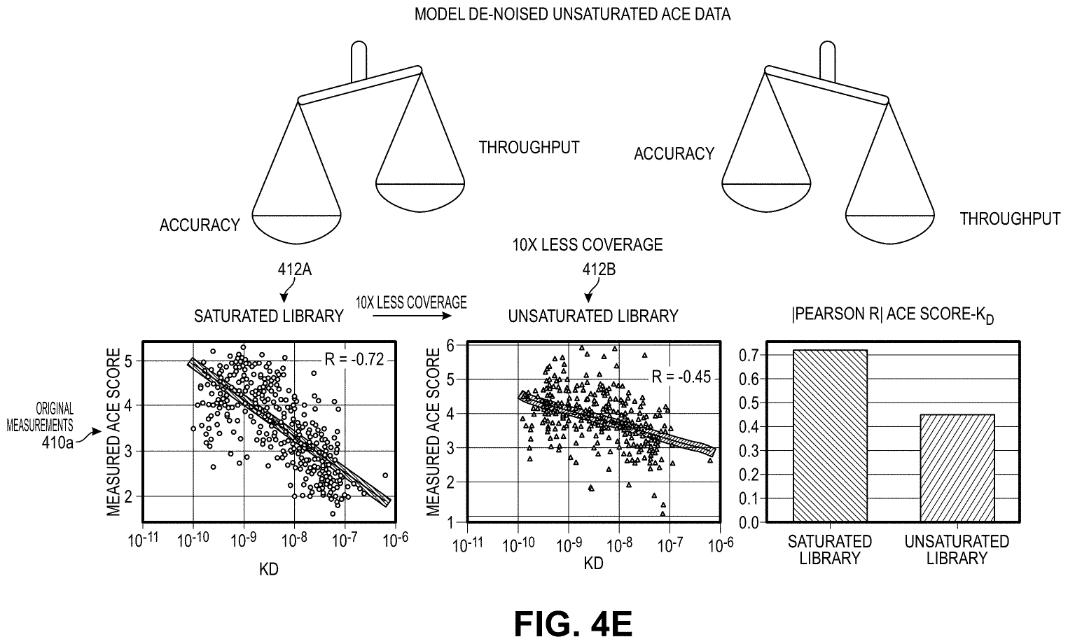

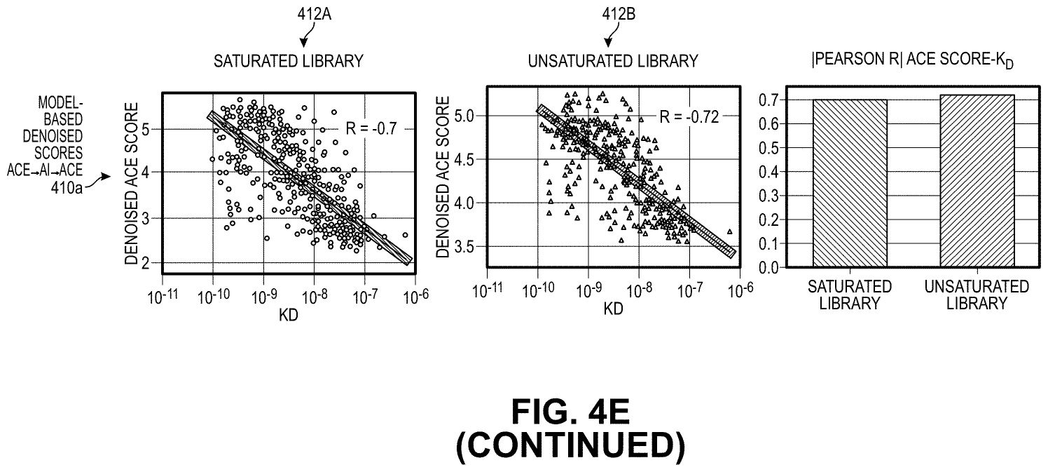

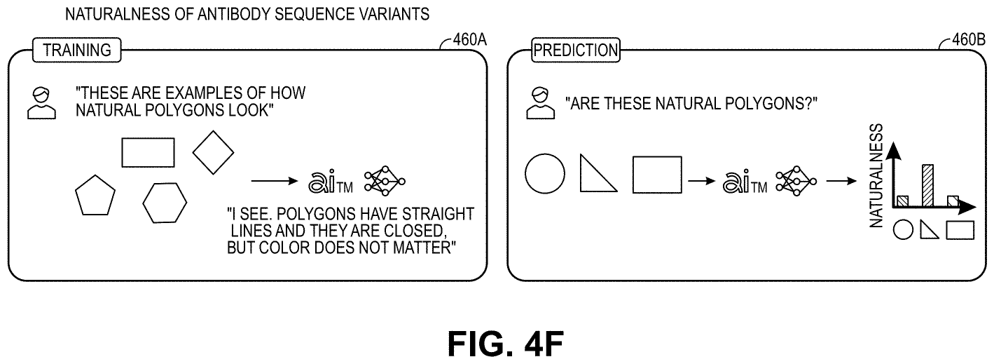

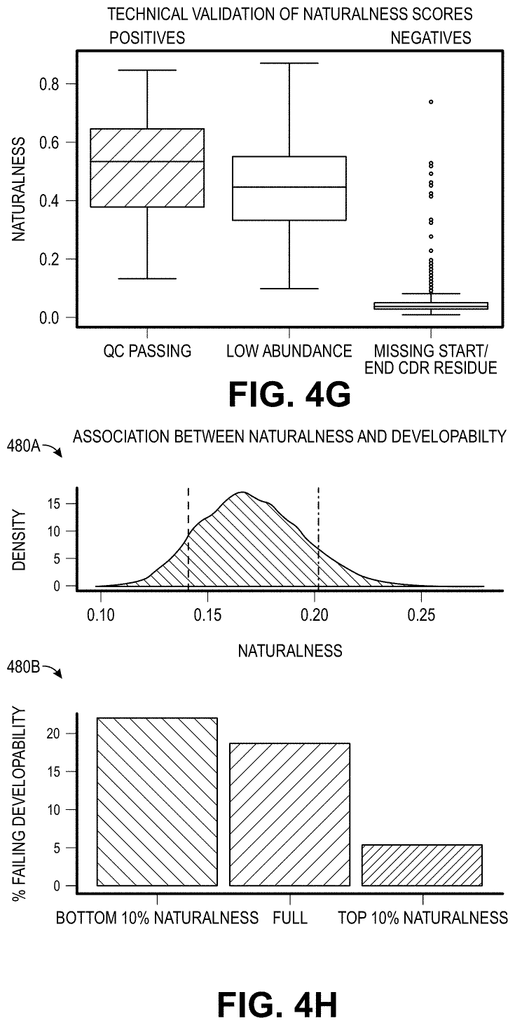

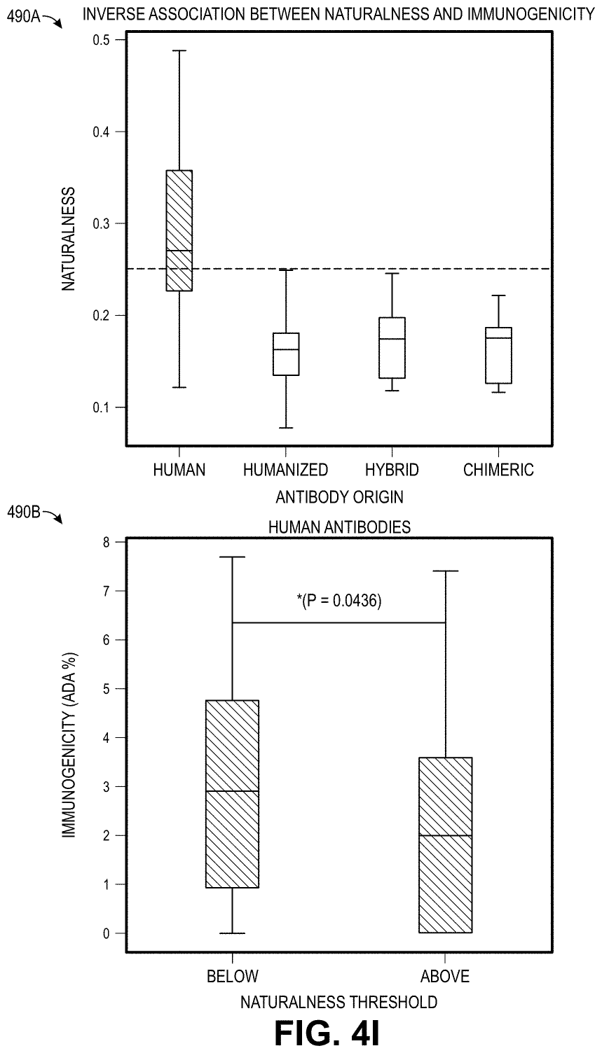

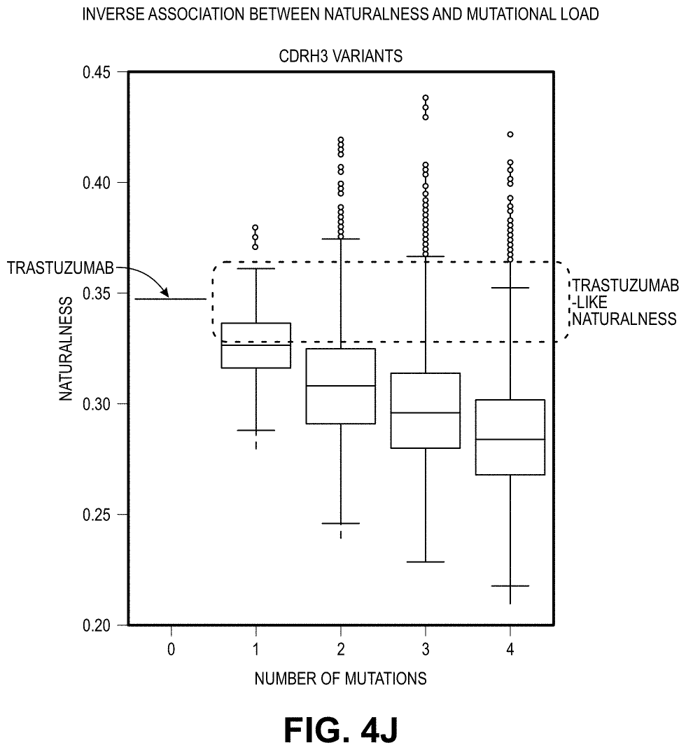

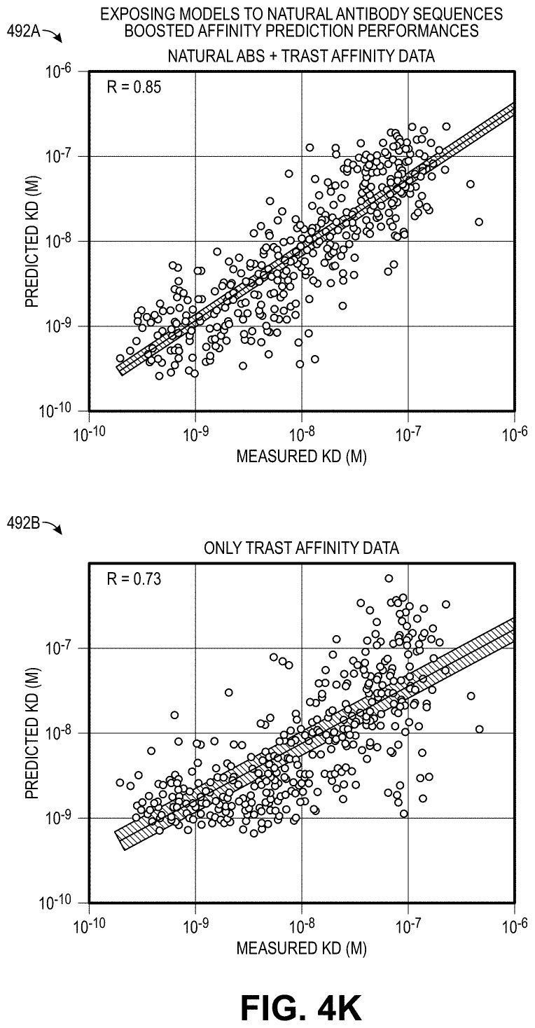

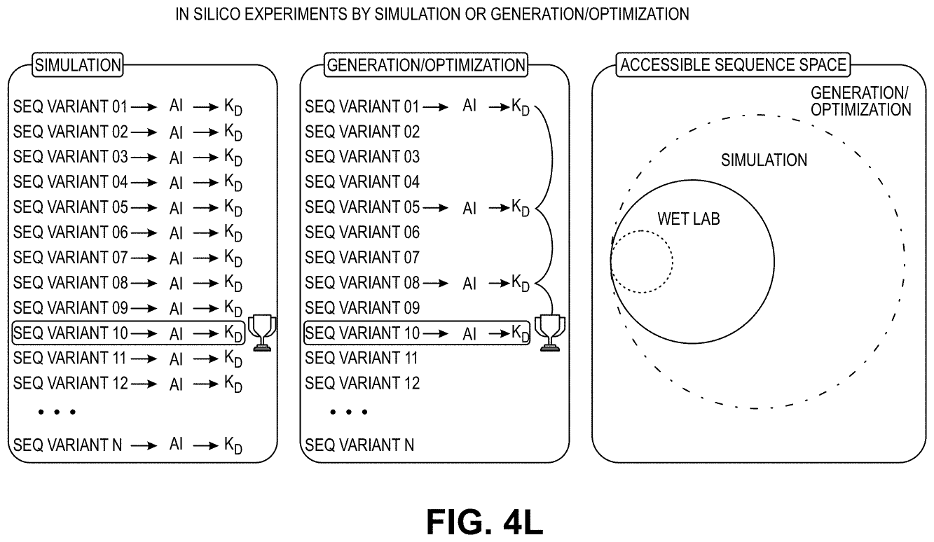

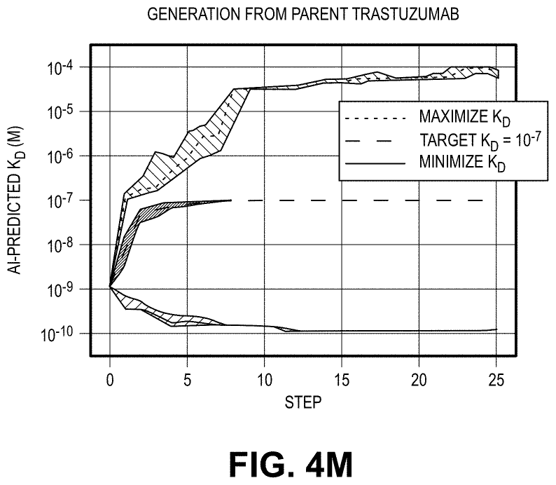

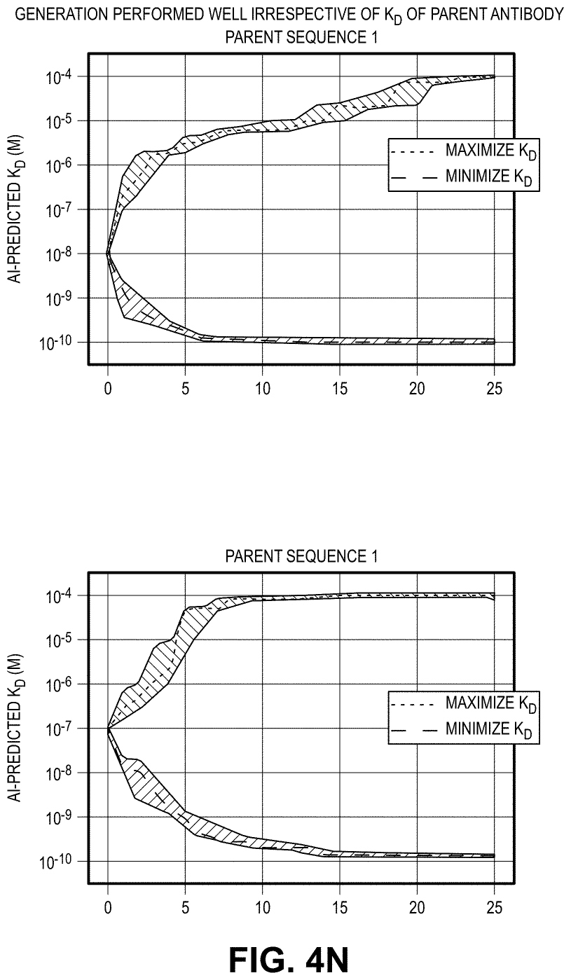

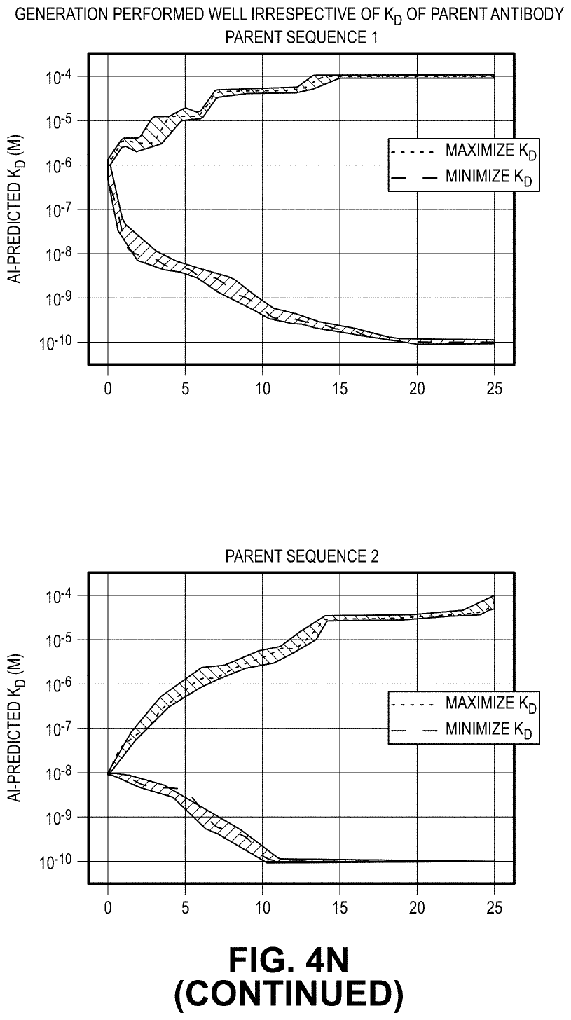

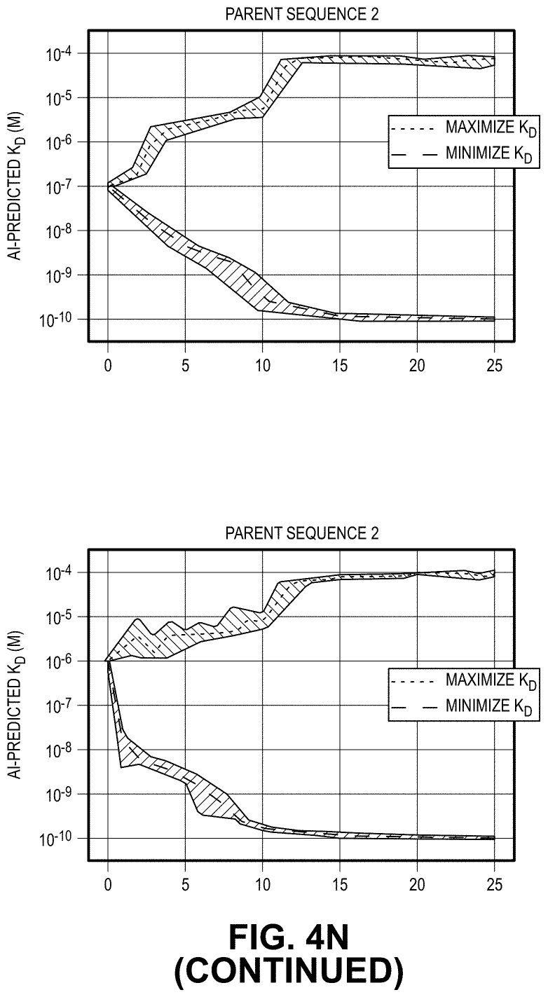

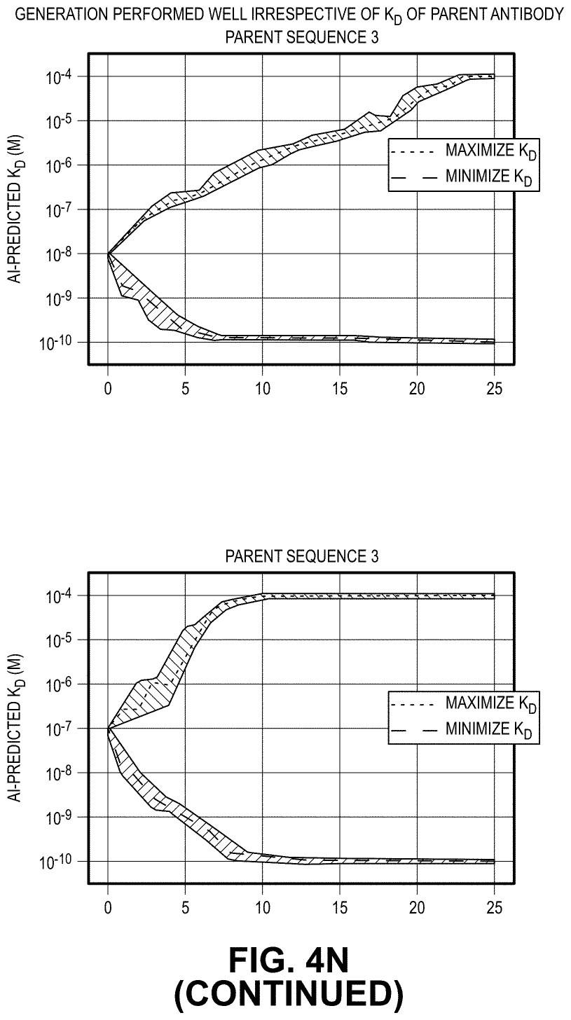

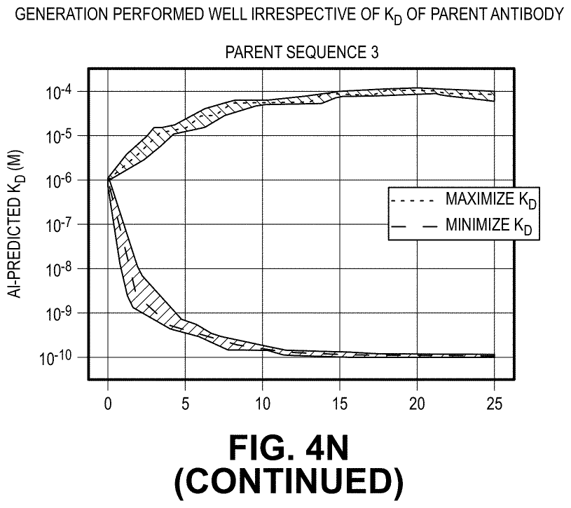

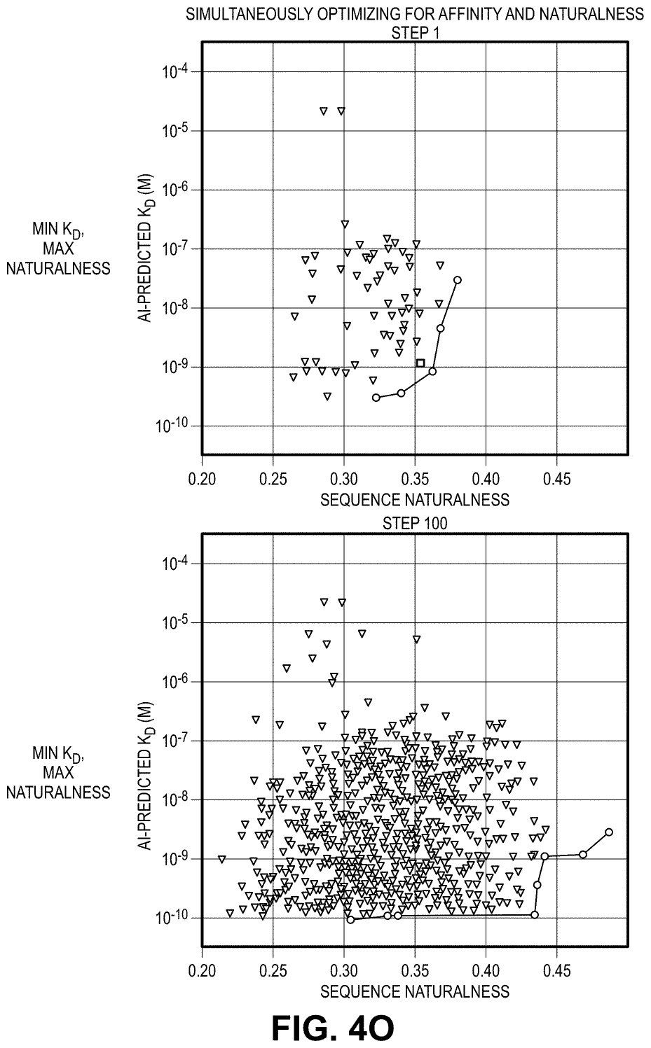

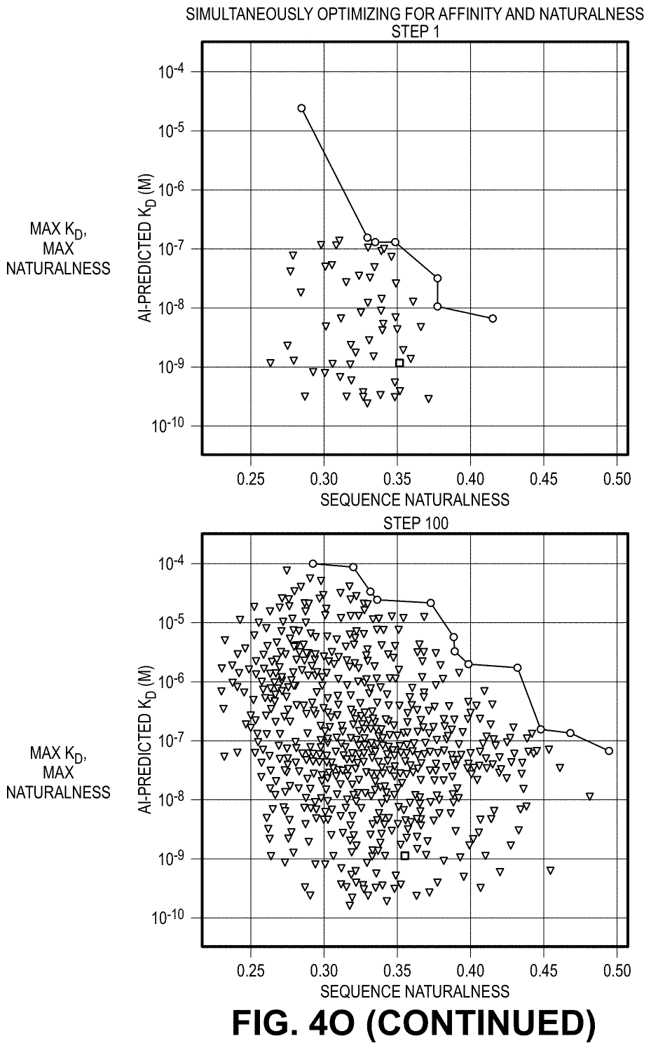

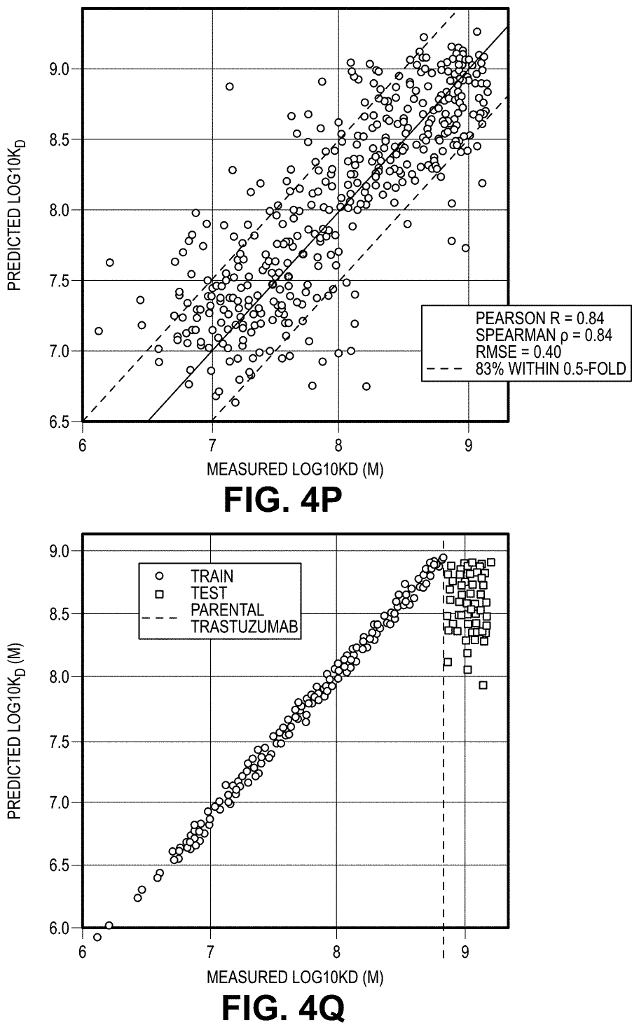

FIG. 4A depicts an example data flow diagram depicting training and predicting of biomolecule sequence variants of interest, according to some aspects. In some aspects, the data flow diagram of FIG. 4A may correspond to the method 200 of FIG. 2.

Generally, FIG. 4A depicts a goal 402 of identifying a higher-affinity monoclonal antibody, relative to a wild-type monoclonal antibody. FIG. 4A also depicts a workflow overview 404 that shows lab assay measurements being input into AI models (e.g., the one or more models trained as discussed in FIG. 2) to generate one or more predictions. FIG. 4A includes an additional level of detail showing that assayed biomolecules may be, for example a SoluPro® strain and/or a library of one or more sequence variants (e.g., a trastuzumab Fab CDRH3 variants). A method may include a proprietary primary screening that ranks the input variants by affinity (e.g., using an ACE Assay™), rescreens the input to increase accuracy in a given KD range of interest and a training of an AI model to screen unseen variants in silico.

FIG. 4B depicts an example block flow diagram for performing the assay of FIG. 4A, according to some aspects. Strains expressing unique antibody sequence variants may be added to fix and permeabilized cells, and probes may be added (blocks 1 and 2). Such a SoluPro® strain may include labeled antigen reports on affinity and labeled scaffold-binding protein reports specifically on titer. The strains may be screened and sorted by flow cytometry (block 3). Next-generation sequencing may be performed, and ACE affinity scores may be generated (blocks 4 and 5).

FIG. 4C depicts an example affinity prediction chart, according to some aspects. In FIG. 4C, observed correlation between model-predicted and SPR-measured K_{D}s of trastuzumab CDRH3 sequence variants binding to Her2 are shown, spanning nearly four orders of magnitude, with an R Pearson correlation coefficient of 0.85.

FIG. 4D depicts an example affinity prediction validation chart, according to some aspects. FIG. 4D shows 20 sequence variants (trastuzumab and 15 antibodies predicted to bind to Her2 more strongly than trastuzumab and four antibodies predicted to be slightly weaker binders relative to trastuzumab) already validated by mid-throughput SPR, undergoing additional secondary validation by low-throughput BLI. The correlation coefficient in this case is R=0.94.

### Exemplary Denoising

There is rich literature for using deep learning for denoising in the field of single-cell RNA-seq. However, denoising has not been used in the field of antibody engineering.

A model used to perform denoising may be the same from an architecture/algorithm standpoint to the one used in Example 1, except that instead of training using Carterra SPR sequences, the model may be trained using ACE data. Pre-training with natural antibody sequences is the same. The model input may be ACE-derived affinity scores having different degrees of accuracy. The higher the coverage (number of cells over number of unique variants), the more accurate the scores. Correlations of ˜negative 0.7 between ACE scores and Carterra K_{D}s have been observed, but when coverage goes down, such correlations decrease. Thus, feeding ACE data to the model as training data comprising sequence variants and respective ACE scores is performed. The model may then predict the ACE scores of the same sequence variants used for training. Model predictions will not be identical to the training set, because the model tries to generalize. We refer to these predictions as “denoised” scores, and empirical testing has shown that such scores correlated better with SPR K_{D}s than original (hard measured) ACE scores. Thus, denoising is used unconventionally in the present techniques to maintain or improve the accuracy of predictions (e.g., affinity) while enabling throughput to be increased. Measurements taken in an assay (e.g., an ACE assay) may be correlated with affinity, as shown in FIG. 4E.

FIG. 4E depicts exemplary denoised data charts, according to some aspects. FIG. 4E depicts original measurements (row 410a) and model-based denoised scores (row 410b), in addition to saturated libraries (col. 412a) and unsaturated libraries (col. 412b). ACE libraries are saturated when sorting and sequencing capacities greatly exceed library size (i.e., have high coverage). Saturated libraries generally yield the highest accuracy in terms of correlation of ACE scores with SPR-derived K_{D}. When coverage is lower, libraries become unsaturated and accuracy degrades. The present techniques may include model training using sequence variants and ACE scores from unsaturated libraries, and models that predict ACE scores for the same sequence variants used in training. Such model-derived ACE scores of training sequence variants (i.e., denoised ACE scores) correlated better with SPR-derived K_{D}s than hard measurements. By contrast, no model-provided accuracy boost was observed when libraries were fully saturated.

A compromise always exists between assay throughput and assay accuracy. Adding more sequence variants, to identify more interesting potential sequences comes at the cost of decreases in accuracy. This is because generally a smaller library will have more redundant measurements, which means that the same sequences can be resampled multiple times, eliminating error.

The model for performing denoising may be trained the same way as other models described herein, e.g. using ACE training data. However, the predictions may no longer be unseen sequence variants (i.e., sequences that were absent from the training data set). Rather, the predicted sequence variants are the same sequence variants used in the training data set. Rather than predicting unseen variants, in the denoising context, predictions are of the training data.

Denoising provides significant benefits, in the depicted example of FIG. 4E restoring the correlation coefficient in the chart of the unsaturated library at row 410b, column 412b to its corresponding saturated chart at row 410b, column 412b. Essentially, this result means that accuracy can be preserved, even while enabling throughput to be greatly increased. The model denoises inaccurate measurements to make them more accurate, in the sense that they correlate better with ground truth (i.e., SPR) measurements. Another way to consider the improvement of denoising technique, is as enabling collection and use of more inaccurate data that might otherwise not be of use.

### Exemplary Machine-Learned Naturalness

The present techniques may include feeding natural antibody sequences to teach naturalness to one or more machine learning model. The source of antibody sequences may be those used for pretraining (e.g., OAS database) optionally supplemented with proprietary sequences, such as those from Totient.

FIG. 4F depicts an exemplary conceptual diagram depicting naturalness training and prediction, according to some aspects. In general, “naturalness” is a measure of whether an input resembles training data. This resemblance may be simply expressed in terms of shapes, as in FIG. 4F. That is, during a training phase 460a, a model may be trained (e.g., by the ML model training module 170 of FIG. 1) using examples of polygons. The model may learn that polygons have certain features, such as straight lines, closed geometry. The model may learn that other features are not determinative of whether a given input is a polygon (e.g., color, line thickness, rotation, etc.). The model may be trained to generate a score for each input representing a probability that the input is a polygon. During a prediction phase 460b, the trained model may be used by inputting a collection of individual shapes (as depicted, a circle, a triangle, a line, etc.), to obtain respective naturalness predictions. As shown, the model infers that the triangle is the most polygonal of those inputs.

The sequence variants tested in the training set may be generated by combinatorially listing all possible sequences upon defining mutational load and positions to be mutated, having all of them or a subsample (for example, randomly picked) synthesized and then tested in the lab. Affinity measurements (ACE scores and/or SPR KDs) can then be fed to the model.

Of course, “naturalness” may be expressed differently, depending upon what inputs are being used to train a model, and the characteristics that determine such similarity may be less intuitive. For example, a similar process can be applied to biomolecules of interest (e.g., to antibody sequences). Such models may be trained using training data comprising many (e.g., millions or more) examples of antibody sequences. These sequences may be from one or more species. Once trained, such models can score the naturalness of previously unseen sequences, such as variants of a parent antibody. In particular, such trained models may be used to adjudge the naturalness of new antibodies generated purely in silico. This is helpful for many reasons, among these that a company seeking to design therapeutics may find it highly beneficial to eliminate antibodies that cannot be used in humans.

FIG. 4G depicts exemplary naturalness score validation charts, according to some aspects. As discussed, models may be trained to determine naturalness scores of biomolecules such as antibodies. The scores derived from such models may behave as expected in technical validations. For example, as shown in FIG. 4G, different sequences may be visually displayed according to their respective naturalness. In this example, a number of antibodies from OAS database that pass a range of quality control (QC) filters (“positives”) are shown in a distribution. QC failures (e.g., those whose annotation indicates a missing start and/or end CDR residue(s) (“negatives”) are shown in a near-zero naturalness score distribution. Scores for low-abundance sequences were also computed using the modeling techniques described herein, which are expected to encompass rare but genuine antibodies as well as sequencing errors. Sequencing errors should be randomly distributed, thereby seldom affecting conserved antibody residues, which would penalize naturalness the most. Consistently, the naturalness distribution of low-abundance antibodies shows only a minor downward shift compared to QC-passing antibodies.

For sequences failing QC filters, “low abundance” means an abundance of 1 count across the dataset. “Missing start/end CDR residues” means that the antibody sequence annotation (typically done using a tool called ANARCI) misses the start or the end residue of one CDR. The OAS filters are described as follows. From the whole unpaired dataset, studies are excluded when they have overlapping samples (e.g., ‘Bonsignori et al., 2016’, ‘Halliley et al., 2015’, ‘Thomqvist et al., 2018’). Diseases may also be excluded: ‘Light Chain Amyloidosis’, ‘CLL’. B-types may be excluded: ‘Immature-B-Cells’, ‘Pre-B-Cells’. As for the sequences themselves, those may be excluded that: have stop codons; are marked as non-productive or out-of-frame; have unconserved cysteine sites; have j_identity<50; have no AAs in FWR2 or FWR3; have more than 37 AAs in CDR3; are missing first two or last two positions on any CDR, according to IMGT. After that, for model training, sequences may be excluded that have a cumulative redundancy of 1.

FIG. 4H depicts exemplary naturalness/developability correlation charts, according to some aspects. To analyze the relationship between antibody naturalness and developability, the present techniques may be used to score the naturalness of a number (e.g., 5000) of hits by enrichment of the final panning round of a published phage display library (Liu et al, Bioinformatics 36:2126 (2020)) (chart 480a). The same sequences may also be analyzed using the Therapeutic Antibody Profiler (Raybould et al, PNAS 116:4025 (2019)), recording the percentage that received at least one amber or red developability flag or could not be modeled at all (developability failures, chart 480b). The top and bottom 10% sequences by naturalness may then be compared, and a depletion and enrichment of developability failures observed, respectively, indicating an association between naturalness and developability. It will be appreciated by those of ordinary skill in the art that other features may be compared and, potentially correlated (e.g., aggregation, viscosity, thermostability, oxidation, etc.).

FIG. 4I depicts exemplary naturalness and immunogenicity correlation charts, according to some aspects. In some aspects, the relationship between antibody naturalness and immunogenicity may be explored using the present modeling techniques. For example, a model may score the naturalness of therapeutic antibodies administered to humans (phase I, II, III or clinically approved) binned by origin (Marks et al, Bioinformatics 37:4041 (2021)) using a CDR-only model. As shown, fully human antibodies may yield higher naturalness scores than other classes of antibodies (chart 490a). A threshold of naturalness above which no humanized, chimeric or hybrid (humanized+chimeric) antibody sequences could be found may be defined. The reported fractions of patients that developed anti-drug antibody (ADA) responses (Marks et al, Bioinformatics 37:4041 (2021)) to fully human antibodies, split according to the previously defined naturalness threshold may be analyzed, to observe lower immunogenicity to human antibodies when naturalness was above threshold (chart 490b), suggesting that naturalness is inversely associated with immunogenicity. This is an important result, because despite potential confounding factors, the ability to identify naturalness above a threshold may assist in identifying drug candidates that are less likely to be rejected by the human immune system.

FIG. 4J depicts exemplary naturalness and mutational load correlation charts, according to some aspects. In some aspects, the present techniques may include scoring the naturalness of trastuzumab variants as a function of CDRH3 mutational load. As mutational load increases, median naturalness may decrease. This observation suggests that a larger and larger fraction of random samples of the combinatorial sequence space might fail downstream development as more mutations are introduced, given the previously discussed association between developability and immunogenicity. As a consequence, model-guided optimization of naturalness might be a superior strategy, opposed to screening random samples of antibody variants.

FIG. 4K depicts exemplary charts of affinity prediction improvement when enriching with naturalness data, according to some aspects. Feeding models with examples of antibody sequences not only enabled the computation of naturalness scores; it also boosted the accuracy of affinity predictions via transfer learning. This enables observed correlation between predicted and SPR-measured K_{D}s using a model trained with both unlabeled natural antibody sequences and affinity measurements of trastuzumab variants (chart 492a, also depicted in FIG. 4C) or only the latter (chart 492b).

FIG. 4L depicts exemplary conceptual diagrams of in silico sequence variant generation and optimization, according to some aspects. Since models trained with affinity measurements of trastuzumab variants predicted the affinities of unseen variants (sequences not present in the training set, FIG. 4C), screening experiments may be simulated in silico. However, naïve simulation would involve exhaustively predicting the K_{D}s of every possible sequence variant given a defined mutational load, which could become inefficient with large sequence spaces. As an alternative, K_{D}s might be optimized at the cost of just a fraction of computations using generative techniques.

To find a sequence variant that is very good according to two criteria (e.g., naturalness and affinity) a genetic algorithm may be used in conjunction with the deep learning models described herein, to generatively find the best sequence variant, without the need to generate sequences combinatorially. It should be appreciated that the present techniques may be used to maximize K_{D}, minimize K_{D} or to find a particular K_{D} value.

FIG. 4M depicts an exemplary chart of affinity prediction from trastuzumab, according to some aspects. FIG. 4M depicts progressive optimization of K_{D}s (minimization, maximization or tuning to a specific value) driven by a model trained with affinity measurements of trastuzumab CDRH3 sequence variants, from a starting point of a trastuzumab sequence.

FIG. 4N depicts exemplary affinity prediction charts from different parent antibodies, according to some aspects. FIG. 4N depicts progressive optimization of K_{D}s (minimization or maximization) driven by a model trained with affinity measurements of trastuzumab CDRH3 sequence variants, from multiple starting points of trastuzumab variants whose respective K_{D}s span 3 orders of magnitude.

FIG. 4O depicts exemplary visualizations of optimizing for affinity and naturalness, according to some aspects. FIG. 4O depicts co-optimization of K_{D}s (minimization, left, or maximization, right) and naturalness (maximization) of trastuzumab CDRH3 sequence variants. The starting point (black dot) was the trastuzumab sequence.

As shown above, the present techniques include deep learning models that are predictive of binding affinity. In some aspects, the present techniques include training a number (e.g., three) of affinity prediction models based on HT and SPR data: one using only the SPR, one using only HT data, and one using both in a multi-task setting. In such aspects, model performance may be evaluated, as directed to addressing two questions. First, self-prediction, or how well do the models recapitulate the data that was used to supervise its training (cross-validation)? And second, K_{D}-prediction, or how well do the models predict the actual KD (as measured by SPR)? Empirical has shown the models discussed herein to be strongly predictive for both self-prediction and true K_{D}-prediction, over a significant number (e.g., four) orders of magnitude, as depicted in the following table and in FIG. 4P, showing performance statistics for Trastuzumab affinity predictions (e.g., wherein the data points that have SPR measurements also have HT measurements, resulting in a total combined training set size of 5689):

| TRAIN | TEST | DATASET | | | % w/t |
|---|---|---|---|---|---|
| DATA | DATA | SIZE | PEARSON | RMSE | 0.5-FOLD |
| HT | HT | 5689 | 0.81 | 0.52 | 74% |
| HT | SPR | 5689 | 0.71 | — | — |
| SPR | SPR | 500 | 0.80 | 0.45 | 78% |
| HT + SPR | HT | 5689 (+500)* | 0.80 | 0.54 | 71% |
| HT + SPR | SPR | 5689 (+500)* | 0.84 | 0.40 | 80% |

The HT model for K_{D}-prediction may be similar to the predictive power of the laboratory measured data. In some aspects, out of the three models, the combined, multi-task model showed the best K_{D}-prediction performance.

As discussed with respect to conventional techniques, the development of a candidate biomolecule (e.g., antibody) into a therapeutic drug is a complex process with a high degree of risk, especially as relates to modeling these risks. The present techniques enable using examples of antibodies in natural systems to model productive patterns and mitigate these issues. For example, the present techniques may include employing the above-described pre-training techniques (e.g., based on natural OAS sequences) to evaluate new sequences for “naturalness.” This naturalness measure may then be used as an additional measure for in silico optimization.

In some aspects, to determine the utility of naturalness scores, the present techniques may evaluate independent measures of therapeutic outcomes (e.g., the Therapeutic Antibody Profiler (TAP) (Raybould et al., 2019), which reports on five criteria for antibody developability). In some aspects, the present techniques demonstrate a strong association between naturalness and TAP on sequences from a phage display library (Liu, G., Zeng, H., Mueller, J., Carter, B., Wang, Z., Schilz, J., Horny, G., Birnbaum, M. E., Ewert, S., and Gifford, D. K. Antibody complementarity determining region design using high-capacity machine learning. Bioinformatics, 36(7):2126-2133, November 2019a. doi: 10.1093/bioinformatics/btz895. URL https://doi. org/10.1093/bioinformatics/btz895.), with less than half as many sequences failing one or more developability criteria in the high naturalness domain (10th percentile) versus the low naturalness domain (90th percentile) (7.6% vs. 17.8%). In another example, a second evaluation may using sequences from a study on production titers in the HEK-293 cell line for clinical-stage antibodies (Jain, T., Sun, T., Durand, S., Hall, A., Houston, N. R., Nett, J. H., Sharkey, B., Bobrowicz, B., Caffry, I., Yu, Y., Cao, Y., Lynaugh, H., Brown, M., Baruah, H., Gray, L. T., Krauland, E. M., Xu, Y., Väsquez, M., and Wittrup, K. D. Biophysical properties of the clinical-stage antibody landscape. Proceedings of the National Academy of Sciences, 114(5):944-949, January 2017. doi: 10.1073/pnas.1616408114. URL https://doi.org/10.1073/pnas.1616408114.).

In that case, empirical evidence has shown that sequences in the high naturalness domain showed increased production titers by more than a third as compared to the low naturalness domain (180 mg/L vs. 130 mg/L). In yet another example, naturalness may be evaluated against reported immunogenicity measures for 217 therapeutic antibodies compiled by Marks et al. (Marks, C., Hummer, A. M., Chin, M., and Deane, C. M. Humanization of antibodies using a machine learning approach on large-scale repertoire data. Bioinformatics, 37(22):4041-4047, June 2021. doi: 10.1093/bioinformatics/btab434. URL https://doi.org/10.1093/bioinformatics/btab434). Empirical evidence has shown that the top quartile of natural sequences were half as likely to be immunogenic as compared to the bottom quartile (median ADA immunogenicity of 2.6% vs. 5.4%).

Furthermore, the present techniques have demonstrated improved antibodies both by screening, and by model-guided design. For example, an initial screen consisting of high-throughput enrichment followed by SPR has identified several sequences with binding affinity greater than the wild-type (n=87). This has enabled the present SPR model to predict novel sequences in this range. The present techniques may enable identification of strong binders, even for K_{D} values that surpassed anything seen in the laboratory assays used to train them.

The present techniques may include result testing, by setting aside all data with measured affinity values higher than wild-type Trastuzumab into a hold-out set, and then training a model using the remaining data, and predicting the affinity of both the train and hold-out sets. In some aspects, some models may not be able to make accurate K_{D} predictions of the held-out data points (as these were out of the distribution the model had seen). However, some models may place these points near the top of the prediction range, as shown in FIG. 4Q, enabling virtual screening of the sequence space and expansion of the prediction range with additional laboratory experiments. In support of the practical value of our SPR model, a number of sequences predicted to have greater than the wild-type binding affinity were empirically tested. SPR screening confirmed that 76% of these sequences were greater than wild-type and 94% of the predictions were within 0.5-fold of their measured values.

### Exemplary Results

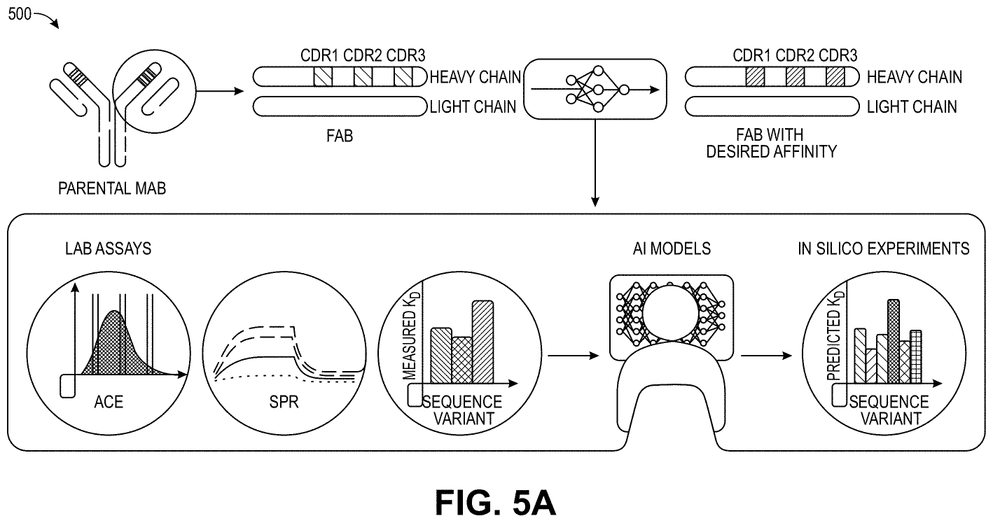

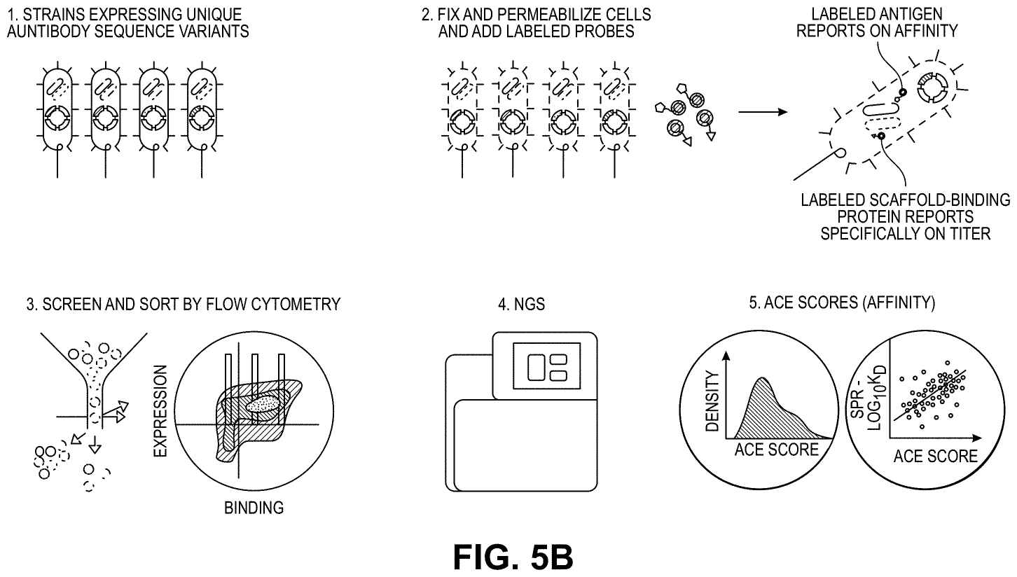

FIG. 5A depicts an exemplary AI-augmented antibody optimization diagram 500, according to some aspects. FIG. 5A shows that deep learning models fed with qaACE and/or SPR measurements can quantitatively predict affinities of novel sequence variants, thereby enabling the in silico design of antibodies with desired binding properties. Deep language models can predict binding affinity of sequence variants. The present techniques hypothesized that Artificial Intelligence (AI) could learn the mapping between variants of a biological sequence (such as an antibody) and quantitative readouts (such as binding affinity) from experimental data. With this capability, AI models could be used to simulate experiments in silico for novel sequence variants, thereby accessing a larger sequence space to identify more and better variants with desired properties in a fraction of the time and cost, as depicted in FIG. 5A.

Training of deep learning models generally requires large, high-quality datasets. To generate high-throughput measurements of antibody binding affinities, the present techniques developed and incorporated the Activity-specific Cell-Enrichment (ACE) assay (or qaACE assay), as shown for example below in FIG. 5B, a Fluorescence-Activated Cell Sorting (FACS) and Next-Generation Sequencing (NGS) method of binning antibody variants based on affinity. In some aspects, the assay is an improved version of prior work [s20]. qaACE leverages intracellular soluble overexpression of folded antibodies in the SoluPro™ E. coli B Strain. Cells expressing antibody variants are fixed, permeabilized and stained with fluorescently-labeled antigen and scaffold probes that enable simultaneous discrimination of cells based on affinity and titer of variants. Variant libraries are sorted and binned based on these signals. Then, the collected DNA sequences are amplified via PCR and sequenced.

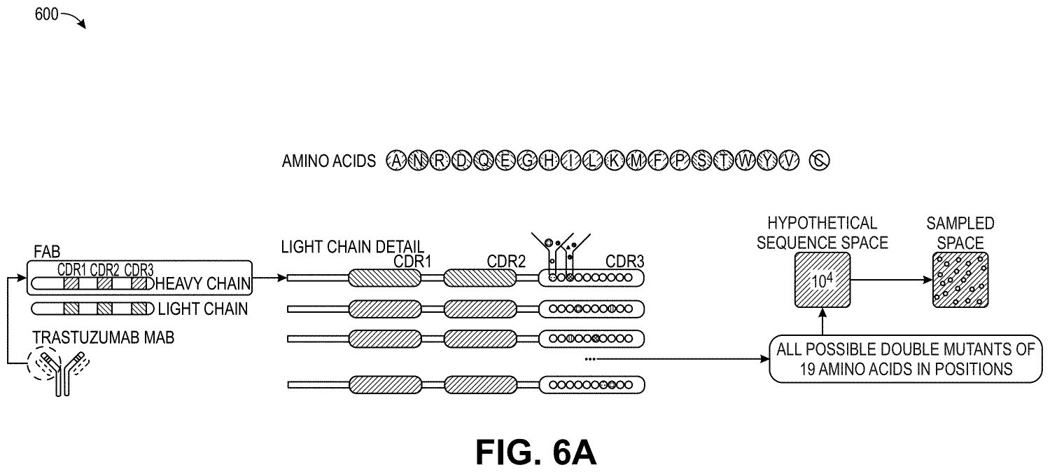

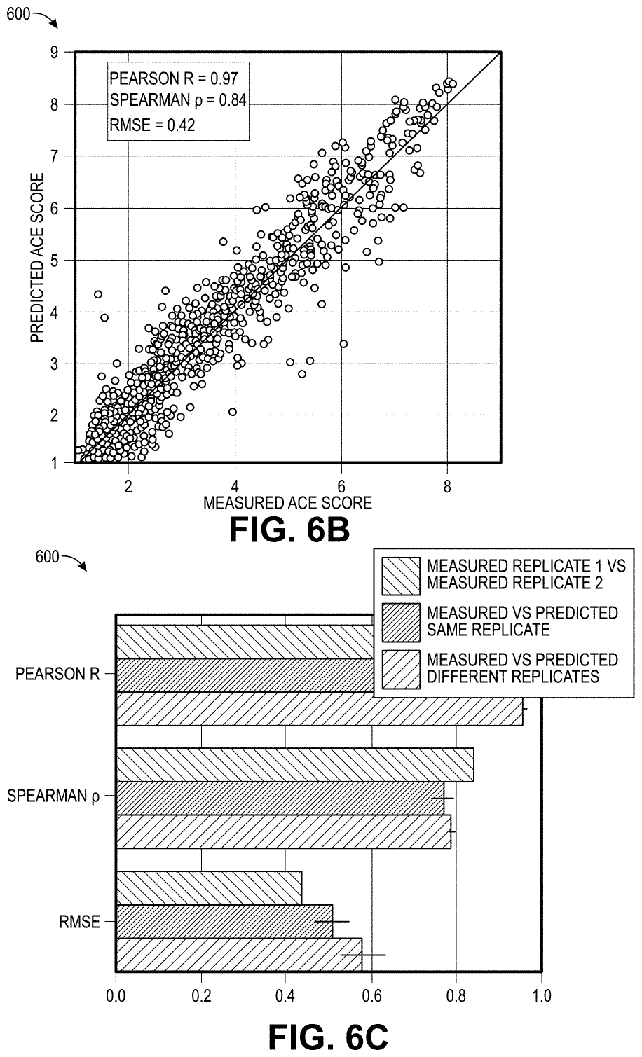

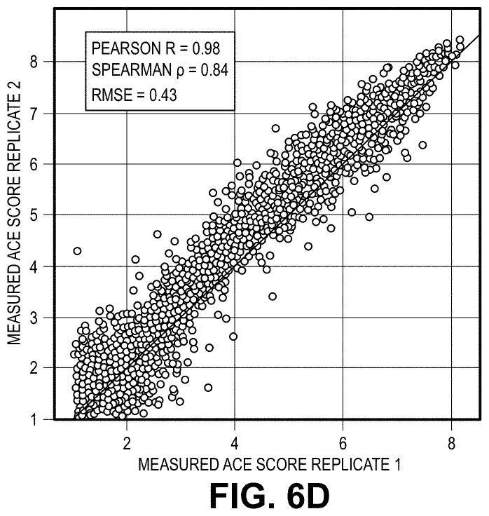

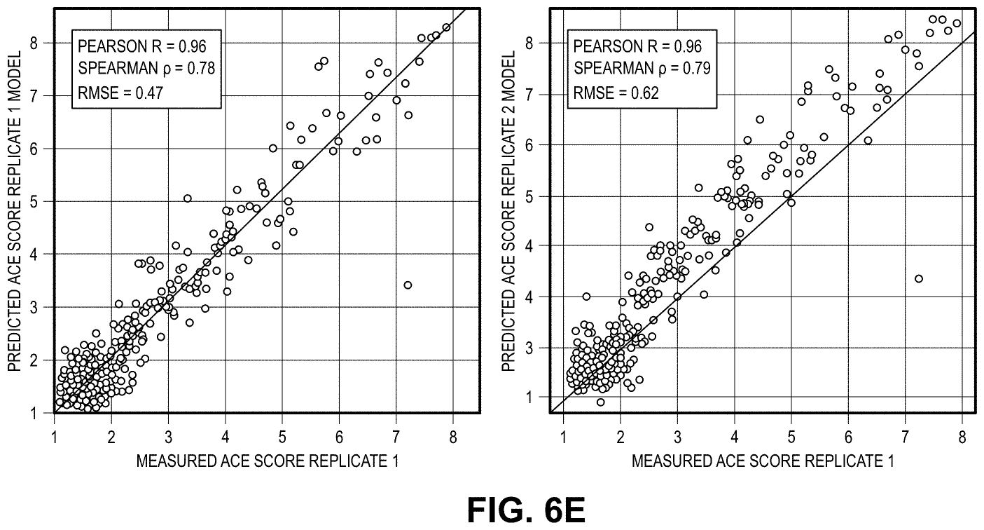

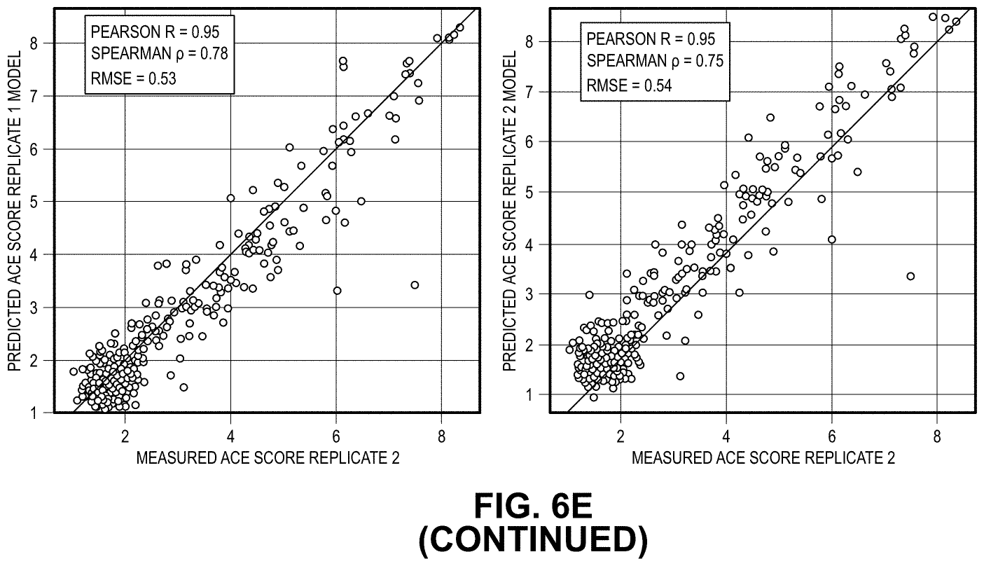

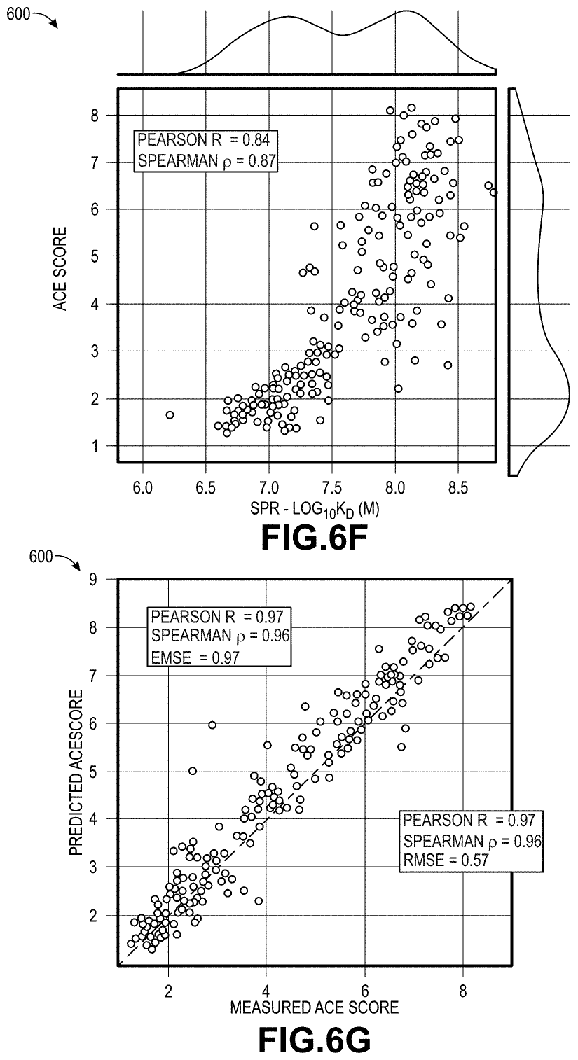

ACE scores are calculated from sequencing read counts (See Methods, infra). qaACE affinity scores are proportional to binding affinities and are highly correlated with surface plasmon resonance (SPR) K_{D} measurements, as discussed below with respect to FIG. 6F. In order to assess whether the sequence-affinity relationship can be modeled and predicted, the present techniques include generating variants of the HER2-binding antibody trastuzumab in Fragment antigen-binding (Fab) format. Mutagenesis of CDRH2 and CDRH3 was prioritized as these regions accommodate the highest density of paratope residues, both in general and for trastuzumab. Across this study, up to five simultaneous amino acid substitutions were introduced randomly in the parent antibody, in up to two CDRs, allowing all natural amino acids except cysteine (excluded to avoid potential disulfide bond-related liabilities).

The following table summarizes the datasets used to train models in this example:

| Dataset | | trast-1 | trast-2 | trast-3 |
|---|---|---|---|---|
| Screening | | ACE | SPR | ACE |
| Mutated CDRH2 positions | | — | — | 10 (55-66) |
| Mutated CDRH3 positions | | 8 (107-114) | 8 (107-114) | 10 (107-116) |
| Mutational load | | Up to double | Up to double | Up to triple |
| | | mutations | mutations | mutations |
| Allowed natural AAs | | 19 (no Cys) | 19 (no Cys) | 19 (no Cys) |
| Combinatorial space | | 9,217 | 9,217 | 6,710,401 |
| # Measured Variants | | 8,932 | 215 | 52,596 |
| Design | | Random* | Uniform** | Random, |
| | | | | stratified* |
| | 0 | 1 | 1 | 1 |
| Number of | 1 | 142 | 23 | 315 |
| of Muta- | 2 | 8,789 | 191 | 4,054 |
| tions in | 3 | — | — | 44,704 |
| AA Variants | 4 | — | — | 1,992 |
| | 5 | — | — | 1,530 |

The above dataset table depicts Trastuzumab variant datasets; in particular, characteristics of datasets used to train and evaluate models. Positions hosting substitutions (IMGT numbering), number of simultaneous substitutions (mutational load) and allowed amino acids (all except cysteine) determine the combinatorial complexity of the sequence space. A subset of sequences was sampled from the combinatorial sequence space according to the indicated design strategy to build libraries for screening by qaACE or SPR. The numbers of QC-passing amino acid sequence variants upon screening and analysis are shown, broken down by mutational load. * Random sampling of combinatorial space. ** Uniform sampling by affinity from the trast-1 dataset. *** Random sampling of combinatorial space per mutational load bin, with defined prevalence ratios of mutational load bins. Quadruple and quintuple mutants were used only to assess the performance of predictions from models trained with up to triple mutants.

In addition to high-throughput (HT) qaACE data, the present techniques also leveraged low-throughput, but highly accurate SPR K_{D} readouts to assess binding affinity. SPR was used for (i) targeted re-screening of sequence variants upon primary screening with ACE; and (ii) to validate model predictions.

As a proof of concept for this workflow, the present techniques include a library containing all sequence variants with up to two mutations across eight positions of trastuzumab CDRH3. FIG. 6A depicts a diagram of this library. FIG. 6A illustrates the combinatorial mutagenesis strategy of the trast-1 dataset: up to double mutants in 8 positions of the CDRH3 of trastuzumab, screened using ACE.

FIG. 6B depicts Predictive performance of a model trained on qaACE scores of variants from 90% of trast-1, evaluated on the remaining 10% of sequences. Using the qaACE assay, the present techniques measured the binding affinity of 8,932 variants (97% of the combinatorial space) to create the trast-1 dataset in the above table. The present techniques trained a deep language model using 90% of the trast-1 dataset and evaluated the model predictions using the remaining 10% of hold-out data. The measured and predicted qaACE scores for the hold-out dataset were highly correlated, indicating that the language model could predict binding affinity with high accuracy, as shown in FIG. 6B.

FIG. 6C depicts a comparative analysis of replicate qaACE measurements and qaACE scores predicted from models trained on individual qaACE replicates, according to some aspects. Any deviation of regression metrics from the theoretical optimum (1 for correlation, 0 for 97 RMSE) is contributed by both inaccuracy in predictions and inaccuracy in measurements. To disentangle these two effects, the present techniques may consider the agreement between measurement replicates using the same metrics previously used to assess the predictive performance of the models disclosed herein as shown, for example, in FIG. 6D. In particular, evaluating the model performance relative to the agreement of measurement replicates indicated that most of the error between predictions and measurements could be attributed to experimental noise, as shown in FIG. 6C and in FIG. 6E.

The hold-out set evaluated in FIGS. 6A-B was randomly drawn from the trast-1 dataset. Therefore, training and hold-out sets had similar distributions of qaACE scores, with a prevalence of low-affinity binders due to the detrimental effect of most mutations. This design of training and hold-out sets addressed the question of whether models can simulate experiments in silico. However, a more challenging test would require assessing predictions using a hold-out set uniformly distributed with respect to binding affinities. This hold-out set would be enriched in stronger binders relative to the training set. To reduce the prevalence of weak binders in our new hold-out set, the present techniques sampled >200 sequences from the trast-1 dataset. The sampled sequences were rescreened by SPR to create the trast-2 dataset shown in the above dataset table.

FIG. 6F depicts a correlation between qaACE affinity score and log-transformed SPR K_{D} measurements, according to some aspects. In particular, FIG. 6F includes a plot showing qaACE scores from trast-1 for sequence variants intersecting with trast-2. Empirical study observed strong agreement between qaACE scores and SPR-derived −log₁₀ K_{D} values of trast-2 sequences, as shown in FIG. 6F, as confirmed the near-uniform distribution of this dataset.

FIG. 6G depicts predictive performance against a hold-out set uniformly distributed with respect to binding affinity (ACE scores from trast-1 for sequences shown in panel FIG. 6F), according to some aspects. As shown in FIG. 6G, the present techniques may include using the trast-2 sequences as a hold-out set for models trained with trast-1 qaACE scores, which confirmed strong predictive performances.

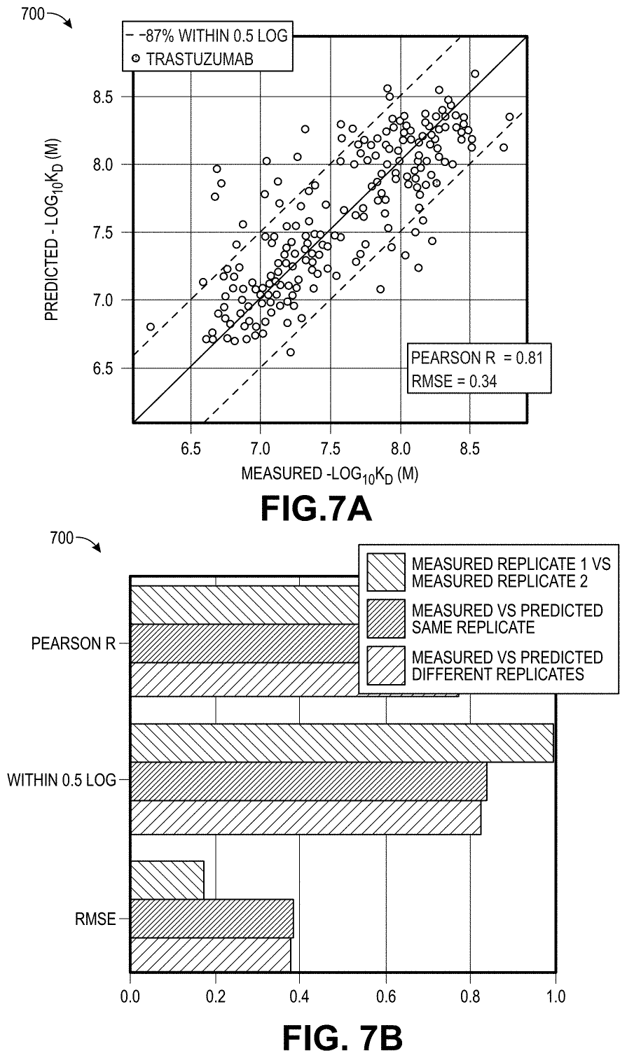

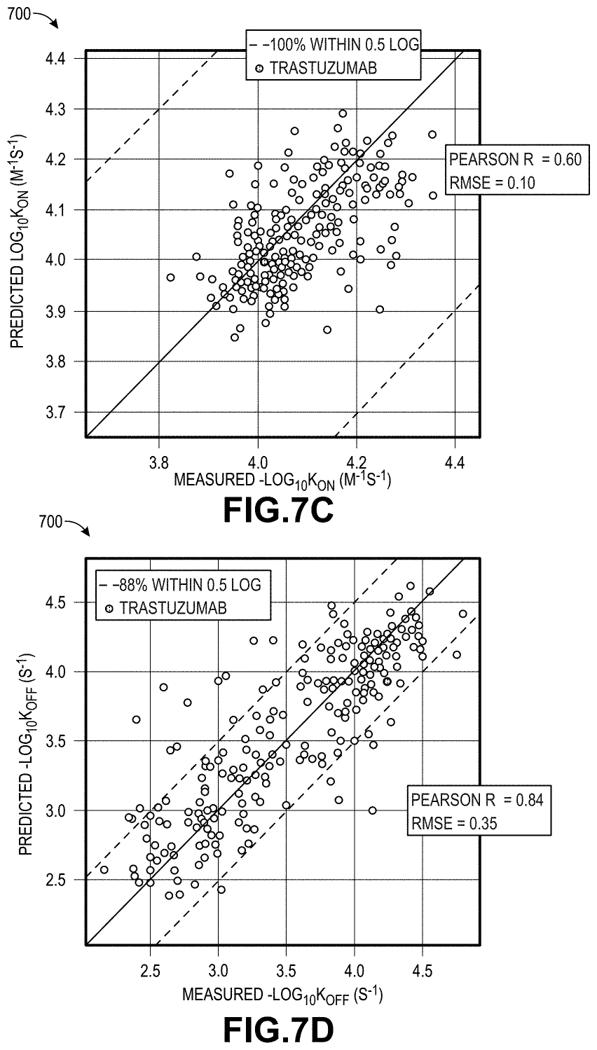

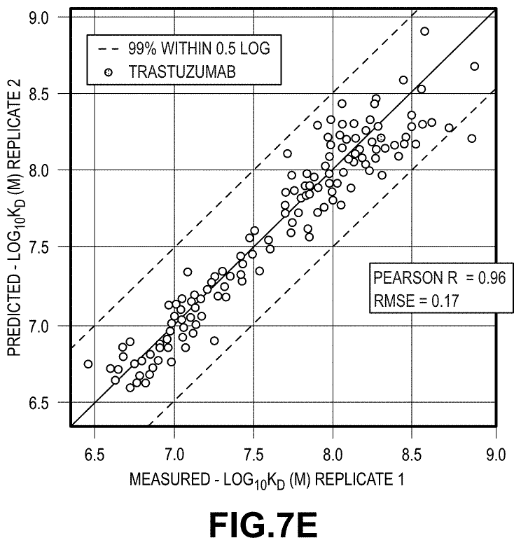

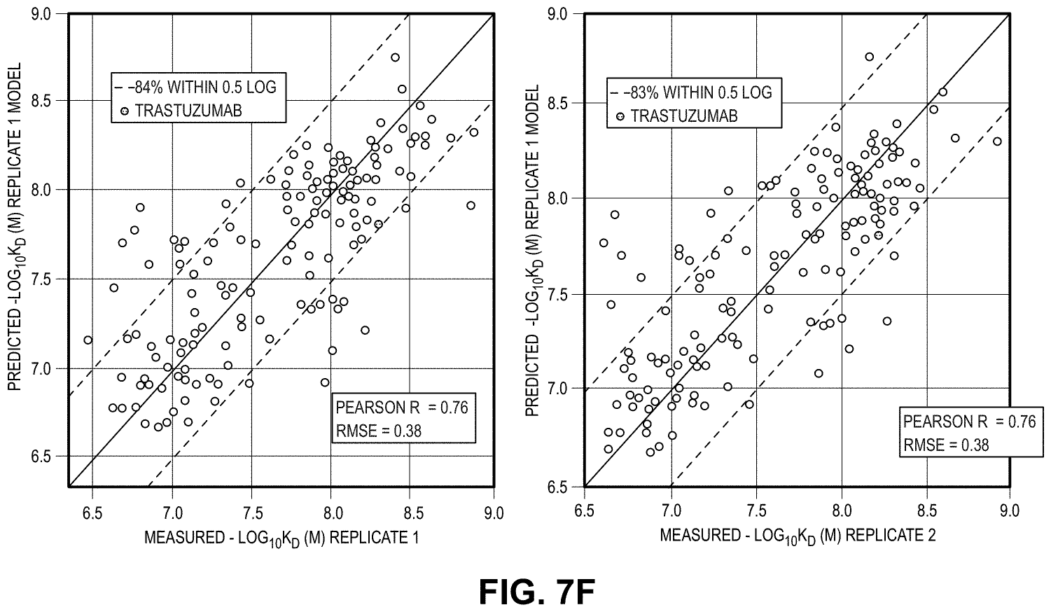

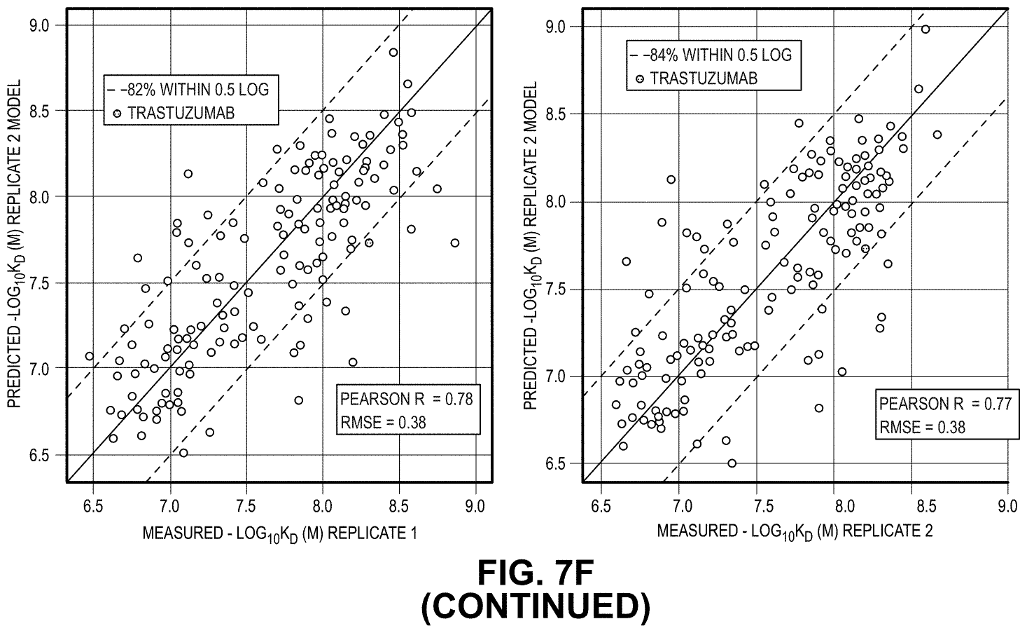

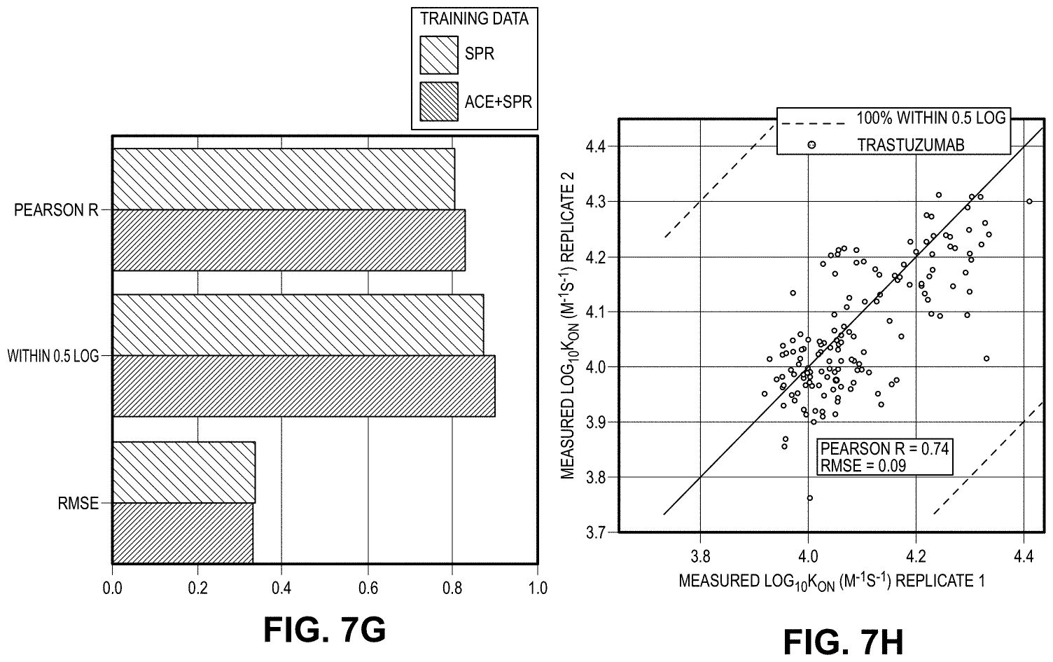

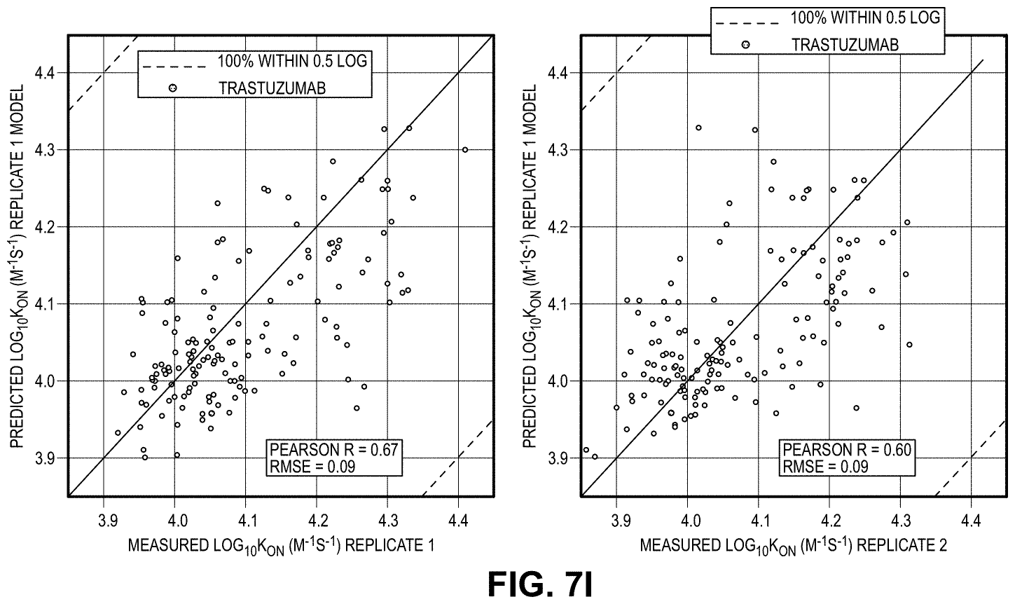

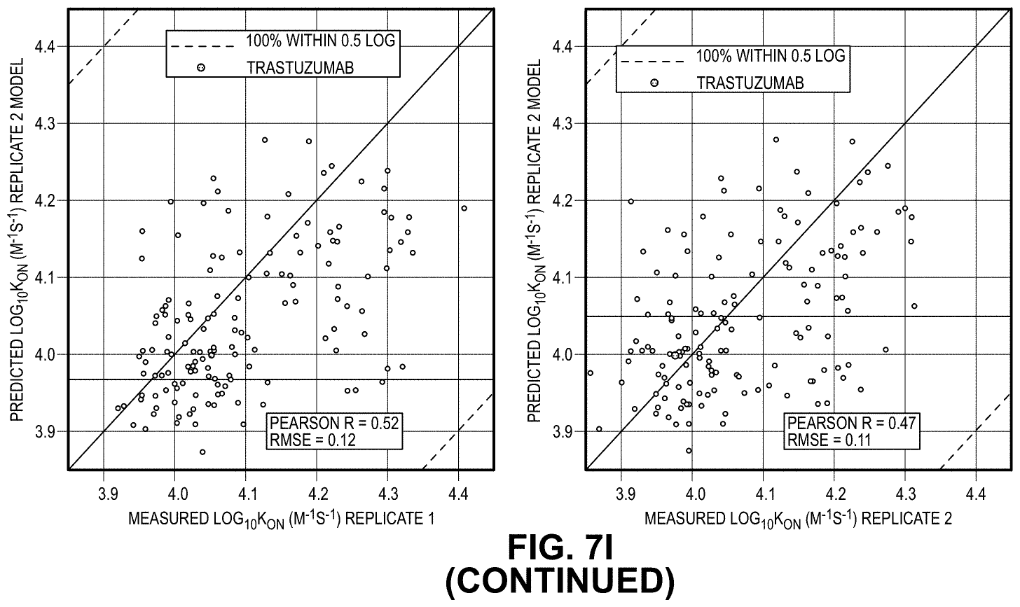

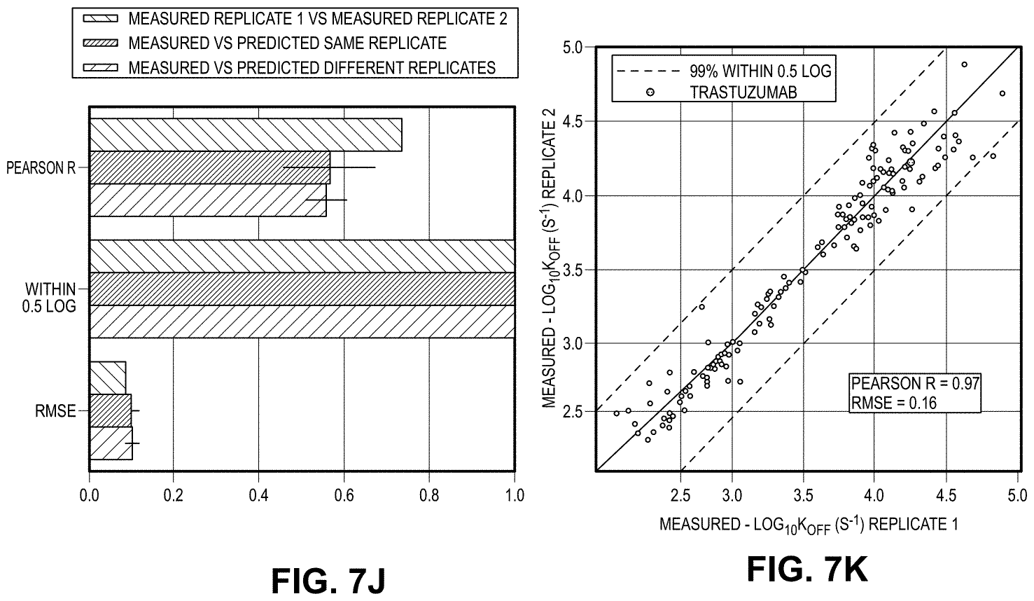

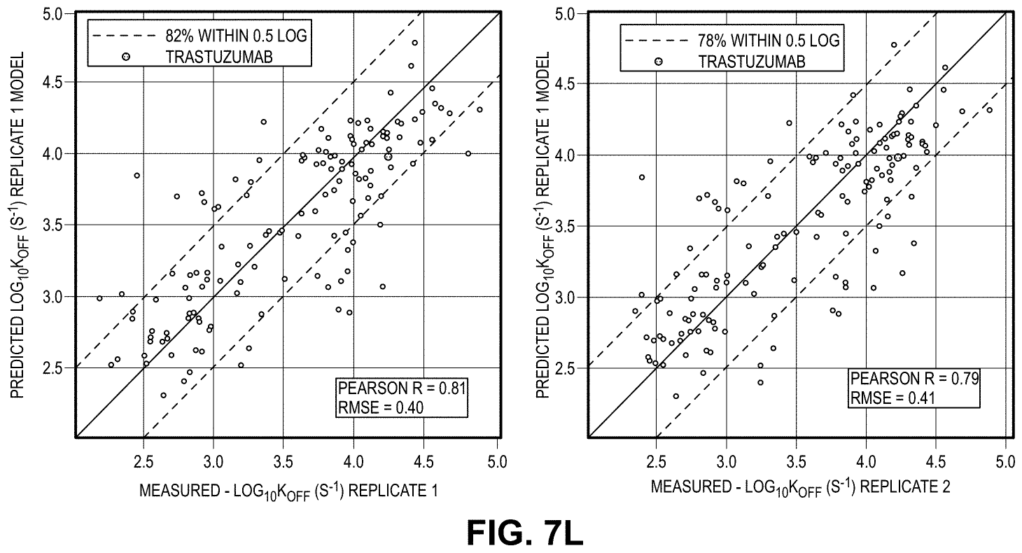

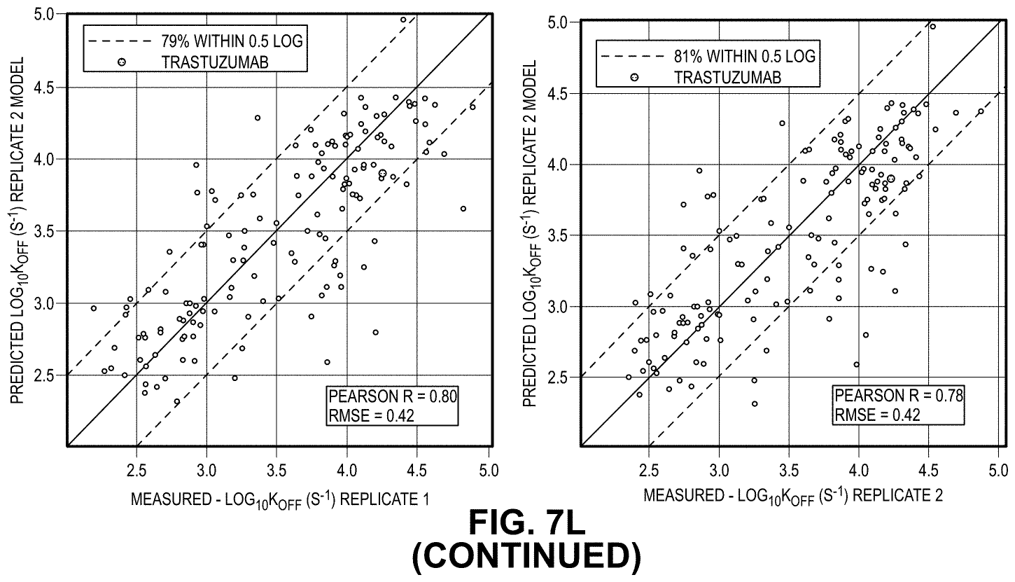

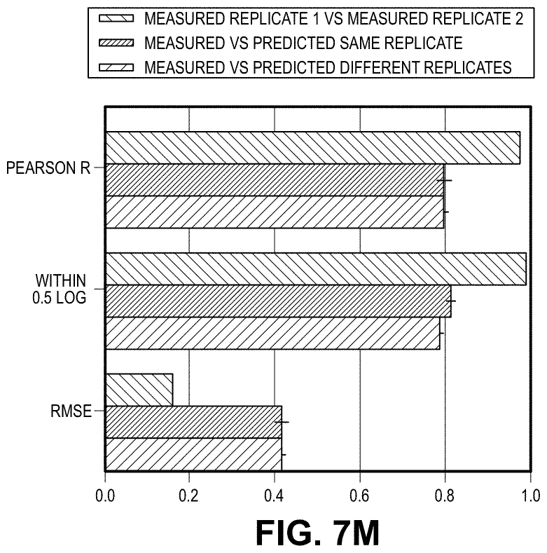

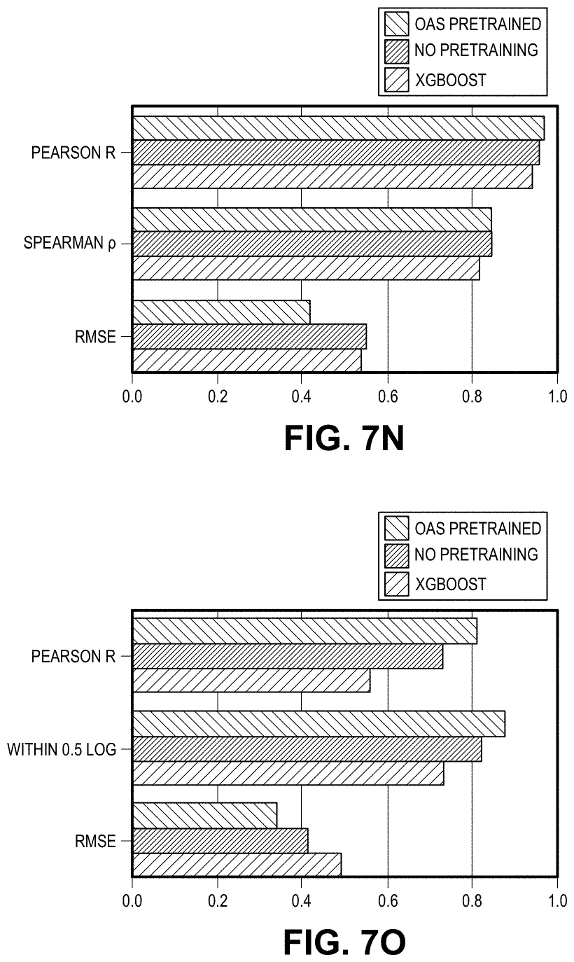

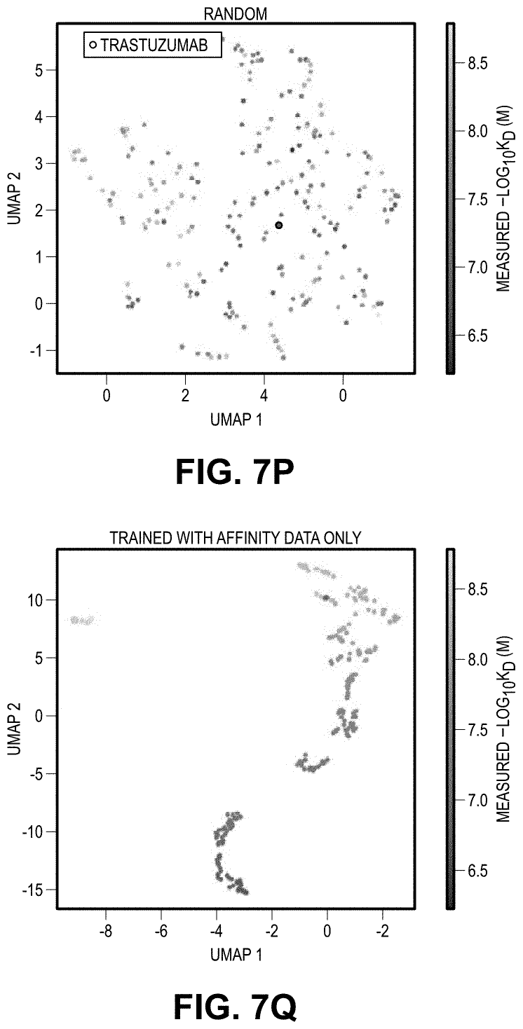

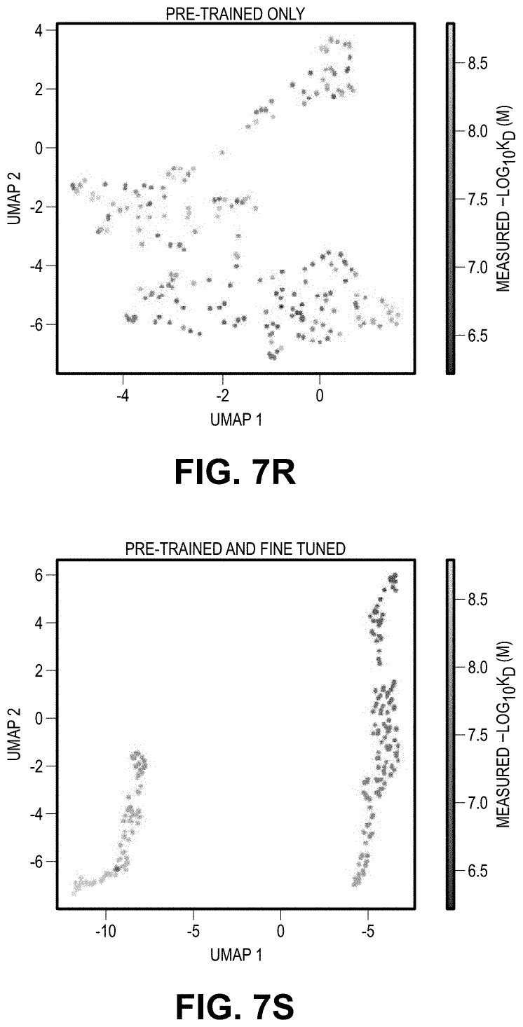

In general, the present techniques demonstrate that deep language models trained with the SPR-generated trast-2 dataset quantitatively predict antibody binding affinity, as shown in FIGS. 7A-7D, wherein performance is evaluated by pooled 10-fold cross-validation. In particular, FIG. 7A depicts predictions from a model trained on SPR-measured −log₁₀ K_{D} values; FIG. 7B depicts comparative analysis of replicate −log₁₀ K_{D} measurements and −log₁₀ K_{D} predicted from models trained on individual SPR replicates; FIG. 7C depicts predictions from a model trained on log₁₀ kₒₙ values; and FIG. 7D depicts predictions from a model trained on −log₁₀ k_{off} values.

Since SPR measurements are collected in some aspects, as with the trast-2 dataset depicted in FIG. 6F, the present techniques may be used to investigate whether this dataset alone was sufficient to train a deep language model to directly predict binding coefficients. Due to the relatively small size of the dataset (n=215), all models may be trained using 10-fold cross-validation and model performance was evaluated using pooled out-of-fold predictions. For example, the present techniques were used to first train a model to predict −log₁₀ K_{D} values, and found that the correlation between measured and predicted values was slightly lower than observed with the high-throughput trast-1 dataset, as shown in FIG. 7A. However, 87% of predicted binding affinities deviated by less than half of a log from their respective measured values. Similar to trast-1, the present techniques may include, for example, evaluating the trast-2 results relative to the best possible performance defined as the degree of agreement between measurement replicates, as shown in FIG. 7B and in FIGS. 7E and 7F).

In addition to equilibrium binding constants, SPR provides association (k.) and dissociation (k_{off}) coefficients. Models trained to predict these coefficients also performed well, as shown in FIGS. 7C and 7D; and in FIGS. 7H-7J and FIG. 7K-7M, opening the possibility for the present AI-based techniques to aid the specific engineering of association and dissociation properties, in addition to the overall binding affinity. FIGS. 7H-7J depict comparisons of measured and predicted log₁₀ kₒₙ values between replicates in the trast-2 SPR dataset. FIGS. 7K-7M depict comparisons of measured and predicted −log₁₀ k_{f} values between replicates in the trast-2 SPR dataset. Note that the lower correlation coefficient observed for k. is due to the small range of observed variation. Consistently, agreement of measurement replicates is also lower for kₒₙ than for k_{off}, which further underscores the need to consider measurement noise when assessing prediction performances.

Finally, the present techniques allow determining whether a model simultaneously trained with two affinity data types can improve the performance of a model only fed with a single data type. To this aim, for example, the present techniques were used to train a model to predict −log₁₀ K_{D} values using both qaACE (trast-1) and SPR (trast-2) data in a multi-task setting. Empirical study found this model to slightly out-perform the model trained only on trast-2 SPR data, as shown in FIG. 7G. FIG. 7G may depict models trained using SPR data from trast-2, or co-trained using both ACE (trast-1) and SPR (trast-2) data; wherein models were evaluated using 10-fold cross-validation, predicting the −log₁₀ K_{D} values of the sequences in each out-of-fold validation set; and/or wherein predictions were combined across folds and compared against SPR-measured −log₁₀ K_{D} values.

In some aspects, all models trained on the trast-1 and trast-2 datasets are deep language models pre-trained on immunoglobulin sequences from the OAS database (see Methods, infra). In aspects, these models may be compared against baselines, either using a 90:10 train:hold-out split from the trast-1 dataset or a pooled 10-fold cross-validation from the trast-2 dataset. For example, the present techniques may be used to first train a deep language model with an identical architecture but no pre-training (i.e., randomly-initialized weights) to evaluate the impact of transfer learning. Second, an XGBoost model may be trained to determine if deep language models boosted predictive accuracy relative to “shallow” machine learning. In some aspects, the pre-trained model out-performed both baselines 147 for both the trast-1 and trast-2 datasets, as shown in FIG. 7N and FIG. 7O, with a stronger benefit seen for the smaller trast-2 dataset, in line with previous observations [23].

For example, FIGS. 7N and 7O depict a comparison of pre-trained language model performance against baselines, wherein the two predictive performance of the OAS pre-trained deep language model was compared against two baselines: (1) a deep language model with identical architecture but randomly-initialized weights, and (2) an XGBoost model. In some aspects, models were trained and evaluated using a 90:10 train:hold-out split of ACE scores from the trast-1 dataset, or 10-fold cross-validation with −log₁₀ K_{D} values from the trast-2 dataset.

To understand why pre-training improves model performance, the present techniques may be used to inspect model embeddings from all combinations of pre-training vs. no pre-training, and fine-tuning vs. no fine-tuning. Even without fine-tuning, embeddings from OAS pre-training appear to have structure with distinct patches enriched for high (or low) binding affinities. This organization simplifies subsequent fine-tuning with binding data, such that the model weights can be more easily updated to provide enhanced binding affinity predictions, as shown in in FIGS. 7P-7S, which generally depict structure of model embeddings relative to binding affinities, wherein embeddings were computed with a forward pass using sequences from the trast-2 dataset, reduced to two dimensions with UMAP and graphically coded by measuring binding affinity.

### Improved Antibody Variants Discovered by Model-Guided Design

As discussed above, the present techniques demonstrate AI prediction performances using hold-out sets and cross-validation. The present techniques further demonstrate using models to design sets of sequences with desired binding properties followed by validation with dedicated SPR experiments. For example, in an aspect, the present techniques may train a model trained on the trast-2 dataset with designing sequences spanning two orders of magnitude of equilibrium dissociation constants (referred to herein as design set A).

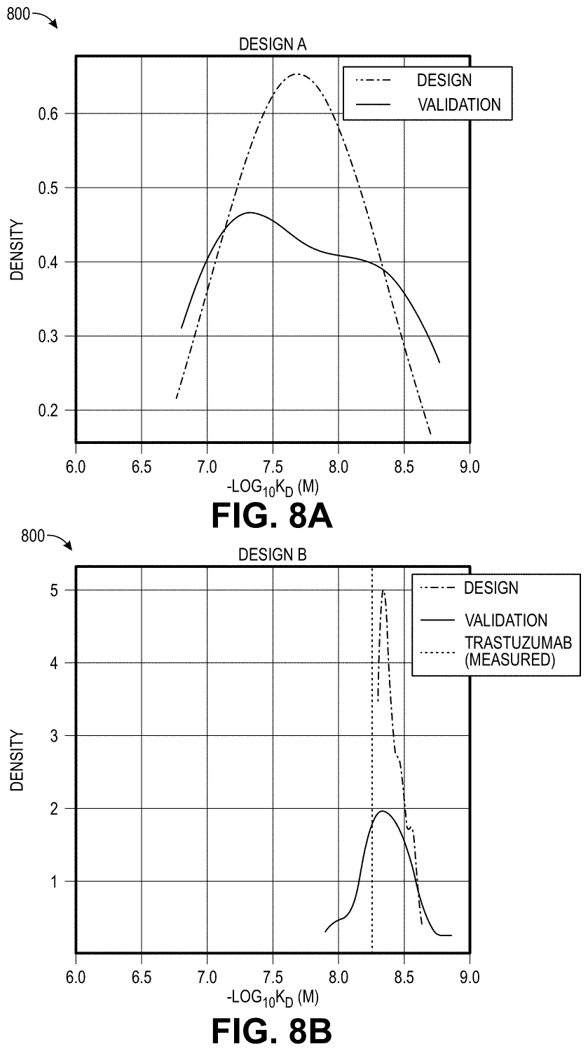

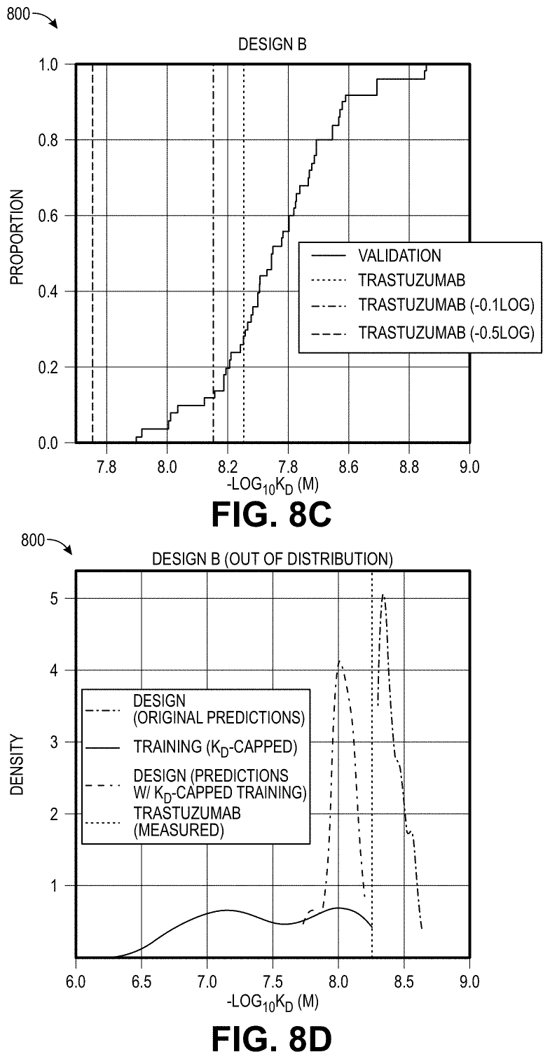

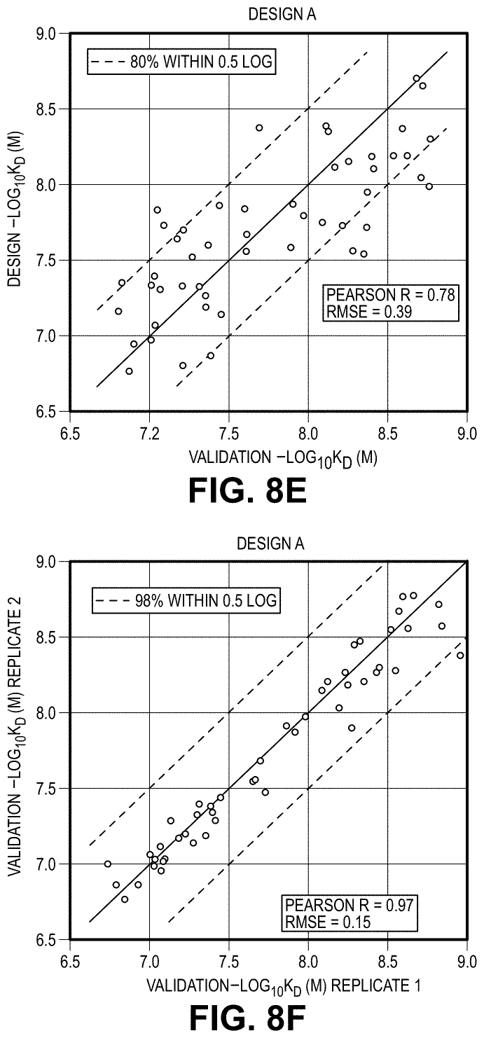

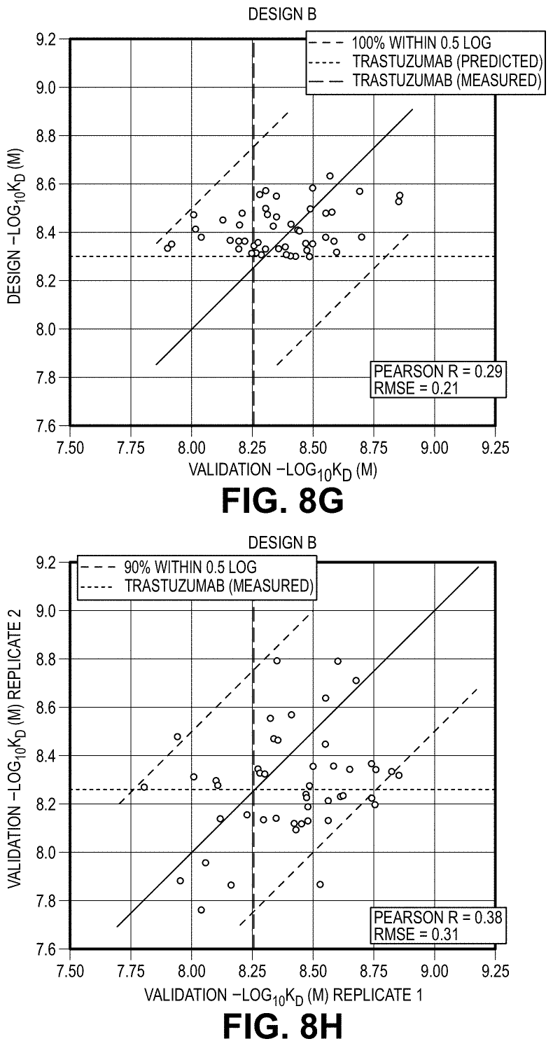

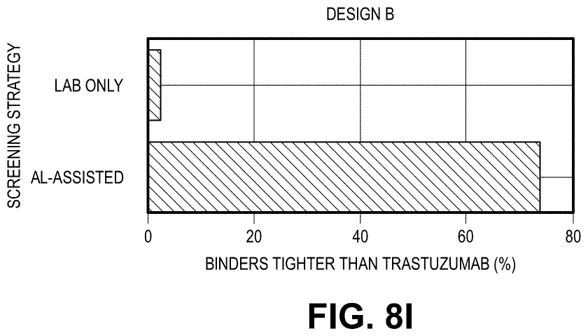

Generally, model-enabled design involves exhaustive predictions in the combinatorial sequence space, followed by sampling sequences with predicted binding affinities consistent with requirements. FIG. 8A-8D depict deep language models trained with the SPR-generated trast-2 dataset can design unseen sequence variants that validate in independent SPR experiments, according to some aspects. FIGS. 8E-8H depict deep language models trained with the SPR-generated trast-2 dataset can design unseen sequence variants that validate in independent SPR experiments, such as those shown in FIGS. 8A-8D. Empirically, the present techniques found excellent agreement between predictions and validations for design set A, as shown in FIGS. 8A, 8E and 8F).

The present techniques may further be used to demonstrate a more challenging design, asking for variants with binding stronger than trastuzumab (referred to herein as design set B). As for the previous design, for example, the present techniques were used to validate 50 sequences by SPR, finding that 74% of variants were indeed tighter binders than the parental antibody, as shown in (FIGS. 8B-C and in the following table, and that 100% complied with the design specification within a tolerance of less than 0.5 log, as shown in FIG. 8C and FIG. 8G:

| | | Predicted | |
|---|---|---|---|
| | | -log₁₀ K_{D} | Validated |
| SEQ IDS | CDRH3 | (M) | -log₁₀ K_{D} (M) |
| SEQ ID NO: 28 | SRWWGGGFYAMDY | 8.55 | 8.86 |
| SEQ ID NO: 29 | SRWISDGFYAMDY | 8.53 | 8.85 |
| SEQ ID NO: 30 | SRWWGAGFYAMDY | 8.38 | 8.69 |
| SEQ ID NO: 31 | SRWWGAGFYAMDY | 8.57 | 8.69 |
| SEQ ID NO: 32 | SRWPGIGFYAMDY | 8.31 | 8.59 |
| SEQ ID NO: 33 | SRWGGHGFYAMDY | 8.48 | 8.58 |
| SEQ ID NO: 34 | SRWIRDGFYAMDY | 8.36 | 8.58 |
| SEQ ID NO: 35 | SRWIADGFYAMDY | 8.63 | 8.57 |
| SEQ ID NO: 36 | SRWSGAGFYAMDY | 8.38 | 8.55 |
| SEQ ID NO: 37 | SRWIGSGFYAMDY | 8.48 | 8.55 |
| SEQ ID NO: 38 | SRWGGTGFYVMDY | 8.35 | 8.5 |
| SEQ ID NO: 39 | SRWIGHGFYAMDY | 8.58 | 8.5 |
| SEQ ID NO: 40 | SRWIQDGFYAMDY | 8.5 | 8.49 |
| SEQ ID NO: 41 | SRWAGPGFYAMDY | 8.3 | 8.48 |
| SEQ ID NO: 42 | SRWLGPGFYAMDY | 8.33 | 8.47 |
| SEQ ID NO: 43 | SRWGAIGFYAMDY | 8.35 | 8.47 |
| SEQ ID NO: 44 | SRWAGYGFYAMDY | 8.4 | 8.44 |
| SEQ ID NO: 45 | SRWLGIGFYAMDY | 8.41 | 8.43 |
| SEQ ID NO: 46 | SRWIGNGFYAMDY | 8.4 | 8.43 |
| SEQ ID NO: 47 | SRWGQIGFYAMDY | 8.3 | 8.42 |
| SEQ ID NO: 48 | SRWIGLGFYAMDY | 8.43 | 8.41 |
| SEQ ID NO: 49 | SRWGRTGFYAMDY | 8.3 | 8.4 |
| SEQ ID NO: 50 | SRWIGMGFYAMDY | 8.3 | 8.39 |
| SEQ ID NO: 51 | SRWVGLGFYAMDY | 8.34 | 8.38 |
| SEQ ID NO: 52 | SRWIGGGFYAMDY | 8.46 | 8.35 |
| SEQ ID NO: 53 | SRWVGTGFYAMDY | 8.33 | 8.35 |
| SEQ ID NO: 54 | SRWIGVGFYAMDY | 8.55 | 8.35 |
| SEQ ID NO: 55 | SRWVGGGFYAMDY | 8.42 | 8.33 |
| SEQ ID NO: 56 | SRWSGQGFYAMDY | 8.47 | 8.31 |
| SEQ ID NO: 57 | SRWSGYGFYAMDY | 8.49 | 8.31 |
| SEQ ID NO: 58 | SRWVGIGFYAMDY | 8.57 | 8.3 |
| SEQ ID NO: 59 | SRWGGFGFFAMDY | 8.33 | 8.3 |
| SEQ ID NO: 60 | SRWLGGGFYAMDY | 8.31 | 8.28 |
| SEQ ID NO: 61 | SRWILDGFYAMDY | 8.56 | 8.28 |
| SEQ ID NO: 62 | SRWLGNGFYAMDY | 8.36 | 8.27 |
| SEQ ID NO: 63 | SRWVGRGFYAMDY | 8.31 | 8.26 |
| SEQ ID NO: 64 | SRWGGIGFFAMDY | 8.34 | 8.26 |
| SEQ ID NO: 65 | SRWDGHGFYAMDY | 8.31 | 8.24 |
| SEQ ID NO: 66 | SRWAGSGFYAMDY | 8.36 | 8.21 |
| SEQ ID NO: 67 | SRWIGTGFYAMDY | 8.48 | 8.21 |
| SEQ ID NO: 68 | SRWVIDGFYAMDY | 8.43 | 8.2 |
| SEQ ID NO: 69 | SRWAGGGFYAMDY | 8.33 | 8.19 |
| SEQ ID NO: 70 | SRWAGAGFYAMDY | 8.36 | 8.19 |
| SEQ ID NO: 71 | SRWGGGGFYVMDY | 8.36 | 8.16 |
| SEQ ID NO: 72 | SRWGGYGFFAMDY | 8.45 | 8.13 |
| SEQ ID NO: 73 | SRWGGSGFYSMDY | 8.38 | 8.04 |
| SEQ ID NO: 74 | SRWIGPGFYAMDY | 8.47 | 8.01 |
| SEQ ID NO: 75 | SRWIPDGFYAMDY | 8.41 | 8.01 |
| SEQ ID NO: 76 | SRWGGTGFFAMDY | 8.35 | 7.92 |
| SEQ ID NO: 77 | SRWGGSGFYYMDY | 8.33 | 7.9 |

The above design B sequence variants table lists 50 designed and validated trastuzumab sequence variants from design set B. For reference, the predicted and validated −log₁₀ K_{D}s of parental trastuzumab were 8.3 M and 8.25 M, respectively.

This performance is competitive when compared with replicate measurements; had we selected tighter binders based on experimental measurements from the first replicate, a similar fraction would have lower binding affinities upon a second replicate measurement (FIG. 8H).

Because of the small −log₁₀ K_{D} range of this design, correlation between predictions and measurements was low, as shown in FIG. 8G. However, as similarly observed before in the above-described km modeling, and as also depicted in FIG. 7H-7J and FIG. 8H, whenever the affinity range is narrow, even measurement replicates correlate poorly with each other. In contrast to correlation, other experimental metrics such as RMSE and the fraction of predictions deviating less than 0.5 log from measurements remained in line with previously observed performance, as shown in FIG. 8G. In some aspects, these metrics are generally more informative when working with a set of sequences packed in a narrow affinity range.

The validation rate of design set B compares very favorably against a naive approach to library screening, in which the fraction of binders tighter than trastuzumab is minimal, as shown in FIG. 8I. Specifically, FIG. 8I depicts a chart in which model predictions are shown to strongly enrich for variants with desired binding properties, relative to naive library screening. In FIG. 8I, sequences of interest in design set B may be defined as antibody variants binding more tightly to HER2 than parental trastuzumab (i.e., top binders). FIG. 8C depicts a validation rate of top binders in design set B (i.e., AI-assisted screening) versus a prevalence of top binders in the combinatorial space (i.e., lab-only screening), as estimated by the fraction of top binders in the model predictions for the full combinatorial space, adjusted for the validation rate of design set B.

The strong enrichment provided by model predictions for variants of interest is the key finding enabling in silico experiments and AI-assisted antibody optimization, as shown in FIG. 5.

As mentioned, the model used to design sequence set B may be trained on the trast-2 dataset, which includes some binders stronger than trastuzumab (see FIG. 7A). Thus, in an example, the present techniques may include determining whether a model that was never fed any sequence as extreme (affinity-wise) as those it is tasked to design can still prioritize top binders. This question is of practical value, as it is conceivable that some applications may eventually face a large sequence space and a low prevalence of positives, which would likely result in training sets devoid of positive examples.

To test the performance of the models described herein in out-of-distribution affinity prediction settings, some examples may include dropping any binder tighter than trastuzumab from the trast-2 training set, and then training a model using the remaining data and predicted the affinity of design set B. Experimentally, as expected, a model trained in this way no longer able to make accurate K_{D} predictions for design B. Remarkably, however, the model was still able to place binding affinities of design B variants at the top of its known distribution, as shown in FIG. 8D. This result demonstrates that some of the AI-based aspects of the present techniques would still enable the prioritization of top binders by sampling top-ranking predictions even if laboratory experiments generating training data did not observe the full affinity range.

AI Predictive Performance is Maintained when Scaling to a Larger Sequence Space

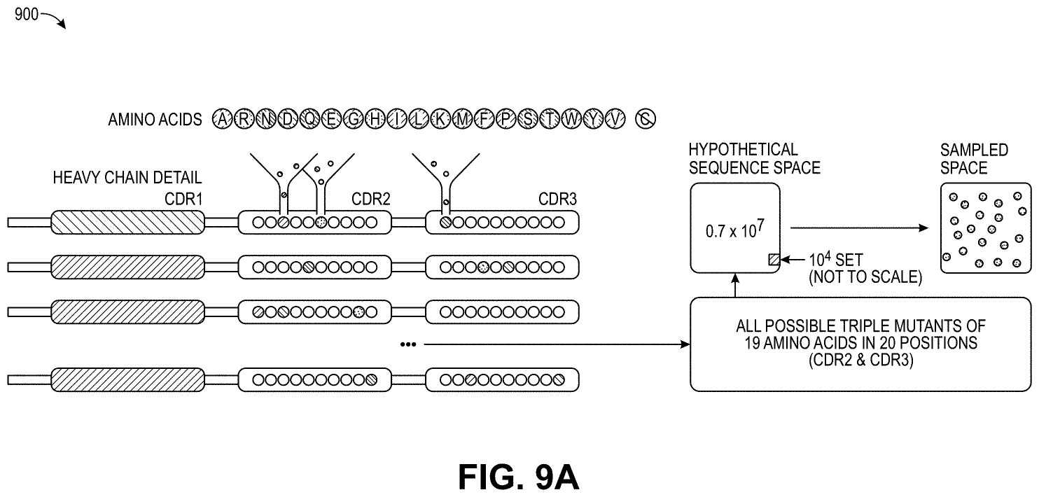

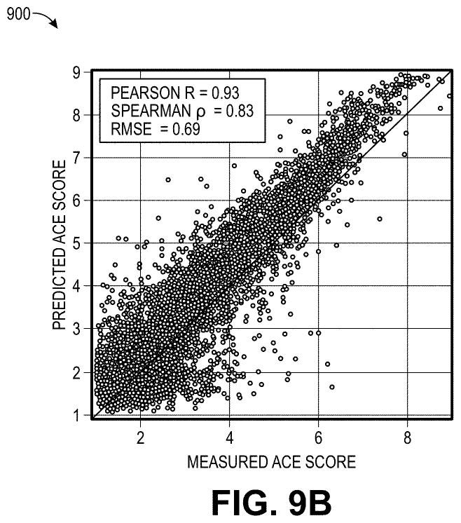

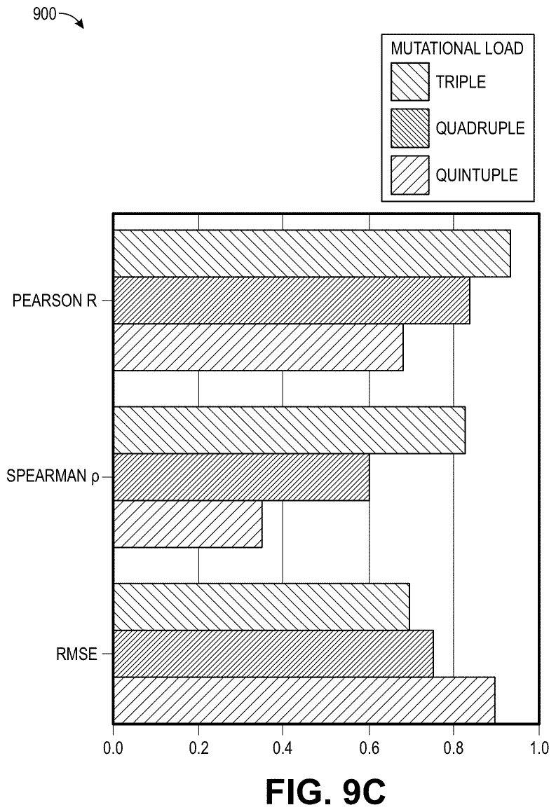

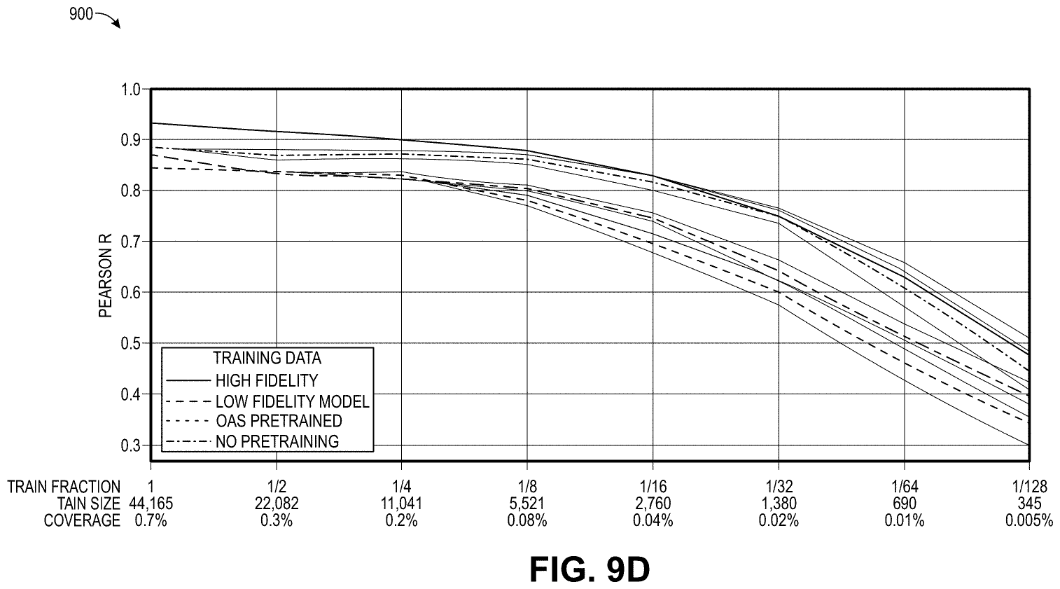

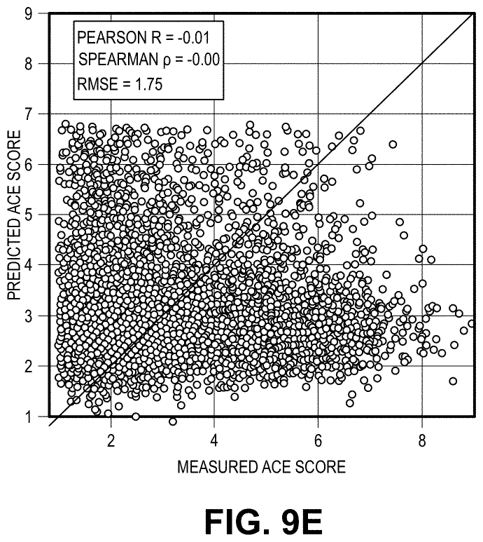

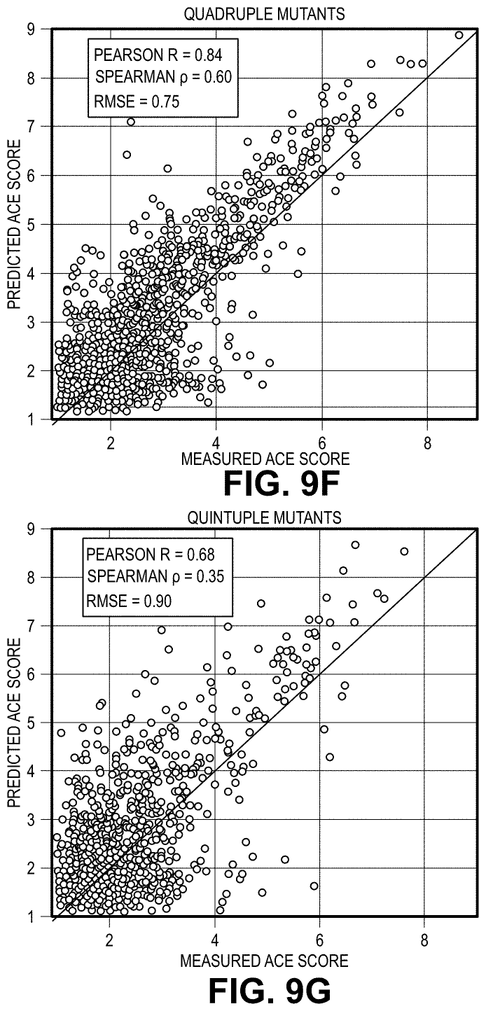

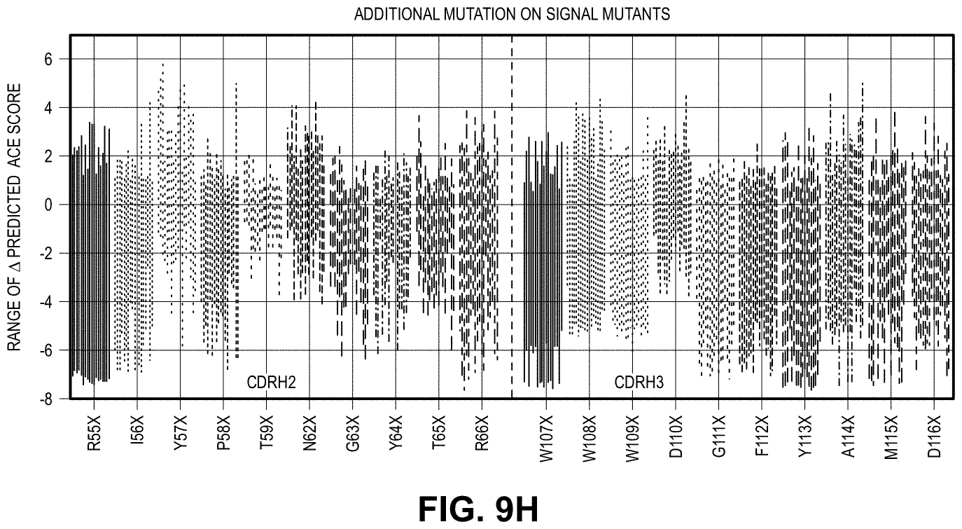

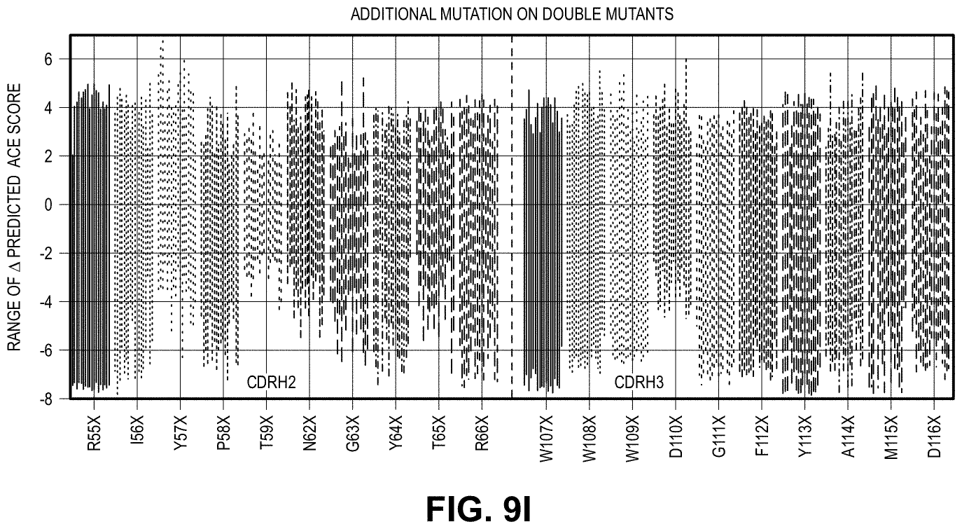

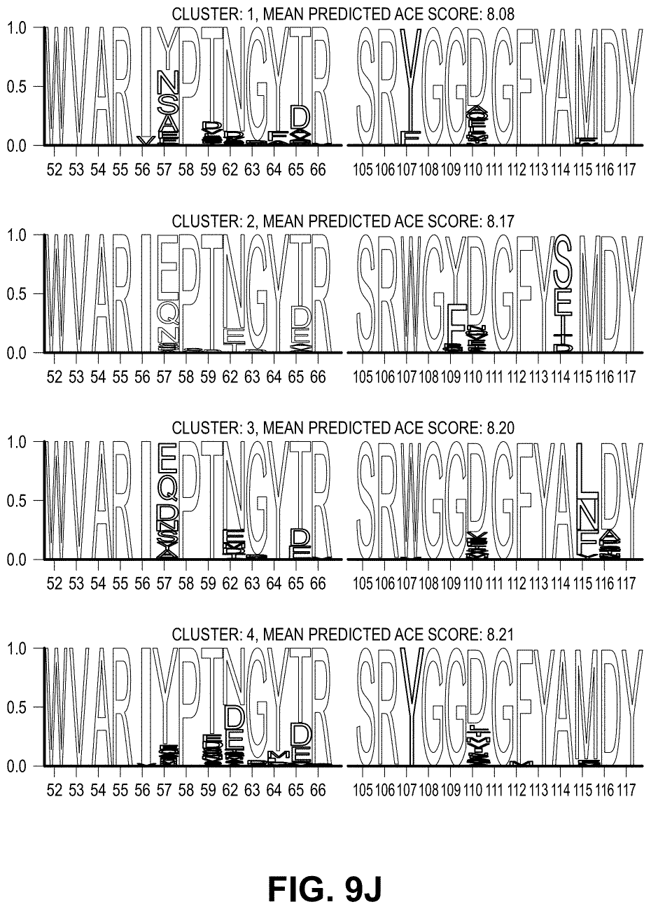

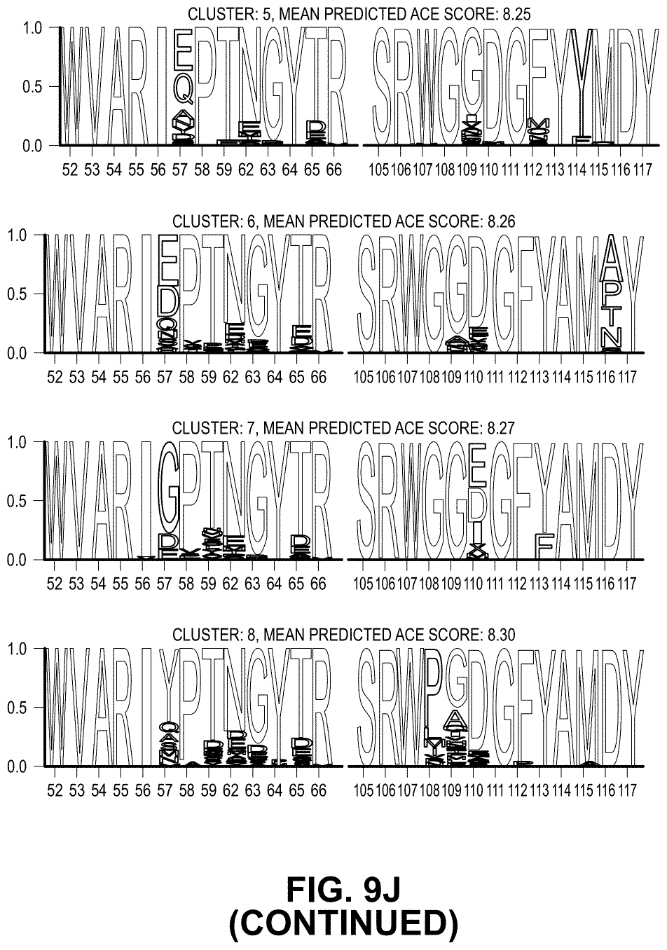

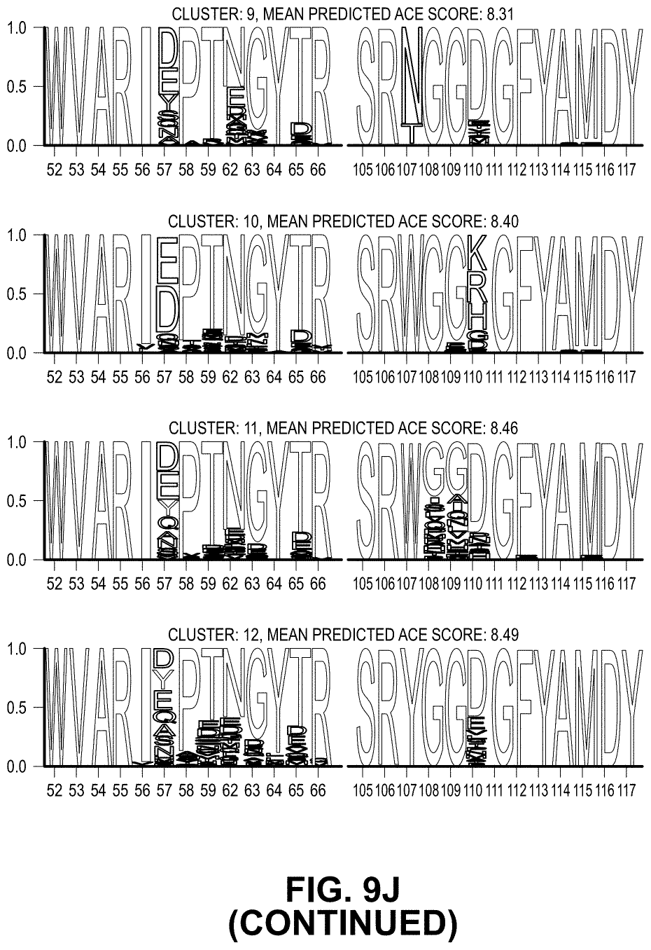

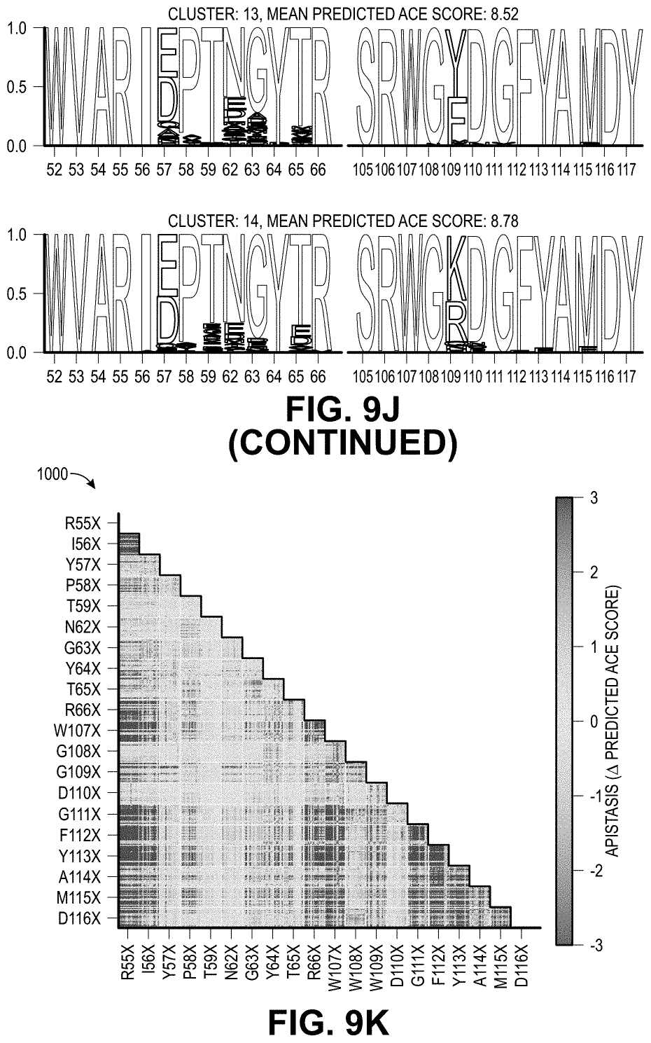

FIGS. 9A-9D depict that high-throughput binding scores from the ACE-generated trast-3 dataset can expand predictive capabilities to a larger mutational space, according to some aspects. In another example, to test the model's performance in a large sequence space, the present techniques may were used to perform combinatorial mutagenesis of up to three mutations over ten amino acids each in the CDRH2 and CDRH3. This example included constructing a library by sampling less than 1% of this sequence space, and measuring the binding affinity of the sampled sequence variants using the qaACE assay, as shown in the above dataset table of trast-3, and in FIG. 9A. This example may further include training a model using 80% of the trast-3 data, and evaluating its performance on the remaining 20% of hold-out sequences. Experimentally, model performance was comparable to the double-mutant library (FIG. 9B).

As a negative control, it was confirmed that a model trained on a dataset with randomly shuffled qaACE scores had no predictive power, as shown in FIG. 9E. Specifically, models trained in this manner are unable to predict ACE scores from randomly shuffled data. Since the trast-3 sequence space was so large, all models were trained and evaluated on only qaACE data, since the high correlation between qaACE scores and −log₁₀ K_{D} values measured by SPR was already demonstrated (see FIG. 6F).

Given the predictive accuracy of the trast-3 model on variants with up to three mutations from the original trastuzumab sequence, an experiment was also conducted to test whether the model could accurately predict the qaACE scores of variants with four or five mutations from trastuzumab (FIG. 9C).

For example, FIG. 9F depicts prediction performance on a set of quadruple mutants, according to some aspects. FIG. 9G depicts prediction performance on a set of quintuple mutants, according to some aspects. In FIG. 9F and FIG. 9G, the model may be trained on the trast-3 dataset of up to triple mutants of the parental trastuzumab sequence, and evaluated on a hold-out set of only quadruple or quintuple mutants, respectively, for example.

As shown, in some aspects, the model may predict qaACE scores of quadruple mutants with slightly lower accuracy than triple mutants, as shown in FIG. 9F. Although the prediction accuracy for quintuple mutants was much lower, as shown for example in FIG. 9G, the model could still be used to discriminate between high and low binders in a classification setting. These results show that the triple mutant model can be extrapolated to quantitatively predict binding scores for up to four simultaneous mutations from the original sequence, and qualitatively predict binding scores for five mutations. As the mutational distance increased, model accuracy degraded, especially for variants with low affinity, as depicted in FIG. 9F and FIG. 9G.

### Deep Language Models are Highly Sample Efficient

Generally, the predictive power of any deep learning model is highly dependent on the quality and quantity of its training data. The trast-3 dataset contains binding affinities for around 50,000 unique antibody sequences, covering 0.7% of the complete combinatorial mutation space for this design, as shown in the above dataset table. To determine the relationship between model performance and the quality and quantity of the training dataset, some examples may include training a cohort of models to predict affinity from a range of dataset sizes sampled from datasets of varying fidelity. as shown in FIG. 9D. In some examples, the original trast-3 dataset may be treated as a high-fidelity dataset, and a low-fidelity dataset may be generated by isolating a single DNA variant for each sequence from a single FACS sort (see Methods, infra). In this example, the size of the training subsets may range from 44,165 sequences (the full 233 training dataset), through 350 sequences ( 1/128 of the full training dataset), and models may be evaluated on a common hold-out validation dataset containing 10% of all sequences in the high-fidelity dataset. At each training subset size, the performance of four models may be compared: (1) OAS pretrained models trained on a subset from the high-fidelity dataset; (2) OAS pretrained models trained on a subset from the low-fidelity dataset; (3) randomly-initialized models trained on a subset from the high-fidelity dataset; and (4) randomly initialized models trained on a subset from the low-fidelity subset.

As the size of the training dataset decreased, the model performance degraded. Models trained on low-fidelity data consistently performed poorer than their counterparts trained on high-fidelity data, highlighting the importance of high-quality experimental assays.

Pre-training the model with the OAS dataset usually improved model performance; however, the performance gains from pre-training were reduced either when models were trained using smaller, high-fidelity datasets, or larger, low-fidelity datasets.

Given that the model requires at least 2,760 sequences to maintain a Pearson R correlation above 0.8, it is impractical to model this mutational space using only SPR training data; higher-throughput assays such as qaACE are required. Since the Pearson R correlation remained above 0.8 for all high-fidelity training subsets covering at least 0.4% of the potential search space, the model learned to predict roughly 2,500 sequences for every sequence in the training dataset. Therefore, the deep language models of the present techniques can expand the search space of an experimental dataset by at least orders of magnitude.

### Deep Language Models Provide Interpretable Analysis of the Antibody Binding Landscape

Once trained, AI models can be used as oracles to predict binding affinity scores for all sequences within the combinatorial space matching the design of the training set. Fast and accurate predictions can inform how an antibody would be affected by different engineering strategies and help guide experimental efforts.

To gain insight into the mutational landscape of trastuzumab, some examples may include using the present techniques to exhaustively evaluate the effect of all single, double and triple mutations in CDRH2 and CDRH3. Trastuzumab has a high binding affinity for its target antigen HER2 (−log₁₀ K_{D} of 8.25 M in Fab format, as shown in FIG. 7A. Thus, most mutations were predicted to have a detrimental effect on the binding affinity, as shown in FIG. 5. When considering multiple mutations, empirical testing suggested that most combinations were predicted to have a detrimental effect on the binding affinity, as shown in FIG. 9H and FIG. 9I).

In particular, FIG. 9H and FIG. 9I show that positions 55, 107, 111, 112, and 113, were predicted to have a detrimental effect when mutated and tended to interact epistatically with other mutations, as discussed later. This pointed to a strong contribution to binding affinity from these residues, in agreement with previous alanine scanning and structural studies [22]. For reference, the effect of each individual mutation on trastuzumab is indicated with dots that are identical in both FIG. 9H and FIG. 9I. Mutations at each position include all possible substitutions with natural amino acids except cysteine, sorted alphabetically (i.e., X∈[A, D, E, F, G, H, I, K, L, M, N, P, Q, R, S, T, V, W, Y]).

Analyzing the incremental effects of mutations across variants indicated that positions 59, 62, and 110 were relatively tolerant to mutations, as depicted in FIG. 9H and FIG. 9I. This suggested that they made a relatively small contribution to binding affinity, and may be ideal candidates to optimize for other antibody properties. Some single mutations in CDRH2, such as Y57D/E, N62E or T65D/E, were predicted to increase binding affinity (see FIG. 5). In addition to single mutants, combining multiple mutations may also provide improved high-affinity variants. In fact, as the mutational load increased, the number of predicted high-affinity sequences increased, although their proportion was reduced. For instance, 2 (0.56%) of the single mutants, 192 (0.31%) of the double mutants, and 7,063 (0.11%) of the triple mutants had predicted qaACE scores higher than trastuzumab in the trast-3 dataset.

The present AI-based techniques enable identifying diverse clusters of high-affinity variants of trastuzumab. In an example, the present techniques were used to carry out a clustering analysis of model-derived embeddings of high-affinity sequences (predicted qaACE score >8.0). For example, FIG. 9J depicts sequence logo plots illustrating the composition of high-affinity clusters of embeddings (predicted ACE score >8.0). Clusters may be generated by reducing the dimensionality of embeddings followed by HDBSCAN clustering, and sorting by mean predicted ACE store. A minimum number of sequences per cluster may be required (e.g., 40). The logo plots of FIG. 9J indicate the relative frequency of each specific substitution in the sequences within each cluster. In some aspects, the predictions of binding affinities came from a model trained on the trast-3 dataset.

While the space of triple mutants offered many potential high-affinity candidate sequences, these tended to form compact clusters involving specific substitutions in a few positions, as shown in FIG. 9J. Notably, mutation Y57D/E was observed in several clusters. Also, most high-affinity triple mutants had two or three mutations in the CDRH2 (particularly in positions 57 and 62 or adjacent positions), while fewer solutions involved one mutation in CDRH2 and two mutations in CDRH3. This finding highlights the key role of the CDRH2 region in antigen binding by trastuzumab, as also noted by others [22, 24].

Empirical testing also demonstrated that the impact of a given mutation on binding affinity varied widely with the presence of other mutations in the sequence, a phenomenon known as contingency [25].

FIG. 9H and FIG. 9I depict that a given mutation can have a larger, smaller, or even opposite effect compared to the effect it would have on the parental trastuzumab sequence, depending on the presence of just another single mutation. Further, in the presence of two mutations, the possible range of effects for an additional (third) mutation becomes wider).

In a similar vein, epistasis is the deviation from additivity in the effects of two co-occurring mutations compared to their individual effects [26]. The epistatic interaction between mutations for all double mutants of trastuzumab is depicted in FIG. 9K.

Specifically, FIG. 9K depicts that antagonistic epistasis is commonly found between key paratope residues in trastuzumab. The heatmap of FIG. 9K depicts epistasis effects across all possible pairs of substitutions. Epistasis refers to a deviation from additivity in the effects of two mutations when they are both present. Antagonistic epistasis refers to a smaller-than-expected change in binding affinity when two mutations co-occur. Mutations at each position include all possible substitutions with natural amino acids except cysteine sorted alphabetically (i.e., X∈[A, D, E, F, G, H, I, K, L, M, N, P, Q, R, S, T, V, W, Y]), according to some aspects. sorted alphabetically (i.e., X∈[A, D, E, F, G, H, I, K, L, M, N, P, Q, R, S, T, V, W, Y]), according to some aspects.

Given the negative effect that many mutations had on binding affinity, antagonistic, positive epistasis is often observed (i.e., a double mutant displays a higher binding affinity than expected based on its constituent single mutants). This is particularly evident in pairs of mutations involving positions 55, 107, 111, 112, and 113, which are crucial to the binding affinity of trastuzumab [22]. Epistatic interactions are also highly contingent on the presence of other mutations in the sequence. The complex interaction between mutations directly affects the biochemical properties of antibodies.

Taken together, the diversity of high-affinity sequences and their dilution as a function of mutational load highlights the value of exhaustively evaluating the space of possible variants. Such large-scale evaluation is only feasible with the help of computational models. The models discussed herein, and the results of those models discussed herein, are in excellent agreement with previous functional and structural studies and can provide unique insight on how mutations interact to shape the binding affinity of antibodies. The pervasiveness of epistatic effects also highlights the need for AI models to accurately predict and guide antibody optimization.

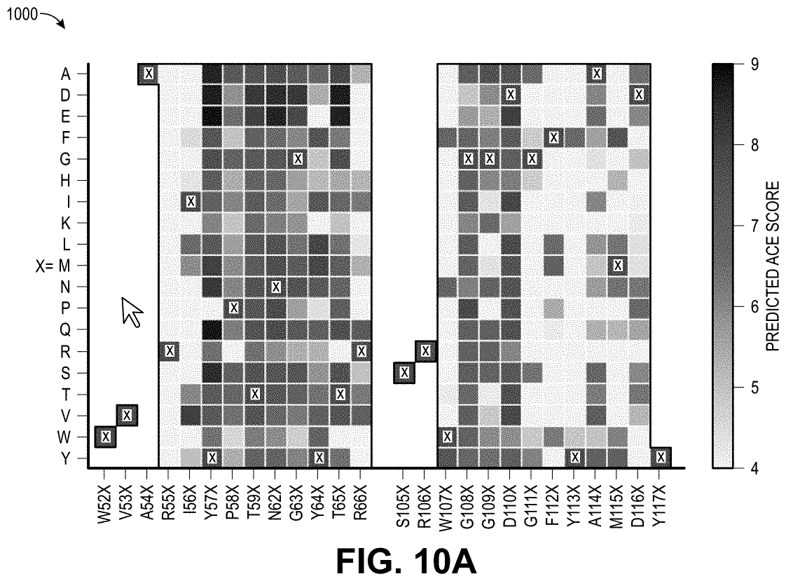

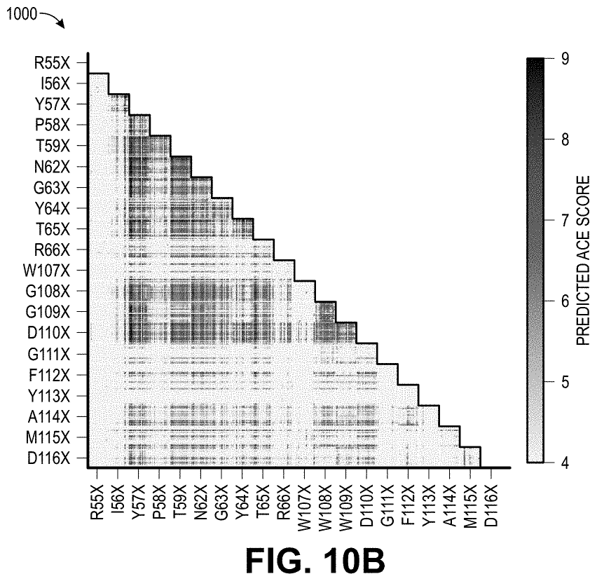

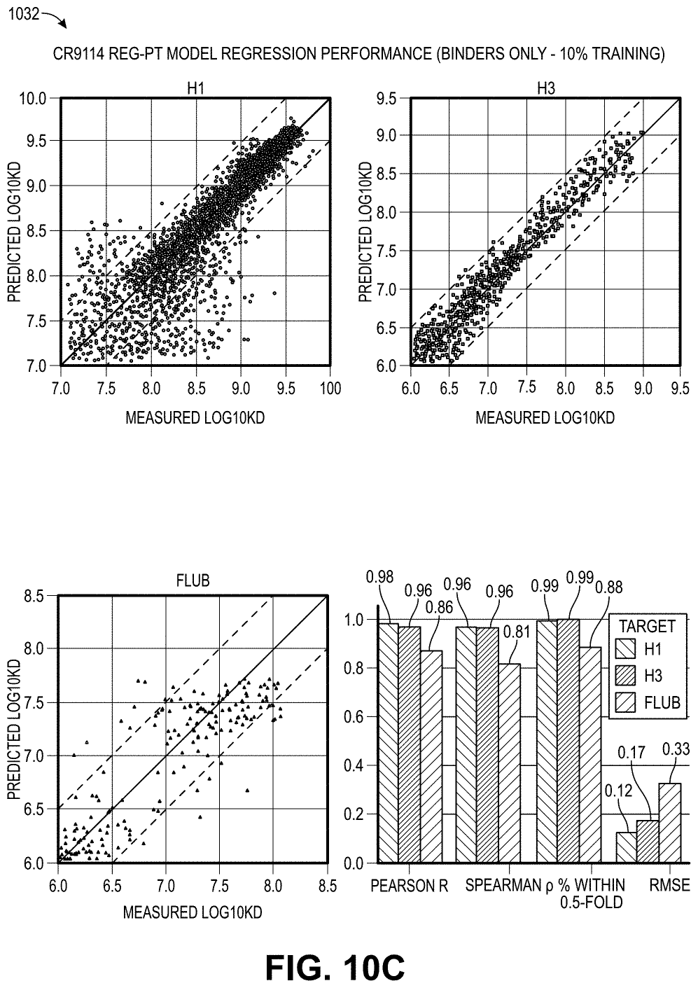

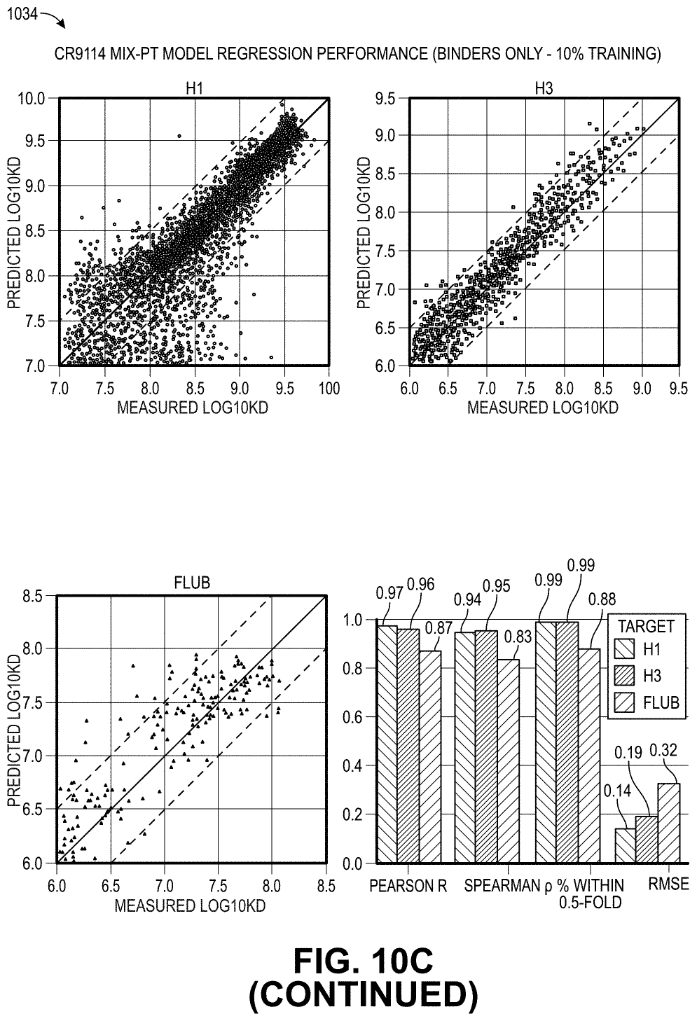

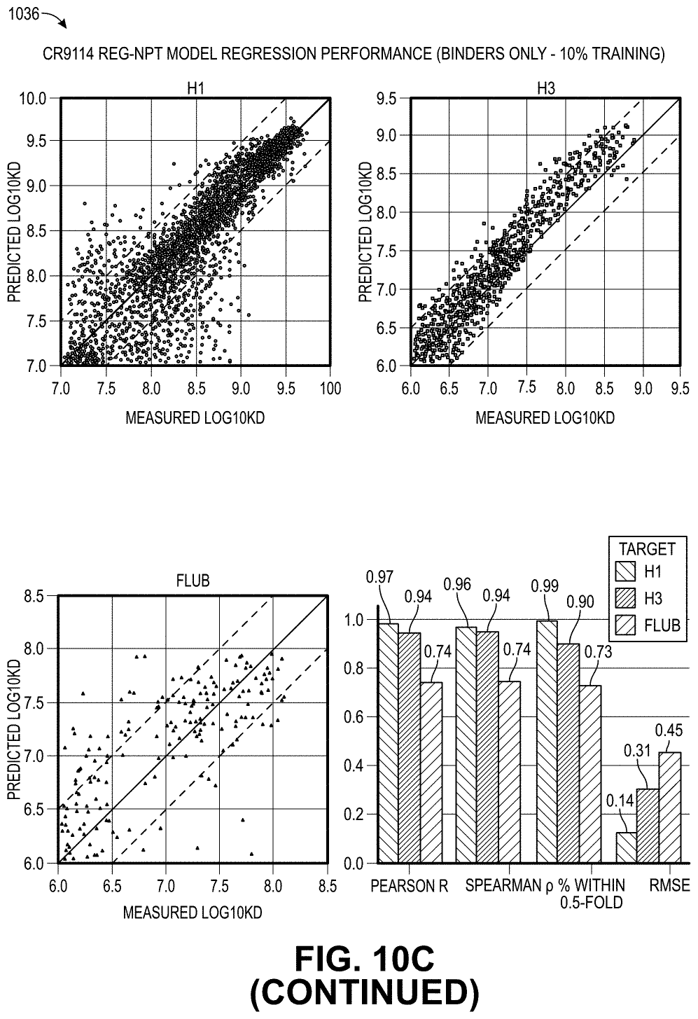

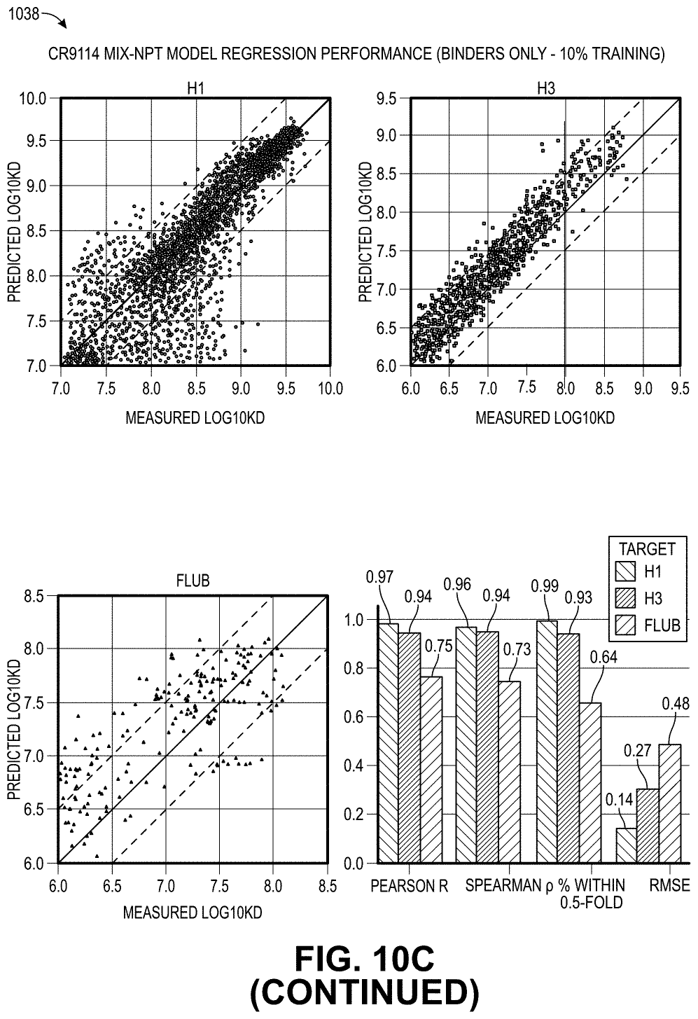

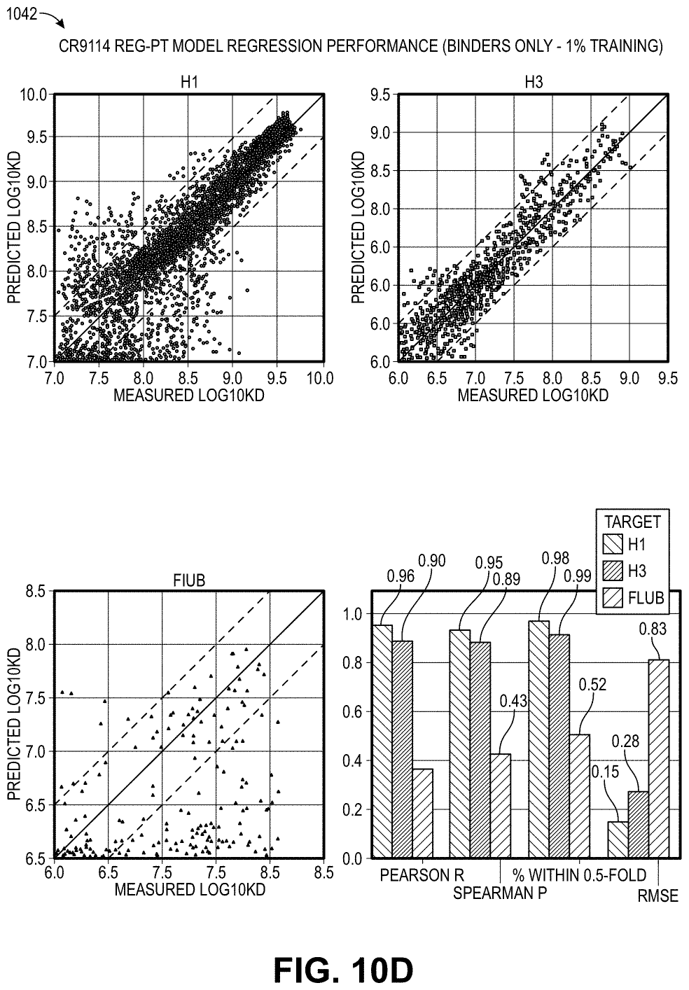

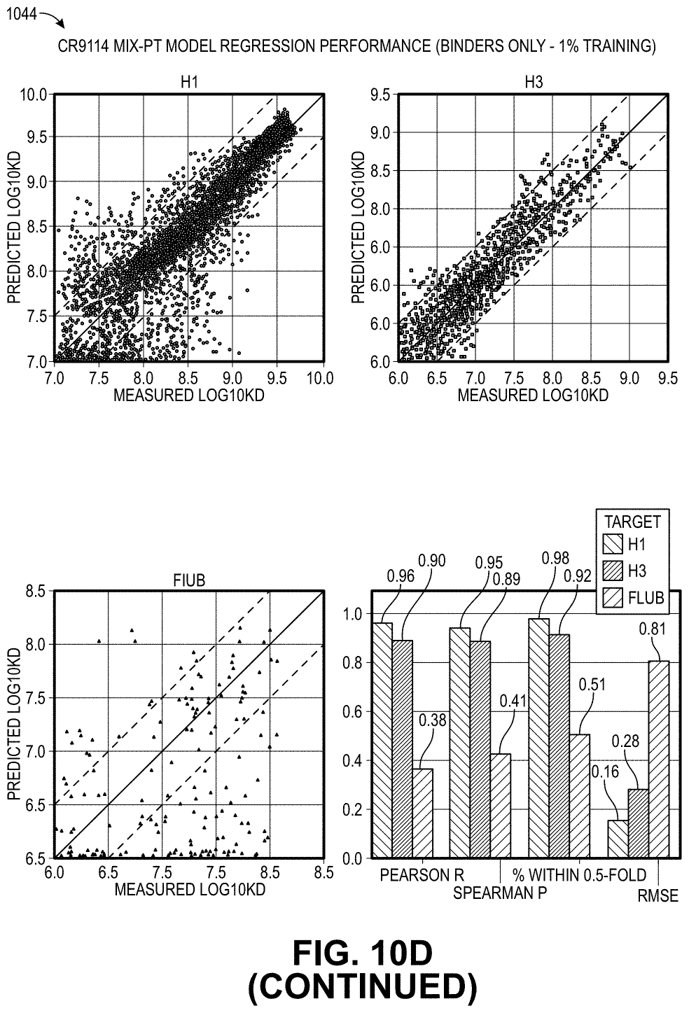

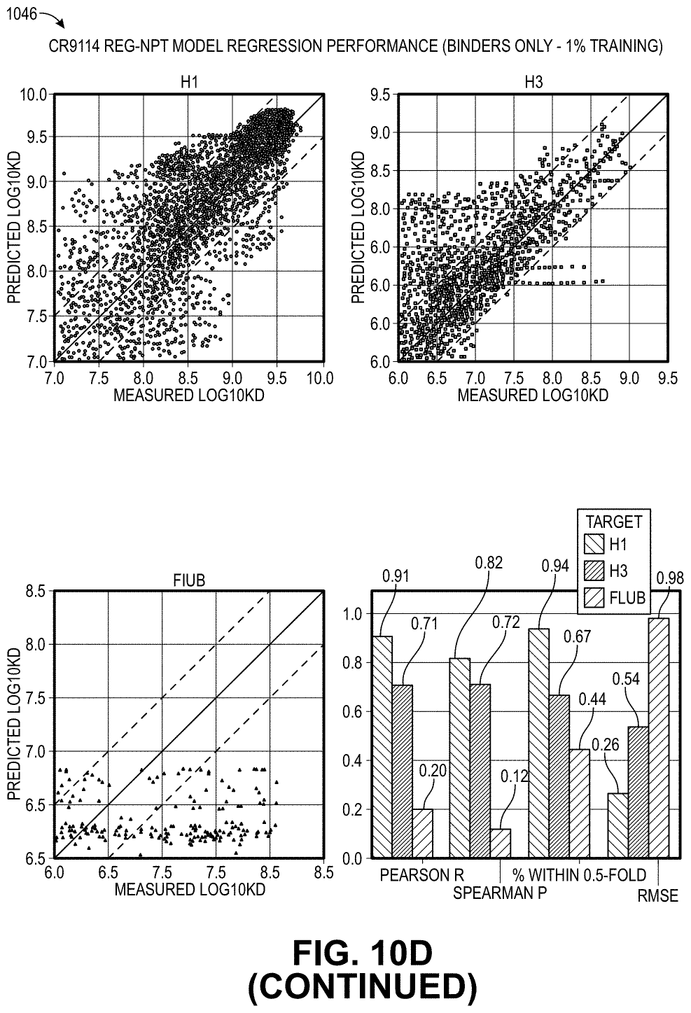

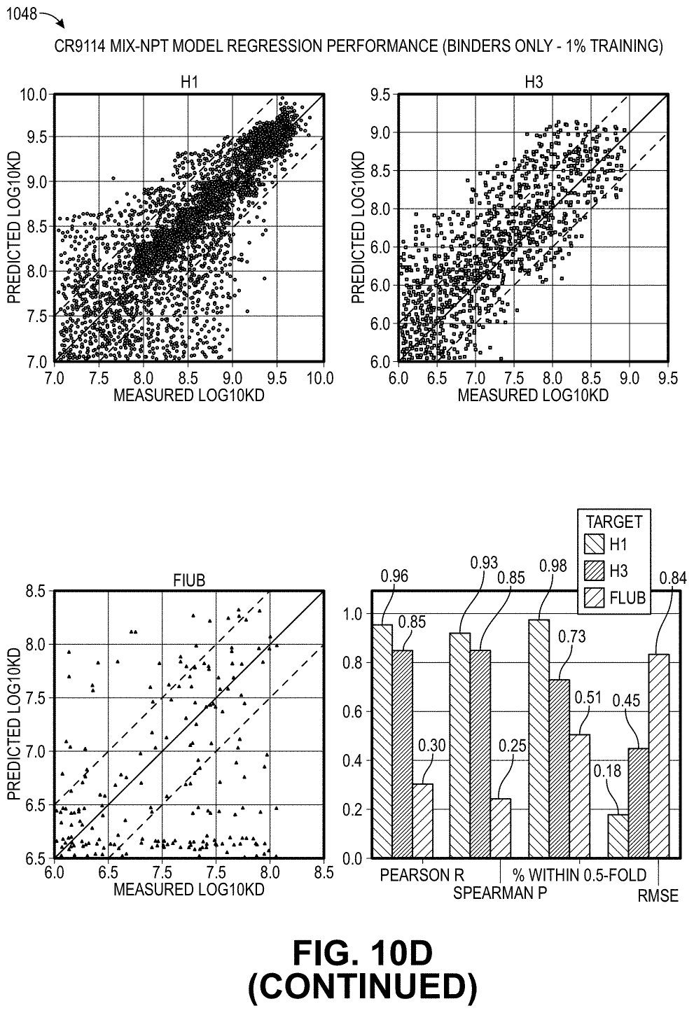

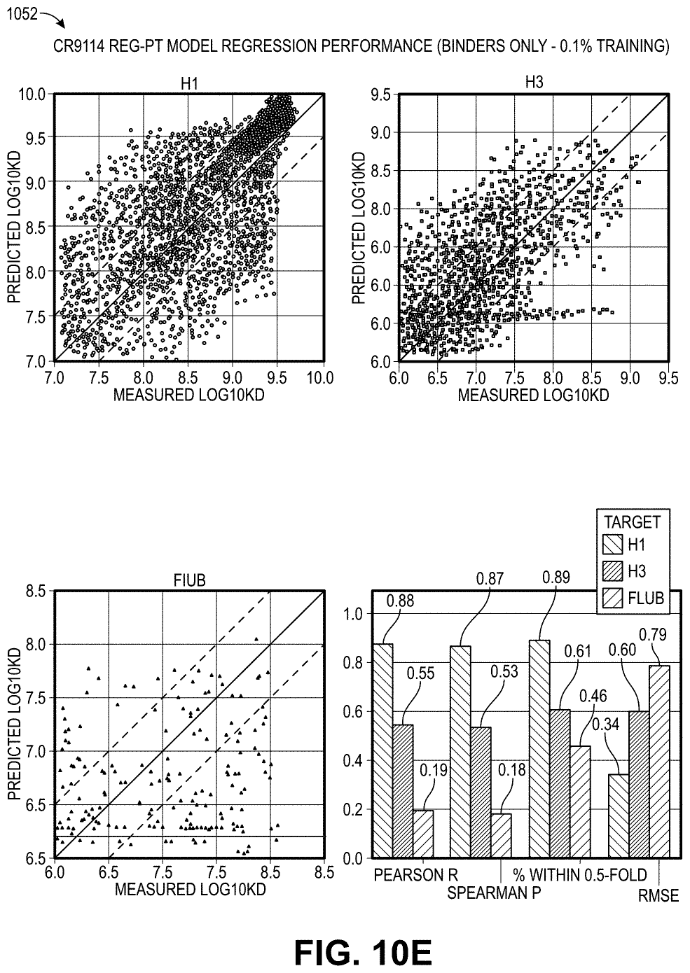

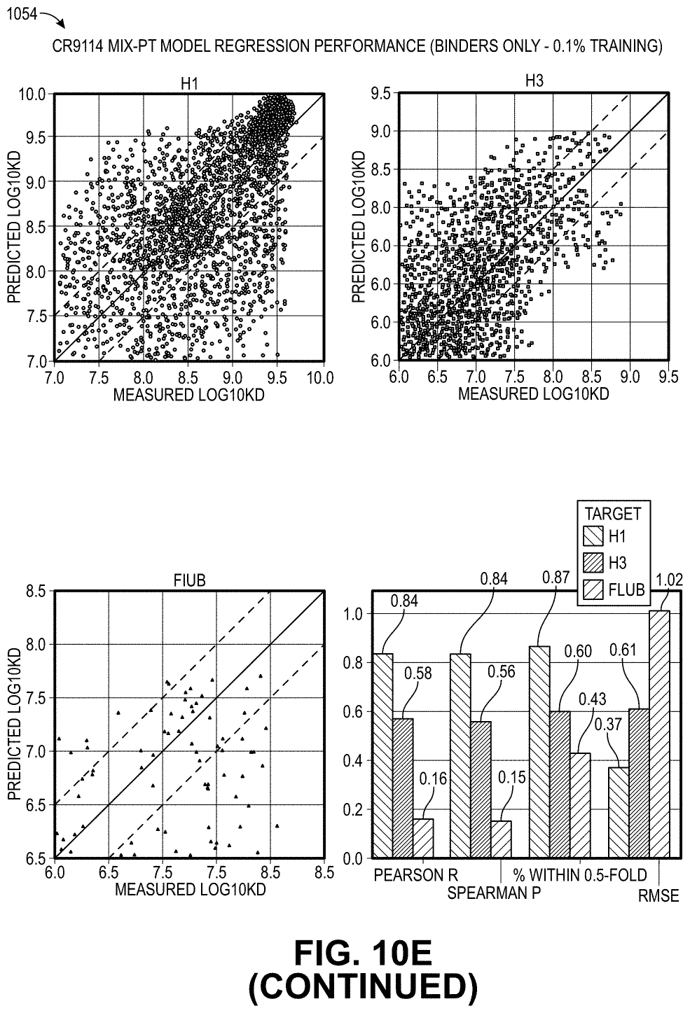

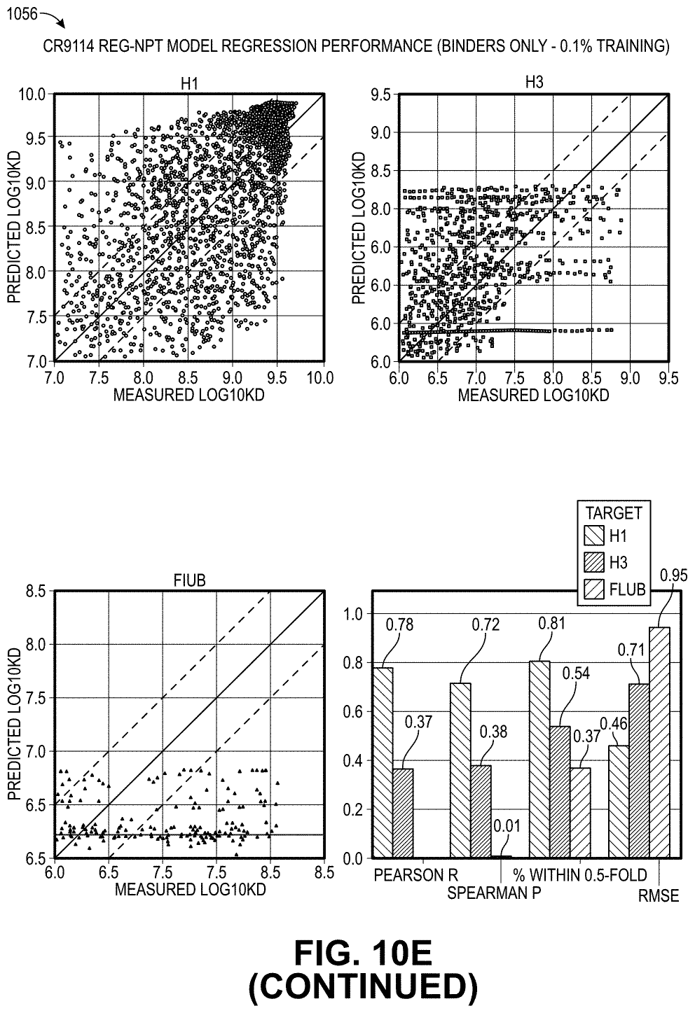

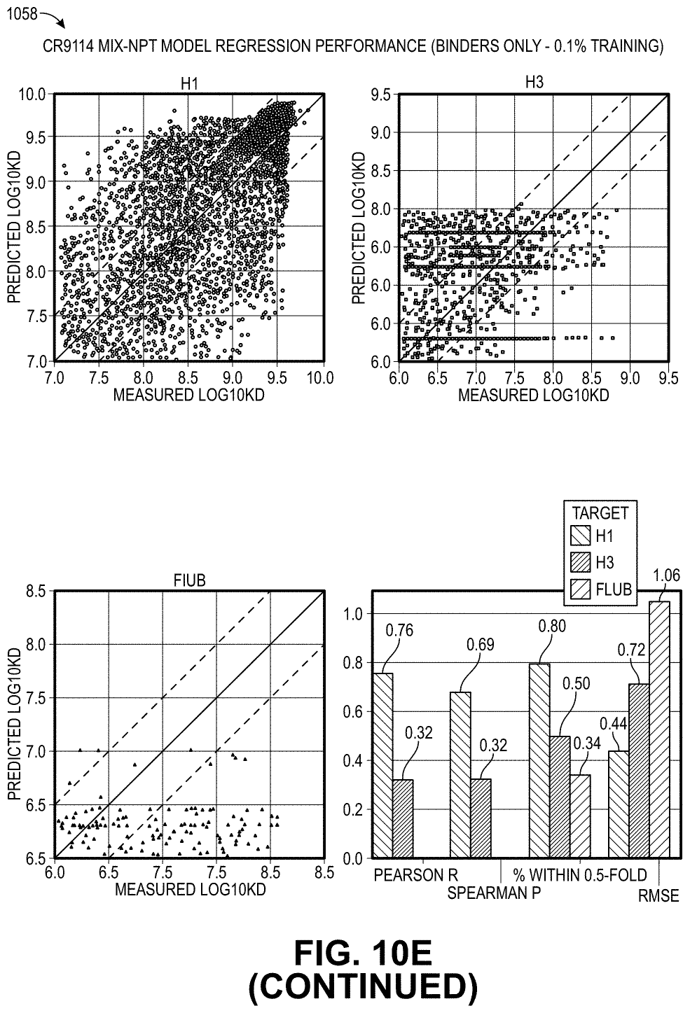

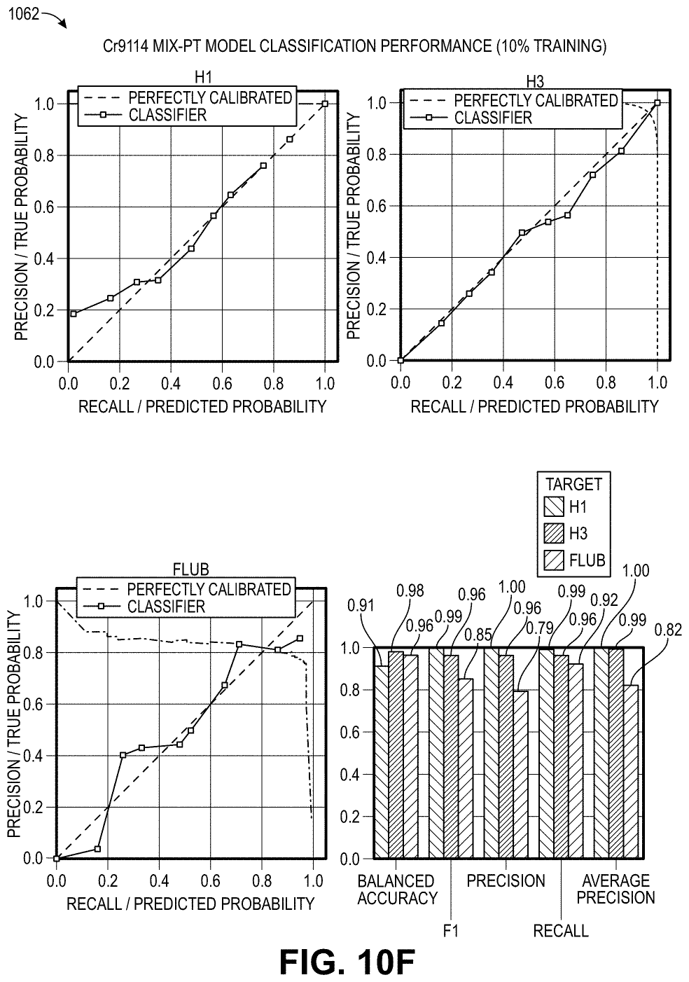

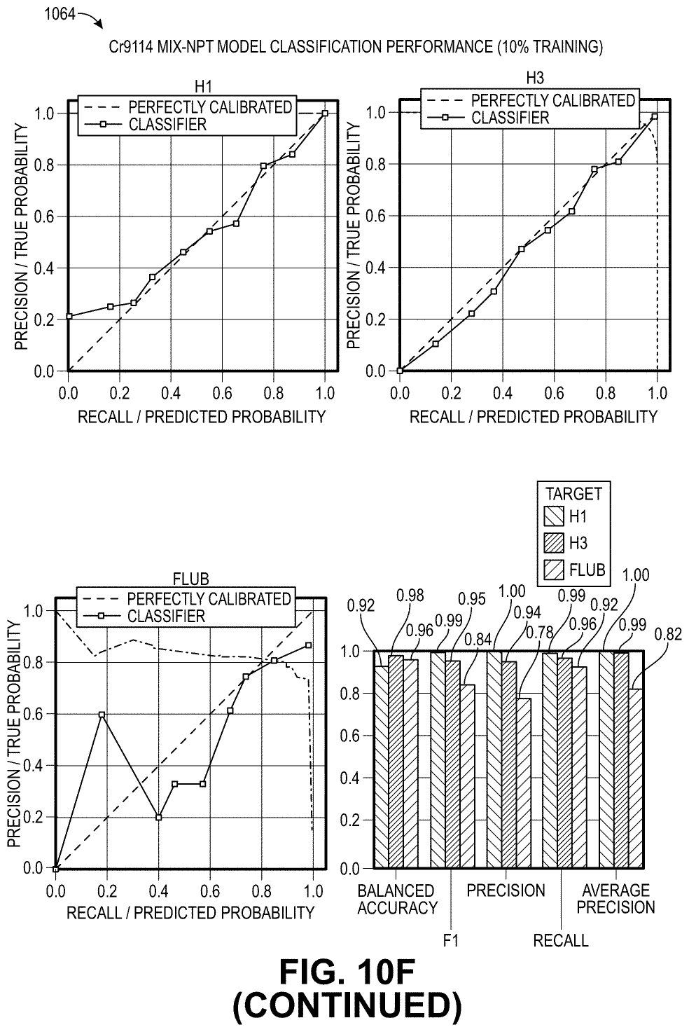

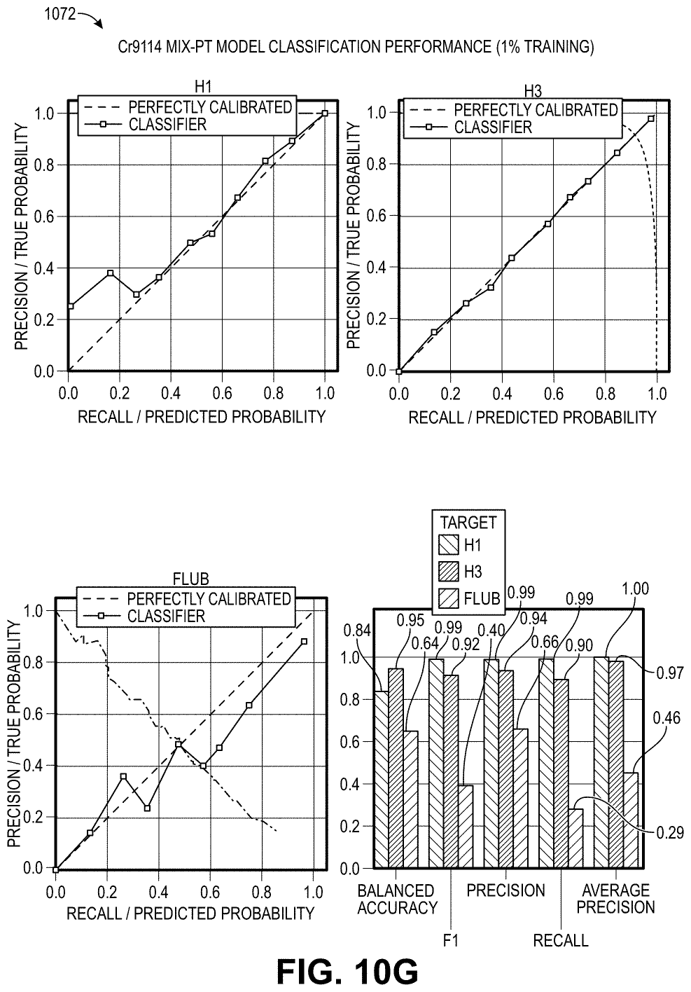

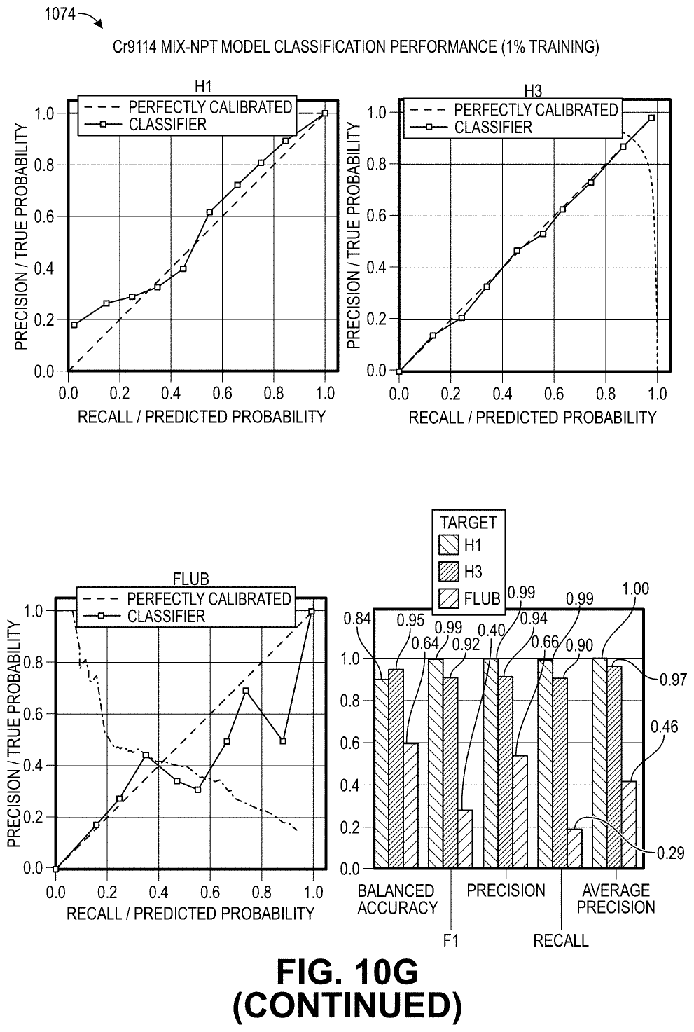

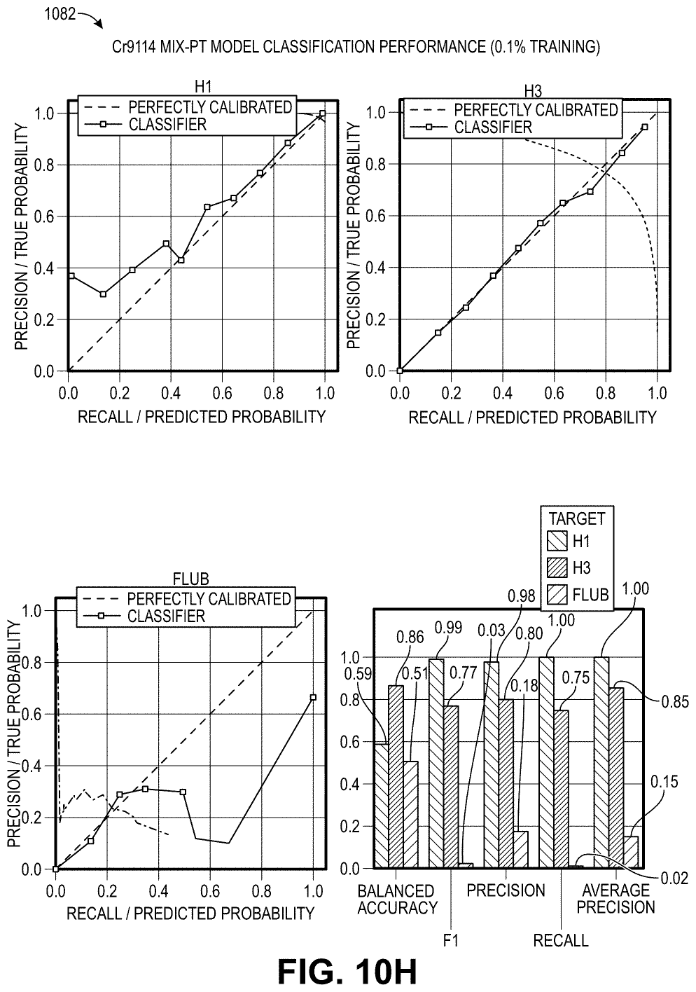

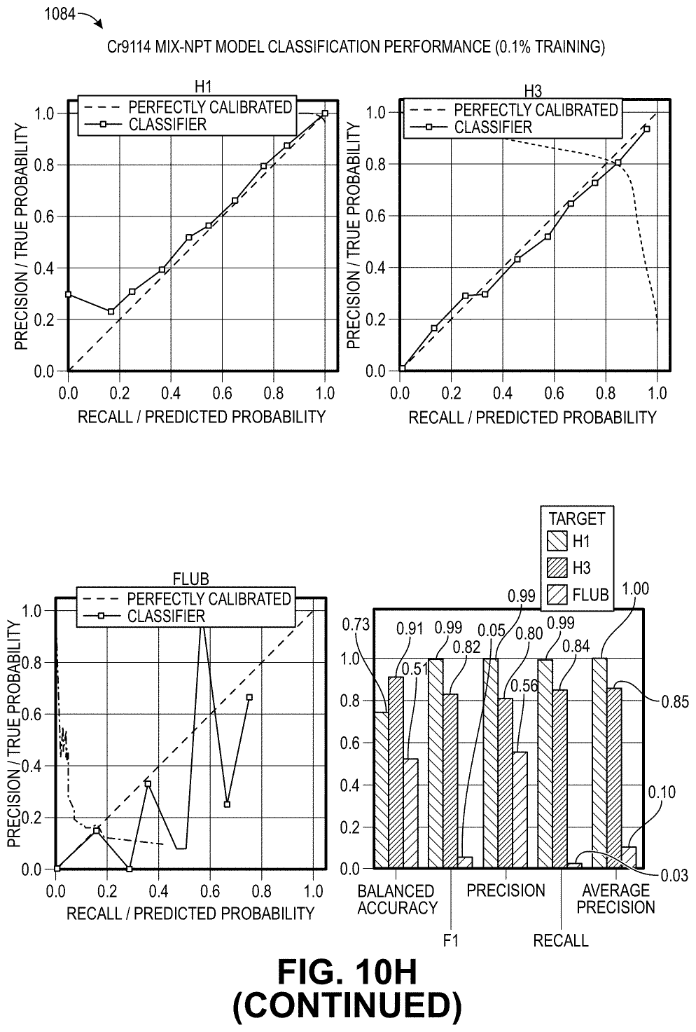

FIG. 10A and FIG. 10B depict global sequence-affinity mapping of trastuzumab variants, according to aspects of the present techniques.

### AI Shows Strong Predictive Performance on a Second Case Study Involving Simultaneous Binding Predictions for Three Antigen Variants

The modeling approach discussed above established with trastuzumab can be readily extended to other antibodies. To demonstrate, in an example, public binding data of variants of the broadly neutralizing(bn)Ab CR9114 may be used (see Supplementary Information, infra) [27]. Since the CR9114 dataset provides binding data for three different influenza subtypes of the target antigen hemagglutinin (HA), the present techniques may be used to extend the model to support multi-task affinity predictions to multiple targets simultaneously. Further, the present techniques may be used to explore the ability of the model to combine classification and regression in a single mixture model, since many of the CR9114 variants lost binding to one or more HA subtypes. And still further, the present techniques may be used to evaluate the impact of the training set size on the model's performance.

| | | Classification | | | Regression | | | | | |
|---|---|---|---|---|---|---|---|---|---|---|
| Training | | Balanced Accuracy | | | RMSE | | | % w/i 0.5-fold | | |
| Size | Model | H1 | H3 | FluB | H1 | H3 | FluB | H1 | H3 | FluB |
| 6509 | Reg-PT | NA | NA | NA | 0.12 | 0.17 | 0.33 | 99% | 99% | 88% |
| (10%) | Mix-PT | 0.91 | 0.98 | 0.96 | 0.14 | 0.19 | 0.32 | 99% | 99% | 88% |
| | Reg-NPT | NA | NA | NA | 0.14 | 0.31 | 0.45 | 99% | 90% | 73% |
| | Mix-NPT | 0.92 | 0.98 | 0.96 | 0.14 | 0.27 | 0.48 | 99% | 93% | 64% |
| 651 | Reg-PT | NA | NA | NA | 0.15 | 0.28 | 0.83 | 98% | 93% | 52% |
| (1%) | Mix-PT | 0.84 | 0.95 | 0.64 | 0.16 | 0.28 | 0.81 | 98% | 92% | 51% |
| | Reg-NPT | NA | NA | NA | 0.26 | 0.54 | 0.98 | 94% | 67% | 44% |
| | Mix-NPT | 0.90 | 0.94 | 0.59 | 0.18 | 0.45 | 0.84 | 98% | 73% | 51% |
| 65 | Reg-PT | NA | NA | NA | 0.34 | 0.60 | 0.79 | 89% | 61% | 46% |
| (0.1%) | Mix-PT | 0.59 | 0.86 | 0.51 | 0.37 | 0.61 | 1.02 | 87% | 60% | 43% |
| | Reg-NPT | NA | NA | NA | 0.46 | 0.71 | 0.95 | 81% | 54% | 37% |
| | Mix-NPT | 0.73 | 0.91 | 0.51 | 0.44 | 0.72 | 1.06 | 80% | 50% | 34% |

The above performance table depicts Joint model affinity prediction performance for CR9114 on multiple influenza strains of the hemagglutinin (HA) antigen. For each training set size (10%, 1%, 0.1% of 65,091) four models were trained (Reg: Regression only model; Mix: Mixture classification/regression model; PT: initialized with pre-trained OAS-model weights; NPT: initialized with random weights). Results are shown for these models using pooled CV. The full CR9114 dataset includes 63,419 (97%) H1, 7,174 (11%) H3, and 198 (0.3%) FluB positive binders.

The above table, and FIGS. 10C-10H, together demonstrate that a single model may be trained using the present techniques to jointly predict affinities of a given antibody sequence against multiple distinct antigen targets.

FIG. 10C depicts regression performance of models trained with 10% of the CR 9114 dataset, according to some aspects. FIG. 10C includes results 1032 of a regression only model, results 1034 of a mixture classification/regression model, results 1036 of a model initialized with pre-trained OAS-model weights and results 1038 initialized with random weights.

FIG. 10D depicts regression performance of models trained with 1% of the CR 9114 dataset, according to some aspects. FIG. 10D includes results 1042 of a regression only model, results 1044 of a mixture classification/regression model, results 1046 of a model initialized with pre-trained OAS-model weights, and results 1048 initialized with random weights.

FIG. 10E depicts regression performance of models trained with 0.1% of the CR 9114 dataset, according to some aspects. FIG. 10E includes results 1052 of a regression only model, results 1054 of a mixture classification/regression model, results 1056 of a model initialized with pre-trained OAS-model weights, and results 1058 initialized with random weights.

FIG. 10C-10E depict results for those models using pooled CV only for positive binders (−log₁₀ K_{D}>B₆ where B_{c} is the lower boundary for each target as determined in the original publication; 7 for H1, and 6 for H3 and FluB). The full CR 9114 dataset includes 63,419 (97%) H1; 7,174 (11%) H3 and 198 (0.3%) FluB positive binders.

FIG. 10F depicts regression performance of mixture models trained with 10% of the CR 9114, according to some aspects. FIG. 10F includes results 1062 of a model initialized with pre-trained OAS-model weights and results 1064 of a model initialized with random weights.

FIG. 10G depicts regression performance of mixture models trained with 1% of the CR 9114, according to some aspects. FIG. 10G includes results 1072 of a model initialized with pre-trained OAS-model weights and results 1074 of a model initialized with random weights.

FIG. 10H depicts regression performance of mixture models trained with 0.1% of the CR 9114, according to some aspects. FIG. 10H includes results 1082 of a model initialized with pre-trained OAS-model weights and results 1084 of a model initialized with random weights.

FIGS. 10F-10H depict results for these models using pooled CV. For each model and target, FIGS. 10F-10H each depict a respective precision-recall curve plot and calibration curve (true probability vs. predicted probability at different scoring bins).

As expected, the predictive power of such a model was lower for the FluB target compared to H1 and H3, since the full dataset contains only 193 positive FluB binders. This left only 19 positive examples when using a training set of 10% and only 1-2 positive examples in training sets of 1% and 0.1% (a minimum of one positive and negative example for each target was required when selecting the cross-validation folds, see Supplementary Information, infra). Nevertheless, even with as little as 19 training examples, 88% of the model's predictions for FluB were within 0.5 log of their measured values when using initial weights pre-trained on the OAS dataset, compared to only 73% when using random initial weights. Using pre-trained weights improved performance in all cases where the number of training examples was below 1,000.

The mixture model was able to perform well on the classification tasks without significant loss of performance on the regression tasks compared to the regression only model. The balanced accuracy of the model's predictions was above 0.84 in all cases where the training set contained at least 7 positive and 7 negative examples, achieving a 0.91 balanced accuracy score on the H3 binding task even with training sets of only 65 variants (7 positive and 58 negative variants on average).

### Naturalness

In general, the development of a candidate antibody into a therapeutic drug is a complex process with a high degree of pre-clinical and clinical risk. This risk is often due to numerous challenges related to production, formulation, efficacy, and adverse reactions. Modeling these risks has been a tremendous challenge for the industry due to the difficulty in obtaining informative and relevant data, particularly at scale.

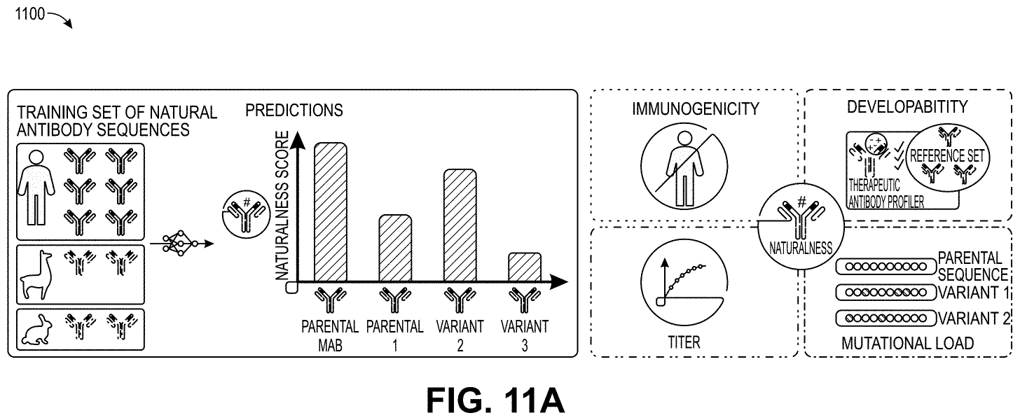

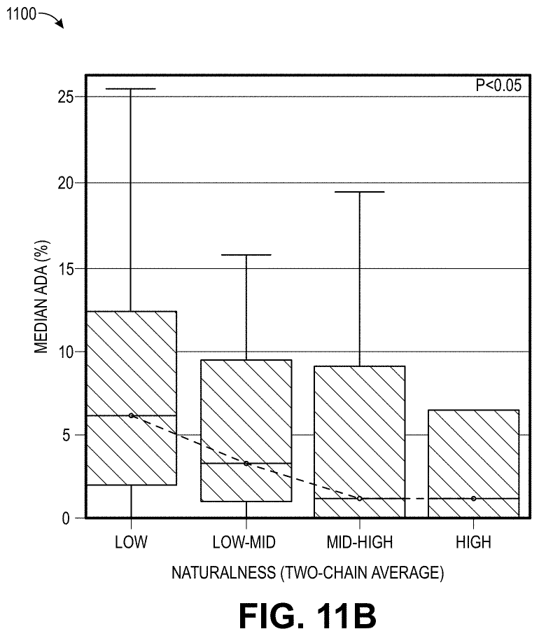

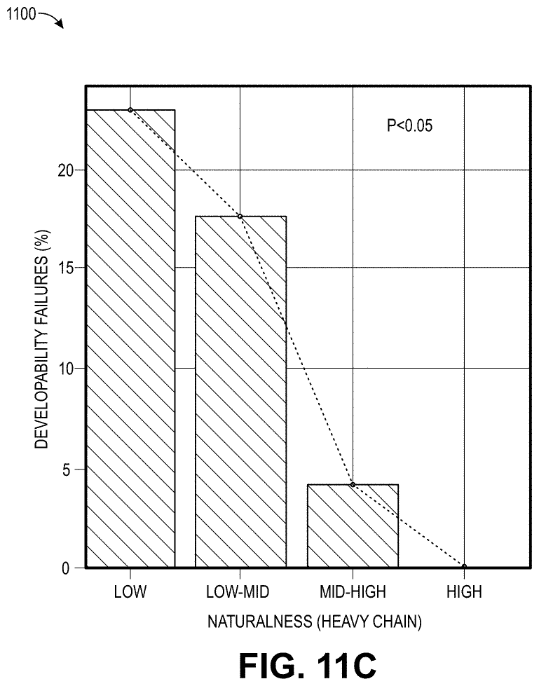

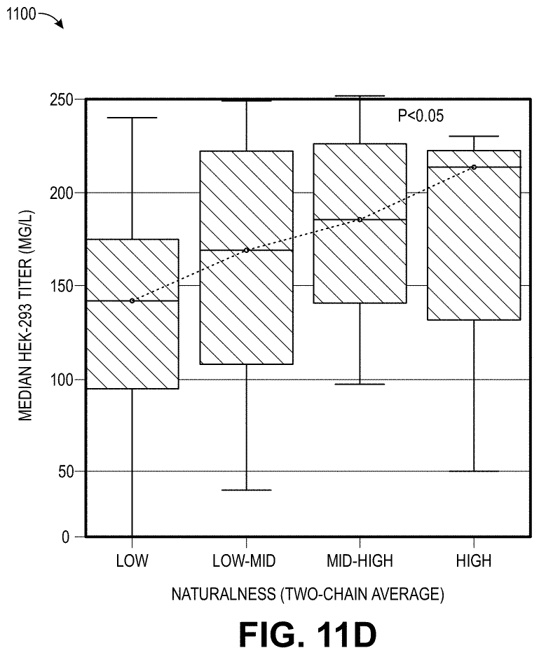

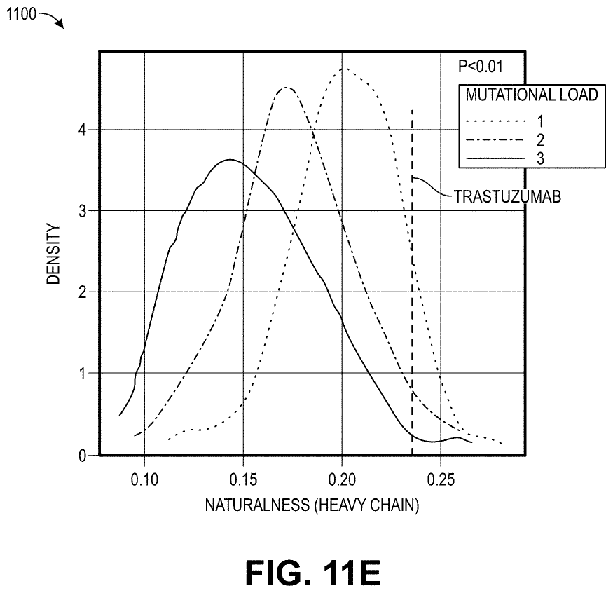

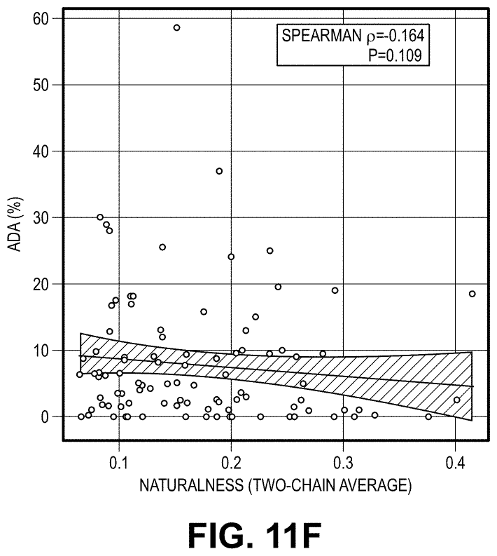

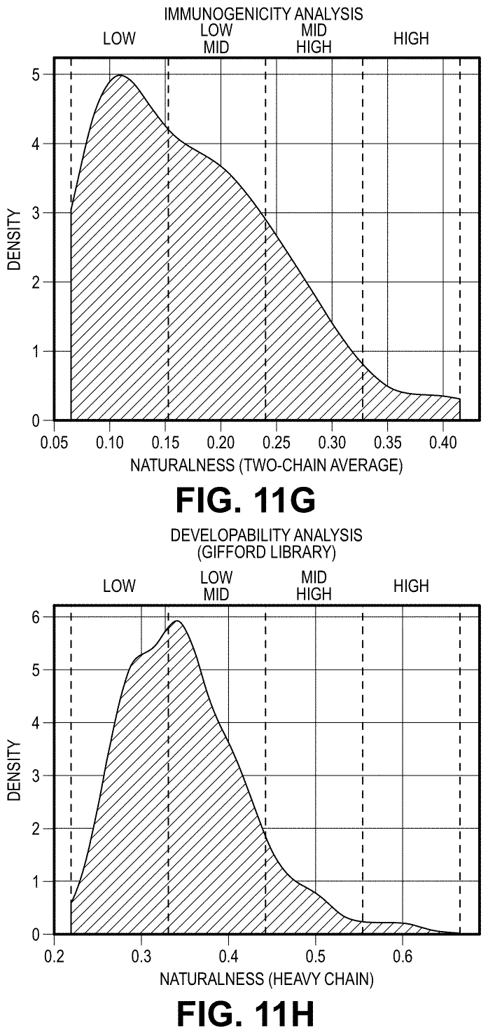

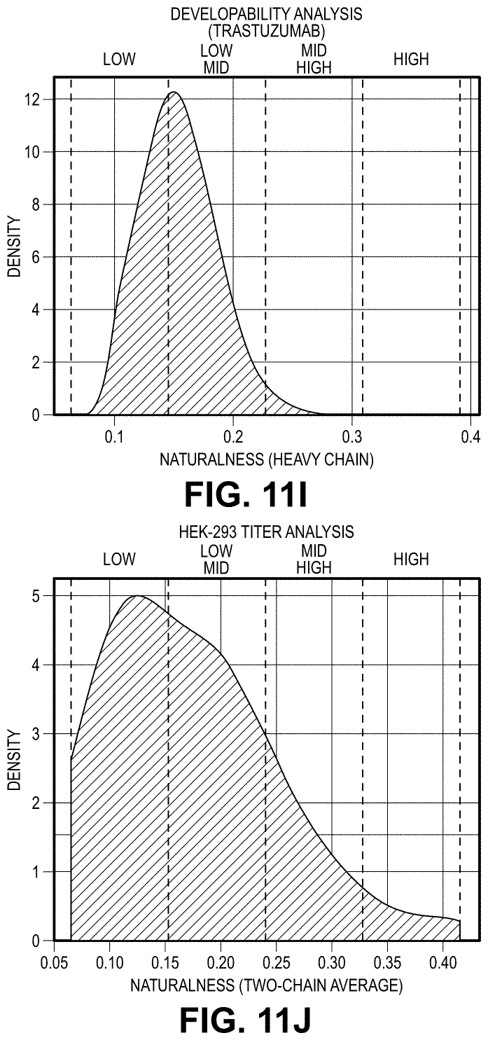

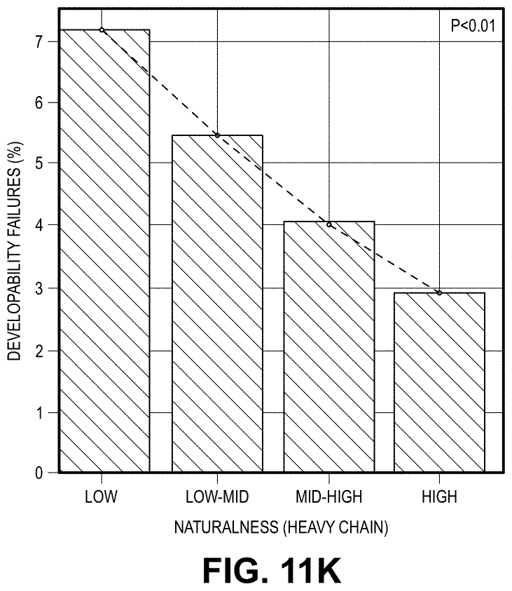

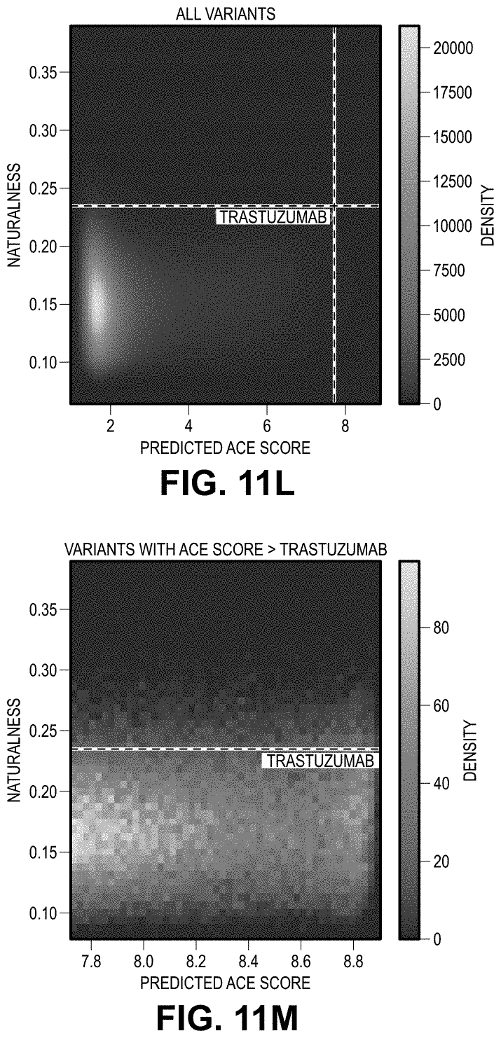

FIG. 11 depicts associations between antibody naturalness, immunogenicity, developability and other properties. In some examples, it was hypothesized that using the present techniques to learn sequence patterns across natural antibodies from different species could be useful to identify and prioritize “human-like” antibody variants, disregarding unnatural sequences and ultimately mitigating drug development risks, as depicted in FIG. 11. To this aim, in an example, language models pre-trained on OAS may be used to evaluate antibody sequences for their naturalness score (see Materials and Methods, infra). In this context, naturalness is a score computed by pre-trained language models that measures how likely it is for a given antibody sequence to be derived from an organism of interest. Thus, naturalness might be used as a guiding metric towards antibody design and engineering.

In FIGS. 11B-11D, the four bins (Low, Low-Mid, Mid-High, High) may correspond to dividing the naturalness range into four parts of equal size (see also FIGS. 11G-11J). P-values may be computed using the Jonckheere-Terpstra test for trends. Datasets in FIGS. 11B and 11D were scored for both chains, whereas datasets in panels 11C and 11E comprised only heavy-chain variants and were consistently scored only by heavy-chain models. To determine the usefulness of naturalness scores, their association with four antibody properties was evaluated. For example, the property of immunogenicity is reflected in FIG. 11B, which was consolidated across numerous studies on clinical-stage antibodies by Marks et al. [28]. A potential confounding factor in a naturalness-immunogenicity association analysis is that some antibodies have a fully human origin, while others are humanized, chimeric or murine.

Scoring antibodies of different origin by naturalness would amount to binning them primarily by species, which is trivial and uninformative. By contrast, scoring antibodies belonging to the same class would amount to genuinely ranking from most natural to least natural. The only two antibody classes in Marks et al. large enough to support a statistical analysis are human and humanized antibodies. In an example, the latter was investigated using the present techniques, because their immunogenicity potential is greater, thereby providing an ideal case study.

A scatterplot between the fraction of patients positive for Anti-Drug Antibodies (ADA) and naturalness reveals a weak, non-significant correlation, as shown in FIG. 11F. However, closer inspection of ADA responses showed that most data points are in the 0-10% range, with a few outliers above 20%. Empirical study and reason suggests that such outliers could blur the relationship between naturalness and immunogenicity, if any. To mitigate the impact of outliers, the present techniques were used to bin naturalness scores and computed the median ADA responses per naturalness bin, as shown in FIG. 11G, for example. As in FIGS. 11B-11D, the ranges in FIGS. 11G-11J (Low, Low-Mid, Mid-High, High) may correspond to dividing the naturalness range into four parts of equal size.

This analysis revealed that the most natural antibodies trigger lower median ADA responses than the least natural antibodies, as depicted in FIG. 11B.

The second antibody property the present techniques were used to consider is developability, which can be estimated with the Therapeutic Antibody Profiler (TAP) [31]. The present techniques were used to compute naturalness scores (with the modeling techniques discussed herein) and developability scores (with TAP) for the heavy-chain sequences from a high-diversity phage display dataset [29] (“Gifford Library”, FIG. 11H) as well as lower-diversity trastuzumab variants (FIG. 11I). For both, empirical review found a strong association between naturalness and TAP-derived developability, as shown in FIGS. 11C and 11K. This result is particularly impressive because naturalness scores were obtained upon training exclusively with examples of naturally occurring antibody sequences, while TAP was calibrated using distributions of five metrics collected from therapeutic antibodies [31]. Therefore, the association between naturalness and TAP-assessed developability suggests that developable antibodies are enriched in human-like antibodies. FIG. 11K depicts the association between naturalness and developability failures of trastuzumab variants with respect to FIG. 11I.

The third property the present techniques were used to investigate is expression levels in mammalian (HEK-293) cells, which have been reported for clinical-stage antibodies by Jain et al. [30]. As for immunogenicity, the dataset comprises several classes of antibodies and the present techniques were used to again focus on humanized antibodies. Empirical results suggested that highly natural antibodies expressed better than antibodies scored less favorably by the modeling techniques used herein, as depicted in FIG. 7D and FIG. 11J.

Finally, the fourth property the present techniques were used to consider is mutational load, which measures the number of amino acid substitutions between a parental antibody sequence and a variant.

For example, the present techniques were used to computed naturalness scores for 6,710,400 single-, double-, and triple-mutant trastuzumab variants, depicted in FIG. 11E. Empirical study of the results found that naturalness was negatively associated with mutational load. This finding is consistent with the common notion that most mutations have detrimental effects and highlights the need to actively optimize naturalness alongside affinity, because introducing mutations into a parental antibody without consideration for naturalness is likely to degrade naturalness.

Sequence Variant Generation with Desired Properties

Generally, antibody optimization can be performed to a limited extent for individual properties using a number of established laboratory approaches. For example, deep mutational scanning has been used to improve the binding affinity of antibody candidates [5]. However, large mutational spaces cannot be exhaustively screened by these methods, limiting the scope of potential improvements. Library screening methods, such as phage display, can overcome this obstacle, but a consequence of selecting for a single property at a time (such as binding) may be the unintended degradation of other properties of interest. For example, the present techniques were used to show that increasing the mutational load results in lower median naturalness, as depicted in FIG. 11D.

To further demonstrate this, the present techniques were used to exhaustively predict the qaACE score and naturalness of all variants with up to three mutations from trastuzumab. Of the 6.7 million variants, just 46,931 (0.7%) had predicted qaACE scores higher than trastuzumab, as shown in FIG. 11L. Even if one were able to experimentally screen for all variants with improved affinity, only 4,003 (8.5%) of these variants had a naturalness score on par or higher than trastuzumab, as shown in FIG. 11M. The dashed lines in FIG. 11L and FIG. 11M indicate the naturalness and/or predicted ACE score of trastuzumab, in the density maps of the fitness landscapes for the exhaustive trast-3 search space.

Randomly screening this space using the approximately 50,000-member trast-3 library yielded only 60 variants with higher qaACE scores and naturalness.

In silico screening provides a way to address this issue by optimizing for multiple properties simultaneously with a designer objective function. The present techniques may include a genetic algorithm (GA) situated on top of the present affinity and naturalness model oracle, that was capable of greatly improving the throughput of the in silico screening process.

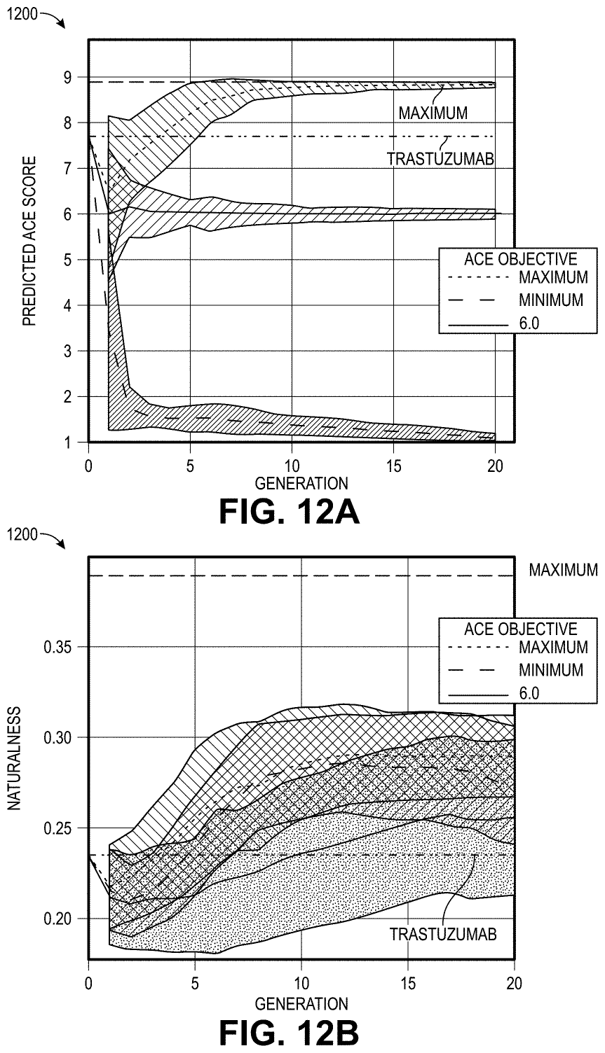

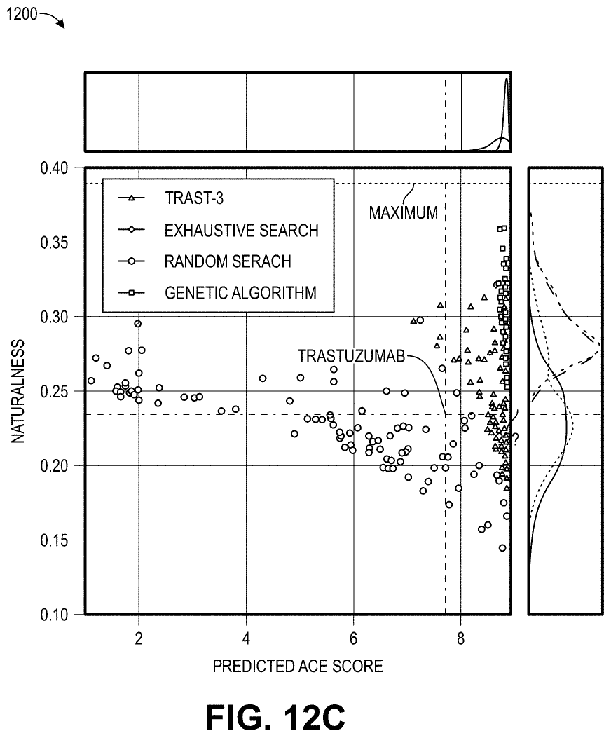

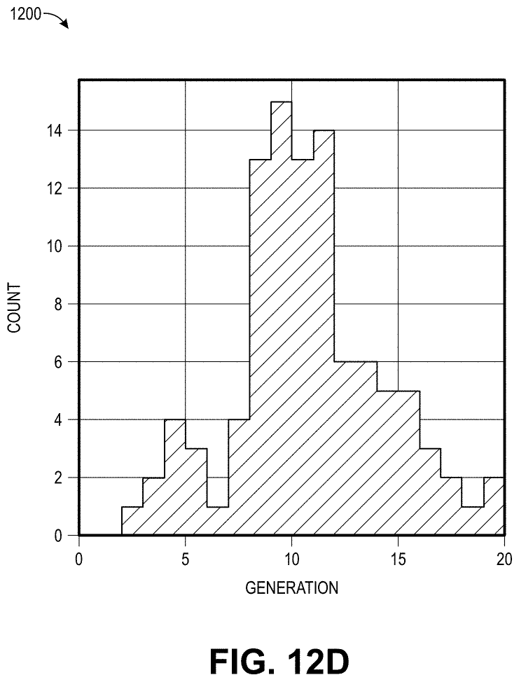

FIG. 12 depicts, generally, that a genetic algorithm can efficiently maximize, minimize, or target specific qaACE scores while maximizing naturalness. As an example, the present techniques may be used to minimize, maximize, or target specific qaACE scores in a search space of over 6.7 million 419 sequence variants—as depicted in FIG. 12A—while simultaneously maximizing naturalness, as shown in FIG. 12B.

In this example, after 20 generations, the GA performed nearly as well as an exhaustive search of the mutational space, as shown in FIG. 12C: 85 of the top 100 variants identified by the GA were among the top 100 variants overall. In addition, all of the top variants identified by the GA were within 5% of the maximum achievable qaACE score (9, resulting from 9 sorting gates) and had 424 higher naturalness scores than trastuzumab. As a baseline, the present techniques may be used to perform a random search by querying the same number of sequences as the GA. In this example, this search was only able to find two sequences with higher qaACE score and naturalness than trastuzumab, as depicted in FIG. 12C.

Unlike an exhaustive search of the mutation space, GA-driven optimization is highly efficient. For example, in each generation, the GA samples 200 new variants, resulting in only 4,000 total sequences sampled across all 20 generations. In addition, over half of the top 100 individuals were selected by the GA in the first 12 generations, as depicted in FIG. 12D. Altogether, these results show that a genetic algorithm built on top of predictive models for binding affinity and naturalness can quickly and efficiently identify a set of top candidates for downstream development. The value of optimization techniques coupled with AI oracles will increase as in silico design is applied to increasingly larger combinatorial sequence spaces.

### Discussion

Generally, deep learning methods have demonstrated rapid progress for the modeling proteins, including sequence, structure, and function. Likewise, protein interactions are receiving increased attention for the purposes of therapeutic design. A key limitation for many of these efforts is the ability to synthesize large libraries of proteins and assess their quantitative attributes. Here we demonstrate that the qaACE assay discussed herein is a powerful complement to deep learning models, providing the throughput and fidelity to accurately model antibody binding affinity with up to four/five mutations in two CDRs (combinatorial space: 108-1010) from a single experiment. The qaACE assay provides advantages over existing methods for large scale antibody variant interrogation such as Tite-Seq [32], SORTCERY [33] and Phage Display [34]. First, qaACE utilizes SoluPro™ E. coli B Strain to solubly express antibodies intracellulariy, avoiding binding artifacts associated with surface display format. Additionally, qaACE leverages genetic tools available for E. coli, enabling faster library generation cycles and increased transformation efficiency compared to other model organisms. Finally, the qaACE assay is a true screening method where all variants are measured regardless of affinity strength, as opposed to selections, such as phage display, where only high affinity binders are preferentially isolated.

The predictive ability of the present deep learning models demonstrated here is possible with the quantitative capability of the improved qaACE assay, which provides two distinct advantages from a modeling standpoint. The first is the expanded capabilities of models trained on quantitative data for overall increased performance and quantitative predictions, which are particularly useful when the goal is to tune the binding affinity rather than simply maximize it. Secondly, quantitative training data also allows for the intelligent selection of sequences for downstream quantification with the lower-throughput gold-standard SPR assay. The random sequence space is enormous and heavily skewed toward deleterious mutations. A common approach to this problem is to bias the mutational library towards specific locations or key mutations, but the strength of epistatic effects identified by these models suggest these approaches provide insufficient coverage. Our pre-quantification step with the improved qaACE assay gives us the opportunity to measure a more uniform distribution without bias, which increases the generalization power of the models.

The diversity of the high-affinity sequences available and their dilution with the mutational load highlights the value of exhaustively and accurately evaluating the space of possible variants. Such large-scale evaluation is only feasible with the help of computational models. The modeling results are in excellent agreement with previous functional and structural studies and can provide unique insights on how different mutations interact to shape the binding affinity landscape of antibodies. The pervasiveness of epistatic effects also highlights the need for highly flexible AI models to accurately predict and guide antibody optimization.

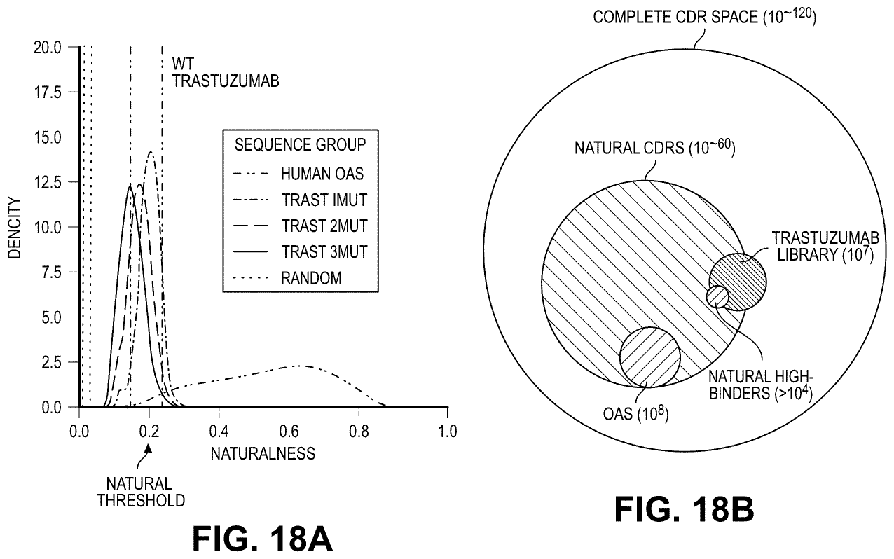

Only a very small fraction of sequences within the enormous combinatoric antibody sequence space have been detected in nature—108 in the OAS database versus more than 10120 possible unique CDR sequences for the longest reported human sequences. The naturalness model presented here can help determine whether a novel sequence belongs to this category, and the present techniques may be used to roughly estimate the size of this natural space as 1060, as shown in FIG. 18A and FIG. 18B.

FIGS. 18A and 18B depict plots and diagrams related to estimating sequence space sizes for heavy-chain human CDRs. For example, in FIG. 18A, one million random sequences may be generated using random amino acids matching a length distribution of OAS. Based on the lower tail of OAs naturalness, a threshold of 0.15 may be chosen for estimating the size of the natural sequence space. In FIG. 18B, circles are not to scale. The size of the total possible sequence space may be estimated from 20 amino acid possibilities across 61 positions (the longest human sequence ni the filtered OAS dataset). The natural CDR space may be roughly approximated by fitting a skew-normal distribution to the random sequences and calculating the fraction that exceeds the naturalness threshold.

While the statistical uncertainty in this calculation is considerable, it is clear that the natural space is much larger than can possibly be screened in a lab or in silico. At the same time, these natural sequences are vanishingly rare in screens of random sequence variants.

The solution presented here is to apply models trained on both naturalness and affinity data targeted to a specific antibody, the intersection of which effectively allows evaluation of a larger whitespace of sequences than can be physically assessed, while also focusing screening on the most relevant ‘natural’ sequences.

The present co-optimization of two antibody properties could be extended to co-optimization of n antibody properties. Training models on multiple affinity datasets unlocks binding predictions for multiple antigens or antigen variants, as was shown here for CR9114. In principle, multi-antigen predictions could facilitate engineering of breadth (co-optimization for antigen escape variants), specificity (co-optimization to reduce binding to undesired members of a protein family, while increasing binding to desired members of the same family) and species cross-reactivity (co-optimization for human and cynomolgus orthologs), just to name a few.

Above, it was demonstrated that pre-training on natural sequences improved the predictive performance of the present models. Likewise, the models can continue to improve with the addition of new data, both with respect to new antibodies and with the addition of new performance or developability attributes. The present techniques may be used to show that naturalness is a useful surrogate of common metrics, and may outperform these associations in practice if the intuitive relationship between naturalness and favorable properties holds. Additional properties could be added alongside data from their respective assays, such as conditional pH binding, effector function, melting temperature, self-aggregation, viscosity and more. For most of these, a single model trained on a strong dataset could serve for diverse antibodies of interest and even improve the power of the binding affinity models through multi-task training.

Importantly, the framework presented herein facilitates tuning an antibody feature toward a desired value, not necessarily limited to selecting for variants at the extremes of a given range. Moreover, while the models presented herein are focused on antibody optimization for target affinity and naturalness features, the approach could in principle be applied to tuning any protein's interaction with its target.

While AI-assisted optimization of biological sequences serves the practical use of reducing therapeutic development time, it does not by itself offer a fully in silico replacement. To this end, fully generative modeling approaches are needed. However, their training and validation faces an even greater data challenge, since the full de novo combinatorial space considered without the anchor of the parental sequence is dramatically larger, and strong selective binders are an infinitesimally small slice of that space.

Structure-based approaches are showing increasing capabilities and may be useful for bridging this gap. The language models presented here offer the possibility of serving as in silico oracles, within their extrapolation range, which can provide generative models with an effective training ground. The synthesis of optimization and full generation may be the next big step in data-driven therapeutic design.

### Materials and Methods

### Libraries

### Cloning

Antibody variants were cloned and expressed in Fab format. To produce qaACE and SPR datasets meant for model training and evaluation (table 1), DNA variants were synthesized spanning CDRH2 and CDRH3 in a single oligonucleotide using ssDNA oligo pools (Twist Bioscience). Codons were randomly selected from the two most common in E. coli B strain [35] for each variant. Two synonymous DNA sequences were synthesized (5 or 10 for parental trastuzumab and positive/negative controls) for each amino acid variant. Amplification of Twist Bioscience ssDNA oligo pools was carried out by PCR according to Twist Bioscience's recommendations with the exception that Platinum SuperFi II DNA polymerase (ThermoFisher) was used in place of KAPA polymerase. Briefly, 20 μL reactions consisted of 1× Platinum SuperFi II Mastermix, 0.3 μM each of forward and reverse primers, and 10 ng oligo pool. Reactions were initially denatured for 3 min at 95° C., followed by 13 cycles of: 95° C. for 20 s; 66° C. for 20 s; 72° C. for 15 s; and a final extension of 72° C. for 1 min. DNA amplification was confirmed by agarose gel electrophoresis, and amplified DNA was subsequently purified (DNA Clean and Concentrate Kit, Zymo Research).

To build libraries meant for SPR validation of model designs in independent experiments, oligonucleotides (59 nt) spanning CDRH3 and the immediate pstream/downstream flanking nucleotides were synthesized by Integrated DNA Technologies (IDT). Codon usage was identical for all variants, except at mutated positions. Olignoucleotides were pooled such that each oligonucleotide was represented in an equimolar fashion within the pool. This single stranded oligonucleotide pool was used directly in cloning reactions (see below) without prior amplification.

To generate linearized vector, a two-step PCR was carried out to split Absci's plasmid vector carrying fab format trastuzumab into two fragments in a manner that provided cloning overlaps of approximately 30 nucleotides (nt) on the 5′ and 3′ ends of the amplified Twist Bioscience libraries, or 18 nt on the 5′ and 3′ ends of IDT oligonucleotides. Vector linearization reactions were digested with DPN1 (New England Bioloabs) and purified from a 0.8% agarose gel (Gel DNA Recovery Kit, Zymo Research) to eliminate parental vector carry through. Cloning reactions consisted of 50 fmol of each purified vector fragment, either 100 fmol purified library (Twist Bioscience) or 10 pmol (IDT) insert, and 1× final concentration NEBuilder HiFi DNA Assembly (New England Biolabs). Reactions were incubated at 50° C. for either two hours (Twist Bioscience libraries) or 25 min (IDT library), and subsequently purified (DNA Clean and Concentrate Kit, Zymo Research). Transformax Epi300 (Lucigen) E. coli were transformed by electroporation (BioRad MicroPulser) with the purified assembly reactions and grown overnight at 30° C. on LB agar plates containing 50 μg/ml kanamycin. The following morning colonies were scraped from LB plates and plasmids were extracted (Plasmid Midi Kit, Zymo Research) and submitted for QC sequencing.

### QC

Antibody variant libraries were amplified by PCR across the CDRH2 and CDRH3 region and sequenced with 2×150 nt reads using the Illumina NextSeq 1000 P2 platform with 20% PhiX. The PCR reaction used 10 nM primer concentration, Q5 2× master mix (NEB) and 1 ng of input DNA diluted in MGH20. Reactions were initially denatured at 98° C. for 3 min; followed by 30 cycles of 98° C. for 10 s, 59° C. for 30 s, 72° C. for 15 s; with a final extension of 72° C. for 2 min.

Sequencing results were analyzed for distribution of mutations, variant representation, library complexity and recovery of expected sequences. Metrics included coefficient of variation of sequence representation, read share of top 1% most prevalent sequences and percentage of designed library sequences observed within the library.

### Activity-Specific Cell-Enrichment (ACE/qaACE) Assay

Antibody Expression In SoluPro™ E. coli B Strain

SoluPro™ E. coli B strain was transformed by electroporation (Bio-Rad MicroPulser). Cells were allowed to recover in 1 ml SOC medium for 90 min at 30° C. with 250 rpm shaking. Recovery outgrowths were centrifuged for 5 min at 8,000 g and the supernatant was removed. Resultant cell pellets were resuspended in 1 ml of induction media (IBM) (4.5 g/L Potassium Phosphate 547 monobasic, 13.8 g/L Ammonium Sulfate, 20.5 g/L yeast extract, 20.5 g/L glycerol, 1.95 g/L Citric Acid) containing inducers and supplements (260 μM Arabinose, 50 μg/mL Kanamycin, 8 mM Magnesium Sulfate, 1 mM Propionate, 1× Korz trace metals) and then added to 100 ml IBM containing inducers and supplements in a 1 L baffled flask. Antibody Fab induction was allowed to proceed at 30° C. with 250 rpm shaking for 24 h. At the end of 24 h, 1 ml aliquots of the induced culture were adjusted to 25% v/v glycerol and stored at −80° C.

### Cell Preparation

High-throughput quantitative selection of antigen-specific Fab-expressing cells was adapted from the approach described in Liu et al. [20]. For staining, an OD600=2 of thawed glycerol stocks from induced cultures were transferred to 0.7 ml matrix tubes, centrifuged at 3300 g for 3 min, and resulting pelleted cells were washed three times with PBS+1 mM EDTA. Washed cells were thoroughly resuspended in 250 μL of 33 mM phosphate buffer (Na2HPO4) by pipetting then fixed by the further addition of 250 μL 32 mM phosphate buffer with 0.5% paraformaldehyde and 0.4% glutaraldehyde. After 40 min incubation on ice, cells were washed three times with PBS, resuspended in lysozyme buffer (20 mM Tris, 50 mM glucose, 10 mM EDTA, 5 μg/ml lysozyme) and incubated for 8 min on ice. Fixed and lysozyme-treated cells were equilibrated in stain buffer by washing 3× in 0.1% saponin buffer (1×PBS, 1 mM EDTA, 0.1% saponin, 1% heat-inactivated FBS).

### Staining

Prior to library staining, the Her2 probe was titrated against the reference strain to determine the 75% effective concentration (EC75). After lysozyme treatment and equilibration, the trast-1 library was resuspended in 250 μL saponin buffer and transferred to a new matrix tube. The trast-3 library was incubated for 20 min in AlphaLISA immunoassay assay buffer (Perkin Elmer; 25 mM HEPES, 0.1% casein, 1 mg/ml dextran—500, 0.5% Triton X-100, and 0.5% kathon) for additional permeabilization prior to equilibration and resuspension in saponin buffer. A 2× concentration of stain reagents—100 nM human HER2:AF647 (Acro Biosystems) and 60 nM anti-kappa light chain:AF488 (BioLegend)—was prepared in saponin buffer, then 250 μL probe solution was transferred to the prepared cells bringing the total stain volume to 500 μL with 50 nM Her2 and 30 nM anti-kappa LC. Libraries were incubated with probe overnight (16 h) with end to end rotation at 4° C. protected from light. After incubation, cells were pelleted, washed 3× with PBS, and then resuspended in 500 μL PBS by thorough pipetting.

### Sorting Libraries

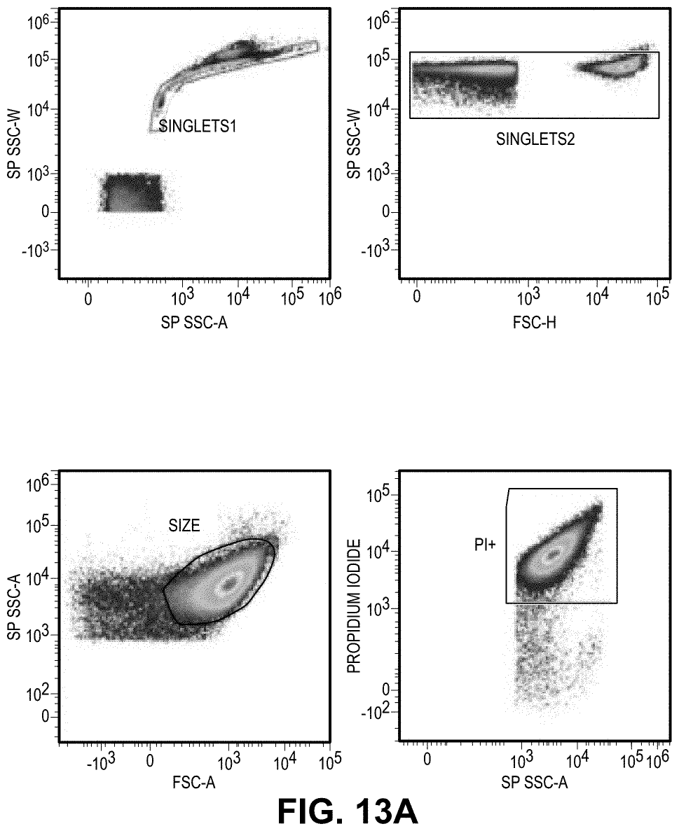

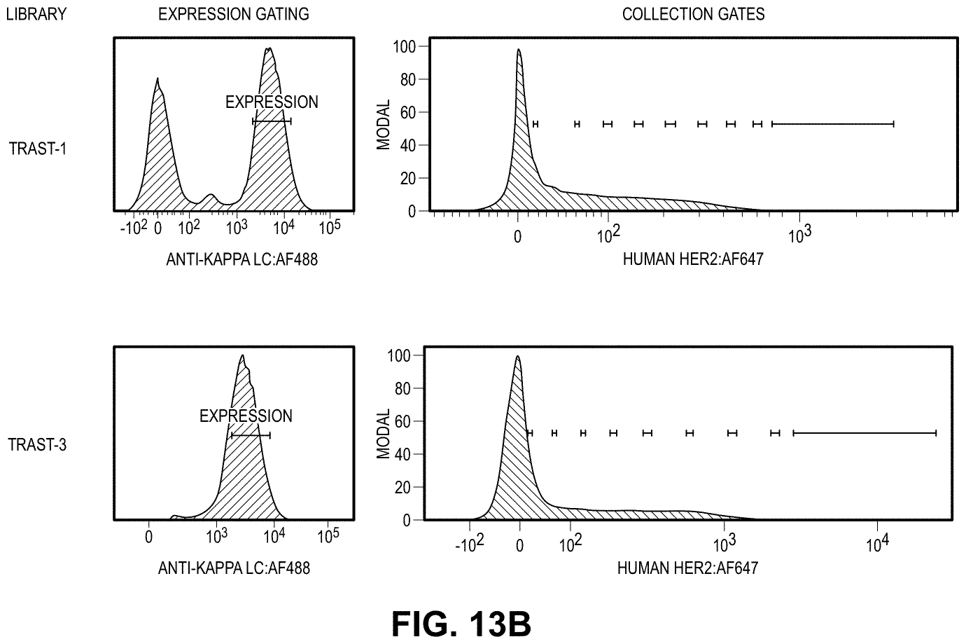

Sorting libraries were sorted on FACSymphony S6 (BD Biosciences) instruments. Immediately prior to sorting, 50 μL prepped sample was transferred to a flow tube containing 1 mL PBS+3 μL propidium iodide. Aggregates, debris, and impermeable cells were removed with singlets, size, and PI+parent gating. To reduce expression bias, an additional parent gate was set on the mid 65% of peak expression positive cells. Collection gates were drawn to evenly sample the log range of binding signal. The far right gate was set to collect the brightest 10,000 events over the allotted sort time, estimated by including the 5 brightest events for every 65,000 in the expression parent gate. Seven additional gates were then set to fractionate the positive binding signal, and one gate collected the binding negative population, as shown in FIGS. 13A and FIG. 13B.

Specifically, FIG. 13A depicts a representative parent gating for all ACE sorts, according to some aspects. The two singlets gates were drawn to exclude SoluPro™ aggregate regions previously identified by dual fluorescence of GFP and mCherry reporter strains, and propidium iodide was used to exclude unpermeabilized cells. FIG. 13B depicts a specific expression and collecting gating for each ACE library sort, according to some aspects. A parent gate containing approximately 65% of the expression positive cells and centered over the peak expression signal was drawn prior to setting collection gates on the probe-specific binding signal.

Libraries were sorted simultaneously on two instruments with photomultipliers adjusted to normalize fluorescence intensity, and the collected events were processed independently as technical replicates.

### Next-Generation Sequencing

Cell material from various gates was collected in a diluted PBS mixture (VWR), in 1.5 mL tubes (Eppendorf). Post sort samples were spun down at 3,800 g and tube volume was normalized to 20 μl. Amplicons for sequencing were generated from one of two methods. The first method amplifies the CDRH2 and CDRH3 region via a two-phase PCR, using collected cell material directly as template. During the initial PCR phase, unique molecular identifiers (UMIs) and partial Illumina adapters were added to the CDRH2 and CDRH3 amplicon via 4 PCR cycles. The second phase PCR added the remaining portion of the Illumina sequencing adapter and the Illumina i5 and i7 sample indices. The initial PCR reaction used 1 nm UMI primer concentration, Q5 2× master mix (NEB) and 20 μl of sorted cell material input suspended in diluted PBS (VWR). Reactions were initially denatured at 98° C. for 3 min, followed by 4 cycles of 98° C. for 10 s; 59° C. for 30 s; 72° C. for 30 s; with a final extension of 72° C. for 2 min. Following the initial PCR, 0.5 μM of the secondary sample index primers were added to each reaction tube. Reactions were then denatured at 98° C. for 3 min, followed by 29 cycles of 98° C. for 10 s; 62° C. for 30 s; 72° C. for 15 s; with a final extension of 72° C. for 2 min. The second method amplifies the CDRH2 and CDRH3 region without the addition of UMIs. This single phase PCR used 10 nM primer concentration, Q5 2× master mix (NEB) and 20 μl of sorted cell material input suspended in diluted PBS (VWR). Reactions were initially denatured at 98° C. for 3 min, followed by 30 cycles of 98° C. for 10 s; 59° C. for 30 s; 72° C. for 15 s; with a final extension of 72° C. for 2 min. After amplification by either method samples were run on a 2% agarose gel at 75 V for 60 min and the proper length band was excised and purified using the Zymoclean Gel DNA Recovery Kit (Zymo Research). Resulting DNA samples were quantified by Qubit fluorometer (Invitrogen), normalized and pooled. Pool size was verified via Tapestation 1000 HS and was sequenced on an Illumina NextSeq 1000 P2 (2×150 nt) with 20% PhiX.

### ACE Assay Analysis

In order to produce quantitative binding scores from reads, the following processing and quality control steps were performed:
1. Paired-end reads were merged using FLASH2 [36] with the maximum allowed overlap set according to the amplicon size and sequencing reads length (150 bases for all the libraries described herein).
2. If UMIs were added during amplification, the downstream UMI tag (last 8 bases) was moved to the beginning of the read, and the UMI Collapse tool [37] was used in FASTQ mode to remove any PCR duplicates. Only fully identical sequences were considered to be duplicates and error correction was not performed at this stage.
3. Primers were removed from both ends of the merged read using cutadapt tool [38], and reads were discarded where primers were not detected.
4. Reads were aggregated across all FACS sorting gates and aligned to the reference sequence (parental version of the amplicon) in amino acid space. Alignment was performed using the Needleman-Wunsch algorithm implemented in Biopython [39], with the following parameters: PairwiseAligner, mode=global, match_score=5, mismatch_score=−4, open_gap_score=−20, extend_gap_score=−1. Parameters were chosen by manual inspection across a number of processed libraries.
5. Reads were then discarded if (1) the mean base quality was below 20, or (2) a sequence (in DNA space) was seen in fewer than 10 reads across all gates (or in less than 10 unique molecules following UMI deduplication, when available).
6. The procedure also flagged: (1) sequences that align to the reference with a low score (defined as less then 0.6 of the score obtained by aligning the reference to itself); (2) sequences containing stop codons outside of the region of interest and (3) sequences containing frame-shifting insertions or deletions. Flagged sequences were not included in any mutation-related statistics, but were used for count normalization for binding score calculations. FastQC [40] and MultiQC [41] were used to generate sequencing quality control metrics.
7. For each gate, the prevalence of each sequence (read/UMI count relative to the total number of reads/UMIs from all sequences in that gate) was normalized to 1 million 672 counts.
8. The binding score (ACE score) was assigned to each unique DNA sequence by taking a weighted average of the normalized counts across the sorting gates. For all experiments, weights were assigned linearly using an integer scale: the gate capturing the lowest fluorescence signal was assigned a weight of 1, the next lowest gate was assigned a weight of 2, etc.
9. Any detected sequence which was not present in the originally designed and 679 synthesized library was dropped.
10. For each unique amino acid variant, qaACE scores from synonymous DNA sequences were averaged.
11. qaACE scores were averaged across independent FACS sorts, dropping sequences for which the standard deviation of replicate measurements was greater than 1.25. An amino acid variant was retained only if at least three independent QC-passing observations were collected between synonymous DNA variants and replicate FACS sorts.

### Surface Plasmon Resonance (SPR)

Antibody Expression in SoluPro™ E. coli B Strain

Individual SoluPro™ E. coli B strain colonies expressing antibody Fab variants were inoculated in LB media in 96-well deep blocks (Labcon) and grown at 30° C. for 24 h to create seed cultures for inducing expression. Seed cultures were then inoculated in IBM containing inducers and supplements in 96-well deep block and additionally grown at 30° C. for 24 h. Post induction samples were transferred to 96-well plates (Greiner Bio-One), pelleted and lysed in 50 μL lysis buffer (1× BugBuster protein extraction reagent containing 0.01 KU Benzonase Nuclease and 1× Protease inhibitor cocktail). Plates were incubated for 15-20 min at 30° C. then centrifuged to remove insoluble debris. After lysis samples were adjusted with 200 μL SPR running buffer (10 mM HEPES, 150 mM NaCl, 3 mM EDTA, 0.01% w/v Tween-20, 0.5 mg/mL BSA) to a final volume of 260 μL and filtered into 96-well plates. Lysed samples were then transferred from 96-well plates to 384-well plates for high-throughput SPR using a Hamilton STAR automated liquid handler. Colonies were prepared in two sets of independent replicates prior to lysis and each replicate was measured in two separate experimental runs. In some instances, single replicates were used, as indicated.

### SPR Experiments

High-throughput SPR experiments were conducted on a microfluidic Carterra LSA SPR instrument using SPR running buffer (10 mM HEPES, 150 mM NaCl, 3 mM EDTA, 0.01% w/v Tween-20, 0.5 mg/mL BSA) and SPR wash buffer (10 mM HEPES, 150 mM NaCl, 3 mM EDTA, 0.01% w/v Tween-20). Carterra LSA SAD200M chips were pre-functionalized with 20 μg/mL biotinylated antibody capture reagent for 600 s prior to conducting experiments. Lysed samples in 384-well blocks were immobilized onto 25/74 chip surfaces for 600 s followed by a 60 s washout step for baseline stabilization. Antigen binding was conducted using the non-regeneration kinetics method with a 300 s association phase followed by a 900 s dissociation phase. For analyte injections, six leading blanks were introduced to create a consistent baseline prior to monitoring antigen binding kinetics. After the leading blanks, five concentrations of HER2 extracellular domain antigen (ACRO Biosystems, prepared in three-fold serial dilution from a starting concentration of 500 nM), were injected into the instrument and the time series response was recorded. In most experiments, measurements on individual DNA variants were repeated four times. Typically each experiment run consisted of two complete measurement cycles (ligand immobilization, leading blank injections, analyte injections, chip regeneration) which provided two duplicate measurement attempts per clone per run. In most experiments, technical replicates measured in separate runs further doubled the number of measurement attempts per clone to four.

### Sensorgram Baseline Subtraction

Sensorgrams were generated from raw data using the Carterra Kinetics GUI software application provided with the Carterra LSA instrument. Sensorgram response values vs. time for 384 regions of interest (ROIs) on the Carterra chip were corrected using a double-referencing and alignment technique implemented by the Carterra manufacturer. This technique incorporates both the time-synchronous response of an interspot reference region adjacent to the ROI, as well as the non-synchronous response from a leading blank buffer injection flowing over the same ROI during an earlier experiment run cycle, to estimate and subtract a background response. Corrected sensorgrams were exported from the Kinetics software package for offline analysis.

### Kinetic Binding Parameters

Kinetic binding parameters were estimated via non-linear regression using a standard 1:1 binding model which was modified by the incorporation of a vector of t_{c} parameters each unique to one analyte concentration. For a single analyte concentration, the association phase model is:

$$R ( t , {c}_{a} ) = \frac{{c}_{a} {R}_{max}}{{c}_{a} + {K}_{D}} [ 1 - {e}^{- ( {c}_{a} {k}_{on} + {k}_{off} ) ( t - {t}_{c} )} ]$$

where
t=time
t_{c}=concentration-dependent time offset
cₐ=analyte concentration
kₒₙ=forward (association) reaction rate constant
k_{off}=backward (dissociation) reaction rate constant
K_{D}=k_{off}/kₒₙ
Rₘₐₓ=asymptotic maximum instrument response.

The additional concentration-dependent time offset parameter t_{c} was needed because of the unique measurement system that Carterra uses, in which successive association phase measurements at each new analyte concentration are attempted before the analyte from the previous phase has fully dissociated, leading to response curves which do not begin from zero response at t=0. The time offset parameters represent the projected time intercept of each association response curve; i.e., the amount of time prior to the start of the association phase, at which the measurement would have had to begin in order to reach the actual observed response at t=0. The dissociation phase was modeled as a standard decaying

exponential curve:

R(t,cₐ)R_{d}e^{-k}^{_{off}}^{(t-t}^{_{d}}^{-t}^{_{c}}⁾

where
t_{d}=start time of dissociation phase measurement
R_{d}=final estimated response value R(t_{d}, cₐ) from association equation.

The regression was conducted using R-language [42] scripts. Minpack.Im [43], an R-ported copy of MINPACK-1 [44] [45], a FORTRAN-based software package which implements the Levenberg-Marquardt [46] [47] non-linear least squares parameter search algorithm, was used to conduct the parameter search.

### Next-Generation Sequencing

To identify the DNA sequence of individual antibody variants evaluated in SPR, NGS was carried out on measured variants. Individual colonies were picked from LB agar plates containing 50 μg/mL Kanamycin (Teknova) into 96 deep well plates containing 1 mL LB media (Teknova). The culture plates were grown overnight in a 30° C. shaker incubator. 200 μl of overnight culture was transferred into new 96 well plates (Labcon) and spun down at 3,500 g. A portion of the pelleted material was transferred into 96 well PCR (Thermo-Fisher) plate via pinner (Fisher Scientific) which contained reagents for performing an initial phase PCR of a two-phase PCR for addition of Illumina adapters and sequencing. Reaction volumes used were 25 μl. During the initial PCR phase partial Illumina adapters were added to CDRH2 and CDRH3 amplicon via 4 PCR cycles. The second phase PCR added the remaining portion of the Illumina sequencing adapter and the Illumina i5 and i7 sample indices. The initial PCR reaction used 0.45 μM UMI primer concentration, 12.5 μl Q5 2× master mix (NEB). Reactions were initially denatured at 98° C. for 3 min, followed by 4 cycles of 98° C. for 10 s; 59° C. for 30 s; 72° C. for 30 s; with a final extension of 72° C. for 2 min. Following the initial PCR, 0.5 μM of the secondary sample index primers were added to each reaction tube. Reactions were then denatured at 98° C. for 3 min, followed by 29 cycles of 98° C. for 10 s; 62° C. for 30 s; 72° C. for 15 s; with a final extension of 72° C. for 2 min. Reactions were then pooled into a 1.5 mL tube (Eppendorf). Pooled samples were size selected with a 1× AMPure XP (Beckman Coulter) bead procedure. Resulting DNA samples were quantified by Qubit fluorometer. Pool size was verified via Tapestation 1000 HS and was sequenced on an Illumina MiSeq Micro (2×150 nt) with 20% PhiX.

After sequencing, amplicon reads were merged corresponding to their sample indices. Merging was performed by custom Python scripts. Scripts merged R1 and R2 reads based on overlapping sequence. Instances of unique amplicon sequences within each sample were counted and tabulated. Next, custom R scripts were applied to calculate sequence frequency ratios and Levenshtein distance between dominant and secondary sequences observed within samples. These calculations were used for quality filtering downstream to ensure clonal SPR measurements. The dominant sequence within each sample was then combined with companion Carterra SPR measurements.

### QC

SPR fits were excluded if any of the following criteria was satisfied:
less than 3 analyte concentrations providing usable fits;
handling errors as noted by operator;
non-physical fits (such as an upward-sloping dissociation-phase signal, even after sensorgram baseline subtraction);
non-convergent fits
a value of −log₁₀ K_{D}≤8.5 coupled with an estimated signal-to-noise ratio, for the highest analyte concentration ca included in the fit (typically 500 nM), of less than 10;
a value of −log₁₀ K_{D}>8.5 coupled with an estimated signal-to-noise ratio, for the highest analyte concentration included in the fit, of less than 70;
a t_{c} value, for the highest analyte concentration included in the fit, such that t_{c}<−300 s or t_{c}>0 s;
failed NGS;
non-clonal sequence (dominant sequence less than 100 times as abundant as secondary sequence when the Levenshtein distance between the two is greater than 2); and/or
sequence does not match any designed variant in the synthesized oligo pool (within a sequence identity tolerance to accommodate sequencing errors).

K_{D} and k_{off} were −log₁₀ transformed, while kₒₙ was log₁₀ transformed. Distributions of kinetic parameters were visually inspected for absence of significant batch effects. Multiple measurements of the same antibody variant (usually (a) duplicate serial measurements of the same clone in the same SPR run; (b) technical replicates of the same clone from duplicate 384-well plates measured in separate runs; (c) two DNA variants with identical translation, when available; and (d) independent clones of a variant) were averaged in log space. Variants whose −log₁₀ K_{D} measurements showed a coefficient of variation greater than 5% upon aggregation were dropped.

### Observed Antibody Space (OAS) Database Processing

The OAS database [48] of unpaired immunoglobulin chains was downloaded on Feb. 1, 2022. From the full database, the following exclusions were applied to the raw OAS data: first, studies whose samples come from another study in the database (Author field Bonsignori et al., 2016, Halliley et al., 2015, Thornqvist et al., 2018); second, studies originating from immature B cells (BType field Immature-B-Cells and Pre-B-Cells) and B cell-associated cancers (Disease field Light Chain Amyloidosis, CLL); and finally, sequences were excluded if any of the following criteria was met:
Sequence contains a stop codon
Sequence is non-productive
V and J segments are out of frame
Framework region 2 is missing
Framework region 3 is missing
CDR3 is longer than 37 amino acids
J segment sequence identity with closest germline is less than 50%
Sequence is missing an amino acid at the beginning or at the end of any CDR
Conserved cysteine residue is missing
Locus does not match chain type

From the resulting sequences, and for each of the two (heavy/light) chains, two types of subsequences were extracted: “CDR” and “near-full length (NF)”. In CDR datasets, we extracted only the CDR1, CDR2 and CDR3 segments as defined by the union of the IMGT [49] and Martin [50] labeling schemes. In NF datasets, we included IMGT positions 21 through 128 (127 for light chains and for heavy chains from rabbits and camels).

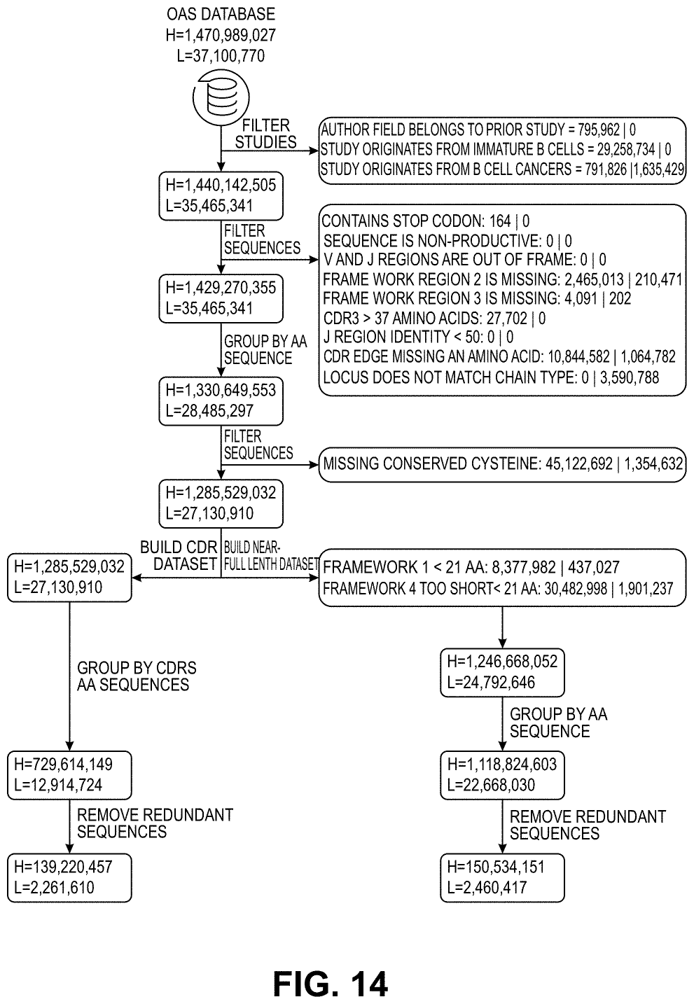

In all four datasets, duplicated sequences were removed, while tabulating the redundancy information (i.e. the number of times a specific sequence was observed in each study). Sequences with a redundancy of one (i.e., observed only once in a single study) were dropped on the grounds of insufficient evidence of genuine biological sequence as opposed to sequencing errors. FIG. 14 depicts a flow diagram depicting a computer-implemented method 1400 with the number of sequences filtered out and retained after each pre-processing step, according to some aspects. The method 1400 may include building four databases by processing the OAS dataset. The method 1400 may include building the databases by, for each of the two chains, heavy and light, extracting two subsets of sequences (CDR-only and near-full length antibody sequence), as discussed above. The method 1400 may include using models trained on CDR datasets for binding affinity and naturalness predictions, with the exception of the CR 9144 case study for which models trained on near-full length datasets may be used due to the location of mutated positions. The numbers denoted “H” and “L” in FIG. 14 depict, respectively, unique heavy and light chain sequences filtered out or retained at each step. The method 1400 may be performed by a computing device, such as the computing device 102 of FIG. 1.

### Model Architecture

Protein language models have shown great promise across a variety of protein engineering tasks [17, 51-55]. In some aspects, the present architecture is based on the RoBERTa model [56] and its PyTorch implementation within the Hugging Face framework [57]. In some aspects, the model contains 16 hidden layers, with 12 attention heads per layer. In some aspects, the hidden layer size is 768 and the intermediate layer size is 3072. In total, the model may contain 114 million parameters. In a pilot study, larger and smaller models were tested, and their respective losses compared in both a masked language modeling task and a regression task. It was observed that smaller models underperformed whereas larger models did not provide significant performance boost, confirming that the selected model size was appropriate. However, it will be appreciated by those of ordinary skill in the art that differently-configured and differently-parameterized models may be used in the present techniques, to achieve the aims of the present techniques.

### Model Training

Pre-Training with OAS Antibody Sequences

In some aspects, models for predicting binding affinity presented herein may be derived from RoBERTa architectures pre-trained on immunoglobulin sequences from the four datasets resulting from the OAS database processing (see Observed Antibody Space (OAS) database processing above). Thus, four or more models may be trained with heavy or light chain, CDR or NF sequences. In some aspects, training sequences contain species tokens (e.g. h for human, m for mouse, etc.) for conditioning the language model [58]. In addition, input sequences to CDR models may contain CDR-delimiting tokens so that the originally discontinuous CDR segments may be concatenated into a single input sequence.

In some aspects, CDR models may be used for all binding affinity and naturalness predictions, except for the CR9114 case study for which NF models may be used due to some framework substitutions present in the dataset.

In some aspects, model training may be performed in a self-supervised manner [48], following a dynamic masking procedure, as described in Wolf et al. [56], whereby 15% of the tokens in a sequence are randomly masked with a special [MASK] token. For masking, the DataCollatorForLanguageModeling class from the Hugging Face framework may be used which, unlike Wolf et al. [56], simply masks all randomly selected tokens. Training may be performed using the LAMB optimizer [59] with ϵ of 10⁻⁶, weight decay of 0.003 and a clamp value of 10, for instance. In some aspects, the maximum learning rate used was 10⁻³ with linear decay and 1000 steps of warm-up, dropout probability of 0.2, weight decay of 0.01, and a batch size of 416. The models may be trained for a maximum of 10 epochs in some aspects.

Fine-Tuning with Affinity Data

Transfer learning may be used in some aspects to leverage the OAS-pre-trained model by adding a dense hidden layer with a number of nodes (e.g., 768) followed by a projection layer with the required number of outputs. All layers may remain unfrozen to update all model parameters during training. Training may be performed with the AdamW optimizer [60], with a learning rate of, for example, 10⁻⁵, a weight decay of 0.01, a dropout probability of 0.2, a linear learning rate decay with 100 warm up steps, a batch size of 64, and mean-squared error (MSE) as the loss function.

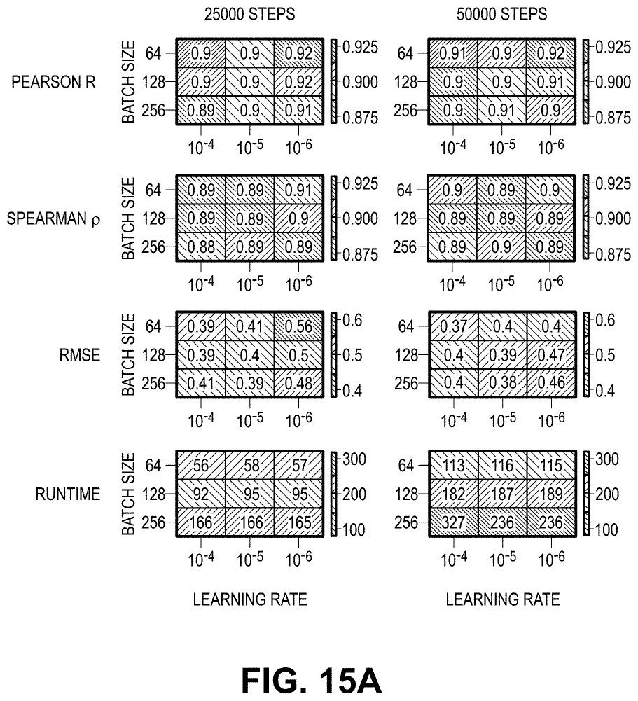

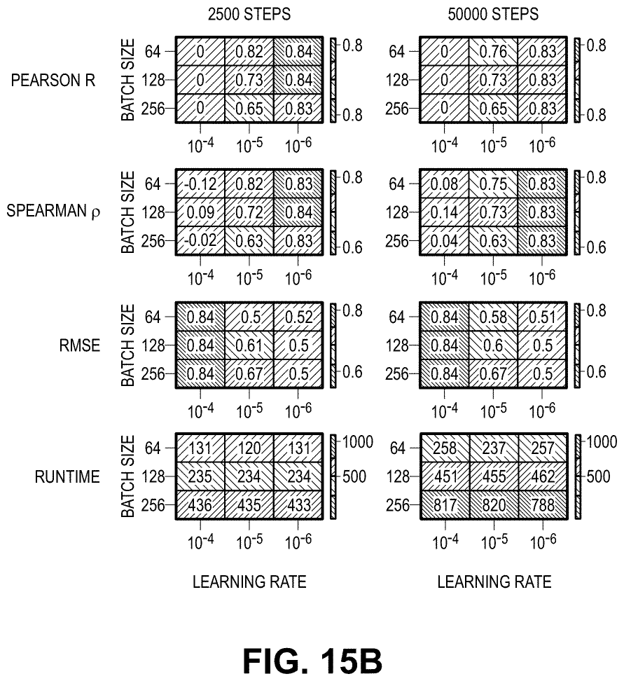

In some aspects, the present models may be trained for 25,000 steps. The number of steps, batch size, and learning rate for all runs were determined through a hyperparameter sweep using a pilot dataset. For example, a grid search was run across three learning rates (10⁻⁴, 10⁻⁵, 10⁻⁶), three batch sizes (64, 128, 256), and two numbers of steps (25,000; 50,000). Each hyperparameter set may be used to fine-tune the OAS pre-trained RoBERTa model, for example using a 90:10 train:hold-out split from a pilot dataset, as shown in FIG. 15A, and from a subset of 500 randomly selected sequences from the pilot dataset, as shown in FIG. 15B. The hyperparameter sweep of the pilot dataset of FIGS. 15A and 15B may include three metrics of predictive accuracy on the test set for each model, along with the time required to train the model. To minimize model training time while maintaining model performance, the final hyperparameters may be, for example, 10⁻⁵ for learning rate, a batch size of 64, and 25,000 training steps.

Co-Training with qaACE and SPR Data

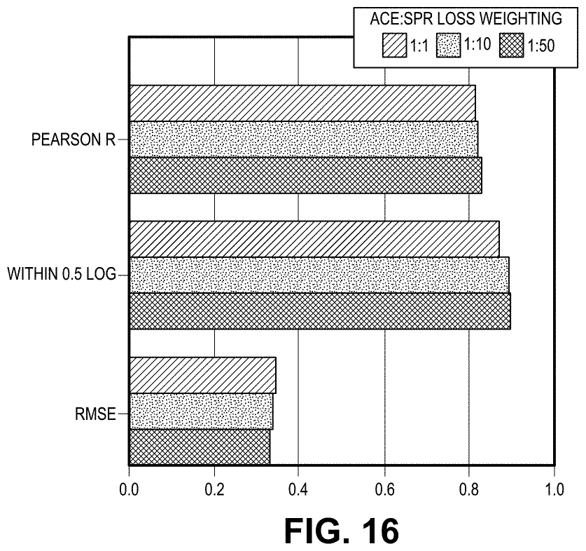

In some aspects, a model may be utilized to predict both qaACE and SPR values from sequences, using a weighted sum of the mean squared errors for each regression task as the loss function. For example, a sweep across weights showed that a 1:50 weighting towards SPR provided a highest combined accuracy, as shown in FIG. 16. In some aspects, models may be evaluated using pooled out-of-fold predictions in a 10-fold cross-validation setting, and using data from both ACE and SPR experiments simultaneously, using a weighted sum to combine the loss from each dataset. Out-of-fold predictions may be pooled with 10-fold cross-validation to compare against −log₁₀ K_{D} values measured by SPR.

### Model Characterization

### Baselines

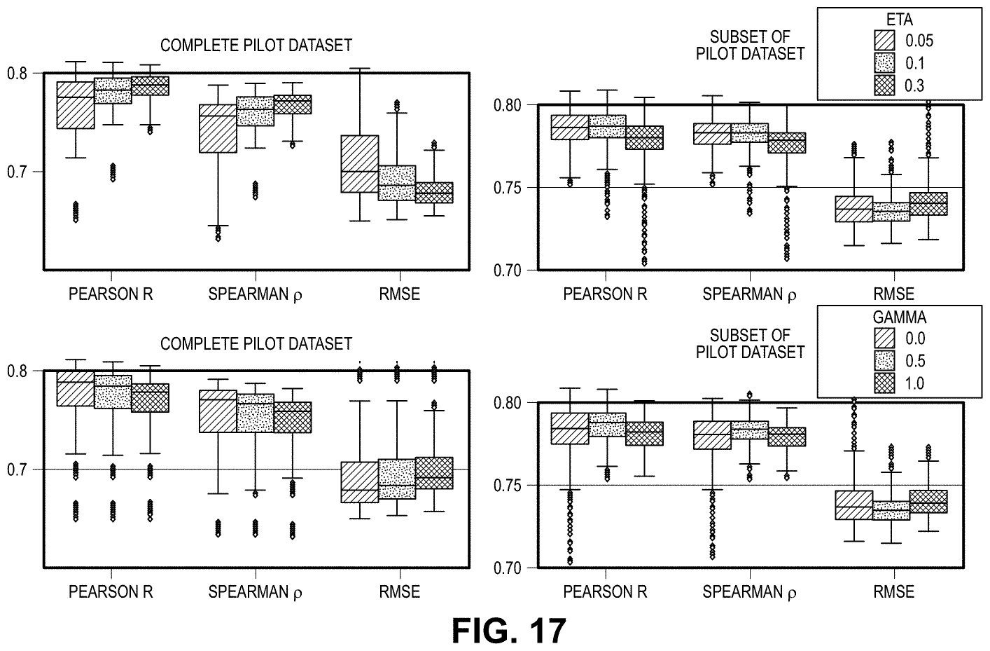

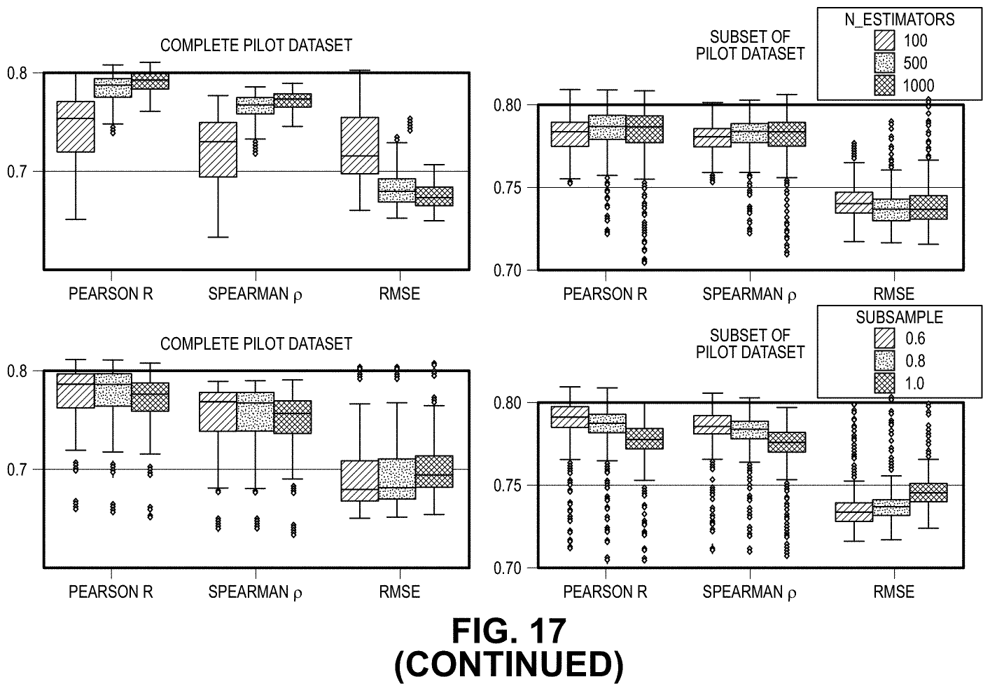

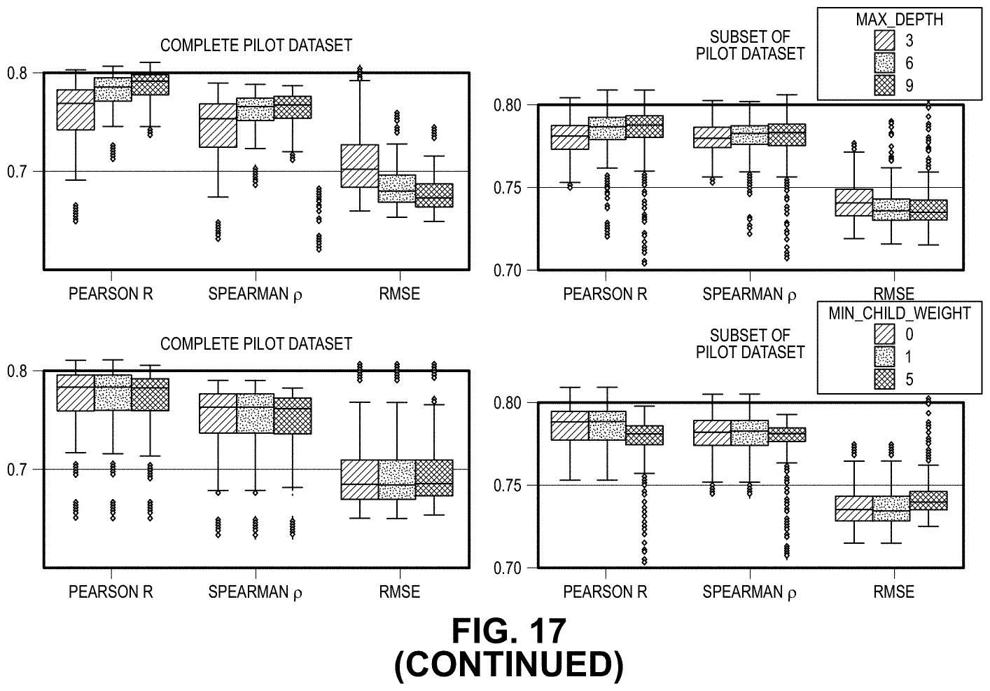

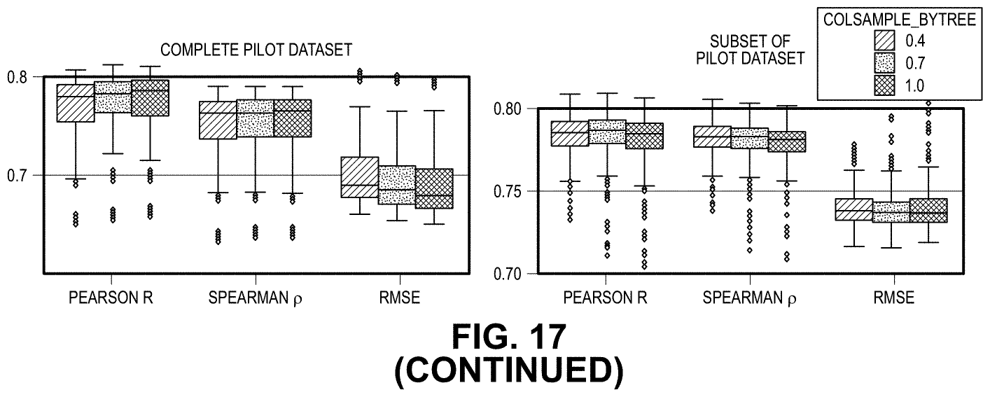

To assess the effectiveness of fine-tuning a pre-trained model, two baselines may be evaluated. First, an XGBoost [61] model may be implemented using a one-hot encoding of amino acids. For example, as depicted in FIG. 17, the following seven XGBoost hyperparameters may be selected using a grid search on a pilot dataset: eta=0.5, gamma=0, n_estimators=1000, subsample=0.6, max_depth=9, min_child_weight=1, col_sample_by_tree=1. FIG. 17 depicts test set accuracy of 10-fold cross-validation across each of the seven optimized XGBoost hyperparameters. An exhaustive grid search may be performed across a number (e.g., three) of values for each hyperparameter using both the complete pilot dataset, and a subset of the pilot dataset containing a number (e.g., 500) of selected sequences.

In some aspects, default values may be used for other hyperparameters. Second, a RoBERTa model with the same architecture as the pre-trained models may be trained with affinity data starting from randomly initialized weights with no OAS pretraining.

### Out-of-Distribution Predictions of Binding Affinity

To evaluate the predictive power for binding affinities outside of the distribution seen in the training set, the present techniques may include fine-tuning a model by excluding any variant with −log₁₀ K_{D} higher than that of parental trastuzumab from the training set. Further, the model may be tasked with predicting affinities of a set of sequences highly enriched in binders stronger than trastuzumab as validated by SPR.

### Assessing the Size and Fidelity of Training Data

In some aspects, models may be trained using subsets of different sizes from datasets of varying fidelity. The trast-3 dataset was treated as the high-fidelity dataset. The low-fidelity dataset was generated by isolating a single DNA variant for each sequence from a single FACS sort, using the same preprocessing workflow. Each training dataset may be split (e.g., evenly split into 1, 2, 4, 8, 16, 32, 64 and 128 subsets, respectively). Each training subset may be used to both directly train a model with randomly initialized weights, and to fine-tune the OAS pre-trained model. A common hold-out dataset containing 10% of data from the original trast-3 dataset may be used to evaluate all models, regardless of data source or training set size, in some aspects. These sequences may be removed from both datasets before constructing the training subsets.

### Embeddings

Embeddings may be generated by taking the mean pool of activations from the last hidden layer of the model, head excluded. The resulting size of the embedding of each sequence may be, for example, 768. The dimensionality of embeddings may be reduced with the Uniform Manifold Approximation and Projection (UMAP) algorithm as implemented in the RAPIDS library [62], for example.

In a first investigation, embeddings were compared from four different models, resulting from presence or absence of OAS pre-training and presence or absence of binding affinity fine-tuning using the trast-2 dataset. In a second investigation, embeddings were leveraged to cluster variants close in internal representation space. To this aim, dimensionality-reduced embeddings were filtered to retain only strong binders based on predicted qaACE scores and 3D embeddings were clustered using HDBSCAN [63], with a minimum cluster size of 40 sequences. Sequence logo plots for each cluster may be generated using Logomaker [64].

### Epistasis

Epistatic interactions between mutations may be assessed by considering the predicted affinity scores for the double mutant, the constituent single mutants, and the parental antibody sequence. Specifically, the epistatic effect between two mutations, m₁ and m₂, may be calculated as:

Epistasis(m₁,m₂)=(y_{1,2}−y_{wt})−(y₁−y_{wt})−(y₂−y_{wt})

where yᵢ denotes the predicted ACE score for the mutant with mutation(s) i, or the parental sequence in the case of y_{wt}.

where yᵢ denotes the predicted qaACE score for the mutant with mutation(s) i, or the parental sequence in the case of y_{wt}.

### Antibody Naturalness

The naturalness nₛ of a sequence may be defined as the inverse of its pseudo-perplexity according to the definition by Salazar et al. [65] for masked language models (MLMs). Recall that, for a sequence S with N tokens, the pseudo-likelihood that a MLM with parameters Θ assigns to this sequence is given by:

PLL(S)=Σₜ₌₁^{|S|}P_{MLM}(t|S_{\t};Θ)

The pseudo-perplexity is obtained by first normalizing the pseudo-likelihood by the sequence length and then applying the negative exponentiation function:

$$PPPL ( S ) = exp ( - \frac{1}{\vert S \vert} PLL ( S ) )$$

Thus, the sequence naturalness is:

$${n}_{s} = \frac{1}{PPPL ( S )} = exp ( \frac{1}{\vert S \vert} PLL ( S ) )$$

Naturalness scores may be computed using the two pre-trained models described above (see Pre-training with OAS antibody sequences). Several antibody properties (immunogenicity, developability, expression level, and mutational load) may be analyzed to investigate a potential relationship with sequence naturalness. For datasets whose members exhibit variation in both chains (immunogenicity and expression level), the reported naturalness score may be the average of the individual heavy- and light-chain scores. For datasets whose members exhibit variation only in the heavy chain (developability and mutational load), only the heavy-chain naturalness score may be computed. In some aspects, naturalness scores are reported in all cases from models trained on CDR datasets (see Pre-training with OAS antibody sequences, supra).

### Naturalness Association Plots

To assess the relationship between naturalness and antibody properties of interest, the present techniques may make use of naturalness association plots. To construct such plots, a 2-column table may be considered in which each row contains a pair of (antibody naturalness, antibody property value). The following procedure may then be followed:
Bin naturalness values into four equally spaced intervals (low, low-mid, mid-high, high) resulting from dividing the naturalness range by 4.
For each naturalness interval, take the property values of antibodies whose naturalness falls within the interval and aggregate the values by median (continuous properties) or fraction of positives (binary properties). Aggregates (Y axis) by naturalness interval (X axis) are then plotted as a box plot (continuous properties) or bar plot (binary).
Compute the Jonckheere-Terpstra test for increasing or decreasing trends and record the p-value.
Add a dashed line connecting the medians (for box plots) or the heights (for bar plots).

For boxplots, the whisker parameter may be set to 1.5 and outliers may not be plotted. Generally, the rationale for using medians and binning to aggregate continuous variables is to reduce the impact of outliers and noisy data points.

### Immunogenicity

The present techniques may include obtaining immunogenic responses, reported as percent of patients with anti-drug antibody (ADA) responses, from Marks et al. [28]. Of all classes of antibodies (human, humanized, chimeric, hybrid, mouse), only humanized antibodies may be included in some analysis aspects because (1) inter-class comparisons are trivial, amounting to simple species discrimination, while intra-class comparisons are both challenging and practically relevant; (2) humanized antibodies represent the largest class (n=97) in this dataset, thereby providing the greatest statistical power; and (3) compared to the second largest class in the dataset, which is human antibodies, humanized antibodies have in principle more immunogenic potential due to the animal origin of their CDRs, thereby providing a practically relevant case study to assess the degree of human naturalness achieved upon engineering/humanization.

### Developability

Sequence developability may be defined as a binary variable indicating whether an antibody sequence fails at least one of the developability flags computed by the Therapeutic Antibody Profiler (TAP) tool [31]. See the TAP analysis subsection, supra, for a detailed definition of these flags.

The present techniques may include scoring hits (positive enrichment in round 3 compared to round 2) heavy chain sequences (n=882) from the phage display library described in Liu et al. [29], which we refer to as the Gifford Library). The present techniques may also include analyzing trastuzumab variants with up to 3 simultaneous amino-acid replacements in 10 positions of CDRH2 and 10 positions of CDRH3 (according to the same mutagenesis strategy of the trast-3 dataset).

### Processing of the Gifford Library Dataset

The present techniques may include obtaining phage display data from the Gifford Library described in Liu et al. [29]. Specifically, the raw FASTQ files for rounds 2 (E1_R2) and 3 (E1_R3) of enrichment may be downloaded from the NIH's Sequence Read Archive (SRA) under accession number SRP158510. The guidelines for processing the data as per Liu et al. [29] may then be followed. First, the flanking DNA sequences of TATTATTGCGCG (SEQ ID NO: 26) and TGGGGTCAA may be used to pull the CDRH3 sequences. Then, sequences that included N or cannot be translated (divisible by 3) may be excluded. Next, DNA sequences may be translated into protein sequences, and dropped if they contained a premature stop codon. Then, CDRH3 sequences shorter than 8 or longer than 20 amino acids may be filtered out. Lastly, the number of occurrences of each unique sequence may be determined, and sequences occurring less than 6 times considered noise and dropped/excluded, in some aspects.

### Construction of Scaffold Sequences in the Gifford Library Dataset

A full-length antibody sequence may be required for analysis with the NF language models. However, the raw sequencing data from Liu et al. [29] only contains CDRH3 and the original study provides scaffold gene names, not sequences. Therefore, in some aspects of the present techniques, antibody sequences may be reconstructed from the gene names provided. For the heavy chain, the present techniques may use the IGHV3-23 germline and performed the following modifications: if the CDRH3 ended with DY, append the IGHJ4 sequence to IGHV3-23, but if the CDRH3 ended with DV, use IGHJ6 instead, as per Liu et al. [29]. The heavy chain genes may also be cross-validated with IgBlast.

The resulting scaffold sequences are reported below, where the _CDR3_ placeholder was replaced with the selected NGS-derived CDRH3 sequences:

>HC_DY
(SEQ ID NO: 1)
EVQLVESGGGLVQPGGSLRLSCAASGFTFSSYAMSWVRQAPGKGLEWVSA
ISGSGGSTYYADSVKGRFTISRDNS-KNTLYLQMNSLRAEDTAVYYC__
CDR3__DYWGQGTLVTVSS
>HC_DV 1016
(SEQ ID NO: 27)
EVQLVESGGGLVQPGGSLRLSCAASGFTFSSYAMSWVRQAPGKGLEWVSA
ISGSGGSTYYADSVKGRFTISRDNS-KNTLYLQMNSLRAEDTAVYYC__
CDR3__DVWGKGTTVTVSS

### TAP Analysis

The present techniques may use the Therapeutic Antibody Profiler (TAP), described in Raybould et al. [66] to calculate developability scores. A commercially-licensed virtual machine image of the tool may be used (e.g., last updated on Feb. 7 2022). TAP calculates five developability metrics: Total CDR Length, Patches of Surface

Hydrophobicity (PSH), Patches of Positive Charge (PPC), Patches of Negative Charge (PNC), and Structural Fv Charge Symmetry Parameter (SFvCSP). Furthermore, it generates flags for whether or not the metric is acceptable relative to a reference set of therapeutic antibodies. Metrics that fell outside the reference distribution may be flagged as “red”, whereas metrics that fall within the most extreme 5% of the distribution may be “amber”, and metrics that fall in the main body of the distribution past the 5% threshold may be “green” and acceptable.

Using TAP, sequence hits from the Gifford Library dataset as well as trastuzumab variants may be analyzed. TAP flags may be used determine if an antibody has acceptable developability scores, and an antibody variant considered a failure if at least one of the TAP flags was not green.

### Antibody Expression in HEK-293 Cells

In some aspects, clinical-stage antibody expression levels in HEK-293 cells may be collected from Jain et al. [30]. The dataset may be heterogeneous with regard to antibody type (e.g., human, humanized, chimeric, etc). For the same reasons illustrated for immunogenicity, the present techniques may focus on humanized antibodies (n=67).

In addition to HEK-293 titer, Jain et al. reported additional biophysical measurements.

In testing, associations between naturalness score and biophysical parameters other than titer were not specifically identified. However, those of ordinary skill in the art will appreciate that a dataset of clinical-stage antibodies is necessarily already biased towards antibodies endowed with favorable properties, meaning that distributions of biophysical parameters are strongly depleted of poorly performing antibodies. The availability of positive but not negative examples severely limits the ability to detect associations between biophysical parameters and other metrics such as naturalness.

### Mutational Load

Mutational load may be defined as the number of amino acid substitutions in an antibody variant compared with its parental sequence. The present techniques may analyze the distribution of naturalness scores across 6,710,401 trastuzumab variants with mutational load between 1 and 3 (10 positions in CDRH2 and 10 positions in CDRH3, allowing all natural amino acids except cysteine). The present techniques may include assessing the statistical significance of differences in naturalness score distributions by mutational load using the Jonckheere-Terpstra test for trends.

### Genetic Algorithms

To generate sequence variants with desired properties (e.g., high/low/target qaACE score and high naturalness), the present techniques may include a genetic algorithm (GA) using, for example, a tailored version of the DEAP library in Python [67]. In this GA, each individual sequence variant may be reduced to its CDR representation described above (union of IMGT and Martin definitions). Each GA run may be initialized from a single trastuzumab sequence. The predicted qaACE and naturalness scores of each sequence may be evaluated using the models described above. A cyclical select-reproduce-mutate-cull process was applied to the starting sequence pool that is common in μ+λ GAs [68].

Each offspring pool may contain, for example, the original 100 parents, along with 200 new, unique 1062 individuals. Of the offspring, 30% were created from a single point mutation of a parent (excluding cysteine), and 70% were created from two-point crossovers between two parents.

For example, since the GA is initialized from a single sequence, the first offspring pool contained 299 individuals, all of which were created using single point mutations from trastuzumab. In some aspects, all sequences may be constrained to remain within the trast-3 library computational space (up to triple mutants in 10 positions in CDRH2 and CDRH3, respectively). If a unique offspring could not be produced within these constraints, a randomly generated individual within the constraints may be added to the offspring pool. Tournament selection without replacement (tournament size=3) may be performed to cull the population (size=300) and select the individuals for the next generation (size=100).

This process represented one “generation” of the GA. The GA may be run for a number of generations (e.g., 20). To properly balance between the qaACE score and naturalness objectives, the fitness objective may be defined as:

$$Fitness = \frac{{( naturalness )}^{5}}{\vert ACE score - ACE target \vert}$$

To test the generative capabilities of the present techniques models, the GA may be run in the following configurations:
Target qaACE score=9 (Maximize qaACE score), while maximizing naturalness
Target qaACE score=1 (Minimize qaACE score), while maximizing naturalness
Target qaACE score=6, while maximizing naturalness
   For example, since the GA queries 300 individuals in the first generation, and 200 individuals in each subsequent generation, the GA queries 4,100 (non-unique) sequences across 20 generations.

As a baseline, the present techniques may include randomly selecting 4,100 sequences from the full mutational search space, and selecting the top 100 individuals with the highest fitness as described above. The fitness function may also be used to identify the top 100 individuals from the exhaustive search of the mutational space, and from the trast-3 dataset.

### REFERENCES

1. S. M. Paul, D. S. Mytelka, C. T. Dunwiddie, C. C. Persinger, B. H. Munos, S. R. Lindborg, and A. L. Schacht, “How to improve R&D productivity: the pharmaceutical industry's grand challenge,” Nature Reviews Drug Discovery, vol. 9, pp. 203-214, February 2010.
2. S. Yamaguchi, M. Kaneko, and M. Narukawa, “Approval success rates of drug candidates based on target, action, modality, application, and their combinations,” Clinical and Translational Science, vol. 14, pp. 1113-1122, April 2021.
3. J. P. Hughes, S. Rees, S. B. Kalindjian, and K. L. Philpott, “Principles of early drug discovery,” British Journal of Pharmacology, vol. 162, pp. 1239-49, March 2011.
4. J. Ministro, A. Manuel, and J. Goncalves, “Therapeutic antibody engineering and selection strategies,” Advances in biochemical engineering/biotechnology, vol. 171, pp. 55-86, November 2019.
5. K. R. Hanning, M. Minot, A. K. Warrender, W. Kelton, and S. T. Reddy, “Deep mutational scanning for therapeutic antibody engineering,” Trends in Pharmacological Sciences, vol. 43, no. 2, pp. 123-135, 2022.
6. T. Kuramochi, T. Igawa, H. Tsunoda, and K. Hattori, “Humanization and simultaneous optimization of monoclonal antibody,” Methods in Molecular Biology, vol. 1060, pp. 123-137, 2014.
7. C. Schneider, A. Buchanan, B. Taddese, and C. M. Deane, “DLAB-Deep learning methods for structure-based virtual screening of antibodies,” Bioinformatics, vol. 38, pp. 377-383, September 2021.
8. A. Khan, A. I. Cowen-Rivers, D.-G.-X. Deik, A. Grosnit, K. Dreczkowski, P. A. Robert, V. Greiff, R. Tutunov, D. Bou-Ammar, J. Wang, and H. Bou-Ammar, “AntBO: Towards real-world automated antibody design with combinatorial bayesian optimisation,” arXiv:2201.12570 [q-bio.BM], 2022.
9. W. Jin, J. Wohlwend, R. Barzilay, and T. S. Jaakkola, “Iterative refinement graph neural network for antibody sequence-structure co-design,” arXiv:2110.04624 [q-bio.BM], 2022.
10. W. Jin, D. Barzilay, and T. Jaakkola, “Antibody-antigen docking and design via hierarchical structure refinement,” in Proceedings of the 39th International Conference on Machine Learning (K. Chaudhuri, S. Jegelka, L. Song, C. Szepesvari, G. Niu, and S. Sabato, eds.), vol. 162 of Proceedings of Machine Learning Research, pp. 10217-10227, PMLR, 17-23 Jul. 2022.
11. S. Luo, Y. Su, X. Peng, S. Wang, J. Peng, and J. Ma, “Antigen-specific antibody design and optimization with diffusion-based generative models,” bioRxiv doi: 10.1101/2022.7.10.499510, 2022.
12. S. P. Mahajan, J. A. Ruffolo, R. Frick, and J. Gray, “Hallucinating structure-conditioned antibody libraries for target-specific binders,” BioRxiv doi: 10.1101/2022.6.6.494991, 2022.
13. J. J. Jeffrey A. Ruffolo, Jeremias Sulam, “Antibody structure prediction using interpretable deep learning,” Patterns, vol. 3, p. 100406, February 2022.
14. R. W. Shuai, J. A. Ruffolo, and J. J. Gray, “Generative language modeling for antibody design,” bioRxiv doi: 10.1101/2021.12.13.472419, 2021.
15. D. M. Mason, S. Friedensohn, C. R. Weber, C. Jordi, B. Wagner, S. M. Meng, R. A. Ehling, L. Bonati, J. Dahinden, P. Gainza, B. E. Correia, and S. T. Reddy, “Optimization of therapeutic antibodies by predicting antigen specificity from antibody sequence via deep learning,” Nature Biomedical Engineering, pp. 600-612, April 2021.
16. K. Saka, T. Kakuzaki, S. Metsugi, D. Kashiwagi, K. Yoshida, M. Wada, H. Tsunoda, and R. Teramoto, “Antibody design using LSTM based deep generative model from phage display library for affinity maturation,” Scientific Reports, vol. 11, p. 5852, March 2021.
17. E. C. Alley, G. Khimulya, S. Biswas, M. AlQuraishi, and G. M. Church, “Unified rational protein engineering with sequence-only deep representation learning,” Nature Methods, vol. 12, pp. 1315-1322, March 2019.
18. Z. Ren, J. Li, F. Ding, Y. Zhou, J. Ma, and J. Peng, “Proximal exploration for model-guided protein sequence design,” bioRxiv doi 10.1101/2022.4.12.487986, 2022.
19. L. A. Rabia, A. A. Desai, H. S. Jhajj, and P. M. Tessier, “Understanding and overcoming trade-offs between antibody affinity, specificity, stability and solubility,” Biochemical engineering journal, vol. 137, pp. 365-374, September 2018.
20. J. Liu, “Activity-specific cell enrichment,” Patent Publication No. WO 2021/146626, 22.7.2021.
21. R. Akbar, P. A. Robert, M. Pavlovid, J. R. Jeliazkov, I. Snapkov, A. Slabodkin, C. R. Weber, L. Scheffer, E. Miho, I. H. Haff, D. T. T. Haug, F. Lund-Johansen, Y. Safonova, G. K. Sandve, and V. Greiff, “A compact vocabulary of paratope-epitope interactions enables predictability of antibody-antigen binding,” Cell Reports, vol. 34, p. 108856, March 2021.
22. J. Bostrom, S.-F. Yu, D. Kan, B. A. Appleton, C. V. Lee, K. Billeci, W. Man, F. Peale, S. Ross, C. Wiesmann, and G. Fuh, “Variants of the antibody herceptin that interact with HER2 and VEGF at the antigen binding site,” Science, vol. 323, pp. 1610-1614, March 2009.
23. S. Biswas, G. Khimulya, E. C. Alley, K. M. Esvelt, and G. M. Church, “Low-n protein engineering with data-efficient deep learning,” Nature Methods, vol. 18, pp. 389-396, April 2021.
24. A. Burkovitz, O. Leiderman, I. Sela-Culang, G. Byk, and Y. Ofran, “Computational identification of antigen-binding antibody fragments,” The Journal of Immunology, vol. 190, pp. 2327-2334, January 2013.
25. V. C. Xie, J. Pu, B. P. Metzger, J. W. Thornton, and B. C. Dickinson, “Contingency and chance erase necessity in the experimental evolution of ancestral proteins,” eLife, vol. 10, June 2021.
26. P. C. Phillips, “Epistasis—the essential role of gene interactions in the structure and evolution of genetic systems,” Nature Reviews Genetics, vol. 9, pp. 855-867, November 2008.
27. A. M. Phillips, K. R. Lawrence, A. Moulana, T. Dupic, J. Chang, M. S. Johnson, I. Cvijovic, T. Mora, A. M. Walczak, and M. M. Desai, “Binding affinity landscapes constrain the evolution of broadly neutralizing anti-influenza antibodies,” eLife, vol. 10, p. e71393, September 2021.
28. C. Marks, A. M. Hummer, M. Chin, and C. M. Deane, “Humanization of antibodies using a machine learning approach on large-scale repertoire data,” Bioinformatics, vol. 37, pp. 4041-4047, June 2021.
29. G. Liu, H. Zeng, J. Mueller, B. Carter, Z. Wang, J. Schilz, G. Horny, M. E. Birnbaum, S. Ewert, and D. K. Gifford, “Antibody complementarity determining region design using high-capacity machine learning,” Bioinformatics, vol. 36, pp. 2126-2133, November 2019.
30. T. Jain, T. Sun, S. Durand, A. Hall, N. R. Houston, J. H. Nett, B. Sharkey, B. Bobrowicz, I. Caffry, Y. Yu, Y. Cao, H. Lynaugh, M. Brown, H. Baruah, L. T. Gray, E. M. Krauland, Y. Xu, M. Vasquez, and K. D. Wittrup, “Biophysical properties of the clinical-stage antibody landscape,” Proceedings of the National Academy of Sciences, vol. 114, pp. 944-949, January 2017.
31. M. I. J. Raybould, C. Marks, K. Krawczyk, B. Taddese, J. Nowak, A. P. Lewis, A. Bujotzek, J. Shi, and C. M. Deane, “Five computational developability guidelines for therapeutic antibody profiling,” Proceedings of the National Academy of Sciences, vol. 116, pp. 4025-4030, February 2019.
32. R. M. Adams, T. Mora, A. M. Walczak, and J. B. Kinney, “Measuring the sequence-affinity landscape of antibodies with massively parallel titration curves,” eLife, vol. 5, p. e23156, December 2016.
33. L. L. Reich, S. Dutta, and A. E. Keating, “SORTCERY-a high-throughput method to affinity rank peptide ligands,” Journal of Molecular Biology, vol. 427, pp. 2135-50, June 2015.
34. C. E. Z. Chan, A. P. C. Lim, P. A. MacAry, and B. J. Hanson, “The role of phage display in therapeutic antibody discovery,” International Immunology, vol. 26, pp. 649-657, August 2014.
35. I. T. Nakamura Y, Gojobori T, “Codon usage tabulated from international DNA sequence databases: status for the year 2000,” Nucleic Acids Research, vol. 28, p. 292, January 2000.
36. T. Magod and S. L. Salzberg, “FLASH: fast length adjustment of short reads to improve genome assemblies,” Bioinformatics, vol. 27, pp. 2957-2963, September 2011.
37. L. D., “Algorithms for efficiently collapsing reads with unique molecular identifiers,” PeerJ, vol. 7, p. e8275, December 2019.
38. M. Martin, “Cutadapt removes adapter sequences from high-throughput sequencing reads,” EMBnet.joumal, vol. 17, May 2011.
39. P. J. Cock, T. Antao, J. T. Chang, B. A. Chapman, C. J. Cox, A. Dalke, I. Friedberg, T. Hamelryck, F. Kauff, B. Wilczynski, and M. J. L. de Hoon, “Biopython: freely available python tools for computational molecular biology and bioinformatics,” Bioinformatics, vol. 25, pp. 1422-1423, June 2009.
40. S. Andrews, “FastQC. A quality control tool for high throughput sequence data.” Babraham Bioinformatics, Babraham Institute, Cambridge, United Kingdom, https://www.bibsonomy.org/bibtex/2b6052877491828ab53d3449be9b293b3/ozborn, 2010.
41. P. Ewels, M. Magnusson, S. Lundin, and M. Käller, “MultiQC: summarize analysis results for multiple tools and samples in a single report,” Bioinformatics, vol. 32, pp. 3047-3048, June 2016.
42. R Core Team, “R: A language and environment for statistical computing.” R Foundation for Statistical Computing, Vienna, Austria, https://www.R-project.org, 2021.
43. T. V. Elzhov, K. M. Mullen, A.-N. Spiess, and B. Bolker, minpack.lm: R Interface to the Levenberg-Marquardt Nonlinear Least-Squares Algorithm Found in MINPACK, Plus Support for Bounds. https://cran.r-project.org/web/packages/minpack.lm/minpack.lm.pdf, 2022.
44. J. J. Moré, “The Levenberg-Marquardt algorithm: Implementation and theory,” in Lecture Notes in Mathematics, pp. 105-116, Springer Berlin Heidelberg, 1978.
45. J. J. Moré, B. S. Garbow, and K. E. Hillstrom, Implementation Guide for MINPACK-1. https://www.osti.gov/biblio/5171554, 1980.
46. K. Levenberg, “A method for the solution of certain non-linear problems in least squares,” Quarterly of applied mathematics, vol. 2, pp. 164-168, July 1944.
47. D. W. Marquardt, “An algorithm for least-squares estimation of nonlinear parameters,” Journal of the society for Industrial and Applied Mathematics, vol. 11, no. 2, pp. 431-441, 1963.
48. T. H. Olsen, F. Boyles, and C. M. Deane, “Observed Antibody Space: A diverse database of cleaned, annotated, and translated unpaired and paired antibody sequences,” Protein Science, vol. 31, pp. 141-146, January 2022.
49. M.-P. Lefranc, C. Pommié, Q. Kaas, E. Duprat, N. Bosc, D. Guiraudou, C. Jean, M. Ruiz, I. Da Piédade, M. Rouard, E. Foulquier, V. Thouvenin, and G. Lefranc, “IMGT unique numbering for immunoglobulin and T cell receptor constant domains and Ig superfamily C-like domains,” Developmental & Comparative Immunology, vol. 29, pp. 185-203, March 2005.
50. K. Abhinandan and A. C. Martin, “Analysis and improvements to Kabat and structurally correct numbering of antibody variable domains,” Molecular Immunology, vol. 45, pp. 3832-3839, August 2008.
51. R. Rao, N. Bhattacharya, N. Thomas, Y. Duan, X. Chen, J. Canny, P. Abbeel, and Y. S. Song, “Evaluating Protein Transfer Learning with TAPE,” in Neural Information Processing Systems, vol. 32, pp. 9689-9701, Cold Spring Harbor Laboratory, June 2019.
52. A. Elnaggar, M. Heinzinger, C. Dallago, G. Rihawi, Y. Wang, L. Jones, T. Gibbs, T. Feher, C. Angerer, M. Steinegger, D. Bhowmik, and B. Rost, “ProtTrans: Towards Cracking the Language of Life's Code Through Self-Supervised Deep Learning and High Performance Computing,” bioRxiv doi: 10.48550/arXiv.2007.06225, 2020.
53. A. Rives, J. Meier, T. Sercu, S. Goyal, Z. Lin, J. Liu, D. Guo, M. Ott, C. L. Zitnick, J. Ma, and R. Fergus, “Biological structure and function emerge from scaling unsupervised learning to 250 million protein sequences,” Proceedings of the National Academy of Sciences, vol. 118, no. 15, p. e2016239118, 2021.
54. J. Meier, R. Rao, R. Verkuil, J. Liu, T. Sercu, and A. Rives, “Language models enable zero-shot prediction of the effects of mutations on protein function,” in Advances in Neural Information Processing Systems (A. Beygelzimer, Y. Dauphin, P. Liang, and J. W. Vaughan, eds.), vol. 34, pp. 29287-29303, 2021.
55. R. M. Rao, J. Liu, R. Verkuil, J. Meier, J. Canny, P. Abbeel, T. Sercu, and A. Rives, “MSA transformer,” in Proceedings of the 38th International Conference on Machine Learning (M. Meila and T. Zhang, eds.), vol. 139 of Proceedings of Machine Learning Research, pp. 8844-8856, PMLR, July 2021.
56. Y. Liu, M. Ott, N. Goyal, J. Du, M. Joshi, D. Chen, O. Levy, M. Lewis, L. Zettlemoyer, and V. Stoyanov, “RoBERTa: A robustly optimized BERT pretraining approach,” arXiv:1907.11692 [cs.CL], 2019.
57. T. Wolf, L. Debut, V. Sanh, J. Chaumond, C. Delangue, A. Moi, P. Cistac, T. Rault, R. Louf, M. Funtowicz, J. Davison, S. Shleifer, P. von Platen, C. Ma, Y. Jemite, J. Plu, C. Xu, T. L. Scao, S. Gugger, M. Drame, Q. Lhoest, and A. M. Rush, “Huggingface's transformers: State-of-the-art natural language processing,” arXiv:1910.03771 [cs.CL], 2019.
58. N. S. Keskar, B. McCann, L. R. Varshney, C. Xiong, and R. Socher, “CTRL: A conditional transformer language model for controllable generation,” arXiv:1909.05858 [cs.CL], 2019.
59. Y. You, J. Li, S. Reddi, J. Hseu, S. Kumar, S. Bhojanapalli, X. Song, J. Demmel, K. Keutzer, and C.-J. Hsieh, “Large batch optimization for deep learning: Training bert in 76 minutes,” arXiv:1904.00962 [cs.LG], 2019.
60. I. Loshchilov and F. Hutter, “Fixing weight decay regularization in Adam,” https://openreview.net/forum?id=rk6qdGgCZ, 2018.
61. T. Chen and C. Guestrin, “XGBoost: A scalable tree boosting system,” in Proceedings of the 22nd ACM SIGKDD International Conference on Knowledge Discovery and Data Mining, KDD '16, (New York, N.Y., USA), pp. 785-794, ACM, 2016.
62. R. D. Team, RAPIDS: Collection of Libraries for End to End GPU Data Science, 2018.
63. R. J. G. B. Campello, D. Moulavi, and J. Sander, “Density-based clustering based on hierarchical density estimates,” in Advances in Knowledge Discovery and Data Mining, pp. 160-172, Springer Berlin Heidelberg, 2013.
64. A. Tareen and J. B. Kinney, “Logomaker: beautiful sequence logos in Python,” Bioinformatics, vol. 36, pp. 2272-2274, December 2019.
65. J. Salazar, D. Liang, T. Q. Nguyen, and K. Kirchhoff, “Masked language model scoring,” arXiv:1910.14659 [cs.CL], 2019.
66. M. I. J. Raybould, C. Marks, K. Krawczyk, B. Taddese, J. Nowak, A. P. Lewis, A. Bujotzek, J. Shi, and C. M. Deane, “Five computational developability guidelines for therapeutic antibody profiling,” Proceedings of the National Academy of Sciences, vol. 116, pp. 4025-4030, February 2019.
67. F.-A. Fortin, F.-M. De Rainville, M.-A. Gardner, M. Parizeau, and C. Gagné, “DEAP: Evolutionary algorithms made easy,” Journal of Machine Learning Research, vol. 13, pp. 2171-2175, July 2012.
68. H. Beyer and H. Schwefel, “Evolution strategies—a comprehensive introduction,” Natural Computing, vol. 1, pp. 3-52, March 2002.

### Supplementary Information

### Tite-Seq CR9114 Dataset

### Dataset Processing

The Tite-seq CR9114 dataset [27] includes affinity data for 65,091 variants of the CR9114 bnAb heavy chain against three different influenza hemagglutinin (HA) antigen subtypes (H1, H3, and FluB). Each variant includes binary mutations in up to 16 positions based on the difference between the CR9114 germline and somatic sequences.

Out of the 65,091 variants, 63,419 (97%) bind to H1, 7174 (11%) bind to H3, and 198 (0.3%) bind to FluB. The present techniques may include downloading downloaded the dataset from https://cdn.elifesciences.org/articles/71393/elife-71393-fig1-data1-v3.csv. The amino acid sequence of each variant was inferred from the binary mutation information using a custom python script, using the germline

SEQ ID NO: 24
( [symbol] [symbol] VS [symbol] SWVRQAPGQGLEWMGGIIPIFGTANYAQKFQGRVTITADK-
STSTAYMELSSLRSEDTAVYYCARHGNYYYYYGMDVWGQGTTVTVSS)
and
somatic
SEQ ID NO: 25 [symbol] [symbol]
VSCKASGGT [symbol] YAISWVRQAPGQGLEWMGGI [symbol] PIFG [symbol] YAQKFQGRVTI [symbol] AD [symbol] [symbol]
TAYME L [symbol] SL [symbol] SEDTAVY [symbol] CARHGNYYYY [symbol] GMDVWGQGTTVTVSS)

sequences (16 somatic mutations highlighted in red, and trimmed sequences struck through). The first 19 amino acids may be trimmed for compatibility with the NF Heavy model (starting from the 21st amino acid in the IMGT numbering scheme).

### Model Architecture

The NF Heavy model may be used, initialized with weights trained on the OAS dataset (PT) as well as a model initialized with random weights (NPT). To support predictions for all three antigen targets, a sum of the mean squared errors may be used in some aspects for each regression task as the loss function for the regression only models (Reg). In addition, models may be trained using a mixture-model combining classification and regression tasks in a joint model (Mix). For example, the loss function for the mixture model may be defined as:

$$\begin{matrix}ℓ ( x , y ) = L = {\sum }_{c = 1}^{C} {L}_{c}^{Reg} + {w}^{Cls} {L}_{c}^{Cls} , \\ {ℓ}_{c}^{Reg} ( x , y ) = {L}_{c}^{Reg} = \frac{1}{N} {\sum }_{n = 1}^{N} {l}_{n , c}^{Reg} , \\ {l}_{n , c}^{Reg} = {[ {x}_{n , c}^{Reg} - ( σ ( {x}_{n , c}^{Cls} ) \cdot {y}_{n , c} + ( 1 - σ ( {x}_{n , c}^{Cls} ) ) \cdot {B}_{c} ) ]}^{2} , \\ {ℓ}_{c}^{Cls} ( x , y ) = {L}_{c}^{Cls} = \frac{1}{N} {\sum }_{n = 1}^{N} {l}_{n , c}^{Cls} , \\ {l}_{n , c}^{Cls} = - [ {p}_{c} {y}_{n , c}^{Cls} \cdot log σ ( {x}_{n , c}^{Cls} ) + ( 1 - {y}_{n , c}^{Cls} ) \cdot \\ log ( 1 - σ ( {x}_{n , c}^{Cls} ) ) ] , \\ {y}_{n , c}^{Cls} = \{ \begin{matrix}0 , & if {y}_{n , c} \leq {B}_{c} ; \\ 1 , & if {y}_{a , c} > {B}_{c}\end{matrix}\end{matrix} ,$$

where C is number of targets (e.g., 3), N is the number of training examples in a batch (e.g., 256),
y_{n,c} is the measured affinity of sample n to target c, X_{n,c}^{Cls} is the predicted binding classification logits score of sample n to target c, X_{n,c}^{Reg} c is the predicted affinity of sample n to target c given that it is binding c, w^{Cis} is the classification loss weight (0.1 for models trained with 10% and 0.1% of the dataset, and 0.01 for models trained with 1% of the dataset), B_{c} is the lower boundary for each target as determined in the original publication (e.g., 7 for H1, and 6 for H3 and FluB), σ is the logistic sigmoid function, and pc is the positive weight score. For example, p_{c} may be calculated dynamically for each training set as the number of negative examples for class c divided by the number of positive examples for class c in the training set (it is set to 1 in cases where there are no positive examples).

### Model Training

In some aspects, the present techniques may include training four types of models (Reg-PT, Reg-NPT, Mix-PT, and Mix-NPT) using three training set sizes (10%, 1%, and 0.1% of 65,091), each using 10 cross-validation folds. For the 1% and 0.1% experiments, 10 folds may be randomly selected requiring each fold to include at least one positive and one negative example for each target in the training set. To support early-stopping and classifier calibration 10% of each test set may be allocated as a separate validation set. In some aspects, transfer learning may be used to leverage the OAS-pretrained model by adding a dense hidden layer with a number of nodes (e.g., 768) followed by a projection layer with the required number of outputs. All layers may remain unfrozen to update all model parameters during training. Training may be performed with the AdamW optimizer with a learning rate of (e.g.) 10₋₅, a weight decay of 0.01, a dropout probability of 0.2, a linear learning rate decay with 100 warm up steps, and a batch size of 256. All models were trained until the validation set loss stopped improving for a number of epochs (e.g., 50, 250, 2500) for training sizes (e.g., 10%, 1%, and 0.1%) respectively.

### Model Evaluation

Unlike typical cross-validation experiments the training sets may be smaller than the test sets and therefore each variant may be present in multiple test sets. For each variant predictions may be randomly selected from a single model instead of using the mean predicted value to avoid introducing an ensemble effect. During inference on the test set the predicted regression values may be calculated as:

ŷ_{c}^{Reg}(x)=max{σ(x_{c}^{Cls})·x_{c}^{Reg}+(1−σ(x_{c}^{Cls}))·B_{c},B_{c}}

Raw classification logits may be converted to probabilities and calibrated using the CalibratedClassifierCV class of scikit-learn using cv=“prefit” and method=“isotonic,” for example Classification metrics may be calculated using scikit-learn functions balanced_accuracy_score, f1_score, precision_score, recall_score, and average_precision_score, in some aspects. Specifically, Balanced Accuracy may be defined as

$$\frac{1}{2} ( \frac{TP}{TP + FN} + \frac{TN}{TN + FP} )$$

and Average Precision is defined as

Σₙ(Rₙ−Rₙ₋₁)Pₙ
where Pₙ and Rₙ are the precision and recall at the nth threshold of the precision-recall curve.

### Antibodies

The term “antibody” as used herein refers to whole antibodies that interact with (e.g., by binding, steric hindrance, stabilizing/destabilizing, spatial distribution) an epitope on a target antigen. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies, chimeric antibodies, single-chain Fvs (scFv), disulfide-linked Fvs (sdFv), Fab fragments, F (ab′) fragments, and anti-idiotypic (anti-Id) antibodies (including, e.g., anti-Id antibodies to antibodies of the invention), and epitope-binding fragments of any of the above. The antibodies can be of any isotype (e.g., IgG, IgE, IgM, IgD, IgA and IgY), class (e.g., IgG1, IgG2, IgG3, IgG4, IgA1 and IgA2) or subclass. The antibody or epitope-binding fragments may be, or be a component of, a multi-specific molecule.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

The phrase “antibody fragment”, as used herein, refers to one or more portions of an antibody that retain the ability to specifically interact with (e.g., by binding, steric hindrance, stabilizing/destabilizing, spatial distribution) a target epitope. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies.

As described herein, antibodies may include biologically active derivatives or variants or fragments. As used herein “biologically active derivative” or “biologically active variant” includes any derivative or variant of an antibody having substantially the same functional and/or biological properties of said antibody (e.g., a WT antibody), such as binding properties, and/or the same structural basis, such as a peptidic backbone or a basic polymeric unit, including framework regions.

An “analog,” such as a “variant” or a “derivative,” is an antibody substantially similar in structure and having the same biological activity, albeit in certain instances to a differing degree, to a naturally-occurring antibody or a WT antibody or another reference antibody as will be understood by those of skill in the art. For example, an antibody variant refers to an antibody sharing substantially similar structure and having the same biological activity as a reference antibody. Variants or analogs differ in the composition of their amino acid sequences compared to the reference antibody from which the analog is derived, based on one or more mutations involving (i) deletion of one or more amino acid residues at one or more termini of the antibody and/or one or more internal regions of the antibody sequence (e.g., fragments), (ii) insertion or addition of one or more amino acids at one or more termini (typically an “addition” or “fusion”) of the antibody and/or one or more internal regions (typically an “insertion”) of the antibody sequence or (iii) substitution of one or more amino acids for other amino acids in the antibody sequence. By way of example, a “derivative” is a type of analog and refers to an antibody sharing the same or substantially similar structure as a reference antibody that has been modified, e.g., chemically.

In some embodiments, the variants or sequence variants are mutants wherein 1, 2, 3, 4, 5, 6 or more amino acids within one or more CDR are mutated relative to a reference antibody. In some embodiments, CDRs on the light chain, heavy chain, or both heavy and light chain, are mutated. In some embodiments, one or more framework amino acid residues are mutated relative to a reference antibody.

In substitution variants, one or more amino acid residues, e.g., in a CDR region, of an antibody are removed and replaced with alternative residues. In one aspect, the substitutions are conservative in nature and conservative substitutions of this type are well known in the art. Alternatively, the disclosure embraces substitutions that are also non-conservative. Exemplary conservative substitutions are described in Lehninger, [Biochemistry, 2nd Edition; Worth Publishers, Inc., New York (1975), pp. 71-77].

Antibodies contemplated herein include full-length antibodies, biologically active subunits or fragments of full length antibodies, as well as biologically active derivatives and variants of any of these forms of therapeutic proteins. Thus, antibodies include those that (1) have an amino acid sequence that has greater than about 60%, about 65%, about 70%, about 75%, about 80%, about 85%, about 90%, about 91%, about 92%, about 93%, about 94%, about 95%, about 96%, about 97%, about 98% or about 99% or greater amino acid sequence identity, over a region of at least about 25, about 50, about 100, about 200, about 300, about 400, or more amino acids, to a reference antibody (e.g., encoded by a referenced nucleic acid or an amino acid sequence described herein). According to the present disclosure, the term “recombinant protein” or “recombinant antibody” includes any protein obtained via recombinant DNA technology. In certain embodiments, the term encompasses antibodies as described herein.

In some embodiment, the antibodies or antibody variants described herein are expressed from one or more expression construct and/or in a cell or strains as described herein.

Exemplary wild-type or reference antibodies include commercially available or other known antibodies, including therapeutic monoclonal antibodies. Reference antibodies according to the present disclosure may include any antibodies now known or later developed, including those that are not clinically and/or commercially available.

### Cells and Expression Constructs

### Cells

Antibodies of the present disclosure, including wild-type (WT) antibodies and variant antibodies, are produced in some embodiments in cells. Cells comprising one or more of the expression constructs described herein are contemplated in various embodiments of the present disclosure.

Prokaryotic host cells. In some embodiments of the disclosure, expression constructs designed for expression of gene products, including fusion proteins as described herein, are provided in host cells, such as prokaryotic host cells. Prokaryotic host cells can include archaea (such as Haloferax volcanii, Sulfolobus solfataricus), Gram-positive bacteria (such as Bacillus subtilis, Bacillus licheniformis, Brevibacillus choshinensis, Lactobacillus brevis, Lactobacillus buchneri, Lactococcus lactis, and Streptomyces lividans), or Gram-negative bacteria, including Alphaproteobacteria (Agrobacterium tumefaciens, Caulobacter crescentus, Rhodobacter sphaeroides, and Sinorhizobium meliloti), Betaproteobacteria (Alcaligenes eutrophus), and Gammaproteobacteria (Acinetobacter calcoaceticus, Azotobacter vinelandii, Escherichia coli, Pseudomonas aeruginosa, and Pseudomonas putida). Preferred host cells include Gammaproteobacteria of the family Enterobacteriaceae, such as Enterobacter, Erwinia, Escherichia (including E. coli), Klebsiella, Proteus, Salmonella (including Salmonella typhimurium), Serratia (including Serratia marcescans), and Shigella.

Eukaryotic host cells. Many additional types of host cells can be used for the expression systems of the present disclosure, including eukaryotic cells such as yeast (Candida shehatae, Kluyveromyces lactis, Kluyveromyces fragilis, other Kluyveromyces species, Pichia pastoris, Saccharomyces cerevisiae, Saccharomyces pastorianus also known as Saccharomyces carlsbergensis, Schizosaccharomyces pombe, Dekkera/Brettanomyces species, and Yarrowia lipolyticd); other fungi (Aspergillus nidulans, Aspergillus niger, Neurospora crassa, Penicillium, Tolypocladium, Trichoderma reesia); insect cell lines (Drosophila melanogaster Schneider 2 cells and Spodoptera frugiperda Sf9 cells); and mammalian cell lines including immortalized cell lines (Chinese hamster ovary (CHO) cells, HeLa cells, baby hamster kidney (BHK) cells, monkey kidney cells (COS), human embryonic kidney (HEK, 293, or HEK-293) cells, and human hepatocellular carcinoma cells (Hep G2)). The above host cells are available from the American Type Culture Collection.

As described in WO/2017/106583, incorporated by reference in its entirety herein, producing gene products such as therapeutic proteins at commercial scale and in soluble form is addressed by providing suitable host cells capable of growth at high cell density in fermentation culture, and which can produce soluble gene products in the oxidizing host cell cytoplasm through highly controlled inducible gene expression. Host cells of the present disclosure with these qualities are produced by combining some or all of the following characteristics. (1) The host cells are genetically modified to have an oxidizing cytoplasm, through increasing the expression or function of oxidizing polypeptides in the cytoplasm, and/or by decreasing the expression or function of reducing polypeptides in the cytoplasm. Specific examples of such genetic alterations are provided herein. Optionally, host cells can also be genetically modified to express chaperones and/or cofactors that assist in the production of the desired gene product(s), and/or to glycosylate polypeptide gene products. (2) The host cells comprise one or more expression constructs designed for the expression of one or more gene products of interest; in certain embodiments, at least one expression construct comprises an inducible promoter and a polynucleotide encoding a gene product to be expressed from the inducible promoter. (3) The host cells contain additional genetic modifications designed to improve certain aspects of gene product expression from the expression construct(s). In particular embodiments, the host cells (A) have an alteration of gene function of at least one gene encoding a transporter protein for an inducer of at least one inducible promoter, and as another example, wherein the gene encoding the transporter protein is selected from the group consisting of araE, araE, araG, araH, rhaT, xylF, xylG, and xylH, or particularly is araE, or wherein the alteration of gene function more particularly is expression of araE from a constitutive promoter; and/or (B) have a reduced level of gene function of at least one gene encoding a protein that metabolizes an inducer of at least one inducible promoter, and as further examples, wherein the gene encoding a protein that metabolizes an inducer of at least one said inducible promoter is selected from the group consisting of araA, araB, araD, prpB, prpD, rhaA, rhaB, rhaD, xylA, and xylB; and/or (C) have a reduced level of gene function of at least one gene encoding a protein involved in biosynthesis of an inducer of at least one inducible promoter, which gene in further embodiments is selected from the group consisting of scpA/sbm, argK/ygfD, scpB/ygfG, scpC/ygfH, rmlA, rmlB, rmlC, and rmlD.

Host Cells with Oxidizing Cytoplasm. The expression systems of the present disclosure are designed to express gene products; in certain embodiments of the disclosure, the gene products are expressed in a host cell. Examples of host cells are provided that allow for the efficient and cost-effective expression of gene products, including components of multimeric products. Host cells can include, in addition to isolated cells in culture, cells that are part of a multicellular organism, or cells grown within a different organism or system of organisms. In certain embodiments of the disclosure, the host cells are microbial cells such as yeasts (Saccharomyces, Schizosaccharomyces, etc.) or bacterial cells, or are gram-positive bacteria or gram-negative bacteria, or are E. coli, or are an E. coli B strain, or are E. coli (B strain) EB0001 cells (also called E. coli ASE(DGH) cells), or are E. coli (B strain) EB0002 cells. In growth experiments with E. coli host cells having oxidizing cytoplasm, specifically the E. coli B strains SHuffle® Express (NEB Catalog No. C3028H) and SHuffle® T7 Express (NEB Catalog No. C3029H) and the E. coli K strain SHuffle® T7 (NEB Catalog No. C3026H), these E. coli B strains with oxidizing cytoplasm are able to grow to much higher cell densities than the most closely corresponding E. coli K strain (WO/2017/106583).

Alterations to host cell gene functions. Certain alterations can be made to the gene functions of host cells comprising inducible expression constructs, to promote efficient and homogeneous induction of the host cell population by an inducer. Preferably, the combination of expression constructs, host cell genotype, and induction conditions results in at least 75% (more preferably at least 85%, and most preferably, at least 95%) of the cells in the culture expressing gene product from each induced promoter, as measured by the method of Khlebnikov et al. described in Example 9 of WO/2017/106583. For host cells other than E. coli, these alterations can involve the function of genes that are structurally similar to an E. coli gene, or genes that carry out a function within the host cell similar to that of the E. coli gene. Alterations to host cell gene functions include eliminating or reducing gene function by deleting the gene protein-coding sequence in its entirety, or deleting a large enough portion of the gene, inserting sequence into the gene, or otherwise altering the gene sequence so that a reduced level of functional gene product is made from that gene. Alterations to host cell gene functions also include increasing gene function by, for example, altering the native promoter to create a stronger promoter that directs a higher level of transcription of the gene, or introducing a missense mutation into the protein-coding sequence that results in a more highly active gene product. Alterations to host cell gene functions include altering gene function in any way, including for example, altering a native inducible promoter to create a promoter that is constitutively activated. In addition to alterations in gene functions for the transport and metabolism of inducers, as described herein with relation to inducible promoters, and/or an altered expression of chaperone proteins, it is also possible to alter the reduction-oxidation environment of the host cell.

Host cell reduction-oxidation environment. In bacterial cells such as E. coli, proteins that need disulfide bonds are typically exported into the periplasm where disulfide bond formation and isomerization is catalyzed by the Dsb system, comprising DsbABCD and DsbG. Increased expression of the cysteine oxidase DsbA, the disulfide isomerase DsbC, or combinations of the Dsb proteins, which are all normally transported into the periplasm, has been utilized in the expression of heterologous proteins that require disulfide bonds (Makino et al., Microb Cell Fact 2011 May 14; 10: 32). It is also possible to express cytoplasmic forms of these Dsb proteins, such as a cytoplasmic version of DsbA and/or of DsbC (‘cDsbA or ‘cDsbC’), that lacks a signal peptide and therefore is not transported into the periplasm. Cytoplasmic Dsb proteins such as cDsbA and/or cDsbC are useful for making the cytoplasm of the host cell more oxidizing and thus more conducive to the formation of disulfide bonds in heterologous proteins produced in the cytoplasm. The host cell cytoplasm can also be made less reducing and thus more oxidizing by altering the thioredoxin and the glutaredoxin/glutathione enzyme systems directly: mutant strains defective in glutathione reductase (gor) or glutathione synthetase (gshB), together with thioredoxin reductase (trxB), render the cytoplasm oxidizing. These strains are unable to reduce ribonucleotides and therefore cannot grow in the absence of exogenous reductant, such as dithiothreitol (DTT). Suppressor mutations (such as ahpC* and ahpCA, Lobstein et al., Microb Cell Fact 2012 May 8; 11: 56; doi: 10.1186/1475-2859-11-56) in the gene ahpC, which encodes the peroxiredoxin AhpC, convert it to a disulfide reductase that generates reduced glutathione, allowing the channeling of electrons onto the enzyme ribonucleotide reductase and enabling the cells defective in gor and trxB, or defective in gshB and trxB, to grow in the absence of DTT. A different class of mutated forms of AhpC can allow strains, defective in the activity of gamma-glutamylcysteine synthetase (gshA) and defective in trxB, to grow in the absence of DTT; these include AhpC V164G, AhpC S71F, AhpC E173/S71F, AhpC E171Ter, and AhpC dupl62-169 (Faulkner et al., Proc Natl Acad Sci USA 2008 May 6; 105(18): 6735-6740, Epub 2008 May 2). In such strains with oxidizing cytoplasm, exposed protein cysteines become readily oxidized in a process that is catalyzed by thioredoxins, in a reversal of their physiological function, resulting in the formation of disulfide bonds. Other proteins that may be helpful to reduce the oxidative stress effects in host cells of an oxidizing cytoplasm are HPI (hydroperoxidase 1) catalase-peroxidase encoded by E. coli katG and HPII (hydroperoxidase 11) catalase-peroxidase encoded by E. coli katE, which disproportionate peroxide into water and 02 (Farr and Kogoma, Microbiol Rev. 1991 December; 55(4): 561-585; Review). Increasing levels of KatG and/or KatE protein in host cells through induced coexpression or through elevated levels of constitutive expression is an aspect of some embodiments of the disclosure.

Another alteration that can be made to host cells is to express the sulfhydryl oxidase Ervlp from the inner membrane space of yeast mitochondria in the host cell cytoplasm, which has been shown to increase the production of a variety of complex, disulfide-bonded proteins of eukaryotic origin in the cytoplasm of E. coli, even in the absence of mutations in gor or trxB (Nguyen et al, Microb Cell Fact 2011 Jan. 7; 10: 1).

Host cells comprising expression constructs preferably also express cDsbA and/or cDsbC and/or Ervlp; are deficient in trxB gene function; are also deficient in the gene function of either gor, gshB, or gshA; optionally have increased levels of katG and/or katE gene function; and express an appropriate mutant form of AhpC so that the host cells can be grown in the absence of DTT.

Chaperones. In some embodiments, desired gene products are coexpressed with other gene products, such as chaperones, that are beneficial to the production of the desired gene product. Chaperones are proteins that assist the non-covalent folding or unfolding, and/or the assembly or disassembly, of other gene products, but do not occur in the resulting monomeric or multimeric gene product structures when the structures are performing their normal biological functions (having completed the processes of folding and/or assembly). Chaperones can be expressed from an inducible promoter or a constitutive promoter within an expression construct, or can be expressed from the host cell chromosome; preferably, expression of chaperone protein(s) in the host cell is at a sufficiently high level to produce coexpressed gene products that are properly folded and/or assembled into the desired product. Examples of chaperones present in E. coli host cells are the folding factors DnaK/DnaJ/GrpE, DsbC/DsbG, GroEL/GroES, IbpA/IbpB, Skp, Tig (trigger factor), and FkpA, which have been used to prevent protein aggregation of cytoplasmic or periplasmic proteins. DnaK/DnaJ/GrpE, GroEL/GroES, and CIpB can function synergistically in assisting protein folding and therefore expression of these chaperones in combinations has been shown to be beneficial for protein expression (Makino et al., Microb Cell Fact 2011 May 14; 10: 32). When expressing eukaryotic proteins in prokaryotic host cells, a eukaryotic chaperone protein, such as protein disulfide isomerase (PDI) from the same or a related eukaryotic species, is in certain embodiments of the disclosure coexpressed or inducibly coexpressed with the desired gene product.

One chaperone that can be expressed in host cells is a protein disulfide isomerase from Humicola insolens, a soil hyphomycete (soft-rot fungus). An amino acid sequence of Humicola insolens PDI is shown as SEQ ID NO: I of WO/2017/106583; it lacks the signal peptide of the native protein so that it remains in the host cell cytoplasm. The nucleotide sequence encoding PDI was optimized for expression in E. coli; the expression construct for PDI is shown as SEQ ID NO: 2 of WO/2017/106583. SEQ ID NO: 2 contains a GCTAGC NheI restriction site at its 5′ end, an AGGAGG ribosome binding site at nucleotides 7 through 12, the PDI coding sequence at nucleotides 21 through 1478, and a GTCGAC Sail restriction site at its 3′ end. The nucleotide sequence of SEQ ID NO: 2 was designed to be inserted immediately downstream of a promoter, such as an inducible promoter. The NheI and Sail restriction sites in SEQ ID NO: 2 can be used to insert it into a vector multiple cloning site, such as that of the pSOL expression vector (SEQ ID NO: 3 of WO/2017/106583), described in published US patent application US2015353940A1. Other PDI polypeptides can also be expressed in host cells, including PDI polypeptides from a variety of species (Saccharomyces cerevisiae (UniProtKB PI 7967), Homo sapiens (UniProtKB P07237), Mus musculus (UniProtKB P09103), Caenorhabditis elegans (UniProtKB Q 17770 and Q 17967), Arabdopsis thaliana (UniProtKB 048773, Q9XI01, Q9S G3, Q9LJU2, Q9MAU6, Q94F09, and Q9T042), Aspergillus niger (UniProtKB Q12730) and also modified forms of such PDI polypeptides. In certain embodiments of the disclosure, a PDI polypeptide expressed in host cells of the disclosure shares at least 70%, or 80%, or 90%, or 95% amino acid sequence identity across at least 50% (or at least 60%, or at least 70%, or at least 80%, or at least 90%) of the length of SEQ ID NO: I of WO/2017/106583, where amino acid sequence identity is determined according to Example 10 of WO/2017/106583.

Cellular transport of cofactors. When using the expression systems of the disclosure to produce enzymes that require cofactors for function, it is helpful to use a host cell capable of synthesizing the cofactor from available precursors, or taking it up from the environment. Common cofactors include ATP, coenzyme A, flavin adenine dinucleotide (FAD), NAD+/NADH, and heme. Polynucleotides encoding cofactor transport polypeptides and/or cofactor synthesizing polypeptides can be introduced into host cells, and such polypeptides can be constitutively expressed, or inducibly coexpressed with the gene products to be produced by methods of the disclosure.

Glycosylation of polypeptide gene products. Host cells can have alterations in their ability to glycosylate polypeptides. For example, eukaryotic host cells can have eliminated or reduced gene function in glycosyltransferase and/or oligo-saccharyttransferase genes, impairing the normal eukaryotic glycosylation of polypeptides to form glycoproteins. Prokaryotic host cells such as E. coli, which do not normally glycosylate polypeptides, can be altered to express a set of eukaryotic and prokaryotic genes that provide a glycosylation function (DeLisa et al., WO2009089154A2, 2009 Jul. 16).

Available host cell strains with altered gene functions. To create preferred strains of host cells to be used in the expression systems and methods of the disclosure, it is useful to start with a strain that already comprises desired genetic alterations (Table A; WO/2017/106583).

TABLE A
Exemplary host cell strains

| Strain | Genotype | Source |
|---|---|---|
| E, coli | F− mcrA Δ(mrr-hsdRMS-mcrBC) | Invitrogen Life |
| TOP10 | Φ80lacZ ΔM15 Δlacx74 recA1 | Technologies |
| | araD139 Δ(ara-leu)7697 galU galK | Catalog |
| | rpsL (Str^{R}) endA1 | Nos. C404-10, |
| | nupG λ | C4040-3, C4040-6, |
| | | C4040-50, and |
| | | C4040-52 |
| E, coli | Δ(ara-leu)7697 Δlacx74 ΔphoA Pvull | Merck (EMD |
| Origami ™ | phoR araD139 ahpC gale galK rpsL | Millipore |
| 2 | F′[lac⁺ lacI^{q} pro] | Chemicals) |
| | gor522::Tn10 trxB (Str^{R}, | Catalog |
| | Tet^{R}) | No. 71344 |
| E, coli | fhuA2 [Ion] ompT ahpC gal | New England |
| SHuffle ® | λatt::pNEB3-r1-cDsbc (Spec, lacI) | Biolabs Catalog |
| Express | ΔtrxB sulA11 | No. C3028H |
| | R(mcr-73::miniTn10-Tet^{S})2 | |
| | [dem] R(zgb-210::Tn10-Te^{S}) | |
| | endA1 Δgor Δ(mcrC-mrr)114::IS10 | |

### Expression Constructs

In some embodiments of the present disclosure, inducible promoters are contemplated for use with the expression constructs. Exemplary promoters are described herein and are also described in WO/2016/205570, incorporated by reference in its entirety herein. As described herein, the cells comprising one or more expression constructs may optionally include one or more inducible promoters to express antibodies of the present disclosure, including wild-type antibodies and variant antibodies.

Expression Constructs. Expression constructs are polynucleotides designed for the expression of one or more antibodies, and thus are not naturally occurring molecules. Expression constructs can be integrated into a host cell chromosome, or maintained within the host cell as polynucleotide molecules replicating independently of the host cell chromosome, such as plasmids or artificial chromosomes. An example of an expression construct is a polynucleotide resulting from the insertion of one or more polynucleotide sequences into a host cell chromosome, where the inserted polynucleotide sequences alter the expression of chromosomal coding sequences. An expression vector is a plasmid expression construct specifically used for the expression of one or more gene products, such as one or more antibodies. One or more expression constructs can be integrated into a host cell chromosome or be maintained on an extrachromosomal polynucleotide such as a plasmid or artificial chromosome. The following are descriptions of particular types of polynucleotide sequences that can be used in expression constructs for the expression or coexpression of antibodies.

Origins of replication. Expression constructs must comprise an origin of replication, also called a replicon, in order to be maintained within the host cell as independently replicating polynucleotides. Different replicons that use the same mechanism for replication cannot be maintained together in a single host cell through repeated cell divisions. As a result, plasmids can be categorized into incompatibility groups depending on the origin of replication that they contain, as shown in Table 2 of WO/2016/205570. Origins of replication can be selected for use in expression constructs on the basis of incompatibility group, copy number, and/or host range, among other criteria. As described above, if two or more different expression constructs are to be used in the same host cell for the coexpression of multiple antibodies or components of antibodies (e.g., heavy and light chains, including fragments, as described herein), in one embodiment the different expression constructs contain origins of replication from different incompatibility groups: a pMBI replicon in one expression construct and a pl5A replicon in another, for example. The average number of copies of an expression construct in the cell, relative to the number of host chromosome molecules, is determined by the origin of replication contained in that expression construct. Copy number can range from a few copies per cell to several hundred (Table 2 of WO/2016/205570). In one embodiment of the disclosure, different expression constructs are used which comprise inducible promoters that are activated by the same inducer, but which have different origins of replication. By selecting origins of replication that maintain each different expression construct at a certain approximate copy number in the cell, it is possible to adjust the levels of overall production of an antibody component or fragment (e.g., a heavy or light chain) expressed from one expression construct, relative to another antibody component or fragment (e.g., a heavy or light chain) expressed from a different expression construct. As an example, to coexpress subunits A and B of a multimeric protein (including, for example, a heavy chain and a light chain), an expression construct is created which comprises the colEl replicon, the am promoter, and a coding sequence for subunit A expressed from the am promoter: ‘colEl-Para-A.

Another expression construct is created comprising the pl 5A replicon, the am promoter, and a coding sequence for subunit B:‘pl5A-Para-B’. These two expression constructs can be maintained together in the same host cells, and expression of both subunits A and B is induced by the addition of one inducer, arabinose, to the growth medium. If the expression level of subunit A needed to be significantly increased relative to the expression level of subunit B, in order to bring the stoichiometric ratio of the expressed amounts of the two subunits closer to a desired ratio, for example, a new expression construct for subunit A could be created, having a modified pMB 1 replicon as is found in the origin of replication of the pUC9 plasmid (‘pUC9ori’): pUC9ori-Para-A. Expressing subunit A from a high-copy-number expression construct such as pUC9ori-Para-A should increase the amount of subunit A produced relative to expression of subunit B from pl5A-Para-B. In a similar fashion, use of an origin of replication that maintains expression constructs at a lower copy number, such as pSOOI (WO/2016/205570), could reduce the overall level of a gene product expressed from that construct. Selection of an origin of replication can also determine which host cells can maintain an expression construct comprising that replicon. For example, expression constructs comprising the colEl origin of replication have a relatively narrow range of available hosts, species within the Enterobacteriaceae family, while expression constructs comprising the RK2 replicon can be maintained in E. coli, Pseudomonas aeruginosa, Pseudomonas putida, Azotobacter vinelandii, and Alcaligenes eutrophus, and if an expression construct comprises the RK2 replicon and some regulator genes from the RK2 plasmid, it can be maintained in host cells as diverse as Sinorhizobium meliloti, Agrobacterium tumefaciens, Caulobacter crescentus, Acinetobacter calcoaceticus, and Rhodobacter sphaeroides (Kiies and Stahl, Microbiol Rev 1989 December; 53(4): 491-516).

Similar considerations can be employed to create expression constructs for inducible expression or coexpression in eukaryotic cells. For example, the 2-micron circle plasmid of Saccharomyces cerevisiae is compatible with plasmids from other yeast strains, such as pSRI (ATCC Deposit Nos. 48233 and 66069; Araki et al., J Mol Biol 1985 Mar. 20; 182(2): 191-203) and pKDI (ATCC Deposit No. 37519; Chen et al, Nucleic Acids Res 1986 Jun. 11; 14(11): 4471-4481).

Selectable markers. Expression constructs usually comprise a selection gene, also termed a selectable marker, which encodes a protein necessary for the survival or growth of host cells in a selective culture medium. Host cells not containing the expression construct comprising the selection gene will not survive in the culture medium. Typical selection genes encode proteins that confer resistance to antibiotics or other toxins, or that complement auxotrophic deficiencies of the host cell. One example of a selection scheme utilizes a drug such as an antibiotic to arrest growth of a host cell. Those cells that contain an expression construct comprising the selectable marker produce a protein conferring drug resistance and survive the selection regimen. Some examples of antibiotics that are commonly used for the selection of selectable markers (and abbreviations indicating genes that provide antibiotic resistance phenotypes) are: ampicillin (AmpR), chloramphenicol (CmIR or CmR), kanamycin (KanR), spectinomycin (SpcR), streptomycin (StrR), and tetracycline (TetR). Many of the plasmids in Table 2 of WO/2016/205570 comprise selectable markers, such as pBR322 (AmpR, TetR); pMOB45 (CmR, TetR); pACYCIW (AmpR, KanR); and pGBMI (SpcR, StrR). The native promoter region for a selection gene is usually included, along with the coding sequence for its gene product, as part of a selectable marker portion of an expression construct. Alternatively, the coding sequence for the selection gene can be expressed from a constitutive promoter.

In various aspects, suitable selectable markers include, but are not limited to, neomycin phosphotransferase (npt II), hygromycin phosphotransferase (hpt), dihydrofolate reductase (dhfr), zeocin, phleomycin, bleomycin resistance gene (ble), gentamycin acetyltransferase, streptomycin phosphotransferase, mutant form of acetolactate synthase (als), bromoxynil nitrilase, phosphinothricin acetyl transferase (bar), enolpyruvylshikimate-3-phosphate (EPSP) synthase (aro A), muscle specific tyrosine kinase receptor molecule (MuSK-R), copper-zinc superoxide dismutase (sod1), metallothioneins (cup1, MT1), beta-lactamase (BLA), puromycin N-acetyl-transferase (pac), blasticidin acetyl transferase (bls), blasticidin deaminase (bsr), histidinol dehydrogenase (HDH), N-succinyl-5-aminoimidazole-4-carboxamide ribotide (SAICAR) synthetase (ade1), argininosuccinate lyase (arg4), beta-isopropylmalate dehydrogenase (leu2), invertase (suc2), orotidine-5′-phosphate (OMP) decarboxylase (ura3), and orthologs of any of the foregoing.

Inducible promoter. As described herein, there are several different inducible promoters that can be included in expression constructs as part of the inducible coexpression systems of the disclosure. Preferred inducible promoters share at least 80% polynucleotide sequence identity (more preferably, at least 90% identity, and most preferably, at least 95% identity) to at least 30 (more preferably, at least 40, and most preferably, at least 50) contiguous bases of a promoter polynucleotide sequence as defined in Table 1 of WO/2016/205570 by reference to the E. coli K-12 substrain MG1655 genomic sequence, where percent polynucleotide sequence identity is determined using the methods of Example 11 of WO/2016/205570. Under ‘standard’ inducing conditions (see Example 5 of WO/2016/205570), preferred inducible promoters have at least 75% (more preferably, at least 100%, and most preferably, at least 110%) of the strength of the corresponding ‘wild-type’ inducible promoter of E. coli K-12 substrain MG1655, as determined using the quantitative PCR method of De Mey et al. (Example 6 of WO/2016/205570). Within the expression construct, an inducible promoter is placed 5′ to (or ‘upstream of) the coding sequence for the gene product (e.g., antibody or antibody fragment) that is to be inducibly expressed, so that the presence of the inducible promoter will direct transcription of the gene product coding sequence in a 5′ to 3′ direction relative to the coding strand of the polynucleotide encoding the gene product.

Ribosome binding site. For some antibodies or antibody fragments, the nucleotide sequence of the region between the transcription initiation site and the initiation codon of the coding sequence of the gene product that is to be inducibly expressed corresponds to the 5′ untranslated region (‘UTR’) of the mRNA for the polypeptide gene product. Preferably, the region of the expression construct that corresponds to the 5′ UT comprises a polynucleotide sequence similar to the consensus ribosome binding site (RBS, also called the Shine-Dalgamo sequence) that is found in the species of the host cell. In prokaryotes (archaea and bacteria), the RBS consensus sequence is GGAGG or GGAGGU, and in bacteria such as E. coli, the RBS consensus sequence is AGGAGG or AGGAGGU. The RBS is typically separated from the initiation codon by 5 to 10 intervening nucleotides. In expression constructs, the RBS sequence is preferably at least 55% identical to the AGGAGGU consensus sequence, more preferably at least 70% identical, and most preferably at least 85% identical, and is separated from the initiation codon by 5 to 10 intervening nucleotides, more preferably by 6 to 9 intervening nucleotides, and most preferably by 6 or 7 intervening nucleotides. The ability of a given RBS to produce a desirable translation initiation rate can be calculated at the website salis.psu.edu/software/RBSLibraryCalculatorSearchMode, using the RBS Calculator; the same tool can be used to optimize a synthetic RBS for a translation rate across a 100,000+ fold range (Salis, Methods Enzymol 2011; 498: 19-42).

Multiple cloning site. A multiple cloning site (MCS), also called a polylinker, is a polynucleotide that contains multiple restriction sites in close proximity to or overlapping each other. The restriction sites in the MCS typically occur once within the MCS sequence, and preferably do not occur within the rest of the plasmid or other polynucleotide construct, allowing restriction enzymes to cut the plasmid or other polynucleotide construct only within the MCS. Examples of MCS sequences are those in the pBAD series of expression vectors, including pBAD18, pBAD18-Cm, pBAD18-Kan, pBAD24, pBAD28, pBAD30, and pBAD33 (Guzman et al., J Bacteriol 1995 July; 177(14): 4121-4130); or those in the pPRO series of expression vectors derived from the pBAD vectors, such as pPR018, pPR018-Cm, pPR018-Kan, pPR024, pPRO30, and pPR033 (U.S. Pat. No. 8,178,338 B2; May 15 2012; Keasling, Jay). A multiple cloning site can be used in the creation of an expression construct: by placing a multiple cloning site 3′ to (or downstream of) a promoter sequence, the MCS can be used to insert the coding sequence for a gene product to be expressed or coexpressed into the construct, in the proper location relative to the promoter so that transcription of the coding sequence will occur. Depending on which restriction enzymes are used to cut within the MCS, there may be some part of the MCS sequence remaining within the expression construct after the coding sequence or other polynucleotide sequence is inserted into the expression construct. Any remaining MCS sequence can be upstream or, or downstream of, or on both sides of the inserted sequence. A ribosome binding site can be placed upstream of the MCS, preferably immediately adjacent to or separated from the MCS by only a few nucleotides, in which case the RBS would be upstream of any coding sequence inserted into the MCS. Another alternative is to include a ribosome binding site within the MCS, in which case the choice of restriction enzymes used to cut within the MCS will determine whether the RBS is retained, and in what relation to, the inserted sequences. A further alternative is to include a RBS within the polynucleotide sequence that is to be inserted into the expression construct at the MCS, preferably in the proper relation to any coding sequences to stimulate initiation of translation from the transcribed messenger RNA.

Expression from constitutive promoters. Expression constructs of the disclosure can also comprise coding sequences that are expressed from constitutive promoters. Unlike inducible promoters, constitutive promoters initiate continual gene product production under most growth conditions. One example of a constitutive promoter is that of the Tn3 bla gene, which encodes beta-lactamase and is responsible for the ampicillin-resistance (AmpR) phenotype conferred on the host cell by many plasmids, including pBR322 (ATCC 31344), pACYCIW (ATCC 37031), and pBAD24 (ATCC 87399). Another constitutive promoter that can be used in expression constructs is the promoter for the E. coli lipoprotein gene, Ipp, which is located at positions 1755731-1755406 (plus strand) in E. coli K-12 substrain MG1655 (Inouye and Inouye, Nucleic Acids Res 1985 May 10; 13(9): 3101-3110). A further example of a constitutive promoter that has been used for heterologous gene expression in E. coli is the trpLEDCBA promoter, located at positions 1321169-1321133 (minus strand) in E. coli K-12 substrain MG1655 (Windass et al., Nucleic Acids Res 1982 Nov. 11; 10(21): 6639-6657). Constitutive promoters can be used in expression constructs for the expression of selectable markers, as described herein, and also for the constitutive expression of other gene products useful for the coexpression of the desired product. For example, transcriptional regulators of the inducible promoters, such as AraC, PrpR, RhaR, and XylR, if not expressed from a bidirectional inducible promoter, can alternatively be expressed from a constitutive promoter, on either the same expression construct as the inducible promoter they regulate, or a different expression construct. Similarly, gene products useful for the production or transport of the inducer, such as PrpEC, AraE, or Rha, or proteins that modify the reduction-oxidation environment of the cell, as a few examples, can be expressed from a constitutive promoter within an expression construct. Gene products useful for the production of coexpressed gene products, and the resulting desired product, also include chaperone proteins, cofactor transporters, etc.

Signal Peptides. Antibodies or antibody fragments expressed or coexpressed by the methods of the disclosure can contain signal peptides or lack them, depending on whether it is desirable for such gene products to be exported from the host cell cytoplasm into the periplasm, or to be retained in the cytoplasm, respectively. Signal peptides (also termed signal sequences, leader sequences, or leader peptides) are characterized structurally by a stretch of hydrophobic amino acids, approximately five to twenty amino acids long and often around ten to fifteen amino acids in length, that has a tendency to form a single alpha-helix. This hydrophobic stretch is often immediately preceded by a shorter stretch enriched in positively charged amino acids (particularly lysine). Signal peptides that are to be cleaved from the mature polypeptide typically end in a stretch of amino acids that is recognized and cleaved by signal peptidase. Signal peptides can be characterized functionally by the ability to direct transport of a polypeptide, either co-translationally or post-translationally, through the plasma membrane of prokaryotes (or the inner membrane of gram negative bacteria like E. coli), or into the endoplasmic reticulum of eukaryotic cells. The degree to which a signal peptide enables a polypeptide to be transported into the periplasmic space of a host cell like E. coli, for example, can be determined by separating periplasmic proteins from proteins retained in the cytoplasm, using a method such as described in Example 12 of WO/2016/205570.

The following is a description of inducible promoters that can be used in expression constructs for expression or coexpression of gene products, along with some of the genetic modifications that can be made to host cells that contain such expression constructs. Examples of these inducible promoters and related genes are, unless otherwise specified, from Escherichia coli (E. coli) strain MG1655 (American Type Culture Collection deposit ATCC 700926), which is a substrain of E. coli K-12 (American Type Culture Collection deposit ATCC 10798). Table 1 of WO/2016/205570 lists the genomic locations, in E. coli MG1655, of the nucleotide sequences for these examples of inducible promoters and related genes. Nucleotide and other genetic sequences, referenced by genomic location as in Table 1 of WO/2016/205570, are expressly incorporated by reference herein. Additional information about E. coli promoters, genes, and strains described herein can be found in many public sources, including the online EcoliWiki resource, located at ecoliwiki.net.

Arabinose promoter. (As used herein, ‘arabinose’ means L-arabinose.) Several E. coli operons involved in arabinose utilization are inducible by arabinose—araBAD, araC, arciE, and araFGH—but the terms ‘arabinose promoter’ and ‘ara promoter’ are typically used to designate the araBAD promoter. Several additional terms have been used to indicate the E. coli araBAD promoter, such as Para, ParaB, ParaBAD, and PBAD—The use herein of ‘ara promoter’ or any of the alternative terms given above, means the E. coli araBAD promoter. As can be seen from the use of another term, ‘araC-araBAD promoter’, the araBAD promoter is considered to be part of a bidirectional promoter, with the araBAD promoter controlling expression of the araBAD operon in one direction, and the araC promoter, in close proximity to and on the opposite strand from the araBAD promoter, controlling expression of the araC coding sequence in the other direction. The AraC protein is both a positive and a negative transcriptional regulator of the araBAD promoter. In the absence of arabinose, the AraC protein represses transcription from PBAD, but in the presence of arabinose, the AraC protein, which alters its conformation upon binding arabinose, becomes a positive regulatory element that allows transcription from PBAD—The araBAD operon encodes proteins that metabolize L-arabinose by converting it, through the intermediates L-ribulose and L-ribulose-phosphate, to D-xylulose-5-phosphate. For the purpose of maximizing induction of expression from an arabinose-inducible promoter, it is useful to eliminate or reduce the function of AraA, which catalyzes the conversion of L-arabinose to L-ribulose, and optionally to eliminate or reduce the function of at least one of AraB and AraD, as well. Eliminating or reducing the ability of host cells to decrease the effective concentration of arabinose in the cell, by eliminating or reducing the cell's ability to convert arabinose to other sugars, allows more arabinose to be available for induction of the arabinose-inducible promoter. The genes encoding the transporters which move arabinose into the host cell are araE, which encodes the low-affinity L-arabinose proton symporter, and the araFGH operon, which encodes the subunits of an ABC superfamily high-affinity L-arabinose transporter. Other proteins which can transport L-arabinose into the cell are certain mutants of the LacY lactose permease: the LacY(AIWC) and the LacY(AIWV) proteins, having a cysteine or a valine amino acid instead of alanine at position 177, respectively (Morgan-Kiss et al., Proc Natl Acad Sci USA 2002 May 28; 99(11): 7373-7377). In order to achieve homogenous induction of an arabinose-inducible promoter, it is useful to make transport of arabinose into the cell independent of regulation by arabinose. This can be accomplished by eliminating or reducing the activity of the AraFGH transporter proteins and altering the expression of araE so that it is only transcribed from a constitutive promoter. Constitutive expression of araE can be accomplished by eliminating or reducing the function of the native araE gene, and introducing into the cell an expression construct which includes a coding sequence for the AraE protein expressed from a constitutive promoter. Alternatively, in a cell lacking AraFGH function, the promoter controlling expression of the host cell's chromosomal araE gene can be changed from an arabinose-inducible promoter to a constitutive promoter. In similar manner, as additional alternatives for homogenous induction of an arabinose-inducible promoter, a host cell that lacks AraE function can have any functional AraFGH coding sequence present in the cell expressed from a constitutive promoter. As another alternative, it is possible to express both the araE gene and the araFGH operon from constitutive promoters, by replacing the native araE and araFGH promoters with constitutive promoters in the host chromosome. It is also possible to eliminate or reduce the activity of both the AraE and the AraFGH arabinose transporters, and in that situation to use a mutation in the LacY lactose permease that allows this protein to transport arabinose. Since expression of the lacY gene is not normally regulated by arabinose, use of a LacY mutant such as LacY(A177C) or LacY(A177V), will not lead to the ‘all or none’ induction phenomenon when the arabinose-inducible promoter is induced by the presence of arabinose. Because the LacY(A177C) protein appears to be more effective in transporting arabinose into the cell, use of polynucleotides encoding the LacY(A177C) protein is preferred to the use of polynucleotides encoding the LacY(A177V) protein.

Propionate promoter. The ‘propionate promoter’ or ‘prp promoter’ is the promoter for the E. coli prpBCDE operon, and is also called PPϕB. Like the ara promoter, the prp promoter is part of a bidirectional promoter, controlling expression of the prpBCDE operon in one direction, and with the prpR promoter controlling expression of the prpR coding sequence in the other direction. The PrpR protein is the transcriptional regulator of the prp promoter, and activates transcription from the prp promoter when the PrpR protein binds 2-methylcitrate (‘2-MC’). Propionate (also called propanoate) is the ion, CH3CH2COO—, of propionic acid (or ‘propanoic acid’), and is the smallest of the ‘fatty’ acids having the general formula H(CH2),COOH that shares certain properties of this class of molecules: producing an oily layer when salted out of water and having a soapy potassium salt. Commercially available propionate is generally sold as a monovalent cation salt of propionic acid, such as sodium propionate (CH3CH2COONa), or as a divalent cation salt, such as calcium propionate (Ca(CH3CH2COO)2). Propionate is membrane-permeable and is metabolized to 2-MC by conversion of propionate to propionyl-CoA by PrpE (propionyl-CoA synthetase), and then conversion of propionyl-CoA to 2-MC by PrpC (2-methylcitrate synthase). The other proteins encoded by the prpBCDE operon, PrpD (2-methylcitrate dehydratase) and PrpB (2-methylisocitrate lyase), are involved in further catabolism of 2-MC into smaller products such as pyruvate and succinate. In order to maximize induction of a propionate-inducible promoter by propionate added to the cell growth medium, it is therefore desirable to have a host cell with PrpC and PrpE activity, to convert propionate into 2-MC, but also having eliminated or reduced PrpD activity, and optionally eliminated or reduced PrpB activity as well, to prevent 2-MC from being metabolized. Another operon encoding proteins involved in 2-MC biosynthesis is the scpA-argK-scpBC operon, also called the sbm-yg/DGH operon. These genes encode proteins required for the conversion of succinate to propionyl-CoA, which can then be converted to 2-MC by PrpC. Elimination or reduction of the function of these proteins would remove a parallel pathway for the production of the 2-MC inducer, and thus might reduce background levels of expression of a propionate-inducible promoter, and increase sensitivity of the propionate-inducible promoter to exogenously supplied propionate. It has been found that a deletion of sbm-ygfD-ygfG-ygfH-ygfl, introduced into E. coli BL21(DE3) to create strain JSB (Lee and Keasling, Appl Environ Microbiol 2005 November; 71(11): 6856-6862), was helpful in reducing background expression in the absence of exogenously supplied inducer, but this deletion also reduced overall expression from the prp promoter in strain JSB. It should be noted, however, that the deletion sbm-ygfD-ygfG-ygfH-ygfl also apparently affects ygfl, which encodes a putative LysR-family transcriptional regulator of unknown function. The genes sbm-yg/DGH are transcribed as one operon, and ygfl is transcribed from the opposite strand. The 3′ ends of the ygfti and ygfl coding sequences overlap by a few base pairs, so a deletion that takes out all of the sbm-yg/DGH operon apparently takes out ygfl coding function as well. Eliminating or reducing the function of a subset of the sbm-ygfDGH gene products, such as YgfG (also called ScpB, methylmalonyl-CoA decarboxylase), or deleting the majority of the sbm-yg/DGH (or scpA-argK-scpBC) operon while leaving enough of the 3′ end of the ygfli (or scpC) gene so that the expression of ygfl is not affected, could be sufficient to reduce background expression from a propionate-inducible promoter without reducing the maximal level of induced expression.

Rhamnose promoter. (As used herein, ‘rhamnose’ means L-rhamnose.) The ‘rhamnose promoter’ or ‘rha promoter’, or PrhaSR, is the promoter for the E. coli rhaSR operon. Like the ara and prp promoters, the rha promoter is part of a bidirectional promoter, controlling expression of the rhaSR operon in one direction, and with the rhaBAD promoter controlling expression of the rhaBAD operon in the other direction. The rha promoter, however, has two transcriptional regulators involved in modulating expression: RhaR and RhaS. The RhaR protein activates expression of the rhaSR operon in the presence of rhamnose, while RhaS protein activates expression of the L-rhamnose catabolic and transport operons, rhaBAD and rhaT, respectively (Wickstrum et al, J Bacteriol 2010 January; 192(1): 225-232). Although the RhaS protein can also activate expression of the rhaSR operon, in effect RhaS negatively autoregulates this expression by interfering with the ability of the cyclic AMP receptor protein (CRP) to coactivate expression with RhaR to a much greater level. The rhaBAD operon encodes the rhamnose catabolic proteins RhaA (L-rhamnose isomerase), which converts L-rhamnose to L-rhamnulose; RhaB (rhamnulokinase), which phosphorylates L-rhamnulose to form L-rhamnulose-1-P; and RhaD (rhamnulose-1-phosphate aldolase), which converts L-rhamnulose-1-P to L-lactaldehyde and DHAP (dihydroxy acetone phosphate). To maximize the amount of rhamnose in the cell available for induction of expression from a rhamnose-inducible promoter, it is desirable to reduce the amount of rhamnose that is broken down by catalysis, by eliminating or reducing the function of RhaA, or optionally of RhaA and at least one of RhaB and RhaD. E. coli cells can also synthesize L-rhamnose from alpha-D-glucose-1-P through the activities of the proteins RmlA, RmlB, RmlC, and RmlD (also called RfbA, RfbB, RfbC, and RfbD, respectively) encoded by the rmlBDACX (or rfbBDACX) operon. To reduce background expression from a rhamnose-inducible promoter, and to enhance the sensitivity of induction of the rhamnose-inducible promoter by exogenously supplied rhamnose, it could be useful to eliminate or reduce the function of one or more of the RmlA, RmlB, RmlC, and

RmlD proteins. L-rhamnose is transported into the cell by RhaT, the rhamnose permease or L-rhamnose:proton symporter. As noted above, the expression of RhaT is activated by the transcriptional regulator RhaS. To make expression of RhaT independent of induction by rhamnose (which induces expression of RhaS), the host cell can be altered so that all functional RhaT coding sequences in the cell are expressed from constitutive promoters. Additionally, the coding sequences for RhaS can be deleted or inactivated, so that no functional RhaS is produced. By eliminating or reducing the function of RhaS in the cell, the level of expression from the rhaSR promoter is increased due to the absence of negative autoregulation by RhaS, and the level of expression of the rhamnose catalytic operon rhaBAD is decreased, further increasing the ability of rhamnose to induce expression from the rha promoter.

Xylose promoter. (As used herein, ‘xylose’ means D-xylose.) The xylose promoter, or ‘xyl promoter’, or PxyiA, means the promoter for the E. coli xylAB operon. The xylose promoter region is similar in organization to other inducible promoters in that the xylAB operon and the xylFGHR operon are both expressed from adjacent xylose-inducible promoters in opposite directions on the E. coli chromosome (Song and Park, J Bacteriol. 1997 November; 179(22): 7025-7032). The transcriptional regulator of both the PxyiA and PxyiF promoters is XylR, which activates expression of these promoters in the presence of xylose. The xylR gene is expressed either as part of the xylFGHR operon or from its own weak promoter, which is not inducible by xylose, located between the xylH and xylR protein-coding sequences. D-xylose is catabolized by XylA (D-xylose isomerase), which converts D-xylose to D-xylulose, which is then phosphorylated by XylB (xylulokinase) to form D-xylulose-5-P. To maximize the amount of xylose in the cell available for induction of expression from a xylose-inducible promoter, it is desirable to reduce the amount of xylose that is broken down by catalysis, by eliminating or reducing the function of at least XylA, or optionally of both XylA and XylB. The xylFGHR operon encodes XylF, XylG, and XylH, the subunits of an ABC super-family high-affinity D-xylose transporter. The xylE gene, which encodes the E. coli low-affinity xylose-proton symporter, represents a separate operon, the expression of which is also inducible by xylose. To make expression of a xylose transporter independent of induction by xylose, the host cell can be altered so that all functional xylose transporters are expressed from constitutive promoters. For example, the xylFGHR operon could be altered so that the xylFGH coding sequences are deleted, leaving XylR as the only active protein expressed from the xylose-inducible PxyiF promoter, and with the xylE coding sequence expressed from a constitutive promoter rather than its native promoter. As another example, the xylR coding sequence is expressed from the PxyiA or the promoter in an expression construct, while either the xylFGHR operon is deleted and xylE is constitutively expressed, or alternatively an xylFGH operon (lacking the xylR coding sequence since that is present in an expression construct) is expressed from a constitutive promoter and the xylE coding sequence is deleted or altered so that it does not produce an active protein.

Lactose promoter. The term ‘lactose promoter’ refers to the lactose-inducible promoter for the IacZYA operon, a promoter which is also called lacZpl; this lactose promoter is located at ca. 365603-365568 (minus strand, with the NA polymerase binding (‘-35’) site at ca. 365603-365598, the Pribnow box (‘-10’) at 365579-365573, and a transcription initiation site at 365567) in the genomic sequence of the E. coli K-12 substrain MG1655 (NCBI Reference Sequence NC 000913.2, I I-JAN-2012). In some embodiments, inducible coexpression systems of the disclosure can comprise a lactose-inducible promoter such as the IacZYA promoter. In other embodiments, the inducible coexpression systems of the disclosure comprise one or more inducible promoters that are not lactose-inducible promoters.

Alkaline phosphatase promoter. The terms ‘alkaline phosphatase promoter’ and ‘phoA promoter’ refer to the promoter for the phoApsiF operon, a promoter which is induced under conditions of phosphate starvation. The phoA promoter region is located at ca. 401647-401746 (plus strand, with the Pribnow box (‘-10’) at 401695-401701 (Kikuchi et al., Nucleic Acids Res 1981 Nov. 11; 9(21): 5671-5678)) in the genomic sequence of the E. coli K-12 substrain MG1655 (NCBI Reference Sequence NC 000913.3, 16 Dec. 2014). The transcriptional activator for the phoA promoter is PhoB, a transcriptional regulator that, along with the sensor protein PhoR, forms a two-component signal transduction system in E. coli. PhoB and PhoR are transcribed from the phoBR operon, located at ca. 417050-419300 (plus strand, with the PhoB coding sequence at 417,142-417,831 and the PhoR coding sequence at 417,889-419,184) in the genomic sequence of the E. coli K-12 substrain MG1655 (NCBI Reference Sequence NC 000913.3, 16 Dec. 2014). The phoA promoter differs from the inducible promoters described above in that it is induced by the lack of a substance—intracellular phosphate—rather than by the addition of an inducer. For this reason the phoA promoter is generally used to direct transcription of gene products that are to be produced at a stage when the host cells are depleted for phosphate, such as the later stages of fermentation. In some embodiments, inducible coexpression systems of the disclosure can comprise a phoA promoter. In other embodiments, the inducible coexpression systems of the disclosure comprise one or more inducible promoters that are not phoA promoters.

### Affinity Assays

Antibody binding and antibody affinity determination assays are well known in the art.

In one embodiment, an activity-specific cell-enrichment method (ACE/qaACE) can be used to identify host cells that express “active” antibodies rather than “inactive material.” Active antibodies can be distinguished from inactive antibodies by the ability of active antibodies to specifically bind a binding partner molecule (e.g., an antigen or epitope). The ACE assay protocol is described in WO/2021/146626, incorporated by reference herein. It will be appreciated by those of ordinary skill in the art that ACE can not only discriminate between active/inactive in a binary fashion, but can also compute a score that is proportional to affinity. Thus, ACE provides quantitative assay information, not merely binary/Boolean information, which enables the modeling techniques herein to perform regression techniques. This richer modeling represents an advantageous improvement over the limited binary classification of conventional techniques.

In another embodiment, the HiPR Bind assay described in WO/2021/163349 and incorporated by reference herein is used in conjunction with the methods provided herein.

Binding assays, for example assays that measure protein-protein interactions, including antibody-antigen interactions and including measuring binding affinity, are well known in the art. By way of example, Surface plasmon resonance (SPR), Dual polarisation interferometry (DPI), Static light scattering (SLS), Dynamic light scattering (DLS), Flow-induced dispersion analysis (FIDA), Fluorescence polarization/anisotropy, Fluorescence resonance energy transfer (FRET), Bio-layer interferometry (BLI), Isothermal titration calorimetry (ITC), Microscale thermophoresis (MST), Single colour reflectometry (SCORE) are contemplated. Additionally, Bimolecular fluorescence complementation (BiFC), affinity electrophoresis, label transfer, phage display, Tandem affinity purification (TAP), cross-linking, Quantitative immunoprecipitation combined with knock-down (QUICK) and Proximity ligation assay (PLA) are other well-known assays that provide protein-protein interaction information.

In some embodiments, the binding affinities of the antibodies described herein are measured by array surface plasmon resonance (SPR), according to standard techniques (Abdiche, et al. (2016) MAbs 8:264-277). Briefly, antibodies were immobilized on a HC 30M chip at four different densities/antibody concentrations. Varying concentrations (0-500 nM) of antibody target are then bound to the captured antibodies. Kinetic analysis is performed using Carterra software to extract association and dissociation rate constants (kₐ and k_{d}, respectively) for each antibody. Apparent affinity constants (K_{D}) are calculated from the ratio of k_{d}/kₐ. In some embodiments, the Carterra LSA Platform is used to determine kinetics and affinity. In other embodiments, binding affinity can be measured, e.g., by surface plasmon resonance (e.g., BIAcore™) using, for example, the IBIS MX96 SPR system from IBIS Technologies or the Carterra LSA SPR platform, or by Bio-Layer Interferometry, for example using the Octet™ system from ForteBio. In some embodiments, a biosensor instrument such as Octet RED384, ProteOn XPR36, IBIS MX96 and Biacore T100 is used (Yang, D., et al., J. Vis. Exp., 2017, 122:55659).

K_{D} is the equilibrium dissociation constant, a ratio of k_{off}/kₒₙ, between the antibody and its antigen. K_{D} and affinity are inversely related. The K_{D} value relates to the concentration of antibody and so the lower the K_{D} value (lower concentration) and thus the higher the affinity of the antibody. Antibody, including reference antibody and variant antibody, K_{D} according to various embodiments of the present disclosure can be, for example, in the micromolar range (10⁻⁴ to 10⁻⁶), the nanomolar range (10⁻⁷ to 10⁻⁹), the picomolar range (10⁻¹⁰ to 10⁻¹²) or the femtomolar range (10⁻¹³ to 10⁻¹⁵). In some embodiments, antibody affinity of a variant antibody is improved, relative to a reference antibody, by approximately 5, 10, 15, 20, 25, 30, 35, 40, 45, or 50% or more. The improvement may also be expressed relative to a fold change (e.g., 2×, 4×, 6×, or 2-, 3-, 4-, 5-, 6-, 7-, 8-, 9-, 10-fold or more improvement in binding activity, etc.) and/or an order of magnitude (e.g., 10⁷, 10⁸, 10⁹, etc.).

The data generated from the antibodies and assays described herein is, in some embodiments, used to train one or more models, as will be described next.

### Additional Considerations

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible. This is intended to provide support for all such combinations.

### EXAMPLES

### Example 1—Generation of Sequence Variants and Affinity Determination

For example, as depicted in FIG. 2, double mutants (SEQ ID NOs: 2-#) of 8 positions in the CDRH3 of the variable domains of trastuzumab (SEQ ID NOs: 1 and 2; and www.genome.jp/entry/D03257) were first generated.

SEQ ID NO: 1
(Heavy chain)
EVQLVESGGG LVQPGGSLRL SCAASGFNIK DTYIHWVRQA
PGKGLEWVAR IYPTNGYTRY ADSVKGRFTI SADTSKNTAY
LQMNSLRAED TAVYYCSRWG GDGFYAMDYW GQGTLVTVSS
SEQ ID NO: 2
(Light chain)
DIQMTQSPSS LSASVGDRVT ITCRASQDVN TAVAWYQQKP
GKAPKLLIYS ASFLYSGVPSRFSGSRSGTD FTLTISSLQP
EDFATYYCQQ HYTTPPTFGQ GTKVEIK

The variants were screened using an activity-based quantitative assay (WO/2021/146626) against Her2. Individual clones were selected across different gates to allow good representation of variants across a wide K_{D} range (e.g., 10⁻⁶ to 10⁻¹⁰ M). Individual clones were re-screened using Carterra SPR. This yielded about 500 unique sequence variants and associated K_{D}s. A model pre-trained using the OAS data set was further fine-tuned as discussed herein, using transfer learning, using the 500 unique sequence variants and their associated affinity labels.

An excerpt of the 500 unique variants follows:

| SEQ ID NO: | variant | kd |
|---|---|---|
| 3 | WYGQGFYA | 9.25E−11 |
| 4 | WGYDYFYA | 1.69E−10 |
| 5 | WGGDCRYA | 1.98E−10 |
| 6 | WGDNGFYA | 2.18E−10 |
| 7 | WGNDGFSA | 2.18E−10 |
| 8 | WGGPGFYA | 2.33E−10 |
| 9 | WWGSGFYA | 2.39E−10 |
| 10 | WHGTGFYA | 2.40E−10 |
| 11 | RGGDYFYA | 2.40E−10 |
| 12 | WHGYGFYA | 2.40E−10 |
| 13 | WGGDFGYA | 7.20E−7 |
| 14 | WGGDGFQA | 9.23E−7 |
| 15 | WVGDGFIA | 1.26E−6 |
| 16 | WHGDGTYA | 1.79E−6 |
| 17 | WGCDGFSA | 2.28E−6 |
| 18 | WVGDDFYA | 4.89E−6 |
| 19 | SGGDSFYA | 2.15E−5 |
| 20 | WGGDGFRA | 2.20E−5 |
| 21 | WGTDGVYA | 8.72E−5 |
| 22 | WGGDGFTT | 0.000101 |

Ten-fold cross-validation was performed, such that 450 variants were used for training and 50 were used to check prediction accuracy, across 10 independent dataset slices. Upon consolidating the cross-validation data sets, the Pearson correlation coefficient was found to be 0.74, as depicted in block 210 of FIG. 2.

It will be appreciated by those of ordinary skill in art that the present examples may include further modeling, including statistical analysis using available datasets. In some aspects, the goal of the affinity modeling is to predict the affinity of an antibody for its target based on sequence variations in the CDR regions. For example, with respect to Trastuzumab, a combinatorial mutagenesis of up to two mutations over eight amino acids may be performed in the CDRH3, as shown in the above example. Two types of experimental measurements may be measured: lower throughput, but highly accurate surface plasmon resonance (SPR) K_{D} readouts, and higher throughput (HT) but more noisy estimates of K_{D} from a proprietary ACE assay. The present techniques may include choosing samples for the SPR assay for two purposes, i.e., model training and evaluation. The training sequences may be chosen from a group of enriched binders in the HT screen (SPR). Additional sequences may be evaluated based on predictions from the trained SPR model. Performance measures may be based on pooled out-of-fold predictions from 10-fold cross-validation. All measures of K_{D} were based on a log 10 scale, as reflected in the respective RMSE metrics.

Sequence naturalness may be defined, in some aspects, as the inverse of its pseudo-perplexity, as is known for some masked language models:

naturalness=e^{-loss}

See, e.g., Salazar, J., Liang, D., Nguyen, T. Q., and Kirchhoff, K. Masked language model scoring. 2019. doi: 10.48550/ARXIV.1910.14659. URL https://arxiv. org/abs/1910.14659.

In some aspects, measurements of antibody titers in HEK-293 cells across 136 antibodies may be obtained (e.g., from Jain et al., 2017). To compare the relationship between titers and CDR sequence naturalness, the Mann-Whitney U test with a significance threshold of 0.5 may be used.

Antibody developability scores and flags may be evaluated, in some aspects, for sequences from a phage display screening library, expected to have a range of developability potential (e.g., Liu et al., 2019a). For example, the most abundant 5000 sequences may be evaluated on five criteria of developability using the Therapeutic Antibody Profiler (TAP) (Raybould, M. I. J., Marks, C., Krawczyk, K., Tad-dese, B., Nowak, J., Lewis, A. P., Bujotzek, A., Shi, J., and Deane, C. M. Five computational de-velopability guidelines for therapeutic antibody profil¬ing. Proceedings of the National Academy of Sci¬ences, 116(10):4025-4030, 2019. doi: 10.1073/pnas. 1810576116. URL https://www.pnas.org/doi/abs/10.1073/pnas.1810576116.).

In some aspects, immunogenicity levels reported as percent of patients with anti-drug antibody (ADA) responses may be obtained (e.g., from Marks et al., 2021). An analysis may be performed using only humanized antibodies. The analysis may include comparing immunogenicity levels between antibodies considered natural against those considered unnatural by the present modeling techniques, e.g., using the Mann-Whitney U test with a significance threshold of 0.5.

### Example 2—Generation of Sequence Variants and Affinity Determination Using Weak Binding Training Data

Using data from Example 1, the model was trained with the bottom (weaker binders) 450 variants by K_{D}, and then predicted the K_{D} of the top 50 variants (stronger binders). The model correctly predicted that most of these sequence variants were strong binders. However, the model was not able to predict the relative ranking of such strong binders, due to the fact that strong binders have a very narrow K_{D} distribution in which measurement error is greater than the accuracy needed for ranking. In other words, even a second repeat of Carterra measurements just for the top 50 variants is likely not to yield the same ranking as seen in the original Carterra measurements. Because the model is trained on experimental data and experimental data does not have the resolution to rank variants in a narrow K_{D} range, the model inherits that inability. Nevertheless the model is able to predict which binders are in the strongest bin of K_{D} affinity.

### Example 3—Generating Biomolecules of Interest Using Denoising and Naturalness

Examples and explanations of denoising and naturalness are shown in further detail in FIG. 4A-FIG. 4O. An antibody against a target of interest resulting from library screening, immunization and/or humanization campaigns may exhibit suboptimal properties, such as insufficient binding affinity, thereby requiring lead optimization. Structure-guided engineering is a powerful approach to improve antibodies, but it is time-consuming and it prompts the experimental validation of a limited set of solutions. By contrast, deep mutagenesis coupled with screening or selection allows exploration of a larger sequence space, thereby potentially yielding more and better variants. However, most mutations degrade binding rather than improving it, leading to reduced screening efficiency. Moreover, the combinatorics of explorable sequence space grows exponentially with mutational load, exceeding capacity of experimental assays by many orders of magnitude. Finally, with most approaches, variant libraries can only be screened for one property at a time, making it difficult to simultaneously optimize for multiple properties.

Lead antibodies require optimization of binding affinity and other properties. Traditional engineering approaches are time-consuming and explore only a subset of the solution sequence space. To address these challenges, we assist antibody development with AI, leveraging synthetic biology data generation. In some cases, machine learning and Artificial Intelligence (e.g., trained neural networks) using high-quality affinity measurements (K_{D}s or surrogates) of sequence variants of trastuzumab generated using proprietary wet lab assays may be used to predict the binding affinities of unseen sequence variants spanning nearly 4 orders of magnitudes of K_{D}s with high accuracy, resulting in the ability to perform screenings in silico. The quantitative nature of predictions enabled affinity maturation applications—strengthening, weakening or tuning antigen binding to a desired K_{D}. Moreover, by introducing sequences of natural antibodies into our AI models we could compare variants to human antibody repertoires, predicting those that were more likely to have natural antibody properties (high “naturalness” score). Empirical testing found that naturalness scores appear to mitigate downstream issues related to developability and immunogenicity. By using generative techniques, neural networks produced sequence variants optimized for both affinity and naturalness. The ability to restrict affinity maturation to high-naturalness sequence space is critical because median antibody variant naturalness decreased as mutational load increased. In summary, the present developments in artificial intelligence promise not only to accelerate and enhance antibody engineering, but also to enable entirely novel applications that could eventually improve the quality of antibodies. Models trained with affinity measurements of sequence variants (e.g., of trastuzumab) may quantitatively predict the binding strength of unseen variants. Models may score antibody sequences for predicted “naturalness” by comparison with human antibody repertoires. The present techniques may train one or more naturalness machine learning models using multi-species data. High naturalness scores may be associated positively with developability and negatively with immunogenicity. Generative techniques enable optimization for both affinity and naturalness. Naturalness is of high importance in many applications of the present techniques, such as drug discovery, wherein binding affinity is not the only relevant consideration.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

Aspects of the techniques described in the present disclosure may include any of the following aspects, either alone or in combination:
1. A computing system for identifying biomolecule sequence variants of interest, the computing system comprising: one or more processors; and one or more non-transitory computer-readable media having stored thereon: a machine-learned model trained using training data, wherein the training data includes one or more training biomolecule sequence variants, each having a respective measured binding characteristic representing an ability of each to bind to a corresponding respective binding partner, and wherein the machine-learned model is configured to output a predicted biomolecule binding characteristic of an input biomolecule sequence variant; and instructions that, when executed by the one or more processors, cause the computing system to: process one or more biomolecule sequence variants with the machine-learned model to generate one or more predicted binding characteristics, each corresponding to a respective one of the one or more biomolecule sequence variants; analyze the one or more predicted binding characteristics to identify one or more biomolecule sequence variants of interest from among the one or more biomolecule sequence variants, each of the one or more biomolecule sequence variants of interest having a respective one or more desired properties; and provide the one or more biomolecule sequence variants of interest as an output.
2. The computing system of aspect 1, wherein at least one of the one or more training biomolecule sequence variants is an antibody sequence variant, and the corresponding respective binding partner is an antigen; and wherein the one or more biomolecule sequence variants are antibody sequence variants.
3. The computing system of aspect 2, wherein at least one of the one or more training biomolecule sequence variants is an antigen sequence variant, and the corresponding respective binding partner is an antibody; and wherein one or more biomolecule sequence variants are antigen sequence variants.
4. The computing system of aspect 1, wherein the training data includes multi-species sequence data.
5. The computing system of aspect 4, wherein the multi-species sequence data includes at least one of (i) human sequence data, (ii) mouse sequence data, or (iii) camelid sequence data.
6. The computing system of aspect 1, wherein the one or more training biomolecule sequence variants include less than 10% of a total possible variant space, less than 9% of a total possible variant space, less than 8% of a total possible variant space, less than 7% of a total possible variant space, less than 6% of a total possible variant space, less than 5% of a total possible variant space, less than 4% of a total possible variant space, less than 3% of a total possible variant space, less than 2% of a total possible variant space; or less than 1% of a total possible variant space.
7. The computing system of aspect 1, wherein the one or more training biomolecule sequence variants include less than 0.5% of a total possible variant space, less than 0.4% of a total possible variant space, less than 0.3% of a total possible variant space, less than 0.2% of a total possible variant space; or less than 0.1% of a total possible variant space.
8. The computing system of any one of aspects 1 through 7, the one or more non-transitory computer-readable media having stored thereon further instructions that, when executed by the one or more processors, cause the computing system to: obtain at least one of the one or more training biomolecule sequence variants from at least one of: (i) Observed Antibody Space (OAS) database; (ii) Uniref90 protein database; (iii) any Uniref-derived dataset; (iv) a BFD dataset; (v) a Mgnify dataset; (vi) any metagenomic dataset derived from a JGI or EBI compendiums; (vii) any corpus of assembled protein sequences; or (viii) any dataset of natural antibody sequences, which might be obtained by BCR-sequencing or other means.
9. The computing system of any one of aspects 1 through 8, the one or more non-transitory computer-readable media having stored thereon further instructions that, when executed by the one or more processors, cause the computing system to: pre-train the machine-learned model using a self-supervised pre-training objective to analyze the one or more training biomolecule sequence variants, wherein the pre-training includes generating a set of universal model weights.
10. The computing system of aspect 9, wherein the self-supervised pre-training objective is a masked language model objective.
11. The computing system of any one of aspects 1 through 10, the one or more non-transitory computer-readable media having stored thereon further instructions that, when executed by the one or more processors, cause the computing system to: pre-train the machine-learned model in response to determining that a number of the training biomolecule sequence variants in the training data is less than a predetermined threshold.
12. The computing system of any one of aspects 1 through 11, the one or more non-transitory computer-readable media having stored thereon further instructions that, when executed by the one or more processors, cause the computing system to: further train the machine-learned model using data output by at least one binding assay corresponding to an antibody-antigen pair, the antibody-antigen pair corresponding to a set of antibody-antigen-specific weights.
13. The computing system of aspect 12, wherein the at least one binding assay includes at least one of: (i) high-throughput screening, (ii) low-throughput screening, (iii) high accuracy targeted screening, (iv) a surface plasmon resonance (SPR) technique, (v) an isothermal titration calorimetry (ITC) technique, (vi) a biolayer interferometry (BLI) technique, or (vii) a microscale thermophoresis (MST) technique.
14. The computing system of any one of aspects 1 through 13, the one or more non-transitory computer-readable media having stored thereon further instructions that, when executed by the one or more processors, cause the computing system to: re-train the machine-learned model using data output by a different at least one binding assay corresponding to a different antibody-antigen pair, wherein the re-training includes generating a different set of antibody-antigen-specific weights corresponding to the different antibody-antigen pair.
15. The computing system of any one of aspects 1 through 14, the one or more non-transitory computer-readable media having stored thereon further instructions that, when executed by the one or more processors, cause the computing system to: determine at least one respective measured binding characteristic based on an environmental condition.
16. The computing system of aspect 1, wherein the one or more biomolecule sequence variants include a reference antibody.
17. The computing system of aspect 1, wherein the one or more biomolecule sequence variants include at least one of a commercial antibody, a non-commercial antibody, a clinical antibody, a non-clinical antibody, a research-grad antibody, a diagnostic-grade antibody, a publicly-available antibody, an antibody derived from patient samples, a de novo antibody discovered in vivo, a de novo antibody discovered in vitro, or a de novo antibody discovered in silico.
18. The computing system of aspect 1, wherein the one or more biomolecule sequence variants include at least one sequence variant selected from the group consisting of a monoclonal antibody, a human antibody, a humanized antibody, a camelised antibody, a chimeric antibody, single-chain Fvs (scFv), disulfide-linked Fvs (sdFv), Fab fragments, F (ab′) fragments, anti-idiotypic (anti-Id) antibody and epitope-binding fragments of any of the above.
19. The computing system of aspect 1, wherein the one or more non-transitory computer-readable media having stored thereon further instructions that, when executed by the one or more processors, cause the computing system to: generate the one or more biomolecule sequence variants by programmatically mutating one or more amino acids of at least one biomolecule in the one or more biomolecule sequence variants.
20. The computing system of any one of aspects 1 through 19, wherein the one or more non-transitory computer-readable media having stored thereon further instructions that, when executed by the one or more processors, cause the computing system to: generate the one or more biomolecule sequence variants by programmatically mutating one or more regions of the at least one of the one or more biomolecule sequence variants, selected from the group consisting of complementarity determining regions (CDR), heavy chain variable region (VH), light chain variable region (VL), framework (FR), or constant domain of an antibody.
21. The computing system of any one of aspects 1 through 20, wherein the one or more non-transitory computer-readable media having stored thereon further instructions that, when executed by the one or more processors, cause the computing system to: generate the one or more biomolecule sequence variants by programmatically mutating one or more CDR selected from the group consisting of CDR1, CDR2 and CDR3 of the VH.
22. The computing system of any one of aspects 1 through 21, wherein the one or more non-transitory computer-readable media having stored thereon further instructions that, when executed by the one or more processors, cause the computing system to: generate the one or more biomolecule sequence variants by programmatically mutating one or more CDR selected from the group consisting of CDR1, CDR2 and CDR3 of the VL.
23. The computing system of aspect 1, wherein an isotype of at least one of the one or more biomolecule sequence variants is selected from the group consisting of IgG, IgE, IgM, IgD, IgA and IgY.
24. The computing system of aspect 1, wherein at least one of the one or more predicted binding characteristics is expressed as an equilibrium dissociation constant (K_{D}) and is improved by 2-, 3-, 4-, 5-, 6-, 7-, 8-, 9-, or 10-fold or more relative to at least one of the one or more biomolecule sequence variants.
25. The computing system of aspect 1, wherein respective desired properties of at least one variant of interest in the one or more variants of interest include at least one of: (i) an increase in at least one predicted binding equilibrium of the variant of interest; (ii) a decrease in at least one predicted binding equilibrium of the variant of interest; (iii) an upper bound of at least one predicted binding equilibrium of the variant of interest; (iv) a lower bound of at least one predicted binding equilibrium of the variant of interest; (v) an increase in equilibrium toward a first antigen of a first predicted binding equilibrium of the variant of interest and a decrease in equilibrium toward a second antigen of a second predicted binding equilibrium of the variant of interest; (vi) ability of a cytokine sequence of a variant of interest to increase or decrease binding equilibrium towards receptors; (vii) suitability of a variant of interest for use as a next-generation antibody scaffold and/or antibody mimetic scaffold; (viii) ability of a variant of interest in an Fc region of an antibody to bind to an Fc receptor; (ix) a developability of the variant of interest as indicated by tolerability upon administration; or (x) an ability of a protein to interact with another protein.
26. The computing system of aspect 1, wherein the predicted binding characteristics include at least one of: (i) a numerical dissociation constant (Kd); (ii) a surrogate/correlate to Kd; (iii) a numerical association constant (Ka); or (iv) a surrogate/correlate to Ka.
27. The computing system of aspect 1, wherein the non-transitory computer-readable media stores at least one of: (i) an artificial neural network; (ii) a transformer neural network; (iii) a convolutional neural network; (iv) a recurrent neural network; (v) a deep learning network; (vi) an autoencoder; (vii) a regression model; (viii) a plug-and-play language model; (iv) a generative model; or (x) a genetic algorithm.
28. A computer-implemented method for training a machine learning model to identify biomolecule sequence variants of interest, the method comprising: generating one or more biomolecule sequence variants by programmatically mutating a reference biomolecule; receiving screening data including a ranking of the biomolecule sequence variants according to one or more training binding characteristics; and training the machine learning model using the screening data to predict one or more desired binding characteristics of an input biomolecule sequence variant.
29. The computer-implemented method of aspect 28, further comprising: receiving rescreening data corresponding to the biomolecule sequence variants to amplify the one or more training binding characteristics; and further training the machine learning model using the rescreening data to improve accuracy of the machine learning model.
30. The computer-implemented method of aspect 28, wherein the training binding characteristics include binding affinity (KD).
31. The computer-implemented method of aspect 28, wherein the screening data is received from one or both of (i) a human experimenter, and (ii) an assay device.
32. The computer-implemented method of aspect 28, wherein the one or more biomolecule sequence variants includes an antibody or an antigen.
33. A computing system for improving accuracy and throughput via predictive denoising, the computing system comprising: one or more processors; and one or more non-transitory computer-readable media having stored thereon: a machine-learned model trained using training data, wherein the training data includes one or more training biomolecule sequence variants, each having a respective measured binding characteristic representing an ability of each to bind to a corresponding respective binding partner, and wherein the machine-learned model is configured to output a predicted denoised biomolecule binding characteristic of one or more training biomolecule sequence variants; and instructions that, when executed by the one or more processors, cause the computing system to: process the one or more training biomolecule sequence variants with the machine-learned model to generate one or more denoised predicted binding characteristics, each corresponding to a respective one of the one or more training biomolecule sequence variants; and provide the one or more training biomolecule sequence variants and respective denoised predicted binding characteristics as output.
34. The computing system of aspect 33, wherein the training biomolecule sequence variants include one or more unsaturated sequence variants.
35. The computing system of aspect 33, wherein the each respective measured binding characteristic representing an ability of the each to bind to the corresponding respective binding partner is determined via an ACE assay™.
36. A computing system for predicting a naturalness of a biomolecule sequence variant, the computing system comprising: one or more processors; and one or more non-transitory computer-readable media having stored thereon: a machine-learned model trained using training data, wherein the training data includes one or more training biomolecule sequence variants, and wherein the machine-learned model is configured to output a respective predicted naturalness characteristic of one or more biomolecule sequence variants; and instructions that, when executed by the one or more processors, cause the computing system to: process one or more input biomolecule sequence variants with the machine-learned model to generate a respective predicted naturalness characteristic for each of the one or more input biomolecule sequence variants; and provide at least one of the predicted naturalness characteristics as output.
37. The computing system of aspect 36, the non-transitory computer-readable media having stored thereon further instructions that, when executed by the one or more processors, cause the computing system to: compare the respective predicted naturalness characteristic for each of the one or more input biomolecule sequence variants to one or both of (i) published phage data and (ii) a Therapeutic Antibody Profiler to determine one or more correlations between at least one respective naturalness characteristic and a developability characteristic.
38. The computing system of any one of aspects 36 through 37, the non-transitory computer-readable media having stored thereon further instructions that, when executed by the one or more processors, cause the computing system to: generate origin-binned data by comparing the respective predicted naturalness characteristic for each of the one or more input biomolecule sequence variants to published naturalness of therapeutic antibodies administered to humans in phase I, phase II, phase III or clinical phase using a CDR-only model; and determine an immunogenicity scoring by splitting the origin-binned data according to whether patients developed an anti-drug antibody response to fully human antibodies.
39. The computing system of any one of aspects 36 through 38, the non-transitory computer-readable media having stored thereon further instructions that, when executed by the one or more processors, cause the computing system to: score naturalness of a sequence variant as a function of CDRH3 mutational load.
40. The computing system of aspect any one of aspects 36 through 39, the non-transitory computer-readable media having stored thereon further instructions that, when executed by the one or more processors, cause the computing system to: score a naturalness of trastuzumab.
41. The computing system of aspect any one of aspects 36 through 40, the non-transitory computer-readable media having stored thereon further instructions that, when executed by the one or more processors, cause the computing system to: process the one or more biomolecule sequence variants with the machine-learned model to generate the one or more predicted binding characteristics and analyze the one or more predicted binding characteristics to identify one or more biomolecule sequence variants of interest from among the one or more biomolecule sequence variants using a generative technique, to avoid exhaustively predicting affinity of every possible sequence variant in a sequence space.
42. The computing system of aspect any one of aspects 36 through 41, the non-transitory computer-readable media having stored thereon further instructions that, when executed by the one or more processors, cause the computing system to: process the one or more biomolecule sequence variants with the machine-learned model to generate the one or more predicted binding characteristics based on a respective predicted naturalness of the one or more biomolecule sequence variants.
43. The computing system of any one of aspects 36 through 42, the non-transitory computer-readable media having stored thereon further instructions that, when executed by the one or more processors, cause the computing system to: analyze the one or more predicted binding characteristics to identify the one or more biomolecule sequence variants of interest from among the sequence variants by minimizing affinity and maximizing naturalness.
44. The computing system of any one of aspects 36 through 43, the non-transitory computer-readable media having stored thereon further instructions that, when executed by the one or more processors, cause the computing system to: analyze the one or more predicted binding characteristics to identify the one or more biomolecule sequence variants of interest from among the sequence variants by maximizing affinity of the one or more biomolecule sequence variants and minimizing naturalness of the one or more biomolecule sequence variants.

1. A computing system for identifying biomolecule sequence variants of interest, the computing system comprising:
one or more processors; and
one or more non-transitory computer-readable media having stored thereon:
a machine-learned model trained using training data,
wherein the training data includes one or more training biomolecule sequence variants, each having a respective measured binding characteristic representing an ability of each to bind to a corresponding respective binding partner, and
wherein the machine-learned model is configured to output a predicted biomolecule binding characteristic of an input biomolecule sequence variant; and
instructions that, when executed by the one or more processors, cause the computing system to:
process one or more biomolecule sequence variants with the machine-learned model to generate one or more predicted binding characteristics, each corresponding to a respective one of the one or more biomolecule sequence variants;
analyze the one or more predicted binding characteristics to identify one or more biomolecule sequence variants of interest from among the one or more biomolecule sequence variants, each of the one or more biomolecule sequence variants of interest having a respective one or more desired properties; and
provide the one or more biomolecule sequence variants of interest as an output.

2. The computing system of claim 1, wherein one or both of:
(i) at least one of the one or more training biomolecule sequence variants is an antibody sequence variant, and the corresponding respective binding partner is an antigen; and the one or more biomolecule sequence variants are antibody sequence variants; and
(ii) at least one of the one or more training biomolecule sequence variants is an antigen sequence variant, and the corresponding respective binding partner is an antibody; and the one or more biomolecule sequence variants are antigen sequence variants.

3. The computing system of claim 1,
wherein the training data includes multi-species sequence data comprising at least one of (i) human sequence data, (ii) mouse sequence data, (iii) camelid sequence data, or (iv) sequence data corresponding to another species.

4. (canceled)

5. The computing system of claim 1, the one or more non-transitory computer-readable media having stored thereon further instructions that, when executed by the one or more processors, cause the computing system to:
obtain at least one of the one or more training biomolecule sequence variants from at least one of:
(i) Observed Antibody Space (OAS) database;
(ii) Uniref90 protein database;
(iii) any Uniref-derived dataset;
(iv) a BFD dataset;
(v) a Mgnify dataset;
(vi) any metagenomic dataset derived from a JGI or EBI compendiums;
(vii) any corpus of assembled protein sequences; or
(viii) any dataset of natural antibody sequences, which might be obtained by BCR-sequencing or other means.

6. The computing system of claim 1, the one or more non-transitory computer-readable media having stored thereon further instructions that, when executed by the one or more processors, cause the computing system to:
at least one of
(i) pre-train the machine-learned model using a self-supervised pre-training objective to analyze the one or more training biomolecule sequence variants, wherein the pre-training includes generating a set of universal model weights,
(ii) pre-train the machine-learned model using a self-supervised pre-training objective to analyze the one or more training biomolecule sequence variants, wherein the pre-training includes generating a set of universal model weights, wherein the self-supervised pre-training objective is a masked language model objective,
(iii) pre-train the machine-learned model in response to determining that a number of the training biomolecule sequence variants in the training data is less than a predetermined threshold; or
(iv) further train the machine-learned model using data output by at least one binding assay corresponding to an antibody-antigen pair, the antibody-antigen pair corresponding to a set of antibody-antigen-specific weights.

7. The computing system of claim 6,
wherein the at least one binding assay includes at least one of:
(i) high-throughput screening,
(ii) low-throughput screening,
(iii) high accuracy targeted screening,
(iv) a surface plasmon resonance (SPR) technique,
(v) an isothermal titration calorimetry (ITC) technique,
(vi) a biolayer interferometry (BLI) technique, or
(vii) a microscale thermophoresis (MST) technique.

8. The computing system of claim 7, the one or more non-transitory computer-readable media having stored thereon further instructions that, when executed by the one or more processors, cause the computing system to:
re-train the machine-learned model using data output by a different at least one binding assay corresponding to a different antibody-antigen pair,
wherein the re-training includes generating a different set of antibody-antigen-specific weights corresponding to the different antibody-antigen pair.

9. The computing system of claim 1, the one or more non-transitory computer-readable media having stored thereon further instructions that, when executed by the one or more processors, cause the computing system to:
determine at least one respective measured binding characteristic based on an environmental condition.

10. The computing system of claim 1, wherein the one or more biomolecule sequence variants include at least one of:
a reference antibody,
a commercial antibody,
a non-commercial antibody,
a clinical antibody,
a non-clinical antibody,
a research-grade antibody,
a diagnostic-grade antibody,
a publicly-available antibody,
an antibody derived from patient samples,
a de novo antibody discovered in vivo,
a de novo antibody discovered in vitro,
a de novo antibody discovered in silico.

11. The computing system of claim 1, wherein the one or more biomolecule sequence variants include at least one sequence variant selected from the group consisting of a monoclonal antibody, a human antibody, a humanized antibody, a camelised antibody, a chimeric antibody, single-chain Fvs (scFv), disulfide-linked Fvs (sdFv), Fab fragments, F (ab′) fragments, anti-idiotypic (anti-Id) antibody and epitope-binding fragments of any of the above.

12. The computing system of claim 1, wherein the one or more non-transitory computer-readable media having stored thereon further instructions that, when executed by the one or more processors, cause the computing system to:
generate the one or more biomolecule sequence variants by programmatically mutating:
(i) one or more amino acids of at least one biomolecule in the one or more biomolecule sequence variants;
(ii) one or more regions of the at least one of the one or more biomolecule sequence variants, selected from the group consisting of complementarity determining regions (CDR), heavy chain variable region (VH), light chain variable region (VL), framework (FR), or constant domain of an antibody;
(iii) one or more CDR selected from the group consisting of CDR1, CDR2 and CDR3 of the VH; or
(iv) one or more CDR selected from the group consisting of CDR1, CDR2 and CDR3 of the VL.

13. The computing system of claim 1, wherein an isotype of at least one of the one or more biomolecule sequence variants is selected from the group consisting of IgG, IgE, IgM, IgD, IgA and IgY.

14. The computing system of claim 1, wherein at least one of the one or more predicted binding characteristics is expressed as an equilibrium dissociation constant (K_{D}) and is improved by 2-, 3-, 4-, 5-, 6-, 7-, 8-, 9-, or 10-fold or more relative to at least one of the one or more biomolecule sequence variants.

15. The computing system of claim 1, wherein respective desired properties of at least one variant of interest in the one or more variants of interest include at least one of:
(i) an increase in at least one predicted binding equilibrium of the variant of interest;
(ii) a decrease in at least one predicted binding equilibrium of the variant of interest;
(iii) an upper bound of at least one predicted binding equilibrium of the variant of interest;
(iv) a lower bound of at least one predicted binding equilibrium of the variant of interest;
(v) an increase in equilibrium toward a first antigen of a first predicted binding equilibrium of the variant of interest and a decrease in equilibrium toward a second antigen of a second predicted binding equilibrium of the variant of interest;
(vi) ability of a cytokine sequence of a variant of interest to increase or decrease binding equilibrium towards receptors;
(vii) suitability of a variant of interest for use as a next-generation antibody scaffold and/or antibody mimetic scaffold;
(viii) ability of a variant of interest in an Fc region of an antibody to bind to an Fc receptor;
(ix) a developability of the variant of interest as indicated by tolerability upon administration; or
(x) an ability of a protein to interact with another protein.

16. The computing system of claim 1, wherein the predicted binding characteristics include at least one of:
(i) a numerical dissociation constant (Kd);
(ii) a surrogate/correlate to Kd;
(iii) a numerical association constant (Ka); or
(iv) a surrogate/correlate to Ka.

17. The computing system of claim 1, wherein the non-transitory computer-readable media stores at least one of:
(i) an artificial neural network;
(ii) a transformer neural network;
(iii) a convolutional neural network;
(iv) a recurrent neural network;
(v) a deep learning network;
(vi) an autoencoder;
(vii) a regression model;
(viii) a plug-and-play language model;
(iv) a generative model; or
(x) a genetic algorithm.

18. A computer-implemented method for training a machine learning model to identify biomolecule sequence variants of interest, the method comprising:
generating one or more biomolecule sequence variants by programmatically mutating a reference biomolecule;
receiving screening data including a ranking of the biomolecule sequence variants according to one or more training binding characteristics; and
training the machine learning model using the screening data to predict one or more desired binding characteristics of an input biomolecule sequence variant.

19. The computer-implemented method of claim 18, further comprising:
receiving rescreening data corresponding to the biomolecule sequence variants to amplify the one or more training binding characteristics; and
further training the machine learning model using the rescreening data to improve accuracy of the machine learning model.

20. The computer-implemented method of claim 18, wherein the training binding characteristics include binding affinity (KD).

21. The computer-implemented method of claim 18, wherein the screening data is received from one or both of (i) a human experimenter, and (ii) an assay device.

22. The computer-implemented method of claim 18, wherein the one or more biomolecule sequence variants includes an antibody or an antigen.

23. A computing system for improving accuracy and throughput via predictive denoising, the computing system comprising:
one or more processors; and
one or more non-transitory computer-readable media having stored thereon:
a machine-learned model trained using training data,
wherein the training data includes one or more training biomolecule sequence variants, each having a respective measured binding characteristic representing an ability of each to bind to a corresponding respective binding partner, and
wherein the machine-learned model is configured to output a predicted denoised biomolecule binding characteristic of one or more training biomolecule sequence variants; and
instructions that, when executed by the one or more processors, cause the computing system to:
process the one or more training biomolecule sequence variants with the machine-learned model to generate one or more denoised predicted binding characteristics, each corresponding to a respective one of the one or more training biomolecule sequence variants; and
provide the one or more training biomolecule sequence variants and respective denoised predicted binding characteristics as output.

24. The computing system of claim 23, wherein the training biomolecule sequence variants include one or more unsaturated sequence variants.

25. The computing system of claim 23, wherein the each respective measured binding characteristic representing an ability of the each to bind to the corresponding respective binding partner is determined via an ACE assay.

26. A computing system for predicting a naturalness of a biomolecule sequence variant, the computing system comprising:
one or more processors; and
one or more non-transitory computer-readable media having stored thereon:
a machine-learned model trained using training data,
wherein the training data includes one or more training biomolecule sequence variants, and
wherein the machine-learned model is configured to output a respective predicted naturalness characteristic of one or more biomolecule sequence variants; and
instructions that, when executed by the one or more processors, cause the computing system to:
process one or more input biomolecule sequence variants with the machine-learned model to generate a respective predicted naturalness characteristic for each of the one or more input biomolecule sequence variants; and
provide at least one of the predicted naturalness characteristics as output.

27. The computing system of claim 26, the non-transitory computer-readable media having stored thereon further instructions that, when executed by the one or more processors, cause the computing system to:
compare the respective predicted naturalness characteristic for each of the one or more input biomolecule sequence variants to one or both of (i) published phage data and (ii) a Therapeutic Antibody Profiler to determine one or more correlations between at least one respective naturalness characteristic and a developability characteristic.

28. The computing system of claim 26, the non-transitory computer-readable media having stored thereon further instructions that, when executed by the one or more processors, cause the computing system to:
generate origin-binned data by comparing the respective predicted naturalness characteristic for each of the one or more input biomolecule sequence variants to published naturalness of therapeutic antibodies administered to humans in phase I, phase II, phase Ill or clinical phase using a CDR-only model; and
determine an immunogenicity scoring by splitting the origin-binned data according to whether patients developed an anti-drug antibody response to fully human antibodies.

29. The computing system of claim 26, the non-transitory computer-readable media having stored thereon further instructions that, when executed by the one or more processors, cause the computing system to:
score at least one of (i) a naturalness of a sequence variant as a function of CDR mutational load, or (ii) a naturalness of a reference antibody sequence.

30. The computing system of claim 26, the non-transitory computer-readable media having stored thereon further instructions that, when executed by the one or more processors, cause the computing system to:
process the one or more biomolecule sequence variants with the machine-learned model to generate the one or more predicted binding characteristics and analyze the one or more predicted binding characteristics to identify one or more biomolecule sequence variants of interest from among the one or more biomolecule sequence variants using a generative technique, to avoid exhaustively predicting affinity of every possible sequence variant in a sequence space.

31. The computing system of claim 30, the non-transitory computer-readable media having stored thereon further instructions that, when executed by the one or more processors, cause the computing system to:
process the one or more biomolecule sequence variants with the machine-learned model to generate the one or more predicted binding characteristics based on a respective predicted naturalness of the one or more biomolecule sequence variants.

32. (canceled)

33. (canceled)
